(19) United States
(12) Patent Application Publication
(10) Pub. No.: US 2024/0100185 A1
(43) Pub. Date: Mar. 28, 2024

(54) COMPOSITIONS AND METHODS FOR THE TARGETING OF PTBP1

(71) Applicant: Scribe Therapeutics Inc., Alameda, CA (US)

(72) Inventors: Susan TOM, Alameda, CA (US); Oleh KRUPA, Oakland, CA (US); Benjamin OAKES, El Cerrito, CA (US); Sean HIGGINS, Alameda, CA (US); Sarah DENNY, San Francisco, CA (US); Brett T. STAAHL, Tiburon, CA (US); Isabel COLIN, Oakland, CA (US); Maroof ADIL, Davis, CA (US)

(21) Appl. No.: 18/255,182

(22) PCT Filed: Dec. 2, 2021
(86) PCT No.: PCT/US2021/061667

§ 371 Date: May 31, 2023

Related U.S. Application Data
(60) Provisional application No. 63/120,879, filed on Dec. 3, 2020.

(51) Int. Cl. A61K 48/00; A61P 25/16; C12N 9/22; C12N 15/113; C12N 15/90

(52) U.S. Cl. CPC A61K 48/005; A61P 25/16; C12N 9/22; C12N 15/113; C12N 15/907; C12N 2310/20; C12N 2800/80

(57) ABSTRACT

Provided herein are Class 2 Type V CRISPR systems comprising CRISPR-Cas polypeptides, (e.g., CasX:gRNA systems comprising CasX polypeptides), guide nucleic acids (gRNA), and optionally donor template nucleic acids, useful in the modification of a PTBP1 gene. The systems are also useful in methods for reprogramming certain eukaryotic cells into functional neurons by the knocking down or knocking out the PTBP1 gene in those cells. Also provided are methods of using such systems in methods of treatment of a subject with a PTBP1-related disease.

### CROSS-REFERENCE TO RELATED APPLICATIONS

This application claims priority to U.S. provisional patent application No. 63/120,879, filed on Dec. 3, 2020, the contents of which are incorporated by reference in their entirety herein.

### INCORPORATION BY REFERENCE OF SEQUENCE LISTING

This application contains a Sequence Listing which has been submitted in ASCII format via EFS-WEB and is hereby incorporated by reference in its entirety. Said ASCII copy, created on Dec. 1, 2021 is named SCRB_029_SeqList_ST25.txt and is 11.8 MB in size.

### BACKGROUND

Polypyrimidine tract-binding protein, also known as PTB or hnRNP I, is a group of three RNA-binding proteins that play a role in the regulation of alternative splicing events, but are also involved in alternative 3′ end processing, mRNA stability and RNA localization (Keppetipola N., et al. Neuronal regulation of pre-mRNA splicing by polypyrimidine tract binding proteins, PTBP1 and PTBP2. Crit Rev Biochem Mol Biol 47:360 (2012)). Genes whose alternative splicing are known to be regulated by PTB include α-TM, β-TM, α-actinin, c-src, γ-2 GABAA, CLCB, NMDA, FGFR-1 and -2, fibronectin, CASP-2, tau, and CT/CGRP (Wollerton, M C, et al. Autoregulation of Polypyrimidine Tract Binding Protein by Alternative Splicing Leading to Nonsense-Mediated Decay. Molecular Cell 13(1):91 (2004)). PTBP1 and its brain-specific homologue polypyrimidine tract-binding protein 2 (PTBP2, also known as nPTB) regulate neural precursor cell differentiation. In the mammalian fetal brain, PTBP1 and PTBP2 are expressed at high levels and then both transcripts decrease in the mature adult brain where staining patterns become mutually exclusive: PTBP1 in glial cells and PTBP2 mostly in neurons (Cheung, H C, et al. Splicing factors PTBP1 and PTBP2 promote proliferation and migration of glioma cell lines. Brain 132:2277 (2009)). In neurons, the loss of PTBP1 is accompanied by the up-regulation of the homologous protein PTBP2 (Boutz P L, et al. A post-transcriptional regulatory switch in polypyrimidine tract-binding proteins reprograms alternative splicing in developing neurons. Genes & Development 21:1636 (2007)). Studies have shown that depleting PTBP1 mRNA in astrocytes can convert these cells to functional neurons and that such a treatment can be applied to the substantia nigra of mice models of Parkinson's disease in order to convert astrocytes to dopaminergic neurons that innervate into and repopulate endogenous neural circuits, restoring motor function in these mice (Qian, H., et al. Reversing a model of Parkinson's disease with in situ converted nigral neurons. Nature 582:550 (2020)). The ability to convert astrocytes or other cells in the glial lineage into neurons would have utility in the treatment or prevention of multiple neurological diseases or the amelioration of neuronal injury due to trauma or other causes.

Increased expression of PTBP1 has been observed in a number of cancers, such as ovarian tumors, glioblastomas, bladder cancer, colon cancer and breast cancer (Li, X, et al. PTBP1 promotes tumorigenesis by regulating apoptosis and cell cycle in colon cancer. Cancer 105(12):1193 (2018); Bielli, P, et al. The Splicing Factor PTBP1 Promotes Expression of Oncogenic Splice Variants and Predicts Poor Prognosis in Patients with Non-muscle-Invasive Bladder Cancer. Clin Cancer Res; 24(21):5422 (2018)). PTBP1 is involved in almost all steps of mRNA regulation including alternative splicing metabolism during tumorigenesis due to its RNA-binding activity (Wang, Z, et al. High expression of PTBP1 promote invasion of colorectal cancer by alternative splicing of cortactin. Oncotarget. 8:36185 (2017)). Glioma is the most common type of malignant primary brain tumor, with high recurrence and lethality rates. The treatment and prognosis of severely ill patients with glioma have shown no significant improvements despite advances in surgery, radiation therapy, and chemotherapy. Genetic-based mechanisms to control gliomagenesis include the alternative expression of core genes (signal transducer and activator of transcription 3 [STAT3], as well as RNA-binding proteins (RBPs), which can bind to single- or double-stranded RNAs, also participate in regulating gliomagenesis (Uren P J, et al. RNA-Binding Protein Musashi1 Is a Central Regulator of Adhesion Pathways in Glioblastoma. Mol. Cell. Biol. 35: 2965 (2015)). Natural antisense transcripts (NATs) are a class of RNA molecules that are complementary to their paired RNA transcripts. It was found that PTB-AS, a novel natural antisense transcript (NAT) transcribed from the reverse strand of the PTBP1 gene, partially overlaps with the 3′ UTR of the PTBP1 mRNA and that its knockdown significantly inhibited glioma proliferation (in vitro and in vivo) and migration (Zhu, L., et al. PTB-AS, a Novel Natural Antisense Transcript, Promotes Glioma Progression by Improving PTBP1 mRNA Stability with SND1. Mol Ther. 27(9):P1621 (2019)). PTBP1 is aberrantly overexpressed in glioma and knock-down of this factor slowed cell proliferation (Cheung, H C, et al. Splicing factors PTBP1 and PTBP2 promote proliferation and migration of glioma cell lines. Brain 132:2277 (2009)). Thus, it represents a target for genetic editing.

The advent of CRISPR/Cas systems and the programmable nature of these minimal systems has facilitated their use as a versatile technology for genomic manipulation and engineering. Particular CRISPR proteins are particularly well suited for such manipulation. For example CasX, has compact size and ease of delivery, and the nucleotide sequence encoding the protein is relatively short; an advantage for its incorporation into viral and other vectors for delivery into a cell.

There remains a critical need for developing treatments for neurologic diseases and cancers using such technologies. Provided herein are compositions and methods for targeting PTBP1 to the address the same.

### SUMMARY

The present disclosure provides compositions comprising modified Class 2, Type V CRISPR proteins and guide nucleic acids used in the editing of PTBP1 gene target nucleic acid sequences. The Class 2, Type V CRISPR proteins and guide nucleic acids can be modified for passive entry into target cells. The Class 2, Type V CRISPR proteins and guide nucleic acids are useful in a variety of methods for target nucleic acid modification of PTBP1-related diseases, which methods are also provided.

In one aspect, the present disclosure relates to systems of CasX proteins and guide ribonucleic acids (CasX:gRNA system) and methods used to knock-down or knock-out a PTBP1 gene in order to reduce or eliminate expression of the PTBP1 gene product in subjects having a PTBP1-related disease. In some embodiments, the compositions and methods have utility in subjects having a neurologic disease or injury such as, but not limited to Parkinson's disease, Huntington's disease, Alzheimer's, amyotrophic lateral sclerosis (ALS), traumatic brain injury, and traumatic spinal cord injury. In other embodiments, the compositions and methods have utility in subjects having a cancer in which PTBP1 is overexpressed.

In some embodiments, the CasX:gRNA system gRNA is a gRNA, or a chimera of RNA and DNA, and may be a single-molecule gRNA or a dual-molecule gRNA. In other embodiments, the CasX:gRNA system gRNA has a targeting sequence complementary to a target nucleic acid sequence within the PTBP1 gene or that comprises a sequence having at least about 50%, at least about 60%, at least about 70%, at least about 80%, at least about 85%, at least about 90%, or at least about 95%, or 100% sequence identity to a sequence selected from the group consisting of SEQ ID NOS: 492-2100 and 2286-43569, wherein the targeting sequence comprises 15 to 30 consecutive nucleotides. In other embodiments, the targeting sequence of the gRNA consists of 20 nucleotides. In other embodiments, the targeting sequence consists of 19 nucleotides. In other embodiments, the targeting sequence consists of 18 nucleotides. In other embodiments, the targeting sequence consists of 17 nucleotides. In other embodiments, the targeting sequence consists of 16 nucleotides. In other embodiments, the targeting sequence consists of 15 nucleotides.

In some embodiments, the gRNA has a scaffold comprising a sequence selected from the group consisting of SEQ ID NOS: 4-16, or a sequence having at least about 50%, at least about 60%, at least about 70%, at least about 80%, at least about 90%, at least about 95%, at least about 96%, at least about 97%, at least about 98%, or at least about 99% sequence identity thereto. In other embodiments, the CasX:gRNA system gRNA has a scaffold comprising a sequence selected from the group consisting of SEQ ID NOS: 2101-2285, 43571-43661 and 44045, or a sequence having at least about 50%, at least about 60%, at least about 70%, at least about 80%, at least about 90%, at least about 95%, at least about 96%, at least about 97%, at least about 98%, at least about 99% sequence identity thereto. In other embodiments, the CasX:gRNA system gRNA has a scaffold comprising a sequence selected from the group consisting of SEQ ID NOS: 2238-2285, 43571-43661, 44045 and 44047, or a sequence having at least about 50%, at least about 60%, at least about 70%, at least about 80%, at least about 90%, at least about 95%, at least about 96%, at least about 97%, at least about 98%, at least about 99% sequence identity thereto. In other embodiments, the CasX:gRNA system gRNA has a scaffold comprising a sequence selected from the group consisting of SEQ ID NOS: 2238-2285, 43571-43661, 44045 and 44047, or a sequence having at least about 50%, at least about 60%, at least about 70%, at least about 80%, at least about 90%, at least about 95%, at least about 96%, at least about 97%, at least about 98%, at least about 99% sequence identity thereto.

In some embodiments, the CasX:gRNA systems comprise a CasX variant sequence of SEQ ID NOS: 36-99, 101-148 or 43662-43907, or a sequence having at least about 50%, at least about 60%, at least about 70%, at least about 80%, or at least about 90%, or at least about 95%, or at least about 96%, or at least about 97%, or at least about 98%, or at least about 99% sequence identity thereto. In some embodiments, the CasX:gRNA systems comprise a CasX variant sequence of SEQ ID NOS: 59, 72-99, 101-148, or 43662-43907, or a sequence having at least about 50%, at least about 60%, at least about 70%, at least about 80%, or at least about 90%, or at least about 95%, or at least about 96%, or at least about 97%, or at least about 98%, or at least about 99% sequence identity thereto. In some embodiments, the CasX:gRNA systems comprise a CasX variant sequence of SEQ ID NOS: 132-148 or 43662-43907, or a sequence having at least about 50%, at least about 60%, at least about 70%, at least about 80%, or at least about 90%, or at least about 95%, or at least about 96%, or at least about 97%, or at least about 98%, or at least about 99% sequence identity thereto. In these embodiments, a CasX variant exhibits one or more improved characteristics relative to a reference CasX protein, for example a reference protein of SEQ ID NO:1, SEQ ID NO:2 or SEQ ID NO:3, or to the variant from which it was derived; e.g., CasX 491 or CasX 515. In some embodiments, the CasX protein has binding affinity for a protospacer adjacent motif (PAM) sequence selected from the group consisting of TTC, ATC, GTC, and CTC. In some embodiments, the CasX protein has binding affinity for the PAM sequence that is at least 1.5-fold greater compared to the binding affinity of any one of the CasX proteins of SEQ ID NOS: 1-3 for the PAM sequences selected from the group consisting of TTC, ATC, GTC, and CTC.

In some embodiments of the CasX:gRNA system, the CasX molecule and the gRNA molecule are associated together in a ribonuclear protein complex (RNP). In a particular embodiment, the RNP comprising the CasX variant and the gRNA variant exhibits greater editing efficiency and/or binding of a target DNA sequence when any one of the PAM sequences TTC, ATC, GTC, or CTC is located 1 nucleotide 5′ to the non-target strand sequence having identity with the targeting sequence of the gRNA in a cellular assay system compared to the editing efficiency and/or binding of an RNP comprising a reference CasX protein and a reference gRNA in a comparable assay system. In a particular embodiment of the foregoing, the target DNA sequence is a sequence of the PTBP1 gene.

In some embodiments, the CasX:gRNA system further comprises a donor template comprising a nucleic acid comprising at least a portion of a PTBP1 gene, wherein the PTBP1 gene portion is selected from the group consisting of a PTBP1 exon, a PTBP1 intron, a PTBP1 intron-exon junction, a PTBP1 regulatory element, or combinations thereof, wherein the donor template is used to knock down or knock out the PTBP1 gene. In some cases the donor sequence is a single-stranded DNA template or a single stranded RNA template. In other cases, the donor template is a double-stranded DNA template.

In other embodiments, the disclosure relates to nucleic acids encoding the CasX:gRNA systems of any of the embodiments described herein, as well as vectors comprising the nucleic acids. In some embodiments, the vector is selected from the group consisting of a retroviral vector, a lentiviral vector, an adenoviral vector, an adeno-associated viral (AAV) vector, a herpes simplex virus (HSV) vector, a plasmid, a minicircle, a nanoplasmid, and an RNA vector. In other embodiments, the vector is a CasX delivery particle (XDP) comprising an RNP of a CasX and gRNA of any of the embodiments described herein and, optionally, a donor template nucleic acid.

In other embodiments, the disclosure provides a method of modifying a PTBP1 target nucleic acid sequence of a cell, wherein said method comprises introducing into the cell: a) CasX:gRNA system of any of the embodiments disclosed herein; b) the nucleic acid of any of the embodiments disclosed herein; c) the vector of any of the embodiments disclosed herein; d) the XDP of any of the embodiments disclosed herein; or e) a combination of the foregoing. In some embodiments of the method, the modifying comprises introducing an insertion, deletion, substitution, duplication, or inversion of one or more nucleotides in the PTBP1 target nucleic acid sequence as compared to the wild-type sequence. In some cases, the method further comprises contacting the target nucleic acid with a donor template nucleic acid of any of the embodiments disclosed herein wherein the donor template is inserted into the break sites of the target nucleic acid introduced by the CasX nuclease. In some embodiments of the method, the donor template comprises a nucleic acid comprising at least a portion of a PTBP1 gene but with one or more mutations for knocking out or knocking down the PTBP1 gene. In some cases, the modifying of the target nucleic acid sequence occurs in vitro or ex vivo. In some cases, the modifying of the target nucleic acid sequence occurs in vivo. In some embodiments, the cell is a eukaryotic cell selected from the group consisting of a rodent cell, a mouse cell, a rat cell, a primate cell, and a non-human primate cell. In some embodiments, the cell is a human cell. In some embodiments, the cell is a selected from the group consisting of a microglial cells, astrocytes, oligodendrocytes, and fibroblasts, wherein the cells are reprogrammed into functional neurons. In some embodiments, the cell is an autologous cell derived from a subject with a neurologic disease or injury. In other embodiments, the cell is allogenic, but of the same species as the subject to be treated. In other embodiments, the cell is a cancer cell wherein the cancer is selected from the group consisting of ovarian cancer, glioblastoma, bladder cancer, colon cancer and breast cancer.

In other embodiments, the disclosure provides methods of modifying a target nucleic acid sequence of the PTBP1 gene wherein the target cells are contacted using vectors encoding the CasX protein and one or more gRNAs comprising a targeting sequence complementary to the PTBP1 gene, and optionally further comprises a donor template, wherein the PTBP1 gene is knocked down or knocked out. In some cases, the vector is an Adeno-Associated Viral (AAV) vector selected from AAV1, AAV2, AAV3, AAV4, AAV5, AAV6, AAV7, AAV8, AAV9, AAV10, AAV11, AAV12, AAV-Rh74, AAVRh10, or a hybrid, a derivative or variant thereof. In other cases, the vector is a lentiviral vector. In other embodiments, the disclosure provides methods wherein the target cells are contacted using a vector wherein the vector is a virus-like particle (XDP) comprising an RNP of a CasX and gRNA of any of the embodiments described herein and, optionally, a donor template nucleic acid. In some embodiments of the method, the vector is administered to a subject at a therapeutically effective dose. The subject can be a mouse, rat, pig, non-human primate, or a human. The dose can be administered by a route of administration selected from intraparenchymal, intravenous, intra-arterial, intracerebroventricular, intracisternal, intrathecal, intracranial, lumbar, intraperitoneal, or combinations thereof.

In some embodiments, the disclosure provides populations of cells modified by the methods of any of the embodiments described herein, wherein the cells have been modified such that at least 70%, at least 75%, at least 80%, at least 85%, at least 90%, or at least 95% of the modified cells do not express a detectable level of PTBP1 protein. In other embodiments, the disclosure provides populations of cells modified by the methods of any of the embodiments described herein, wherein the cells have been modified such that the expression of PTBP1 protein is reduced by at least 70%, at least 75%, at least 80%, at least 85%, at least 90%, or at least 95% compared to cells where the PTBP1 gene has not been modified. In some cases, the population of cells is modified in vitro or ex vivo. In some embodiments, the cells are selected from the group consisting of microglial cells, astrocytes, oligodendrocytes, and fibroblasts, and the modification results in the reprogramming of the cells into functional neurons. The cells have utility in the treatment of neurologic diseases or injury and can be administered to a subject using a therapeutically-effective dose. In some cases, the population of modified cells are autologous with respect to the subject to be administered the cells. In other cases, the population of cells are allogeneic with respect to the subject to be administered the cells. In some cases, when administered to the subject, the cells or their progeny persist in the subject for at least one month, two month, three months, four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the modified cells to the subject.

In other embodiments, the disclosure provides a method of treating a PTBP1-related disease in a subject in need thereof, comprising modifying a gene encoding PTBP1 gene in a cell of the subject, the modifying comprising contacting said cell with: a) CasX:gRNA system of any of the embodiments disclosed herein; b) the nucleic acid of any of the embodiments disclosed herein; c) the vector of any of the embodiments disclosed herein; d) the XDP of any of the embodiments disclosed herein; or e) a combination of the foregoing wherein the PTBP1 gene is knocked down or knocked out. In some embodiments, the PTBP1-related disease is a neurologic disease or neurologic injury selected from the group consisting of Parkinson's disease, Huntington's disease, Alzheimer's, amyotrophic lateral sclerosis (ALS), traumatic brain injury, and traumatic spinal cord injury. In other embodiments, the PTBP1-related disease is a cancer selected from the group consisting of ovarian cancer, glioblastoma, bladder cancer, colon cancer and breast cancer. In some cases, the methods of treating a subject with a PTBP1-related disease result in improvement in at least one clinically-relevant parameter. In other cases, the methods of treating a subject with a PTBP1-related disease result in improvement in at least two clinically-relevant parameters.

In other embodiments, the disclosure provides use of the CasX:gRNA systems, nucleic acids, vectors or XDP described herein for treating a PTBP1-related disease in a subject in need thereof. In some embodiments, the use comprises modifying a gene encoding PTBP1 gene in a cell of the subject, the modifying comprising contacting said cell with: a) CasX:gRNA system of any of the embodiments disclosed herein; b) the nucleic acid of any of the embodiments disclosed herein; c) the vector of any of the embodiments disclosed herein; d) the XDP of any of the embodiments disclosed herein; or e) a combination of the foregoing wherein the PTBP1 gene is knocked down or knocked out.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The contents of U.S. provisional application 63/208,855 filed on Jun. 9, 2021, and U.S. provisional application 63/120,879, filed on Dec. 3, 2020, both applications which disclose CasX variants and gRNA variants, are hereby incorporated by reference in their entireties. The contents of international application publications WO 2020/247882, published Dec. 10, 2020, WO 2020/247883, published Dec. 10, 2020, and WO 2021/113772, published Jun. 10, 2021 are hereby incorporated by reference in their entireties.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a graph of the results of an assay for the quantification of active fractions of RNP formed by sgRNA174 and the CasX variants 119, 457, 488 and 491, as described in Example 8. “2” refers to the reference CasX protein of SEQ ID NO: 2, and sequences corresponding to sgRNA174 and the CasX variants are provided in Tables 3 and 4, respectively. Equimolar amounts of RNP and target were co-incubated and the amount of cleaved target was determined at the indicated timepoints. Mean and standard deviation of three independent replicates are shown for each timepoint. The biphasic fit of the combined replicates is shown.

FIG. 2 shows the quantification of active fractions of RNP formed by CasX2 (reference CasX protein of SEQ ID NO:2) and the modified sgRNAs, as described in Example 8. Equimolar amounts of RNP and target were co-incubated and the amount of cleaved target was determined at the indicated timepoints. Mean and standard deviation of three independent replicates are shown for each timepoint. The biphasic fit of the combined replicates is shown.

FIG. 3 shows the quantification of active fractions of RNP formed by CasX 491 and the modified sgRNAs under guide-limiting conditions, as described in Example 8. Equimolar amounts of RNP and target were co-incubated and the amount of cleaved target was determined at the indicated timepoints. The biphasic fit of the data is shown.

FIG. 4 shows the quantification of cleavage rates of RNP formed by sgRNA174 and the CasX variants, as described in Example 8. Target DNA was incubated with a 20-fold excess of the indicated RNP and the amount of cleaved target was determined at the indicated time points. Mean and standard deviation of three independent replicates are shown for each timepoint, except for 488 and 491 where a single replicate is shown. The monophasic fit of the combined replicates is shown.

FIG. 5 shows the quantification of cleavage rates of RNP formed by CasX2 and the sgRNA variants, as described in Example 8. Target DNA was incubated with a 20-fold excess of the indicated RNP and the amount of cleaved target was determined at the indicated time points. Mean and standard deviation of three independent replicates are shown for each timepoint. The monophasic fit of the combined replicates is shown.

FIG. 6 shows the quantification of initial velocities of RNP formed by CasX2 and the sgRNA variants, as described in Example 8. The first two time-points of the previous cleavage experiment were fit with a linear model to determine the initial cleavage velocity.

FIG. 7 shows the quantification of cleavage rates of RNP formed by CasX491 and the sgRNA variants, as described in Example 8. Target DNA was incubated with a 20-fold excess of the indicated RNP at 10° C. and the amount of cleaved target was determined at the indicated time points. The monophasic fit of the timepoints is shown.

FIG. 8 shows the quantification of competent fractions of RNP of CasX variant 515 and 526 complexed with gRNA variant 174 compared to RNP of reference CasX 2 complexed with gRNA 2 using equimolar amounts of indicated RNP and a complementary target, as described in Example 8. The biphasic fit for each time course or set of combined replicates is shown.

FIG. 9 shows the quantification of cleavage rates of RNP of CasX variant 515 and 526 complexed with gRNA variant 174 compared to RNP of reference CasX 2 complexed with gRNA 2 using with a 20-fold excess of the indicated RNP, as described in Example 8.

FIG. 10A shows the quantification of cleavage rates of CasX variants on TTC PAM, as described in Example 5. Target DNA substrates with identical spacers and the indicated PAM sequence were incubated with a 20-fold excess of the indicated RNP at 37° C. and the amount of cleaved target was determined at the indicated time points. Monophasic fit of a single replicate is shown.

FIG. 10B shows the quantification of cleavage rates of CasX variants on CTC PAM, as described in Example 5. Target DNA substrates with identical spacers and the indicated PAM sequence were incubated with a 20-fold excess of the indicated RNP at 37° C. and the amount of cleaved target was determined at the indicated time points. Monophasic fit of a single replicate is shown.

FIG. 10C shows the quantification of cleavage rates of CasX variants on GTC PAM, as described in Example 5. Target DNA substrates with identical spacers and the indicated PAM sequence were incubated with a 20-fold excess of the indicated RNP at 37° C. and the amount of cleaved target was determined at the indicated time points. Monophasic fit of a single replicate is shown.

FIG. 10D shows the quantification of cleavage rates of CasX variants on ATC PAM, as described in Example 5. Target DNA substrates with identical spacers and the indicated PAM sequence were incubated with a 20-fold excess of the indicated RNP at 37° C. and the amount of cleaved target was determined at the indicated time points. Monophasic fit of a single replicate is shown.

FIG. 11A shows the quantification of cleavage rates of RNP of CasX variant 491 and guide 174 on NTC PAMs, as described in Example 5. Timepoints were taken over the course of 10 minutes and the fraction cleaved was graphed for each target and timepoint, but only the first two minutes of the time course are shown for clarity.

FIG. 11B shows the quantification of cleavage rates of RNP of CasX variant 491 and guide 174 on NTT PAMs, as described in Example 5. Timepoints were taken over the course of 10 minutes and the fraction cleaved was graphed for each target and timepoint.

FIG. 12A shows the quantification of cleavage by RNP formed by sgRNA174 and the CasX variants 515 using spacer lengths of 18, 19, or 20 nucleotides, as described in Example 9. Target DNA was incubated with a 20-fold excess of the indicated RNP and the amount of cleaved target was determined at the indicated time points. Mean and standard deviation of three independent replicates are shown for each timepoint. The monophasic fit of the combined replicates is shown.

FIG. 12B shows the quantification of cleavage by RNP formed by sgRNA174 and the CasX variant 526 using spacer lengths of 18, 19, or 20 nucleotides, as described in Example 9. Target DNA was incubated with a 20-fold excess of the indicated RNP and the amount of cleaved target was determined at the indicated time points. Mean and standard deviation of three independent replicates are shown for each timepoint. The monophasic fit of the combined replicates is shown.

FIG. 13 is a schematic showing an example of CasX protein and scaffold DNA sequence for packaging in adeno-associated virus (AAV). The DNA segment between the AAV inverted terminal repeats (ITRs), comprised of a CasX-encoding DNA and its promoter, and scaffold-encoding DNA and its promoter gets packaged within an AAV capsid during AAV production.

FIG. 14 shows the results of an editing assay comparing gRNA scaffolds 229-237 to scaffold 174 in mouse neural progenitor cells (mNPC) isolated from the Ai9-tdtomato transgenic mice, as described in Example 12. Cells were nucleofected with the indicated doses of p59 plasmids encoding CasX 491, the scaffold, and spacer 11.30 (5′ AAGGGGCUCCGCACCACGCC 3′, SEQ ID NO: 44037) targeting mRHO. Editing at the mRHO locus was assessed 5 days post-transfection by NGS, and show that editing with constructs with scaffolds 230, 231, 234 and 235 demonstrated greater editing compared to constructs with scaffold 174 at both doses.

FIG. 15 shows the results of an editing assay comparing gRNA scaffolds 229-237 to scaffold 174 in mNPC cells, as described in Example 12. Cells were nucleofected with the indicated doses of p59 plasmids encoding CasX 491, the scaffold, and spacer 12.7 (5′ CUGCAUUCUAGUUGUGGUUU 3′, SEQ ID NO: 44038) targeting repeat elements preventing expression of the tdTomato fluorescent protein. Editing was assessed 5 days post-transfection by FACS, to quantify the fraction of tdTomato positive cells. Cells nucleofected with scaffolds 231-235 displayed approximately 35% greater editing compared to constructs with scaffold 174 at the high dose, and approximately 25% greater editing at the low dose.

FIG. 16A shows the results of an editing assay comparing gRNA scaffolds 235 to scaffold 174 in ARPE-19 mNPC cells, as described in Example 12. Cells were nucleofected with 1000 ng of AAV-cis plasmids expressing CasX protein 491 and guide variants 174 or 235 with spacer 11.1 targeting the exogenous RHO-GFP locus (5′ AAGGGGCUGCGUACCACACC 3′, SEQ ID NO: 44039) or guide variant 235 with a non-targeting control spacer. Frequency of GFP-cells was assessed by FACS 5 days post-transfection as a readout of indel-induced knock-down of WT RHO-GFP fusion protein. Frequency of RFP-cells was also measured as to assess off target cleavage. Spacer 11.1 harbors 1-bp mismatch to exogenous P23H-RHO. Data (n=3) are presented as mean±SEM.

FIG. 16B shows editing results of cells nucleofected with p590.4910.235.11.1 compared relative to benchmark p59.491.174.11.1 (set to a value of 1.0) in cells nucleofected with 1000 ng of plasmid, with editing improved 3-fold with the 235 gRNA scaffold compared to the 174 gRNA scaffold, as described in Example 12.

FIG. 17A shows the results of AAV-mediated editing assays comparing gRNA scaffold 235 to scaffold 174 and guide 11.30 at the endogenous mouse Rho exon 1 locus in mNPCs, as described in Example 12. FIG. 17A shows the results of editing assays in mNPCs at a 3.0e+5 AAV vg/cell MOI.

FIG. 17B shows the editing results as fold-change in editing levels for scaffold 235 relative to guide 174 (set to 1.0) with spacer 11.30 in cells infected at a 5.0e+5 MOI, as described in Example 12.

FIG. 18 depicts a schematic of the relative locations in the mouse PTBP1 gene that spacers 28.1-28.12 target, as described in Example 14. Locations targeted by spacers are indicated by black bars.

FIG. 19 shows the quantification of average percent editing measured as indel rate detected by NGS at the mouse PTBP1 locus generated by the indicated spacer, as described in Example 14.

FIG. 20 is a bar chart illustrating average editing rates by type of mutation generated (insertion, deletion, or both insertion and deletion) by an individual spacer, as described in Example 14.

FIG. 21 is a bar chart showing average percent editing measured as indel rate generated by the indicated spacer detected by NGS at the mouse PTBP1 locus at the indicated range of MOIs, as described in Example 14.

FIG. 22 is a schematic showing the molecular organization of the AAV construct used to encode, package, and deliver CasX:gNA systems, as described in Example 15.

FIG. 23 depicts the results of an editing assay measured as indel rate detected by NGS at the mouse PTBP1 locus for the indicated AAV-CasX (XAAV) dual-guide systems (28.10-12.7 and NT-12.7) transduced into mouse astrocytes in a series of three-fold dilution of MOI, as described in Example 15.

FIG. 24 illustrates the quantification of tdTomato+ astrocytes detected by flow cytometry four days post-transduction of the indicated XAAV dual-guide systems into primary mouse astrocytes, as described in Example 15.

FIG. 25A shows western blot quantification of PTBP1 levels at the indicated time points in mouse astrocytes treated with XDP-NT or XDP-PTBP1, as described in Example 16. The ratio of PTBP1 level over total protein was normalized to NT control in the graph.

FIG. 25B shows western blot quantification of PTBP1 levels quantified at Day 5, 12, and 21 of treatment, as described in Example 16. The ratio of PTBP1 level over total protein was normalized to NT control in the graph.

FIG. 26A shows western blot quantification of nPTB levels at the indicated time points in mouse astrocytes treated with XDP-NT or XDP-PTBP1, as described in Example 16. The ratio of nPTB level over total protein level was normalized to NT control in the graph.

FIG. 26B is a bar graph for nPTB levels quantified at Day 5, 12, and 21 of treatment, as described in Example 16. The ratio of nPTB level over total protein level was normalized to NT control in the graph.

FIG. 27 shows quantification of percent editing measured as indel rate detected by NGS at the PTBP1 locus in cultured mouse astrocytes that were untreated or treated with XDPs containing spacer targeting either PTBP1 or tdTomato (NT control), as described in Example 17. Editing levels at the PTBP1 locus were assessed at Day 5, 12, and 21 post-XDP treatment. Data are presented as mean±SEM for n=2 replicates.

FIG. 28 is a bar graph showing the quantification of mouse astrocytes stained for the neuronal marker MAP2 at Day 28 post-XDP treatment for the indicated experimental conditions, as described in Example 17. Data are presented as percent of total cells (quantified by DAPI staining) expressing MAP2. Data are mean±SEM for n=2 replicates (*p<0.05, **p<0.01).

FIG. 29A illustrates the quantification of NeuN+/tdTomato+ cells at 3 weeks and 12 weeks post-infection with dual-guide XAAVs harboring either the PTBP1-tdTomato or non-targeting (NT)-tdTomato spacer combination. Data points are presented as a fraction of total tdTomato+ edited cells, as described in Example 18. Data are presented as mean±SEM, with n=2 animals per experimental group (**p<0.01, ***p<0.001).

FIG. 29B illustrates the quantification of Sox9+/tdTomato+ cells at 3 weeks and 12 weeks post-infection with dual-guide XAAVs harboring either the PTBP1-tdTomato or non-targeting (NT)-tdTomato spacer combination. Data points are presented as a fraction of total tdTomato+ edited cells, as described in Example 18. Data are presented as mean±SEM, with n=2 animals per experimental group (**p<0.01, ***p<0.001).

FIG. 30 shows the quantification of average percent editing measured as indel rate generated by the indicated spacer, as detected by NGS at the rat PTBP1 locus, as described in Example 19.

FIG. 31A compares the editing activity for engineered CasX nucleases 491, 668, 672, and 676 at the mouse PTBP1 locus when delivered in vitro via XDPs at various doses, as described in Example 20.

FIG. 31B compares the editing activity for engineered CasX nucleases 491, 668, 672, and 676 at the mouse (FIG. 31A) or rat (FIG. 31B) PTBP1 locus when delivered in vitro via XDPs at various doses, as described in Example 20.

FIG. 32 shows the quantification of average percent editing measured as indel rate generated with the indicated spacer detected by NGS at the human PTBP1 locus, as described in Example 21.

FIG. 33A shows the quantification of percent editing of the human PTBP1 locus measured as indel rate detected by NGS at the PTBP1 locus generated using the two human PTBP1 spacers 30.17 and 30.19 and non-targeting spacer (NT) across the various MOIs, as described in Example 22.

FIG. 33B shows correlation plots between editing events at the human PTBP1 locus and RNA expression of PTBP1 for PTBP1 generated using spacers 30.17 and 30.19 in vitro, as described in Example 22. The expression levels were normalized relative to expressions from samples treated with the non-targeting control.

FIG. 33C show correlation plots between editing events at the human PTBP1 locus and RNA expression of nPTB for PTBP1 generated using spacers 30.17 and 30.19 in vitro, as described in Example 22. The expression levels were normalized relative to expressions from samples treated with the non-targeting control.

FIG. 34A is a representative image of XDP in vivo editing efficiency in a brain section at three weeks post-XDP injection into the substantia nigra, marked by tdTomato fluorescent reporter expression, and biodistribution in the mouse midbrain as described in Example 23.

FIG. 34B shows the fraction of total cells that were edited, based on quantification of tdTomato expression in the brain sections, as described in Example 23. Data are mean±SD for 40 tissue sections across two animals.

FIG. 34C is the quantification of cellular tropism of the XDP quantified by the estimated fraction of all cells or tdTomato+ edited cells within the mouse substantia nigra that were marked NeuN+(neuron) or Sox9+(astrocyte), as described in Example 23. Data are mean±SD for 40 tissue sections across two animals.

### DETAILED DESCRIPTION

While exemplary embodiments have been shown and described herein, it will be obvious to those skilled in the art that such embodiments are provided by way of example only. Numerous variations, changes, and substitutions will now occur to those skilled in the art without departing from the inventions claimed herein. It should be understood that various alternatives to the embodiments described herein may be employed in practicing the embodiments of the disclosure. It is intended that the claims define the scope of the invention and that methods and structures within the scope of these claims and their equivalents be covered thereby.

Unless otherwise defined, all technical and scientific terms used herein have the same meaning as commonly understood by one of ordinary skill in the art to which this invention belongs. Although methods and materials similar or equivalent to those described herein can be used in the practice or testing of the present embodiments, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definitions

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

“Hybridizable” or “complementary” are used interchangeably to mean that a nucleic acid (e.g., RNA, DNA) comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. It is understood that the sequence of a polynucleotide need not be 100% complementary to that of its target nucleic acid sequence to be specifically hybridizable; it can have at least about 70%, at least about 80%, or at least about 90%, or at least about 95% sequence identity and still hybridize to the target nucleic acid sequence. Moreover, a polynucleotide may hybridize over one or more segments such that intervening or adjacent segments are not involved in the hybridization event (e.g., a loop structure or hairpin structure, a ‘bulge’, ‘bubble’ and the like).

A “gene,” for the purposes of the present disclosure, includes a DNA region encoding a gene product (e.g., a protein, RNA), as well as all DNA regions which regulate the production of the gene product, whether or not such regulatory sequences are adjacent to coding and/or transcribed sequences. Accordingly, a gene may include accessory element sequences including, but not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. Coding sequences encode a gene product upon transcription or transcription and translation; the coding sequences of the disclosure may comprise fragments and need not contain a full-length open reading frame. A gene can include both the strand that is transcribed as well as the complementary strand containing the anticodons.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

The term “adjacent to” with respect to polynucleotide or amino acid sequences refers to sequences that are next to, or adjoining each other in a polynucleotide or polypeptide. The skilled artisan will appreciate that two sequences can be considered to be adjacent to each other and still encompass a limited amount of intervening sequence, e.g., 1, 2, 3, 4, 5, 6, 7, 8, 9 or 10 nucleotides or amino acids.

The term “accessory element” is used interchangeably herein with the term “accessory sequence,” and is intended to include, inter alia, polyadenylation signals (poly(A) signal), enhancer elements, introns, posttranscriptional regulatory elements (PTREs), nuclear localization signals (NLS), deaminases, DNA glycosylase inhibitors, additional promoters, factors that stimulate CRISPR-mediated homology-directed repair (e.g. in cis or in trans), activators or repressors of transcription, self-cleaving sequences, and fusion domains, for example a fusion domain fused to a CRISPR protein. It will be understood that the choice of the appropriate accessory element or elements will depend on the encoded component to be expressed (e.g., protein or RNA) or whether the nucleic acid comprises multiple components that require different polymerases or are not intended to be expressed as a fusion protein.

The term “promoter” refers to a DNA sequence that contains a transcription start site and additional sequences to facilitate polymerase binding and transcription. Exemplary eukaryotic promoters include elements such as a TATA box, and/or B recognition element (BRE) and assists or promotes the transcription and expression of an associated transcribable polynucleotide sequence and/or gene (or transgene). A promoter can be synthetically produced or can be derived from a known or naturally occurring promoter sequence or another promoter sequence. A promoter can be proximal or distal to the gene to be transcribed. A promoter can also include a chimeric promoter comprising a combination of two or more heterologous sequences to confer certain properties. A promoter of the present disclosure can include variants of promoter sequences that are similar in composition, but not identical to, other promoter sequence(s) known or provided herein. A promoter can be classified according to criteria relating to the pattern of expression of an associated coding or transcribable sequence or gene operably linked to the promoter, such as constitutive, developmental, tissue-specific, inducible, etc. A promoter can also be classified according to its strength. As used in the context of a promoter, “strength” refers to the rate of transcription of the gene controlled by the promoter. A “strong” promoter means the rate of transcription is high, while a “weak” promoter means the rate of transcription is relatively low.

A promoter of the disclosure can be a Polymerase II (Pol II) promoter. Polymerase II transcribes all protein coding and many non-coding genes. A representative Pol II promoter includes a core promoter, which is a sequence of about 100 base pairs surrounding the transcription start site, and serves as a binding platform for the Pol II polymerase and associated general transcription factors. The promoter may contain one or more core promoter elements such as the TATA box, BRE, Initiator (INR), motif ten element (MTE), downstream core promoter element (DPE), downstream core element (DCE), although core promoters lacking these elements are known in the art.

A promoter of the disclosure can be a Polymerase III (Pol III) promoter. Pol III transcribes DNA to synthesize small ribosomal RNAs such as the 5S rRNA, tRNAs, and other small RNAs. Representative Pol III promoters use internal control sequences (sequences within the transcribed section of the gene) to support transcription, although upstream elements such as the TATA box are also sometimes used. All Pol III promoters are envisaged as within the scope of the instant disclosure.

The term “enhancer” refers to regulatory DNA sequences that, when bound by specific proteins called transcription factors, regulate the expression of an associated gene. Enhancers may be located in the intron of the gene, or 5′ or 3′ of the coding sequence of the gene. Enhancers may be proximal to the gene (i.e., within a few tens or hundreds of base pairs (bp) of the promoter), or may be located distal to the gene (i.e., thousands of bp, hundreds of thousands of bp, or even millions of bp away from the promoter). A single gene may be regulated by more than one enhancer, all of which are envisaged as within the scope of the instant disclosure.

As used herein, a “post-transcriptional regulatory element (PRE),” such as a hepatitis PRE, refers to a DNA sequence that, when transcribed creates a tertiary structure capable of exhibiting post-transcriptional activity to enhance or promote expression of an associated gene operably linked thereto.

As used herein, a “post-transcriptional regulatory element (PTRE),” such as a hepatitis PTRE, refers to a DNA sequence that, when transcribed creates a tertiary structure capable of exhibiting post-transcriptional activity to enhance or promote expression of an associated gene operably linked thereto.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “enhancers” and “promoters”, above).

The term “recombinant polynucleotide” or “recombinant nucleic acid” refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant polypeptide” or “recombinant protein” refers to a polypeptide or protein which is not naturally occurring, e.g., is made by the artificial combination of two otherwise separated segments of amino sequence through human intervention. Thus, e.g., a protein that comprises a heterologous amino acid sequence is recombinant.

As used herein, the term “contacting” means establishing a physical connection between two or more entities. For example, contacting a target nucleic acid with a guide nucleic acid means that the target nucleic acid and the guide nucleic acid are made to share a physical connection; e.g., can hybridize if the sequences share sequence similarity.

“Dissociation constant”, or “K_{d}”, are used interchangeably and mean the affinity between a ligand “L” and a protein “P”; i.e., how tightly a ligand binds to a particular protein. It can be calculated using the formula K_{d}=[L][P]/[LP], where [P], [L] and [LP] represent molar concentrations of the protein, ligand and complex, respectively.

The disclosure provides systems and methods useful for editing a target nucleic acid sequence. As used herein “editing” is used interchangeably with “modifying” and includes but is not limited to cleaving, nicking, deleting, knocking in, knocking out, and the like.

By “cleavage” it is meant the breakage of the covalent backbone of a target nucleic acid molecule (e.g., RNA, DNA). Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events.

The term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant sequence. The term “knock-down” as used herein refers to reduction in the expression of a gene or its gene product(s). As a result of a gene knock-down, the protein activity or function may be attenuated or the protein levels may be reduced or eliminated.

As used herein, “homology-directed repair” (HDR) refers to the form of DNA repair that takes place during repair of double-strand breaks in cells. This process requires nucleotide sequence homology, and uses a donor template to repair or knock-out a target DNA, and leads to the transfer of genetic information from the donor to the target. Homology-directed repair can result in an alteration of the sequence of the target sequence by insertion, deletion, or mutation if the donor template differs from the target DNA sequence and part or all of the sequence of the donor template is incorporated into the target DNA.

As used herein, “non-homologous end joining” (NHEJ) refers to the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

As used herein “micro-homology mediated end joining” (MMEJ) refers to a mutagenic DSB repair mechanism, which always associates with deletions flanking the break sites without the need for a homologous template (in contrast to homology-directed repair, which requires a homologous sequence to guide repair). MMEJ often results in the loss (deletion) of nucleotide sequence near the site of the double-strand break. A polynucleotide or polypeptide has a certain percent “sequence similarity” or “sequence identity” to another polynucleotide or polypeptide, meaning that, when aligned, that percentage of bases or amino acids are the same, and in the same relative position, when comparing the two sequences. Sequence similarity (sometimes referred to as percent similarity, percent identity, or homology) can be determined in a number of different manners. To determine sequence similarity, sequences can be aligned using the methods and computer programs that are known in the art, including BLAST, available over the world wide web at ncbi.nlm.nih.gov/BLAST. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence.

A “vector” or “expression vector” is a replicon, such as plasmid, phage, virus, virus-like particle or cosmid, to which another DNA segment, i.e., an “insert”, may be attached so as to bring about the replication or expression of the attached segment in a cell.

The term “naturally-occurring” or “unmodified” or “wild type” as used herein as applied to a nucleic acid, a polypeptide, a cell, or an organism, refers to a nucleic acid, polypeptide, cell, or organism that is found in nature.

As used herein, a “mutation” refers to an insertion, deletion, substitution, duplication, or inversion of one or more amino acids or nucleotides as compared to a wild-type or reference amino acid sequence or to a wild-type or reference nucleotide sequence.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

A “host cell,” as used herein, denotes a eukaryotic cell, a prokaryotic cell, or a cell from a multicellular organism (e.g., in a cell line), which eukaryotic or prokaryotic cells are used as recipients for a nucleic acid (e.g., an expression vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector.

The term “tropism” ″ as used herein refers to preferential entry of the virus like particle (XDP, sometimes also referred to herein as XDP) into certain cell or tissue type(s) and/or preferential interaction with the cell surface that facilitates entry into certain cell or tissue types, optionally and preferably followed by expression (e.g., transcription and, optionally, translation) of sequences carried by the XDP into the cell.

The terms “pseudotype” or “pseudotyping” as used herein, refers to viral envelope proteins that have been substituted with those of another virus possessing preferable characteristics. For example, HIV can be pseudotyped with vesicular stomatitis virus G-protein (VSV-G) envelope proteins (amongst others, described herein, below), which allows HIV to infect a wider range of cells because HIV envelope proteins target the virus mainly to CD4+ presenting cells.

The term “tropism factor” as used herein refers to components integrated into the surface of an XDP that provides tropism for a certain cell or tissue type. Non-limiting examples of tropism factors include glycoproteins, antibody fragments (e.g., scFv, nanobodies, linear antibodies, etc.), receptors and ligands to target cell markers.

A “target cell marker” refers to a molecule expressed by a target cell including but not limited to cell-surface receptors, cytokine receptors, antigens, tumor-associated antigens, glycoproteins, oligonucleotides, enzymatic substrates, antigenic determinants, or binding sites that may be present in the on the surface of a target tissue or cell that may serve as ligands for an antibody fragment or glycoprotein tropism factor.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

The term “antibody,” as used herein, encompasses various antibody structures, including but not limited to monoclonal antibodies, polyclonal antibodies, multispecific antibodies (e.g., bispecific antibodies), nanobodies, single domain antibodies such as VHH antibodies, and antibody fragments so long as they exhibit the desired antigen-binding activity or immunological activity. Antibodies represent a large family of molecules that include several types of molecules, such as IgD, IgG, IgA, IgM and IgE.

An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2, diabodies, single chain diabodies, linear antibodies, a single domain antibody, a single domain camelid antibody, single-chain variable fragment (scFv) antibody molecules, and multispecific antibodies formed from antibody fragments.

As used herein, “treatment” or “treating,” are used interchangeably herein and refer to an approach for obtaining beneficial or desired results, including but not limited to a therapeutic benefit and/or a prophylactic benefit. By therapeutic benefit is meant eradication or amelioration of the underlying disorder or disease being treated. A therapeutic benefit can also be achieved with the eradication or amelioration of one or more of the symptoms or an improvement in one or more clinical parameters associated with the underlying disease such that an improvement is observed in the subject, notwithstanding that the subject may still be afflicted with the underlying disorder.

The terms “therapeutically effective amount” and “therapeutically effective dose”, as used herein, refer to an amount of a drug or a biologic, alone or as a part of a composition, that is capable of having any detectable, beneficial effect on any symptom, aspect, measured parameter or characteristics of a disease state or condition when administered in one or repeated doses to a subject such as a human or an experimental animal. Such effect need not be absolute to be beneficial.

As used herein, “administering” means a method of giving a dosage of a compound (e.g., a composition of the disclosure) or a composition (e.g., a pharmaceutical composition) to a subject.

A “subject” is a mammal. Mammals include, but are not limited to, domesticated animals, non-human primates, humans, dogs, rabbits, mice, rats and other rodents.

All publications, patents, and patent applications mentioned in this specification are herein incorporated by reference to the same extent as if each individual publication, patent, or patent application was specifically and individually indicated to be incorporated by reference.

### a. General Methods

The practice of the present invention employs, unless otherwise indicated, conventional techniques of immunology, biochemistry, chemistry, molecular biology, microbiology, cell biology, genomics and recombinant DNA, which can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that endpoints are included and that each intervening value, to the tenth of the unit of the lower limit unless the context clearly dictates otherwise, between the upper and lower limit of that range and any other stated or intervening value in that stated range, is encompassed. The upper and lower limits of these smaller ranges may independently be included in the smaller ranges, and are also encompassed, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of ordinary skill in the art to which this invention belongs. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise.

It will be appreciated that certain features of the disclosure, which are, for clarity, described in the context of separate embodiments, may also be provided in combination in a single embodiment. In other cases, various features of the disclosure, which are, for brevity, described in the context of a single embodiment, may also be provided separately or in any suitable sub-combination. It is intended that all combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### b. Systems for Genetic Editing of PTBP1 Genes

In a first aspect, the present disclosure provides systems comprising a Class 2, Type V CRISPR nuclease protein and one or more guide nucleic acids (gRNA), as well as nucleic acids encoding the CRISPR nuclease proteins and gRNA, for use in modifying or editing a PTBP1 gene (referred to herein as the “target nucleic acid”).

As used herein, a “system,” such as the systems comprising a CRISPR nuclease protein and one or more gRNAs the disclosure, as well as nucleic acids encoding the CRISPR nuclease proteins and gRNA and vectors comprising the nucleic acids or CRISPR nuclease protein and one or more gRNAs the disclosure, is used interchangeably with term “composition.”

The PTBP1 gene encodes polypyrimidine tract-binding protein 1, a protein that plays a role in the regulation of alternative splicing events, but is also involved in alternative 3′ end processing, mRNA stability and RNA localization (Keppetipola N., et al. Neuronal regulation of pre-mRNA splicing by polypyrimidine tract binding proteins, PTBP1 and PTBP2. Crit Rev Biochem Mol Biol 47:360 (2012)). The human PTBP1 gene (HGNC:9583) encodes a protein of 531 amino acids having the sequence MDGIVPDIAVGTKRGSDELFSTCVTNGPFIMSSNSASAANGNDSKKFKGDSRSAGVPSRVIHIR KLPIDVTEGEVISLGLPFGKVTNLLMLKGKNQAFIEMNTEEAANTMVNYYTSVTPVLRGQPIYI QFSNHKELKTDSSPNQARAQAALQAVNSVQSGNLALAASAAAVDAGMAMAGQSPVLRIIVENLF YPVTLDVLHQIFSKFGTVLKIITFTKNNQFQALLQYADPVSAQHAKLSLDGQNIYNACCTLRID FSKLTSLNVKYNNDKSRDYTRPDLPSGDSQPSLDQTMAAAFGLSVPNVHGALAPLAIPSAAAAA AAAGRIAIPGLAGAGNSVLLVSNLNPERVTPQSLFILFGVYGDVQRVKILFNKKENALVQMADG NQAQLAMSHLNGHKLHGKPIRITLSKHQNVQLPREGQEDQGLTKDYGNSPLHRFKKPGSKNFQN IFPPSATLHLSNIPPSVSEEDLKVLFSSNGGVVKGFKFFQKDRKMALIQMGSVEEAVQALIDLH NHDLGENHHLRVSFSKSTI (SEQ ID NO:100). The human PTBP1 gene is defined as the sequence that spans chr19:797,075-812,327 (GRCh38/hg38), comprising 15,253 base-pairs in size on the short arm of chromosome 19 and has 16 exons; however the PTBP1 protein results from skipping of exon 9. Alternative splicing of PTB1 exon 2 to 10 has also been observed, leading to a functionally different protein (Wollerton, M C, et al. Autoregulation of Polypyrimidine Tract Binding Protein by Alternative Splicing Leading to Nonsense-Mediated Decay. Molecular Cell 13(1):91 (2004)).

In some embodiments, the disclosure provides systems specifically designed to modify the PTBP1 gene in eukaryotic cells. Generally, any portion of the PTBP1 target nucleic acid can be targeted using the programmable compositions and methods provided herein. In some embodiments, the portion of the PTBP1 gene to be modified is selected from the group consisting of a PTBP1 intron, a PTBP1 exon, a PTBP1 intron-exon junction, a PTBP1 regulatory element, and an intergenic region, or the modification is deletion or mutation of one or more exons.

In some embodiments, the CRISPR nuclease is a Class 2, Type V nuclease. In some embodiments, the Class 2, Type V nuclease is selected from the group consisting of Cas12a, Cas12b, Cas12c, Cas12d (CasY), Cas12j, Cas12k, C2c4, C2c8, C2c5, C2c10, C2c9, CasZ, and CasX. In some embodiments, the disclosure provides systems comprising one or more CasX proteins and one or more guide nucleic acids (gRNA) as a CasX:gRNA system. In some embodiments, the disclosure provides systems comprising one or more CasX variant proteins and one or more guide nucleic acids (gRNA) as a CasX:gRNA system designed to target and edit specific locations in the target nucleic acid sequence. In other embodiments, the CasX:gRNA systems of the disclosure comprise one or more CasX variant proteins, one or more guide nucleic acids (gRNA) and one or more donor template nucleic acids comprising a nucleic acid encoding a portion of a PTBP1 gene. Each of these components and their use in the editing of the PTBP1 gene is described herein, below

In some embodiments, the disclosure provides gene editing pairs of a CasX variant and a gRNA of any of the embodiments described herein that are capable of being bound together prior to their use for gene editing and, thus, are “pre-complexed” as a ribonuclear protein complex (RNP). The use of a pre-complexed RNP confers advantages in the delivery of the system components to a cell or target nucleic acid sequence for editing of the target nucleic acid sequence.

In some embodiments, the functional RNP can be delivered ex vivo to a cell by electrophoresis or by chemical means. In other embodiments, the functional RNP can be delivered either ex vivo or in vivo by a vector in their functional form. The gRNA can provide target specificity to the complex by including a targeting sequence (or “spacer”) having a nucleotide sequence that is complementary to a sequence of the target nucleic acid sequence while the CasX protein of the pre-complexed CasX:gRNA provides the site-specific activity such as cleavage or nicking of the target sequence that is guided to a target site (e.g., stabilized at a target site) within a target nucleic acid sequence by virtue of its association with the gRNA. The CasX proteins and gRNA components of the CasX:gRNA systems and their sequences, features and functions are described more fully, below.

In some cases, the CasX:gRNA systems have utility in the treatment of a subject having a neurologic disease, such as Parkinson's disease, Huntington's disease, Alzheimer's, amyotrophic lateral sclerosis (ALS), traumatic brain injury, traumatic spinal cord injury. In other cases, the CasX:gRNA systems have utility in the treatment of a subject having certain cancers, such as ovarian tumors, glioblastomas, colon cancer and breast cancer. Each of the components of the CasX:gRNA systems and their use in the editing of the target nucleic acids in cells is described more fully, below.

### c. Guide Nucleic Acids of the Systems for Genetic Editing

In another aspect, the disclosure relates to specifically-designed guide ribonucleic acids (gRNA) comprising a targeting sequence complementary to (and are therefore able to hybridize with) a target nucleic acid sequence of a PTBP1 gene that have utility in genome editing of the PTBP1 target nucleic acid in a cell. It is envisioned that in some embodiments, multiple gRNAs are delivered in the systems for the modification of a target nucleic acid. For example, a pair of gRNAs with targeting sequences to different or overlapping regions of the target nucleic acid sequence can be used in order to bind and cleave at two different or overlapping sites within the gene, which is then edited by non-homologous end joining (NHEJ), homology-directed repair (HDR), homology-independent targeted integration (HITI), micro-homology mediated end joining (MMEJ), single strand annealing (SSA) or base excision repair (BER). For example, when an editing event designed to delete one or more exons of the PTBP1 gene is desired, a pair of gRNAs can be used in order to bind and cleave at two different sites 5′ and 3′ of the targeted exon(s) within the PTBP1 gene. In the context of nucleic acids, cleavage refers to the breakage of the covalent backbone of a nucleic acid molecule; either DNA or RNA, by the nuclease. Both single-stranded cleavage and double-stranded cleavage are possible, and double-stranded cleavage can occur as a result of two distinct single-stranded cleavage events. In some embodiments, small indels introduced by the CasX:gRNA systems of the embodiments described herein and cellular repair systems can disrupt the protein reading frame of the PTBP1 gene. In some embodiments, the disclosure provides gRNAs utilized in the CasX:gRNA systems that have utility in genome editing a PTBP1 gene in a eukaryotic cell. The present disclosure provides specifically-designed gRNAs wherein the targeting sequence (or spacer, described more fully, below) of the gRNA is complementary to (and are therefore able to hybridize with) target nucleic acid sequences when used as a component of the gene editing CasX:gRNA systems. Representative, but non-limiting examples of targeting sequences to the PTBP1 target nucleic acid that can be utilized in the gRNA of the embodiments are selected from the group consisting of SEQ ID NOS: 492-2100 and 2286-43569. In some embodiments, the gRNA is a ribonucleic acid molecule (“gRNA”); and in other embodiments, the gRNA is a chimera, and comprises both DNA and RNA. As used herein, the term gRNA covers naturally-occurring molecules, as well as sequence variants.

### d. Reference gRNA and gRNA Variants

As used herein, a “reference gRNA” refers to a CRISPR guide nucleic acid comprising a wild-type sequence of a naturally-occurring gRNA. In some embodiments, a reference gRNA of the disclosure may be subjected to one or more mutagenesis methods, such as the mutagenesis methods described herein, which may include Deep Mutational Evolution (DME), deep mutational scanning (DMS), error prone PCR, cassette mutagenesis, random mutagenesis, staggered extension PCR, gene shuffling, or domain swapping, in order to generate one or more gRNA variants with enhanced or varied properties relative to the reference gRNA. gRNA variants also include variants comprising one or more exogenous sequences, for example fused to either the 5′ or 3′ end, or inserted internally. The activity of reference gRNAs may be used as a benchmark against which the activity of gRNA variants are compared, thereby measuring improvements in function or other characteristics of the gRNA variants. In other embodiments, a reference gRNA may be subjected to one or more deliberate, specifically-targeted mutations in order to produce a gRNA variant, for example a rationally designed variant. Exemplary reference gRNA sequences are provided in Table 2, and include the reference scaffold sequences of SEQ ID NO: 4 and SEQ ID NO: 5.

The gRNAs of the disclosure comprise two segments: a targeting sequence and a protein-binding segment. The targeting segment of a gRNA includes a nucleotide sequence (referred to interchangeably as a guide sequence, a spacer, a targeter, or a targeting sequence) that is complementary to (and therefore hybridizes with) a specific sequence (a target site) within the target nucleic acid sequence (e.g., a target ssRNA, a target ssDNA, a strand of a double stranded target DNA, etc.), described more fully below. The targeting sequence of a gRNA is capable of binding to a target nucleic acid sequence, including a coding sequence, a complement of a coding sequence, a non-coding sequence, and to regulatory elements. The protein-binding segment (or “activator” or “protein-binding sequence”) interacts with (e.g., binds to) a CasX variant protein as a complex, forming an RNP (described more fully, below). The protein-binding segment is alternatively referred to herein as a “scaffold”, which is comprised of several regions, described more fully, below.

In the case of a dual guide RNA (dgRNA), the targeter and the activator portions each have a duplex-forming segment, where the duplex forming segment of the targeter and the duplex-forming segment of the activator have complementarity with one another and hybridize to one another to form a double stranded duplex (dsRNA duplex for a gRNA). When the gRNA is a gRNA, the term “targeter” or “targeter RNA” is used herein to refer to a crRNA-like molecule (crRNA: “CRISPR RNA”) of a CasX dual guide RNA (and therefore of a CasX single guide RNA when the “activator” and the “targeter” are linked together; e.g., by intervening nucleotides). The crRNA has a 5′ region that anneals with the tracrRNA followed by the nucleotides of the targeting sequence. Thus, for example, a guide RNA (dgRNA or sgRNA) comprises a guide sequence and a duplex-forming segment of a crRNA, which can also be referred to as a crRNA repeat. A corresponding tracrRNA-like molecule (activator) also comprises a duplex-forming stretch of nucleotides that forms the other half of the dsRNA duplex of the protein-binding segment of the guide RNA. Thus, a targeter and an activator, as a corresponding pair, hybridize to form a dual guide NA, referred to herein as a “dual guide NA”, a “dual-molecule gRNA”, a “dgRNA”, a “double-molecule guide NA”, or a “two-molecule guide NA”. Site-specific binding and/or cleavage of a target nucleic acid sequence (e.g., genomic DNA) by the CasX variant protein can occur at one or more locations (e.g., a sequence of a target nucleic acid) determined by base-pairing complementarity between the targeting sequence of the gRNA and the target nucleic acid sequence. Thus, for example, and as described more fully, below, the gRNA variants of the disclosure have targeting sequences complementarity to and therefore can hybridize with the target nucleic acid that is adjacent to a sequence complementary to a TC PAM motif or a PAM sequence, such as ATC, CTC, GTC, or TTC. Because the targeting sequence of a guide sequence hybridizes with a sequence of a target nucleic acid sequence, a targeter can be modified by a user to hybridize with a specific target nucleic acid sequence, so long as the location of the PAM sequence is considered. Thus, in some cases, the sequence of a targeter may be a non-naturally occurring sequence. In other cases, the sequence of a targeter may be a naturally-occurring sequence, derived from the gene to be edited. In other embodiments, the activator and targeter of the gRNA are covalently linked to one another (rather than hybridizing to one another) and comprise a single molecule, referred to herein as a “single-molecule gRNA,” “single guide RNA,” a “single-molecule guide RNA,” a “one-molecule guide RNA”, or a “sgRNA”. In some embodiments, the sgRNA includes an “activator” or a “targeter” and thus can be an “activator-RNA” and a “targeter-RNA,” respectively.

Collectively, the assembled gRNAs of the disclosure comprise four distinct regions, or domains: the RNA triplex, the scaffold stem, the extended stem, and the targeting sequence that, in the embodiments of the disclosure is specific for a target nucleic acid and is located on the 3′end of the gRNA. The RNA triplex, the scaffold stem, and the extended stem, together, are referred to as the “scaffold” of the gRNA.

In some embodiments, the gRNA is a ribonucleic acid molecule (“gRNA”), and in other embodiments, the gRNA is a chimera, and comprises both DNA and RNA. As used herein, the term gRNA cover naturally-occurring molecules, as well as sequence variants.

### e. RNA Triplex

In some embodiments of the guide NAs provided herein (including reference sgRNAs), there is a RNA triplex, and the RNA triplex comprises the sequence of a UUU-nX(˜4-15)-UUU (SEQ ID NO: 17) stem loop that ends with an AAAG after 2 intervening stem loops (the scaffold stem loop and the extended stem loop), forming a pseudoknot that may also extend past the triplex into a duplex pseudoknot. The UU-UUU-AAA sequence of the triplex forms as a nexus between the targeting sequence, scaffold stem, and extended stem. In exemplary CasX sgRNAs, the UUU-loop-UUU region is coded for first, then the scaffold stem loop, and then the extended stem loop, which is linked by the tetraloop, and then an AAAG closes off the triplex before becoming the targeting sequence.

### f. Scaffold Stem Loop

In some embodiments of sgRNAs of the disclosure, the triplex region is followed by the scaffold stem loop. The scaffold stem loop is a region of the gRNA that is bound by CasX protein (such as a CasX variant protein). In some embodiments, the scaffold stem loop is a fairly short and stable stem loop. In some cases, the scaffold stem loop does not tolerate many changes, and requires some form of an RNA bubble. In some embodiments, the scaffold stem is necessary for CasX sgRNA function. While it is perhaps analogous to the nexus stem of Cas9 as being a critical stem loop, the scaffold stem of a CasX sgRNA, in some embodiments, has a necessary bulge (RNA bubble) that is different from many other stem loops found in CRISPR/Cas systems. In some embodiments, the presence of this bulge is conserved across sgRNA that interact with different CasX proteins. An exemplary sequence of a scaffold stem loop sequence of a gRNA comprises the sequence CCAGCGACUAUGUCGUAUGG (SEQ ID NO: 14).

### g. Extended Stem Loop

In some embodiments of the CasX sgRNAs of the disclosure, the scaffold stem loop is followed by the extended stem loop. In some embodiments, the extended stem comprises a synthetic tracr and crRNA fusion that is largely unbound by the CasX protein. In some embodiments, the extended stem loop can be highly malleable. In some embodiments, a single guide gRNA is made with a GAAA tetraloop linker or a GAGAAA linker between the tracr and crRNA in the extended stem loop. In some cases, the targeter and activator of a CasX sgRNA are linked to one another by intervening nucleotides and the linker can have a length of from 3 to 20 nucleotides. In some embodiments of the CasX sgRNAs of the disclosure, the extended stem is a large 32-bp loop that sits outside of the CasX protein in the ribonucleoprotein complex. An exemplary sequence of an extended stem loop sequence of a sgRNA comprises the sequence GCGCUUAUUUAUCGGAGAGAAAUCCGAUAAAUAAGAAGC (SEQ ID NO: 15). In some embodiments, the extended stem loop comprises a GAGAAA spacer sequence.

### h. Targeting Sequence

In some embodiments of the gRNAs of the disclosure, the extended stem loop is followed by a region that forms part of the triplex, and then the targeting sequence (or “spacer”) at the 3′ end of the gRNA. The targeting sequence targets the CasX ribonucleoprotein holo complex (i.e., the RNP) to a specific region of the target nucleic acid sequence of the gene to be modified. Thus, for example, gRNA targeting sequences of the disclosure have sequences complementarity to, and therefore can hybridize to, a portion of the PTBP1 gene in a nucleic acid in a eukaryotic cell (e.g., a eukaryotic chromosome, chromosomal sequence, a eukaryotic RNA, etc.) as a component of the RNP when the TC PAM motif or any one of the PAM sequences TTC, ATC, GTC, or CTC is located 1 nucleotide 5′ to the non-target strand sequence complementary to the target sequence. The targeting sequence of a gRNA can be modified so that the gRNA can target a desired sequence of any desired target nucleic acid sequence, so long as the PAM sequence location is taken into consideration. In some embodiments, the gRNA scaffold is 5′ of the targeting sequence, with the targeting sequence on the 3′ end of the gRNA. In some embodiments, the PAM motif sequence recognized by the nuclease of the RNP is TC. In other embodiments, the PAM sequence recognized by the nuclease of the RNP is NTC.

In some embodiments, the targeting sequence of the gRNA has between 14 and 35 consecutive nucleotides. In some embodiments, the targeting sequence has 14, 15, 16, 18, 18, 19, 20, 21, 22, 23 24, 25, 26, 27, 28, 29, 30, 31, 32, 33, 34 or 35 consecutive nucleotides. In some embodiments, the targeting sequence consists of 20 consecutive nucleotides. In some embodiments, the targeting sequence consists of 19 consecutive nucleotides. In some embodiments, the targeting sequence consists of 18 consecutive nucleotides. In some embodiments, the targeting sequence consists of 17 consecutive nucleotides. In some embodiments, the targeting sequence consists of 16 consecutive nucleotides. In some embodiments, the targeting sequence consists of 15 consecutive nucleotides. In some embodiments, the targeting sequence has 14, 15, 16, 17, 18, 19, 20, 21, 22, 23, 24, 25, 26, 27, 28, 29, 30, 31, 32, 33, 34 or 35 consecutive nucleotides and the targeting sequence can comprise 0 to 5, 0 to 4, 0 to 3, or 0 to 2 mismatches relative to the target nucleic acid sequence and retain sufficient binding specificity such that the gRNA of the ribonuclear protein complex (RNP) can form a complementary bond with respect to the target nucleic acid.

Representative, but non-limiting examples of targeting sequences to the PTBP1 target nucleic acid sequence contemplated for use in the gRNA of the disclosure are presented as SEQ ID NOS: 492-2100 and 2286-43569 (see Table 1). In one embodiment, the targeting sequence of the gRNA comprises a sequence having at least about 65%, at least about 75%, at least about 85%, or at least about 95% identity to a sequence selected from the group consisting of SEQ ID NOS: 492-2100 and 2286-43569. In another embodiment, the targeting sequence of the gRNA consists of a sequence selected from the group consisting of SEQ ID NOS: 37971-37979, 38027, 38136, 38137, 38152-38158, 38160, 38162-38174, 38176, 38177, 38181, 38195, 38196, 38198, 38199, 38253-38256, 38259-38267, 38306 and 38311-38353.

In some embodiments, the PAM sequence of the target nucleic acid for the gRNA targeting sequence comprises an ATC. In some embodiments, the gRNA targeting sequence for an ATC PAM comprises a sequence selected from the group consisting of SEQ ID NOS: 492-2100 and 2286-6781, or a sequence that is at least 50% identical, at least 55% identical, at least 60% identical, at least 65% identical, at least 70% identical, at least 75% identical, at least 80% identical, at least 85% identical, at least 90% identical, at least 95% identical to SEQ ID NOS: 492-2100 and 2286-6781. In some embodiments, the gRNA targeting sequence for an ATC PAM of the target nucleic acid is selected from the group consisting of SEQ ID NOS: 492-2100 and 2286-6781. In some embodiments, the PAM sequence of the target nucleic acid comprises CTC. In some embodiments, the gRNA targeting sequence for a CTC PAM of the target nucleic acid comprises a sequence selected from the group consisting of SEQ ID NOS: 16676-35169, or a sequence that is at least 50% identical, at least 55% identical, at least 60% identical, at least 65% identical, at least 70% identical, at least 75% identical, at least 80% identical, at least 85% identical, at least 90% identical, at least 95% identical to SEQ ID NOS: 16676-35169. In some embodiments, the gRNA targeting sequence for a CTC PAM is selected from the group consisting of SEQ ID NOS: 16676-35169. In some embodiments, the PAM sequence of the target nucleic acid comprises GTC. In some embodiments, the gRNA targeting sequences for a GTC PAM of the target nucleic acid comprises a sequence selected from the group consisting of SEQ ID NOS: 6782-16675 or a sequence that is at least 50% identical, at least 55% identical, at least 60% identical, at least 65% identical, at least 70% identical, at least 75% identical, at least 80% identical, at least 85% identical, at least 90% identical, at least 95% identical to SEQ ID NOS: 6782-16675. In some embodiments, the gRNA targeting sequence for a GTC PAM of the target nucleic acid is selected from the group consisting of SEQ ID NOS: 6782-16675. In some embodiments, the PAM sequence comprises TTC. In some embodiments, the gRNA targeting sequence for a TTC PAM of the target nucleic acid comprises a sequence selected from the group consisting of SEQ ID NOS: 35170-43569, or a sequence that is at least 50% identical, at least 55% identical, at least 60% identical, at least 65% identical, at least 70% identical, at least 75% identical, at least 80% identical, at least 85% identical, at least 90% identical, at least 95% identical to SEQ ID NOS: 35170-43569. In some embodiments, the gRNA targeting sequence for a TTC PAM of the target nucleic acid is selected from the group consisting of SEQ ID NOS: 35170-43569. In the foregoing embodiments, thymine (T) nucleotides can be substituted for one or more or all of the uracil (U) nucleotides in any of the targeting sequences such that the gRNA targeting sequence can be a gDNA or a gRNA, or a chimera of RNA and DNA. In some embodiments, a targeting sequence selected from the group consisting of SEQ ID NOS: 492-2100 and 2286-43569 has at least 1, 2, 3, 4, 5, or 6 or more thymine nucleotides substituted for uracil nucleotides. In some embodiments, the targeting sequence of the gRNA comprises a sequence selected from the group consisting of SEQ ID NOS: 492-2100 and 2286-43569 with a single nucleotide removed from the 3′ end of the sequence. In other embodiments, the targeting sequence of the gRNA comprises a sequence selected from the group consisting of SEQ ID NOS: 492-2100 and 2286-43569 with two nucleotides removed from the 3′ end of the sequence. In other embodiments, the targeting sequence of the gRNA comprises a sequence selected from the group consisting of SEQ ID NOS: 492-2100 and 2286-43569 with three nucleotides removed from the 3′ end of the sequence. In other embodiments, the targeting sequence of the gRNA comprises a sequence selected from the group consisting of SEQ ID NOS: 492-2100 and 2286-43569 with four nucleotides removed from the 3′ end of the sequence. In other embodiments, the targeting sequence of the gRNA comprises a sequence selected from the group consisting of SEQ ID NOS: 492-2100 and 2286-43569 with five nucleotides removed from the 3′ end of the sequence. In other embodiments, the targeting sequence of the gRNA comprises a sequence selected from the group consisting of SEQ ID NOS: 37971-37979, 38027, 38136, 38137, 38152-38158, 38160, 38162-38174, 38176, 38177, 38181, 38195, 38196, 38198, 38199, 38253-38256, 38259-38267, 38306 and 38311-38353.

TABLE 1
Targeting Sequences Specific to PTBP1

| SEQ ID NO: | PAM Sequence |
|---|---|
| 492-2100, 2286-6781 | ATC |
| 35170-43569 | TTC |
| 16676-35169 | CTC |
| 6782-16675 | GTC |

By selection of the targeting sequences of the gRNA, defined regions of the target nucleic acid sequence or sequences bracketing a particular location within the target nucleic acid can be modified or edited using the CasX:gRNA systems described herein, including facilitating the insertion of a donor template or excision of a region comprising a mutation of the PTBP1 gene. In some embodiments, the targeting sequence of the gRNA is specific for a portion of a gene encoding a PTBP1 protein. In some embodiments, the targeting sequence of a gRNA is complementary to a PTBP1 exon selected from the group consisting of exon 1, exon 2, exon 3, exon 4, exon 5, exon 6, exon 7, exon 8, exon 9, exon 10, exon 11, exon 12, exon 13, exon 14, exon 15, and exon 16. In a particular embodiment, the targeting sequence of a gRNA is complementary to a PTBP1 exon selected from exon 1, exon 2, or exon 3. In some embodiments, the targeting sequence of a gRNA is specific for a PTBP1 intron. In some embodiments, the targeting sequence of the gRNA is specific for a PTBP1 intron-exon junction. In some embodiments, the targeting sequence of the gRNA has a sequence that hybridizes with a PTBP1 regulatory element, a PTBP1 coding region, a PTBP1 non-coding region, or combinations thereof (e.g., the intersection of two regions). In some embodiments, the targeting sequence of the gRNA is complementary to a sequence comprising one or more single nucleotide polymorphisms (SNPs) of the PTBP1 gene or its complement. SNPs that are within a PTBP1 coding sequence or within a PTBP1 non-coding sequence are both within the scope of the instant disclosure. In other embodiments, the targeting sequence of the gRNA is complementary to a sequence of an intergenic region of the PTBP1 gene.

In some embodiments, the targeting sequence of a gRNA is designed to be specific for a regulatory element that regulates expression of the PTBP1 gene product. Such regulatory elements include, but are not limited to promoter regions, enhancer regions, intergenic regions, 5′ untranslated regions (5′ UTR), 3′ untranslated regions (3′ UTR), conserved elements, and regions comprising cis-regulatory elements. The promoter region is intended to encompass nucleotides within 5 kb of the initiation point of the encoding sequence or, in the case of gene enhancer elements or conserved elements, can be thousands of bp, hundreds of thousands of bp, or even millions of bp away from the encoding sequence of the gene of the target nucleic acid. In the foregoing, the targets are those in which the encoding gene of the target is intended to be knocked out or knocked down such that the PTBP1 protein is not expressed or is expressed at a lower level in a cell.

### i. gRNA Scaffolds

With the exception of the targeting sequence domain, the remaining components of the gRNA are referred to herein as the scaffold. In some embodiments, the gRNA scaffolds are derived from naturally-occurring sequences of reference gRNA. In other embodiments, the gRNA scaffolds are variants of reference gRNA wherein mutations, insertions, deletions or domain substitutions are introduced to confer desirable properties on the gRNA.

In some embodiments, a CasX reference gRNA comprises a sequence isolated or derived from Deltaproteobacter. In some embodiments, a CasX reference guide RNA comprises a sequence isolated or derived from Planctomycetes. In still other embodiments, a CasX reference gRNA comprises a sequence isolated or derived from Candidatus Sungbacteria.

Table 2 provides the sequences of reference gRNAs tracr and scaffold sequences. In some embodiments, the disclosure provides gRNA variant sequences wherein the gRNA has a scaffold comprising a sequence having one or more nucleotide modifications relative to a reference gRNA sequence having a sequence of any one of SEQ ID NOS:4-16 of Table 2. It will be understood that in those embodiments wherein a vector comprises a DNA encoding sequence for a gRNA, or where a gRNA is a chimera of RNA and DNA, that thymine (T) bases can be substituted for the uracil (U) bases of any of the gRNA sequence embodiments described herein, including the sequences of Table 2 and Table 3.

TABLE 2
Reference gRNA sequences

| SEQ | |
|---|---|
| ID NO. | Nucleotide Sequence |
| 4 | ACAUCUGGCGCGUUUAUUCCAUUACUUUGGAGCCAGUCCCAG |
| | CGACUAUGUCGUAUGGACGAAGCGCUUAUUUAUCGGAGAGAA |
| | ACCGAUAAGUAAAACGCAUCAAAG |
| 5 | UACUGGCGCUUUUAUCUCAUUACUUUGAGAGCCAUCACCAGC |
| | GACUAUGUCGUAUGGGUAAAGCGCUUAUUUAUCGGAGAGAAA |
| | UCCGAUAAAUAAGAAGCAUCAAAG |
| 6 | ACAUCUGGCGCGUUUAUUCCAUUACUUUGGAGCCAGUCCCAG |
| | CGACUAUGUCGUAUGGACGAAGCGCUUAUUUAUCGGAGA |
| 7 | ACAUCUGGCGCGUUUAUUCCAUUACUUUGGAGCCAGUCCCAG |
| | CGACUAUGUCGUAUGGACGAAGCGCUUAUUUAUCGG |
| 8 | UACUGGCGCUUUUAUCUCAUUACUUUGAGAGCCAUCACCAGC |
| | GACUAUGUCGUAUGGGUAAAGCGCUUAUUUAUCGGAGA |
| 9 | UACUGGCGCUUUUAUCUCAUUACUUUGAGAGCCAUCACCAGC |
| | GACUAUGUCGUAUGGGUAAAGCGCUUAUUUAUCGG |
| 10 | GUUUACACACUCCCUCUCAUAGGGU |
| 11 | GUUUACACACUCCCUCUCAUGAGGU |
| 12 | UUUUACAUACCCCCUCUCAUGGGAU |
| 13 | GUUUACACACUCCCUCUCAUGGGGG |
| 14 | CCAGCGACUAUGUCGUAUGG |
| 15 | GCGCUUAUUUAUCGGAGAGAAAUCCGAUAAAUAAGAAGC |
| 16 | GGCGCUUUUAUCUCAUUACUUUGAGAGCCAUCACCAGCGACU |
| | AUGUCGUAUGGGUAAAGCGCUUAUUUAUCGGA |

### j. gRNA Variants

In another aspect, the disclosure relates to guide nucleic acid variants (“gRNA variant”), which comprise one or more modifications relative to a reference gRNA scaffold. As used herein, “scaffold” refers to all parts to the gRNA necessary for gRNA function with the exception of the targeting sequence.

In some embodiments, a gRNA variant comprises one or more nucleotide substitutions, insertions, deletions, or swapped or replaced regions relative to a reference gRNA sequence of the disclosure. In some embodiments, a mutation can occur in any region of a reference gRNA to produce a gRNA variant. In some embodiments, the scaffold of the gRNA variant sequence has at least 20%, at least 30%, at least 40%, at least 50%, at least 60%, or at least 70%, at least 80%, at least 85%, at least about 90%, at least about 95%, at least about 96%, at least about 97%, at least about 98%, or at least about 99% identity to the sequence of SEQ ID NO:4 or SEQ ID NO:5.

In some embodiments, a gRNA variant comprises one or more nucleotide changes within one or more regions of the reference gRNA that improve a characteristic relative to the reference gRNA. Exemplary regions include the RNA triplex, the pseudoknot, the scaffold stem loop, and the extended stem loop. In some cases, the variant scaffold stem further comprises a bubble. In other cases, the variant scaffold further comprises a triplex loop region. In still other cases, the variant scaffold further comprises a 5′ unstructured region. In one embodiment, the gRNA variant scaffold comprises a scaffold stem loop having at least 60% sequence identity to SEQ ID NO: 14. In another embodiment, the gRNA variant comprises a scaffold stem loop having the sequence of CCAGCGACUAUGUCGUAGUGG (SEQ ID NO: 20). In another embodiment, the disclosure provides a gRNA scaffold comprising, relative to SEQ ID NO: 5, a C18G substitution, a G55 insertion, a U1 deletion, and a modified extended stem loop in which the original 6 nt loop and 13 most-loop-proximal base pairs (32 nucleotides total) are replaced by a Uvsx hairpin (4 nt loop and 5 loop-proximal base pairs; 14 nucleotides total) and the loop-distal base of the extended stem was converted to a fully base-paired stem contiguous with the new Uvsx hairpin by deletion of the A99 and substitution of G64U. In the foregoing embodiment, the gRNA scaffold comprises the sequence

(SEQ ID NO: 2238)
ACUGGCGCUUUUAUCUGAUUACUUUGAGAGCCAUCACCAGCGACUAUGUC
GUAGUGGGUAAAGCUCCCUCUUCGGAGGGAGCAUCAAAG.

All gRNA variants that have one or more improved functions or characteristics, or add one or more new functions when the variant gRNA is compared to a reference gRNA described herein, are envisaged as within the scope of the disclosure. A representative example of such a gRNA variant is guide 235 (SEQ ID NO: 43577), the utility of which is described in the Examples. In some embodiments, the gRNA variant adds a new function to the RNP comprising the gRNA variant. In some embodiments, the gRNA variant has an improved characteristic selected from: improved stability; improved transcription of the gRNA; improved resistance to nuclease activity; increased productive folding; improved binding affinity to a CasX protein; improved binding affinity to a target DNA when complexed with a CasX protein; improved gene editing when complexed with a CasX protein; improved specificity of editing when complexed with a CasX protein; and improved ability to utilize a greater spectrum of one or more PAM sequences, including ATC, CTC, GTC, or TTC, in the editing of target DNA when complexed with a CasX protein, or any combination thereof. In some cases, the one or more of the improved characteristics of the gRNA variant is at least about 1.1 to about 100,000-fold improved relative to the reference gRNA of SEQ ID NO:4 or SEQ ID NO:5. In other cases, the one or more improved characteristics of the gRNA variant is at least about 1.1, at least about 10, at least about 100, at least about 1000, at least about 10,000, at least about 100,000-fold or more improved relative to the reference gRNA of SEQ ID NO:4 or SEQ ID NO:5. In other cases, the one or more of the improved characteristics of the gRNA variant is about 1.1 to 100,00-fold, about 1.1 to 10,00-fold, about 1.1 to 1,000-fold, about 1.1 to 500-fold, about 1.1 to 100-fold, about 1.1 to 50-fold, about 1.1 to 20-fold, about 10 to 100,00-fold, about 10 to 10,00-fold, about 10 to 1,000-fold, about 10 to 500-fold, about 10 to 100-fold, about 10 to 50-fold, about 10 to 20-fold, about 2 to 70-fold, about 2 to 50-fold, about 2 to 30-fold, about 2 to 20-fold, about 2 to 10-fold, about 5 to 50-fold, about 5 to 30-fold, about 5 to 10-fold, about 100 to 100,00-fold, about 100 to 10,00-fold, about 100 to 1,000-fold, about 100 to 500-fold, about 500 to 100,00-fold, about 500 to 10,00-fold, about 500 to 1,000-fold, about 500 to 750-fold, about 1,000 to 100,00-fold, about 10,000 to 100,00-fold, about 20 to 500-fold, about 20 to 250-fold, about 20 to 200-fold, about 20 to 100-fold, about 20 to 50-fold, about 50 to 10,000-fold, about 50 to 1,000-fold, about 50 to 500-fold, about 50 to 200-fold, or about 50 to 100-fold, improved relative to the reference gRNA of SEQ ID NO:4 or SEQ ID NO:5. In other cases, the one or more improved characteristics of the gRNA variant is about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 40-fold, 45-fold, 50-fold, 55-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 310-fold, 320-fold, 330-fold, 340-fold, 350-fold, 360-fold, 370-fold, 380-fold, 390-fold, 400-fold, 425-fold, 450-fold, 475-fold, or 500-fold improved relative to the reference gRNA of SEQ ID NO:4 or SEQ ID NO:5.

In some embodiments, a gRNA variant can be created by subjecting a reference gRNA to a one or more mutagenesis methods, such as the mutagenesis methods described herein, below, which may include Deep Mutational Evolution (DME), deep mutational scanning (DMS), error prone PCR, cassette mutagenesis, random mutagenesis, staggered extension PCR, gene shuffling, or domain swapping, in order to generate the gRNA variants of the disclosure. The activity of reference gRNAs may be used as a benchmark against which the activity of gRNA variants are compared, thereby measuring improvements in function of gRNA variants. In other embodiments, a reference gRNA may be subjected to one or more deliberate, targeted mutations, substitutions, or domain swaps in order to produce a gRNA variant, for example a rationally designed variant. Exemplary gRNA variants produced by such methods are described in the Examples and representative sequences of gRNA scaffolds are presented in Table 3 SEQ ID NOS: 2101-2285, 43571-43661 and 44045.

In some embodiments, the gRNA variant comprises one or more modifications compared to a reference guide nucleic acid scaffold sequence, wherein the one or more modification is selected from: at least one nucleotide substitution in a region of the gRNA variant; at least one nucleotide deletion in a region of the gRNA variant; at least one nucleotide insertion in a region of the gRNA variant; a substitution of all or a portion of a region of the gRNA variant; a deletion of all or a portion of a region of the gRNA variant; or any combination of the foregoing. In some cases, the modification is a substitution of 1 to 15 consecutive or non-consecutive nucleotides in the gRNA variant in one or more regions. In other cases, the modification is a deletion of 1 to 10 consecutive or non-consecutive nucleotides in the gRNA variant in one or more regions. In other cases, the modification is an insertion of 1 to 10 consecutive or non-consecutive nucleotides in the gRNA variant in one or more regions. In other cases, the modification is a substitution of the scaffold stem loop or the extended stem loop with an RNA stem loop sequence from a heterologous RNA source with proximal 5′ and 3′ ends. In some cases, a gRNA variant of the disclosure comprises two or more modifications in one region. In other cases, a gRNA variant of the disclosure comprises modifications in two or more regions. In other cases, a gRNA variant comprises any combination of the foregoing modifications described in this paragraph.

In some embodiments, a 5′ G is added to a gRNA variant sequence for expression in vivo, as transcription from a U6 promoter is more efficient and more consistent with regard to the start site when the +1 nucleotide is a G. In other embodiments, two 5′ Gs are added to a gRNA variant sequence for in vitro transcription to increase production efficiency, as T7 polymerase strongly prefers a G in the +1 position and a purine in the +2 position. In some cases, the 5′ G bases are added to the reference scaffolds of Table 2. In other cases, the 5′ G bases are added to the variant scaffolds of Table 3, e.g. SEQ ID NOS: 2101-2285, 43571-43661 or 44045.

Table 3 provides exemplary gRNA variant scaffold sequences. In some embodiments, the disclosure provides gRNA a variant scaffold comprising any one of SEQ ID NOS: 2101-2285, 43571-43661, or 44045, or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto. In some embodiments, the disclosure provides gRNA a variant scaffold comprising any one of SEQ ID NOS:2238-2285, 43571-43661, 44045 and 44047, or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto. In some embodiments, the disclosure provides gRNA a variant scaffold comprising any one of SEQ ID NOS: 2281-2285, 43571-43661 or 44045, or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto. It will be understood that in those embodiments wherein a vector comprises a DNA encoding sequence for a gRNA, or where a gRNA is a gDNA or a chimera of RNA and DNA, that thymine (T) bases can be substituted for the uracil (U) bases of any of the gRNA sequence embodiments described herein.

TABLE 3
Exemplary gRNA Scaffold Sequences

| SEQ ID NO: | Name | NUCLEOTIDE SEQUENCE OR DESCRIPTION OF MODIFICATION |
|---|---|---|
| 2101 | ND | phage replication stable |
| 2102 | ND | Kissing loop_b1 |
| 2103 | ND | Kissing loop_a |
| 2104 | ND | 32: uvsX hairpin |
| 2105 | ND | PP7 |
| 2106 | ND | 64: trip mut, extended stem truncation |
| 2107 | ND | hyperstable tetraloop |
| 2108 | ND | C18G |
| 2109 | ND | U17G |
| 2110 | ND | CUUCGG loop |
| 2111 | ND | MS2 |
| 2112 | ND | −1, A2G, −78, G77U |
| 2113 | ND | QB |
| 2114 | ND | 45, 44 hairpin |
| 2115 | ND | U1A |
| 2116 | ND | A14C, U17G |
| 2117 | ND | CUUCGG loop modified |
| 2118 | ND | Kissing loop_b2 |
| 2119 | ND | −76:78, −83:87 |
| 2120 | ND | −4 |
| 2121 | ND | extended stem truncation |
| 2122 | ND | C55 |
| 2123 | ND | trip mut |
| 2124 | ND | −76:78 |
| 2125 | ND | −1:5 |
| 2126 | ND | −83:87 |
| 2127 | ND | = +G28, A82U, −84, |
| 2128 | ND | = +51U |
| 2129 | ND | −1:4, +G5A, +G86, |
| 2130 | ND | = +A94 |
| 2131 | ND | = +G72 |
| 2132 | ND | shorten front, CUUCGG loop modified. extend extended |
| 2133 | ND | A14C |
| 2134 | ND | −1:3, +G3 |
| 2135 | ND | = +C45, +U46 |
| 2136 | ND | CUUCGG loop modified, fun start |
| 2137 | ND | −93:94 |
| 2138 | ND | = +U45 |
| 2139 | ND | −69, −94 |
| 2140 | ND | −94 |
| 2141 | ND | modified CUUCGG, minus U in 1st triplex |
| 2142 | ND | −1:4, +C4, A14C, U17G, +G72, −76:78, −83:87 |
| 2143 | ND | U1C, −73 |
| 2144 | ND | Scaffold uuCG, stem uuCG. Stem swap, t shorten |
| 2145 | ND | Scaffold uuCG, stem uuCG. Stem swap |
| 2146 | ND | = +G60 |
| 2147 | ND | no stem Scaffold uuCG |
| 2148 | ND | no stem Scaffold uuCG, fun start |
| 2149 | ND | Scaffold uuCG, stem uuCG, fun start |
| 2150 | ND | Pseudoknots |
| 2151 | ND | Scaffold uuCG, stem uuCG |
| 2152 | ND | Scaffold uuCG, stem uuCG, no start |
| 2153 | ND | Scaffold uuCG |
| 2154 | ND | = +GCUC36 |
| 2155 | ND | G quadriplex telomere basket+ ends |
| 2156 | ND | G quadriplex M3q |
| 2157 | ND | G quadriplex telomere basket no ends |
| 2158 | ND | 45, 44 hairpin (old version) |
| 2159 | ND | Sarcin-ricin loop |
| 2160 | ND | uvsX, C18G |
| 2161 | ND | truncated stem loop, C18G, trip mut (U10C) |
| 2162 | ND | short phage rep, C18G |
| 2163 | ND | phage rep loop, C18G |
| 2164 | ND | = +G18, stacked onto 64 |
| 2165 | ND | truncated stem loop, C18G, −1 A2G |
| 2166 | ND | phage rep loop, C18G, trip mut (U10C) |
| 2167 | ND | short phage rep, C18G, trip mut (U10C) |
| 2168 | ND | uvsX, trip mut (U10C) |
| 2169 | ND | truncated stem loop |
| 2170 | ND | = +A17, stacked onto 64 |
| 2171 | ND | 3′ HDV genomic ribozyme |
| 2172 | ND | phage rep loop, trip mut (U10C) |
| 2173 | ND | −79:80 |
| 2174 | ND | short phage rep, trip mut (U10C) |
| 2175 | ND | extra truncated stem loop |
| 2176 | ND | U17G, C18G |
| 2177 | ND | short phage rep |
| 2178 | ND | uvsX, C18G, −1 A2G |
| 2179 | ND | uvsX, C18G, trip mut (U10C), −1 A2G, HDV −99 G65U |
| 2180 | ND | 3′ HDV antigenomic ribozyme |
| 2181 | ND | uvsX, C18G, trip mut (U10C), −1 A2G, HDV AA(98:99)C |
| 2182 | ND | 3′ HDV ribozyme (Lior Nissim, Timothy Lu) |
| 2183 | ND | TAC(1:3)GA, stacked onto 64 |
| 2184 | ND | uvsX, −1 A2G |
| 2185 | ND | truncated stem loop, C18G, trip mut (U10C), −1 A2G, HDV −99 G65U |
| 2186 | ND | short phage rep, C18G, trip mut (U10C), −1 A2G, HDV −99 G65U |
| 2187 | ND | 3′ sTRSV WT viral Hammerhead ribozyme |
| 2188 | ND | short phage rep, C18G, −1 A2G |
| 2189 | ND | short phage rep, C18G, trip mut (U10C), −1 A2G, 3′ genomic HDV |
| 2190 | ND | phage rep loop, C18G, trip mut (U10C), −1 A2G, HDV −99 G65U |
| 2191 | ND | 3′ HDV ribozyme (Owen Ryan, Jamie Cate) |
| 2192 | ND | phage rep loop, C18G, −1 A2G |
| 2193 | ND | 0.14 |
| 2194 | ND | −78, G77U |
| 2195 | ND | ND |
| 2196 | ND | short phage rep, −1 A2G |
| 2197 | ND | truncated stem loop, C18G, trip mut (U10C), −1 A2G |
| 2198 | ND | −1, A2G |
| 2199 | ND | truncated stem loop, trip mut (U10C), −1 A2G |
| 2200 | ND | uvsX, C18G, trip mut (U10C), −1 A2G |
| 2201 | ND | phage rep loop, −1 A2G |
| 2202 | ND | phage rep loop, trip mut (U10C), −1 A2G |
| 2203 | ND | phage rep loop, C18G, trip mut (U10C), −1 A2G |
| 2204 | ND | truncated stem loop, C18G |
| 2205 | ND | uvsX, trip mut (U10C), −1 A2G |
| 2206 | ND | truncated stem loop, −1 A2G |
| 2207 | ND | short phage rep, trip mut (U10C), −1 A2G |
| 2208 | ND | 5′HDV ribozyme (Owen Ryan, Jamie Cate) |
| 2209 | ND | 5′HDV genomic ribozyme |
| 2210 | ND | truncated stem loop, C18G, trip mut (U10C), −1 A2G, HDV AA(98:99)C |
| 2211 | ND | 5′env25 pistol ribozyme (with an added CUUCGG loop) |
| 2212 | ND | 5′HDV antigenomic ribozyme |
| 2213 | ND | 3′ Hammerhead ribozyme (Lior Nissim, Timothy Lu) guide scaffold scar |
| 2214 | ND | = +A27, stacked onto 64 |
| 2215 | ND | 5′Hammerhead ribozyme (Lior Nissim, Timothy Lu) smaller scar |
| 2216 | ND | phage rep loop, C18G, trip mut (U10C), −1 A2G, HDV AA(98:99)C |
| 2217 | ND | −27, stacked onto 64 |
| 2218 | ND | 3′ Hatchet |
| 2219 | ND | 3′ Hammerhead ribozyme (Lior Nissim, Timothy Lu) |
| 2220 | ND | 5′ Hatchet |
| 2221 | ND | 5′ HDV ribozyme (Lior Nissim, Timothy Lu) |
| 2222 | ND | 5′ Hammerhead ribozyme (Lior Nissim, Timothy Lu) |
| 2223 | ND | 3′ HH15 Minimal Hammerhead ribozyme |
| 2224 | ND | 5′ RBMX recruiting motif |
| 2225 | ND | 3′ Hammerhead ribozyme (Lior Nissim, Timothy Lu) smaller scar |
| 2226 | ND | 3′ env25 pistol ribozyme (with an added CUUCGG loop) |
| 2227 | ND | 3′ Env-9 Twister |
| 2228 | ND | = +AUUAUCUCAUUACU25 |
| 2229 | ND | 5′ Env-9 Twister |
| 2230 | ND | 3′ Twisted Sister 1 |
| 2231 | ND | no stem |
| 2232 | ND | 5′ HH15 Minimal Hammerhead ribozyme |
| 2233 | ND | 5′ Hammerhead ribozyme (Lior Nissim, Timothy Lu) guide scaffold scar |
| 2234 | ND | 5′ Twisted Sister 1 |
| 2235 | ND | 5′ sTRSV WT viral Hammerhead ribozyme |
| 2236 | ND | 148: = +G55, stacked onto 64 |
| 2237 | ND | 158: 103 + 148(+G55) −99, G65U |
| 2238 | 174 | ACUGGCGCUUUUAUCUGAUUACUUUGAGAGCCAUCACCAGCGACUAUGUCGUAGUGGG |
| | | UAAAGCUCCCUCUUCGGAGGGAGCAUCAAAG |
| 2239 | 175 | ACUGGCGCCUUUAUCUCAUUACUUUGAGAGCCAUCACCAGCGACUAUGUCGUAUGGGU |
| | | AAAGCGCUUACGGACUUCGGUCCGUAAGAAGCAUCAAAG |
| 2240 | 176 | GCUGGCGCUUUUAUCUGAUUACUUUGAGAGCCAUCACCAGCGACUAUGUCGUAGUGGG |
| | | UAAAGCUCCCUCUUCGGAGGGAGCAUCAAAG |
| 2241 | 177 | ACUGGCGCUUUUAUCUGAUUACUUUGAGAGCCAUCACCAGCGACUAUGUCGUAUGGGU |
| | | AAAGCUCCCUCUUCGGAGGGAGCAUCAAAG |
| 2242 | 181 | ACUGGCGCCUUUAUCUGAUUACUUUGAGAGCCAUCACCAGCGACUAUGUCGUAUGGGU |
| | | AAAGCGCUUACGGACUUCGGUCCGUAAGAAGCAUCAAAG |
| 2243 | 182 | ACUGGCGCUUUUAUCUGAUUACUUUGAGAGCCAUCACCAGCGACUAUGUCGUAUGGGU |
| | | AAAGCGCUUACGGACUUCGGUCCGUAAGAAGCAUCAAAG |
| 2244 | 183 | ACUGGCGCUUUUAUCUGAUUACUUUGAGAGCCAUCACCAGCGACUAUGUCGUAGUGGG |
| | | UAAAGCGCUUACGGACUUCGGUCCGUAAGAAGCAUCAAAG |
| 2245 | 184 | ACUGGCGCUUUUAUCUGAUUACUUUGAGAGCCAUCACCAGCGACUAUGUCGUAUUGGG |
| | | UAAAGCUCCCUCUUCGGAGGGAGCAUCAAAG |
| 2246 | 185 | ACUGGCGCUUUUAUCUGAUUACUUUGAGAGCCAUCACCAGCGACUAUGUCGUAUUGGG |
| | | UAAAGCGCUUACGGACUUCGGUCCGUAAGAAGCAUCAAAG |
| 2247 | 186 | ACUGGCGCCUUUAUCAUCAUUACUUUGAGAGCCAUCACCAGCGACUAUGUCGUAUGGG |
| | | UAAAGCGCUUACGGACUUCGGUCCGUAAGAAGCAUCAAAG |
| 2248 | 187 | ACUGGCGCUUUUAUCUGAUUACUUUGAGAGCCAUCACCAGCGACUAUGUCGUAGUGGG |
| | | UAAAGCGCCCUCUUCGGAGGGAAGCAUCAAAG |
| 2249 | 188 | ACUGGCGCUUUUAUCUGAUUACUUUGAGAGCCAUCACCAGCGACUAUGUCGUAGUGGG |
| | | UAAAGCUCACAUGAGGAUCACCCAUGUGAGCAUCAAAG |
| 2250 | 189 | ACUGGCACUUUUACCUGAUUACUUUGAGAGCCAACACCAGCGACUAUGUCGUAGUGGG |
| | | UAAAGCUCCCUCUUCGGAGGGAGCAUCAAAG |
| 2251 | 190 | ACUGGCACUUUUAUCUGAUUACUUUGAGAGCCAUCACCAGCGACUAUGUCGUAGUGGG |
| | | UAAAGCUCCCUCUUCGGAGGGAGCAUCAAAG |
| 2252 | 191 | ACUGGCCCUUUUAUCUGAUUACUUUGAGAGCCAUCACCAGCGACUAUGUCGUAGUGGG |
| | | UAAAGCUCCCUCUUCGGAGGGAGCAUCAAAG |
| 2253 | 192 | ACUGGCGCUUUUACCUGAUUACUUUGAGAGCCAUCACCAGCGACUAUGUCGUAGUGGG |
| | | UAAAGCUCCCUCUUCGGAGGGAGCAUCAAAG |
| 2254 | 193 | ACUGGCGCUUUUAUCUGAUUACUUUGAGAGCCAACACCAGCGACUAUGUCGUAGUGGG |
| | | UAAAGCUCCCUCUUCGGAGGGAGCAUCAAAG |
| 2255 | 195 | ACUGGCACCUUUACCUGAUUACUUUGAGAGCCAACACCAGCGACUAUGUCGUAUGGGU |
| | | AAAGCGCUUACGGACUUCGGUCCGUAAGAAGCAUCAAAG |
| 2256 | 196 | ACUGGCACCUUUAUCUGAUUACUUUGAGAGCCAUCACCAGCGACUAUGUCGUAUGGGU |
| | | AAAGCGCUUACGGACUUCGGUCCGUAAGAAGCAUCAAAG |
| 2257 | 197 | ACUGGCCCCUUUAUCUGAUUACUUUGAGAGCCAUCACCAGCGACUAUGUCGUAUGGGU |
| | | AAAGCGCUUACGGACUUCGGUCCGUAAGAAGCAUCAAAG |
| 2258 | 198 | ACUGGCGCCUUUAUCUGAUUACUUUGAGAGCCAACACCAGCGACUAUGUCGUAUGGGU |
| | | AAAGCGCUUACGGACUUCGGUCCGUAAGAAGCAUCAAAG |
| 2259 | 199 | GCUGGCGCUUUUAUCUGAUUACUUUGAGAGCCAUCACCAGCGACUAUGUCGUAGUGGG |
| | | UAAAGCUCCCUCUUCGGAGGGAGCAUCAAAG |
| 2260 | 200 | GACUGGCGCUUUUAUCUGAUUACUUUGAGAGCCAUCACCAGCGACUAUGUCGUAGUGG |
| | | GUAAAGCUCCCUCUUCGGAGGGAGCAUCAAAG |
| 2261 | 201 | ACUGGCGCCUUUAUCUGAUUACUUUGGAGAGCCAUCACCAGCGACUAUGUCGUAGUGG |
| | | GUAAAGCUCCCUCUUCGGAGGGAGCAUCAAAG |
| 2262 | 202 | ACUGGCGCAUUUAUCUGAUUACUUUGUGAGCCAUCACCAGCGACUAUGUCGUAGUGGG |
| | | UAAAGCUCCCUCUUCGGAGGGAGCAUCAAAG |
| 2263 | 203 | ACUGGCGCCUUUAUCUGAUUACUUUGAGAGCCAUCACCAGCGACUAUGUCGUAGUGGG |
| | | UAAAGCUCCCUCUUCGGAGGGAGCAUCAAAG |
| 2264 | 204 | ACUGGCGCUUUUAUCUGAUUACUUUGGAGAGCCAUCACCAGCGACUAUGUCGUAGUGG |
| | | GUAAAGCUCCCUCUUCGGAGGGAGCAUCAAAG |
| 2265 | 205 | ACUGGCGCAUUUAUCUGAUUACUUUGAGAGCCAUCACCAGCGACUAUGUCGUAGUGGG |
| | | UAAAGCUCCCUCUUCGGAGGGAGCAUCAAAG |
| 2266 | 206 | ACUGGCGCUUUUAUCUGAUUACUUUGUGAGCCAUCACCAGCGACUAUGUCGUAGUGGG |
| | | UAAAGCUCCCUCUUCGGAGGGAGCAUCAAAG |
| 2267 | 207 | ACUGGCGCUUUUAUUCUGAUUACUUUGAGAGCCAUCACCAGCGACUAUGUCGUAGUGG |
| | | GUAAAGCUCCCUCUUCGGAGGGAGCAUCAAAG |
| 2268 | 208 | ACGGCGCUUUUAUCUGAUUACUUUGAGAGCCAUCACCAGCGACUAUGUCGUAGUGGGU |
| | | AAAGCUCCCUCUUCGGAGGGAGCAUCAAAG |
| 2269 | 209 | ACUGGCGCUUUUAUAUGAUUACUUUGAGAGCCAUCACCAGCGACUAUGUCGUAGUGGG |
| | | UAAAGCUCCCUCUUCGGAGGGAGCAUCAAAG |
| 2270 | 210 | ACUGGCGCUUUUAUCUUGAUUACUUUGAGAGCCAUCACCAGCGACUAUGUCGUAGUGG |
| | | GUAAAGCUCCCUCUUCGGAGGGAGCAUCAAAG |
| 2271 | 211 | ACUGGCGCUUUUAUCUGAUUACUUUGAGAGCCAGCACCAGCGACUAUGUCGUAGUGGG |
| | | UAAAGCUCCCUCUUCGGAGGGAGCAUCAAAG |
| 2272 | 212 | ACUGGCGCUGUUAUCUGAUUACUUCGAGAGCCAUCACCAGCGACUAUGUCGUAGUGGG |
| | | UAAAGCUCCCUCUUCGGAGGGAGCAUCGAAG |
| 2273 | 213 | ACUGGCGCUCUUAUCUGAUUACUUCGAGAGCCAUCACCAGCGACUAUGUCGUAGUGGG |
| | | UAAAGCUCCCUCUUCGGAGGGAGCAUCGAAG |
| 2274 | 214 | ACUGGCGCUUGUAUCUGAUUACUCUGAGAGCCAUCACCAGCGACUAUGUCGUAGUGGG |
| | | UAAAGCUCCCUCUUCGGAGGGAGCAUCAGAG |
| 2275 | 215 | ACUGGCGCUUCUAUCUGAUUACUCUGAGAGCCAUCACCAGCGACUAUGUCGUAGUGGG |
| | | UAAAGCUCCCUCUUCGGAGGGAGCAUCAGAG |
| 2276 | 216 | ACUGGCGCUUUGAUCUGAUUACCUUGAGAGCCAUCACCAGCGACUAUGUCGUAGUGGG |
| | | UAAAGCUCCCUCUUCGGAGGGAGCAUCAAGG |
| 2277 | 217 | ACUGGCGCUUUCAUCUGAUUACCUUGAGAGCCAUCACCAGCGACUAUGUCGUAGUGGG |
| | | UAAAGCUCCCUCUUCGGAGGGAGCAUCAAGG |
| 2278 | 218 | ACUGGCGCUGUUAUCUGAUUACUUUGAGAGCCAUCACCAGCGACUAUGUCGUAGUGGG |
| | | UAAAGCUCCCUCUUCGGAGGGAGCAUCAAAG |
| 2279 | 219 | ACUGGCGCUUUUAUCUGAUUACUUUGAGAGCCAUCACCAGCGACUAUGUCGUAGUGGG |
| | | UAAAGCUCCCUCUUCGGAGGGAGCAUCGAAG |
| 2280 | 220 | ACUGGCGCUUUUAUCUGAUUACUUCGAGAGCCAUCACCAGCGACUAUGUCGUAGUGGG |
| | | UAAAGCUCCCUCUUCGGAGGGAGCAUCAAAG |
| 2281 | 221 | ACUGGCACUUCUAUCUGAUUACUCUGAGAGCCAUCACCAGCGACUAUGUCGUAUGGGU |
| | | AAAGCCGCUUACGGACUUCGGUCCGUAAGAGGCAUCAGAG |
| 2282 | 222 | ACUGGCACUUCUAUCUGAUUACUCUGAGAGCCAUCACCAGCGACUAUGUCGUAGUGGG |
| | | UAAAGCUCCCUCUUCGGAGGGAGCAUCAGAG |
| 2283 | 223 | ACUGGCACCUUUAUCUGAUUACUUUGAGAGCCAUCACCAGCGACUAUGUCGUAUGGGU |
| | | AAAGCCGCUUACGGACUUCGGUCCGUAAGAGGCAUCAAAG |
| 2284 | 224 | ACUGGCACUUGUAUCUGAUUACUCUGAGAGCCAUCACCAGCGACUAUGUCGUAUGGGU |
| | | AAAGCCGCUUACGGACUUCGGUCCGUAAGAGGCAUCAGAG |
| 2285 | 225 | ACUGGCACUUGUAUCUGAUUACUCUGAGAGCCAUCACCAGCGACUAUGUCGUAGUGGG |
| | | UAAAGCUCCCUCUUCGGAGGGAGCAUCAGAG |
| 44045 | 226 | ACUGGCGCUUUUAUCUGAUUACUUUGAGAGCCAUCACCAGCGACUAUGUCGUAGUGGG |
| | | UAAAGCUGCACUAUGGGCGCAGCGUCAAUGACGCUGACGGUACAGGCCAGACAAUUAU |
| | | UGUCUGGUAUAGUGCAGCAUCAAAG |
| 43571 | 229 | ACUGGCACUUUUAUCUGAUUACUUUGAGAGCCAUCACCAGCGACUAUGUCGUAUGGGU |
| | | AAAGCGCUUACGGACUUCGGUCCGUAAGAAGCAUCAAAG |
| 43572 | 230 | ACUGGCACUUCUAUCUGAUUACUCUGAGAGCCAUCACCAGCGACUAUGUCGUAUGGGU |
| | | AAAGCGCUUACGGACUUCGGUCCGUAAGAAGCAUCAGAG |
| 43573 | 231 | ACUGGCGCUUCUAUCUGAUUACUCUGAGAGCCAUCACCAGCGACUAUGUCGUAUGGGU |
| | | AAAGCCGCUUACGGACUUCGGUCCGUAAGAGGCAUCAGAG |
| 43574 | 232 | ACUGGCACUUCUAUCUGAUUACUCUGAGCGCCAUCACCAGCGACUAUGUCGUAUGGGU |
| | | AAAGCCGCUUACGGACUUCGGUCCGUAAGAGGCAUCAGAG |
| 43575 | 233 | ACUGGCGCUUCUAUCUGAUUACUCUGAGCGCCAUCACCAGCGACUAUGUCGUAUGGGU |
| | | AAAGCCGCUUACGGACUUCGGUCCGUAAGAGGCAUCAGAG |
| 43576 | 234 | ACUGGCGCUUCUAUCUGAUUACUCUGAGCGCCAUCACCAGCGACUAUGUCGUAUGGGU |
| | | AAAGCGCCUUACGGACUUCGGUCCGUAAGGAGCAUCAGAG |
| 43577 | 235 | ACUGGCGCUUCUAUCUGAUUACUCUGAGCGCCAUCACCAGCGACUAUGUCGUAGUGGG |
| | | UAAAGCCGCUUACGGACUUCGGUCCGUAAGAGGCAUCAGAG |
| 43578 | 236 | ACGGGACUUUCUAUCUGAUUACUCUGAAGUCCCUCACCAGCGACUAUGUCGUAUGGGU |
| | | AAAGCCGCUUACGGACUUCGGUCCGUAAGAGGCAUCAGAG |
| 43579 | 237 | ACCUGUAGUUCUAUCUGAUUACUCUGACUACAGUCACCAGCGACUAUGUCGUAUGGGU |
| | | AAAGCCGCUUACGGACUUCGGUCCGUAAGAGGCAUCAGAG |
| 43580 | 238 | ACUGGCGCUUUUAUCUGAUUACUUUGAGAGCCAUCACCAGCGACUAUGUCGUAGUGGG |
| | | UAAAGCUGCACGGUGGGCGCAGCUUCGGCUGACGGUACACCGUGCAGCAUCAAAG |
| 43581 | 239 | ACUGGCGCUUUUAUCUGAUUACUUUGAGAGCCAUCACCAGCGACUAUGUCGUAGUGGG |
| | | UAAAGCUGCACGGUGGGCGCAGCUUCGGCUGACGGUACACCGGUGGGCGCAGCUUCGG |
| | | CUGACGGUACACCGUGCAGCAUCAAAG |
| 43582 | 240 | ACUGGCGCUUUUAUCUGAUUACUUUGAGAGCCAUCACCAGCGACUAUGUCGUAGUGGG |
| | | UAAAGCUGCACGGUGGGCGCAGCUUCGGCUGACGGUACACCGGUGGGCGCAGCUUCGG |
| | | CUGACGGUACACCGGUGGGCGCAGCUUCGGCUGACGGUACACCGUGCAGCAUCAAAG |
| 43583 | 241 | ACUGGCGCUUUUAUCUGAUUACUUUGAGAGCCAUCACCAGCGACUAUGUCGUAGUGGG |
| | | UAAAGCUGCACGGUGGGCGCAGCUUCGGCUGACGGUACACCGGUGGGCGCAGCUUCGG |
| | | CUGACGGUACACCGGUGGGCGCAGCUUCGGCUGACGGUACACCGGUGGGCGCAGCUUC |
| | | GGCUGACGGUACACCGUGCAGCAUCAAAG |
| 43584 | 242 | ACUGGCGCUUUUAUCUGAUUACUUUGAGAGCCAUCACCAGCGACUAUGUCGUAGUGGG |
| | | UAAAGCUGCACGGUGGGCGCAGCUUCGGCUGACGGUACACCGGUGGGCGCAGCUUCGG |
| | | CUGACGGUACACCGGUGGGCGCAGCUUCGGCUGACGGUACACCGGUGGGCGCAGCUUC |
| | | GGCUGACGGUACACCGGUGGGCGCAGCUUCGGCUGACGGUACACCGUGCAGCAUCAAA |
| | | G |
| 43585 | 243 | ACUGGCGCUUUUAUCUGAUUACUUUGAGAGCCAUCACCAGCGACUAUGUCGUAGUGGG |
| | | UAAAGCUGCACCUAGCGGAGGCUAGGUGCAGCAUCAAAG |
| 43586 | 244 | ACUGGCGCUUUUAUCUGAUUACUUUGAGAGCCAUCACCAGCGACUAUGUCGUAGUGGG |
| | | UAAAGCUGCACCUCGGCUUGCUGAAGCGCGCACGGCAAGAGGCGAGGUGCAGCAUCAA |
| | | AG |
| 43587 | 245 | ACUGGCGCUUUUAUCUGAUUACUUUGAGAGCCAUCACCAGCGACUAUGUCGUAGUGGG |
| | | UAAAGCUGCACCUCUCUCGACGCAGGACUCGGCUUGCUGAAGCGCGCACGGCAAGAGG |
| | | CGAGGGGCGGCGACUGGUGAGUACGCCAAAAAUUUUGACUAGCGGAGGCUAGAAGGAG |
| | | AGAGGUGCAGCAUCAAAG |
| 43588 | 246 | ACUGGCGCUUUUAUCUGAUUACUUUGAGAGCCAUCACCAGCGACUAUGUCGUAGUGGG |
| | | UAAAGCUGCACGGUGCCCGUCUGUUGUGUCGAGAGACGCCAAAAAUUUUGACUAGCGG |
| | | AGGCUAGAAGGAGAGAGAUGGGUGCCGUGCAGCAUCAAAG |
| 43589 | 247 | ACUGGCGCUUUUAUCUGAUUACUUUGAGAGCCAUCACCAGCGACUAUGUCGUAGUGGG |
| | | UAAAGCUGCACAUGGAGAGGAGAUGUGCAGCAUCAAAG |
| 43590 | 248 | ACUGGCGCUUUUAUCUGAUUACUUUGAGAGCCAUCACCAGCGACUAUGUCGUAGUGGG |
| | | UAAAGCUGCACAUGGAGAUGUGCAGCAUCAAAG |
| 43591 | 249 | ACUGGCGCUUUUAUCUGAUUACUUUGAGAGCCAUCACCAGCGACUAUGUCGUAGUGGG |
| | | UAAAGCUUGGGCGCAGCGUCAAUGACGCUGACGGUACAAGCAUCAAAG |
| 43592 | 250 | ACUGGCGCUUUUAUCUGAUUACUUUGAGAGCCAUCACCAGCGACUAUGUCGUAGUGGG |
| | | UAAAGCUGCACUAUGGGCGCAGCGUCAAUGACGCUGACGGUACAGGCCACAUGAGGAU |
| | | CACCCAUGUGGUAUAGUGCAGCAUCAAAG |
| 43593 | 251 | ACUGGCGCUUUUAUCUGAUUACUUUGAGAGCCAUCACCAGCGACUAUGUCGUAGUGGG |
| | | UAAAGCUGCACUAUGGGCGCAGCUCAUGAGGAUCACCCAUGAGCUGACGGUACAGGCC |
| | | ACAUGAGGAUCACCCAUGUGGUAUAGUGCAGCAUCAAAG |
| 43594 | 252 | ACUGGCGCUUUUAUCUGAUUACUUUGAGAGCCAUCACCAGCGACUAUGUCGUAGUGGG |
| | | UAAAGCUGCACUAUGGGCGCAGCGUCAAUGACGCUGACGGUACAGGCCACAUGGCAGU |
| | | CGUAACGACGCGGGUGGUAUAGUGCAGCAUCAAAG |
| 43595 | 253 | ACUGGCGCUUUUAUCUGAUUACUUUGAGAGCCAUCACCAGCGACUAUGUCGUAGUGGG |
| | | UAAAGCUGCACUAUGGGCGCAGCAAACAUGGCAGUCCUAAGGACGCGGGUUUUGCUGA |
| | | CGGUACAGGCCACAUGGCAGUCGUAACGACGCGGGUGGUAUAGUGCAGCAUCAAAG |
| 43596 | 254 | ACUGGCGCUUUUAUCUGAUUACUUUGAGAGCCAUCACCAGCGACUAUGUCGUAGUGGG |
| | | UAAAGCUGCACUAUGGGCGCAGACAUGGCAGUCGUAACGACGCGGGUCUGACGGUACA |
| | | GGCCACAUGAGGAUCACCCAUGUGGUAUAGUGCAGCAUCAAAG |
| 43597 | 255 | ACUGGCGCUUUUAUCUGAUUACUUUGAGAGCCAUCACCAGCGACUAUGUCGUAGUGGG |
| | | UAAAGCUGCACUAAGGAGUUUAUAUGGAAACCCUUAGUGCAGCAUCAAAG |
| 43598 | 256 | ACUGGCGCUUUUAUCUGAUUACUUUGAGAGCCAUCACCAGCGACUAUGUCGUAGUGGG |
| | | UAAAGCUCAGGAAGCACUAUGGGCGCAGCGUCAAUGACGCUGACGGUACAGGCCAGAC |
| | | AAUUAUUGUCUGGUAUAGUGCAGCAGCAGAACAAUUUGCUGAGGGCUAUUGAGGCGCA |
| | | ACAGCAUCUGUUGCAACUCACAGUCUGGGGCAUCAAGCAGCUCCAGGCAAGAAUCCUG |
| | | AGCAUCAAAG |
| 43599 | 257 | ACUGGCGCUUUUAUCUGAUUACUUUGAGAGCCAUCACCAGCGACUAUGUCGUAGUGGG |
| | | UAAAGCUGCACGCCCUGAAGAAGGGCGUGCAGCAUCAAAG |
| 43600 | 258 | ACUGGCGCUUUUAUCUGAUUACUUUGAGAGCCAUCACCAGCGACUAUGUCGUAGUGGG |
| | | UAAAGCUGCACGGCUCGUGUAGCUCAUUAGCUCCGAGCCGUGCAGCAUCAAAG |
| 43601 | 259 | ACUGGCGCUUUUAUCUGAUUACUUUGAGAGCCAUCACCAGCGACUAUGUCGUAGUGGG |
| | | UAAAGCUGCACCCGUGUGCAUCCGCAGUGUCGGAUCCACGGGUGCAGCAUCAAAG |
| 43602 | 260 | ACUGGCGCUUUUAUCUGAUUACUUUGAGAGCCAUCACCAGCGACUAUGUCGUAGUGGG |
| | | UAAAGCUGCACGGAAUCCAUUGCACUCCGGAUUUCACUAGGUGCAGCAUCAAAG |
| 43603 | 261 | ACUGGCGCUUUUAUCUGAUUACUUUGAGAGCCAUCACCAGCGACUAUGUCGUAGUGGG |
| | | UAAAGCUGCACAUGCAUGUCUAAGACAGCAUGUGCAGCAUCAAAG |
| 43604 | 262 | ACUGGCGCUUUUAUCUGAUUACUUUGAGAGCCAUCACCAGCGACUAUGUCGUAGUGGG |
| | | UAAAGCUGCACAAAACAUAAGGAAAACCUAUGUUGUGCAGCAUCAAAG |
| 43605 | 263 | ACUGGCGCUUCUAUCUGAUUACUCUGAGCGCCAUCACCAGCGACUAUGUCGUAGUGGG |
| | | UAAAGCCGCUUACGGACUAUGGGCGCAGCGUCAAUGACGCUGACGGUACAGGCCAGAC |
| | | AAUUAUUGUCUGGUAUAGUCCGUAAGAGGCAUCAGAG |
| 43606 | 264 | ACUGGCGCUUCUAUCUGAUUACUCUGAGCGCCAUCACCAGCGACUAUGUCGUAGUGGG |
| | | UAAAGCCGCUUACGGGUGGGCGCAGCGUCAAUGACGCUGACGGUACAGGCCAGACAAU |
| | | UAUUGUCUGGUACCCGUAAGAGGCAUCAGAG |
| 43607 | 265 | ACUGGCGCUUCUAUCUGAUUACUCUGAGCGCCAUCACCAGCGACUAUGUCGUAGUGGG |
| | | UAAAGCCGCUUACGGUAUGGGCGCAGCGUCAAUGACGCUGACGGUACAGGCCACAUGA |
| | | GGAUCACCCAUGUGGUAUACCGUAAGAGGCAUCAGAG |
| 43608 | 266 | ACUGGCGCUUUUAUCUGAUUACUUUGAGAGCCAUCACCAGCGACUAUGUCGUAGUGGG |
| | | UAAAGCUCCCUAUGGGCGCAGCGUCAAUGACGCUGACGGUACAGGCCACAUGAGGAUC |
| | | ACCCAUGUGGUAUAGGGAGCAUCAAAG |
| 43609 | 267 | ACUGGCGCUUCUAUCUGAUUACUCUGAGCGCCAUCACCAGCGACUAUGUCGUAGUGGG |
| | | UAAAGCCGCUUACGGUAUGGGCGCAGCUCAUGAGGAUCACCCAUGAGCUGACGGUACA |
| | | GGCCACAUGAGGAUCACCCAUGUGGUAUACCGUAAGAGGCAUCAGAG |
| 43610 | 268 | ACUGGCGCUUUUAUCUGAUUACUUUGAGAGCCAUCACCAGCGACUAUGUCGUAGUGGG |
| | | UAAAGCUCCCUAUGGGCGCAGCUCAUGAGGAUCACCCAUGAGCUGACGGUACAGGCCA |
| | | CAUGAGGAUCACCCAUGUGGUAUAGGGAGCAUCAAAG |
| 43611 | 269 | ACUGGCGCUUCUAUCUGAUUACUCUGAGCGCCAUCACCAGCGACUAUGUCGUAGUGGG |
| | | UAAAGCCGCUUACGGUAUGGGCGCAGCGUCAAUGACGCUGACGGUACAGGCCACAUGG |
| | | CAGUCGUAACGACGCGGGUGGUAUACCGUAAGAGGCAUCAGAG |
| 43612 | 270 | ACUGGCGCUUUUAUCUGAUUACUUUGAGAGCCAUCACCAGCGACUAUGUCGUAGUGGG |
| | | UAAAGCUCCCUAUGGGCGCAGCGUCAAUGACGCUGACGGUACAGGCCACAUGGCAGUC |
| | | GUAACGACGCGGGUGGUAUAGGGAGCAUCAAAG |
| 43613 | 27 | ACUGGCGCUUCUAUCUGAUUACUCUGAGCGCCAUCACCAGCGACUAUGUCGUAGUGGG |
| | | UAAAGCCGCUUACGGUAUGGGCGCAGCAAACAUGGCAGUCCUAAGGACGCGGGUUUUG |
| | | CUGACGGUACAGGCCACAUGGCAGUCGUAACGACGCGGGUGGUAUACCGUAAGAGGCA |
| | | UCAGAG |
| 43614 | 272 | ACUGGCGCUUUUAUCUGAUUACUUUGAGAGCCAUCACCAGCGACUAUGUCGUAGUGGG |
| | | UAAAGCUCCCUAUGGGCGCAGCAAACAUGGCAGUCCUAAGGACGCGGGUUUUGCUGAC |
| | | GGUACAGGCCACAUGGCAGUCGUAACGACGCGGGUGGUAUAGGGAGCAUCAAAG |
| 43615 | 273 | ACUGGCGCUUCUAUCUGAUUACUCUGAGCGCCAUCACCAGCGACUAUGUCGUAGUGGG |
| | | UAAAGCCGCUUACGGUAUGGGCGCAGACAUGGCAGUCGUAACGACGCGGGUCUGACGG |
| | | UACAGGCCACAUGAGGAUCACCCAUGUGGUAUACCGUAAGAGGCAUCAGAG |
| 43616 | 274 | ACUGGCGCUUUUAUCUGAUUACUUUGAGAGCCAUCACCAGCGACUAUGUCGUAGUGGG |
| | | UAAAGCUCCCUAUGGGCGCAGACAUGGCAGUCGUAACGACGCGGGUCUGACGGUACAG |
| | | GCCACAUGAGGAUCACCCAUGUGGUAUAGGGAGCAUCAAAG |
| 43617 | 275 | ACUGGCGCUUUUAUCUGAUUACUUUGAGAGCCAUCACCAGCGACUAUG |
| | | UCGUAGUGGGUAAAGCUGCACUAUGGGCGCAGCACCUGAGGAUCACCC |
| | | AGGUGCUGACGGUACAGGCCACCUGAGGAUCACCCAGGUGGUAUAGUG |
| | | CAGCAUCAAAG |
| 43618 | 276 | ACUGGCGCUUUUAUCUGAUUACUUUGAGAGCCAUCACCAGCGACUAUG |
| | | UCGUAGUGGGUAAAGCUGCACUAUGGGCGCAGCGCAUGAGGAUCACCC |
| | | AUGCGCUGACGGUACAGGCCGCAUGAGGAUCACCCAUGCGGUAUAGUG |
| | | CAGCAUCAAAG |
| 43619 | 277 | ACUGGCGCUUUUAUCUGAUUACUUUGAGAGCCAUCACCAGCGACUAUG |
| | | UCGUAGUGGGUAAAGCUGCACUAUGGGCGCAGCGCCUGAGGAUCACCC |
| | | AGGCGCUGACGGUACAGGCCGCCUGAGGAUCACCCAGGCGGUAUAGUG |
| | | CAGCAUCAAAG |
| 43620 | 278 | ACUGGCGCUUUUAUCUGAUUACUUUGAGAGCCAUCACCAGCGACUAUG |
| | | UCGUAGUGGGUAAAGCUGCACUAUGGGCGCAGCGCCUGAGCAUCAGCC |
| | | AGGCGCUGACGGUACAGGCCGCCUGAGCAUCAGCCAGGCGGUAUAGUG |
| | | CAGCAUCAAAG |
| 43621 | 279 | ACUGGCGCUUUUAUCUGAUUACUUUGAGAGCCAUCACCAGCGACUAUG |
| | | UCGUAGUGGGUAAAGCUGCACUAUGGGCGCAGCACAUGAGCAUCAGCC |
| | | AUGUGCUGACGGUACAGGCCACAUGAGCAUCAGCCAUGUGGUAUAGUG |
| | | CAGCAUCAAAG |
| 43622 | 280 | ACUGGCGCUUUUAUCUGAUUACUUUGAGAGCCAUCACCAGCGACUAUG |
| | | UCGUAGUGGGUAAAGCUGCACUAUGGGCGCAGCACAUGAGUAUCAACC |
| | | AUGUGCUGACGGUACAGGCCACAUGAGUAUCAACCAUGUGGUAUAGUG |
| | | CAGCAUCAAAG |
| 43623 | 281 | ACUGGCGCUUUUAUCUGAUUACUUUGAGAGCCAUCACCAGCGACUAUG |
| | | UCGUAGUGGGUAAAGCUGCACUAUGGGCGCAGCACAUGAGAAUCAGCC |
| | | AUGUGCUGACGGUACAGGCCACAUGAGAAUCAGCCAUGUGGUAUAGUG |
| | | CAGCAUCAAAG |
| 43624 | 282 | ACUGGCGCUUUUAUCUGAUUACUUUGAGAGCCAUCACCAGCGACUAUG |
| | | UCGUAGUGGGUAAAGCUGCACUAUGGGCGCAGCCCUUGAGGAUCACCC |
| | | AUGUGCUGACGGUACAGGCCCCUUGAGGAUCACCCAUGUGGUAUAGUG |
| | | CAGCAUCAAAG |
| 43625 | 283 | ACUGGCGCUUUUAUCUGAUUACUUUGAGAGCCAUCACCAGCGACUAUG |
| | | UCGUAGUGGGUAAAGCUGCACUAUGGGCGCAGCACUUGAGGAUCACCC |
| | | AUGUGCUGACGGUACAGGCCACUUGAGGAUCACCCAUGUGGUAUAGUG |
| | | CAGCAUCAAAG |
| 43626 | 284 | ACUGGCGCUUUUAUCUGAUUACUUUGAGAGCCAUCACCAGCGACUAUG |
| | | UCGUAGUGGGUAAAGCUGCACUAUGGGCGCAGCACCUGAGGAUCACCC |
| | | AUGUGCUGACGGUACAGGCCACCUGAGGAUCACCCAUGUGGUAUAGUG |
| | | CAGCAUCAAAG |
| 43627 | 285 | ACUGGCGCUUUUAUCUGAUUACUUUGAGAGCCAUCACCAGCGACUAUGUCGU |
| | | AGUGGGUAAAGCUGCACUAUGGGCGCAGCACAUGAGGAUCACCUAUGUGCUG |
| | | ACGGUACAGGCCACAUGAGGAUCACCUAUGUGGUAUAGUGCAGCAUCAAAG |
| 43628 | 286 | ACUGGCGCUUUUAUCUGAUUACUUUGAGAGCCAUCACCAGCGACUAUGUCGU |
| | | AGUGGGUAAAGCUGCACUAUGGGCGCAGCACAUUAGGAUCACCAAUGUGCUG |
| | | ACGGUACAGGCCACAUUAGGAUCACCAAUGUGGUAUAGUGCAGCAUCAAAG |
| 43629 | 287 | ACUGGCGCUUUUAUCUGAUUACUUUGAGAGCCAUCACCAGCGACUAUGUCGU |
| | | AGUGGGUAAAGCUGCACUAUGGGCGCAGCACAUUAGGAUCACCGAUGUGCUG |
| | | ACGGUACAGGCCACAUUAGGAUCACCGAUGUGGUAUAGUGCAGCAUCAAAG |
| 43630 | 288 | ACUGGCGCUUUUAUCUGAUUACUUUGAGAGCCAUCACCAGCGACUAUGUCGU |
| | | AGUGGGUAAAGCUGCACUAUGGGCGCAGCACAUUAGGAUCACCUAUGUGCUG |
| | | ACGGUACAGGCCACAUUAGGAUCACCUAUGUGGUAUAGUGCAGCAUCAAAG |
| 43631 | 289 | ACUGGCGCUUUUAUCUGAUUACUUUGAGAGCCAUCACCAGCGACUAUGUCGU |
| | | AGUGGGUAAAGCUGCACUAUGGGCGCAGCACAUGAGGAUUACCCAUGUGCUG |
| | | ACGGUACAGGCCACAUGAGGAUUACCCAUGUGGUAUAGUGCAGCAUCAAAG |
| 43632 | 290 | ACUGGCGCUUUUAUCUGAUUACUUUGAGAGCCAUCACCAGCGACUAUGUCGU |
| | | AGUGGGUAAAGCUGCACUAUGGGCGCAGCACAUGAGGAUAACCCAUGUGCUG |
| | | ACGGUACAGGCCACAUGAGGAUAACCCAUGUGGUAUAGUGCAGCAUCAAAG |
| 43633 | 291 | ACUGGCGCUUUUAUCUGAUUACUUUGAGAGCCAUCACCAGCGACUAUGUCGU |
| | | AGUGGGUAAAGCUGCACUAUGGGCGCAGCACAUGAGGAUGACCCAUGUGCUG |
| | | ACGGUACAGGCCACAUGAGGAUGACCCAUGUGGUAUAGUGCAGCAUCAAAG |
| 43634 | 292 | ACUGGCGCUUUUAUCUGAUUACUUUGAGAGCCAUCACCAGCGACUAUGUCGU |
| | | AGUGGGUAAAGCUGCACUAUGGGCGCAGCACAUGAGGACCACCCAUGUGCUG |
| | | ACGGUACAGGCCACAUGAGGACCACCCAUGUGGUAUAGUGCAGCAUCAAAG |
| 43635 | 293 | ACUGGCGCUUUUAUCUGAUUACUUUGAGAGCCAUCACCAGCGACUAUGUCGU |
| | | AGUGGGUAAAGCUGCACUAUGGGCGCAGCAGAUGAGGAUCACCCAUGGGCUG |
| | | ACGGUACAGGCCAGAUGAGGAUCACCCAUGGGGUAUAGUGCAGCAUCAAAG |
| 43636 | 294 | ACUGGCGCUUUUAUCUGAUUACUUUGAGAGCCAUCACCAGCGACUAUGUCGU |
| | | AGUGGGUAAAGCUGCACUAUGGGCGCAGCACAUGGGGAUCACCCAUGUGCUG |
| | | ACGGUACAGGCCACAUGGGGAUCACCCAUGUGGUAUAGUGCAGCAUCAAAG |
| 43637 | 295 | ACUGGCGCUUUUAUCUGAUUACUUUGAGAGCCAUCACCAGCGACUAUGUCGU |
| | | AGUGGGUAAAGCUGCACUAUGGGCGCAGCACAUGAGGAUCACCCAUGUGCUG |
| | | ACGGUACAGGCCACAUGAGGAUCACCCAUGUGGUAUAGUGCAGCAUCAAAG |
| 43638 | 296 | ACUGGCGCUUUUAUCUGAUUACUUUGAGAGCCAUCACCAGCGACUAUGUCGU |
| | | AGUGGGUAAAGCUCACCUGAGGAUCACCCAGGUGAGCAUCAAAG |
| 43639 | 297 | ACUGGCGCUUUUAUCUGAUUACUUUGAGAGCCAUCACCAGCGACUAUGUCGU |
| | | AGUGGGUAAAGCUCGCAUGAGGAUCACCCAUGCGAGCAUCAAAG |
| 43640 | 298 | ACUGGCGCUUUUAUCUGAUUACUUUGAGAGCCAUCACCAGCGACUAUGUCGU |
| | | AGUGGGUAAAGCUCGCCUGAGGAUCACCCAGGCGAGCAUCAAAG |
| 43641 | 299 | ACUGGCGCUUUUAUCUGAUUACUUUGAGAGCCAUCACCAGCGACUAUGUCGU |
| | | AGUGGGUAAAGCUCGCCUGAGCAUCAGCCAGGCGAGCAUCAAAG |
| 43642 | 300 | ACUGGCGCUUUUAUCUGAUUACUUUGAGAGCCAUCACCAGCGACUAUGUCGU |
| | | AGUGGGUAAAGCUCACAUGAGCAUCAGCCAUGUGAGCAUCAAAG |
| 43643 | 301 | ACUGGCGCUUUUAUCUGAUUACUUUGAGAGCCAUCACCAGCGACUAUGUCGU |
| | | AGUGGGUAAAGCUCACAUGAGUAUCAACCAUGUGAGCAUCAAAG |
| 43644 | 302 | ACUGGCGCUUUUAUCUGAUUACUUUGAGAGCCAUCACCAGCGACUAUGUCGU |
| | | AGUGGGUAAAGCUCACAUGAGAAUCAGCCAUGUGAGCAUCAAAG |
| 43645 | 303 | ACUGGCGCUUUUAUCUGAUUACUUUGAGAGCCAUCACCAGCGACUAUGUCGU |
| | | AGUGGGUAAAGCUCCCUUGAGGAUCACCCAUGUGAGCAUCAAAG |
| 43646 | 304 | ACUGGCGCUUUUAUCUGAUUACUUUGAGAGCCAUCACCAGCGACUAUGUCGU |
| | | AGUGGGUAAAGCUCACUUGAGGAUCACCCAUGUGAGCAUCAAAG |
| 43647 | 305 | ACUGGCGCUUUUAUCUGAUUACUUUGAGAGCCAUCACCAGCGACUAUGUCGU |
| | | AGUGGGUAAAGCUCACCUGAGGAUCACCCAUGUGAGCAUCAAAG |
| 43648 | 306 | ACUGGCGCUUUUAUCUGAUUACUUUGAGAGCCAUCACCAGCGACUAUGUCGU |
| | | AGUGGGUAAAGCUCACAUGAGGAUCACCUAUGUGAGCAUCAAAG |
| 43649 | 307 | ACUGGCGCUUUUAUCUGAUUACUUUGAGAGCCAUCACCAGCGACUAUGUCGU |
| | | AGUGGGUAAAGCUCACAUUAGGAUCACCAAUGUGAGCAUCAAAG |
| 43650 | 308 | ACUGGCGCUUUUAUCUGAUUACUUUGAGAGCCAUCACCAGCGACUAUGUCGU |
| | | AGUGGGUAAAGCUCACAUUAGGAUCACCGAUGUGAGCAUCAAAG |
| 43651 | 309 | ACUGGCGCUUUUAUCUGAUUACUUUGAGAGCCAUCACCAGCGACUAUGUCGU |
| | | AGUGGGUAAAGCUCACAUUAGGAUCACCUAUGUGAGCAUCAAAG |
| 43652 | 310 | ACUGGCGCUUUUAUCUGAUUACUUUGAGAGCCAUCACCAGCGACUAUGUCGU |
| | | AGUGGGUAAAGCUCACAUGAGGAUUACCCAUGUGAGCAUCAAAG |
| 43653 | 311 | ACUGGCGCUUUUAUCUGAUUACUUUGAGAGCCAUCACCAGCGACUAUGUCGU |
| | | AGUGGGUAAAGCUCACAUGAGGAUAACCCAUGUGAGCAUCAAAG |
| 43654 | 312 | ACUGGCGCUUUUAUCUGAUUACUUUGAGAGCCAUCACCAGCGACUAUGUCGU |
| | | AGUGGGUAAAGCUCACAUGAGGAUGACCCAUGUGAGCAUCAAAG |
| 43655 | 313 | ACUGGCGCUUUUAUCUGAUUACUUUGAGAGCCAUCACCAGCGACUAUGUCGU |
| | | AGUGGGUAAAGCUCACAUGAGGACCACCCAUGUGAGCAUCAAAG |
| 43656 | 314 | ACUGGCGCUUUUAUCUGAUUACUUUGAGAGCCAUCACCAGCGACUAUGUCGU |
| | | AGUGGGUAAAGCUCAGAUGAGGAUCACCCAUGGGAGCAUCAAAG |
| 43657 | 315 | ACUGGCGCUUUUAUCUGAUUACUUUGAGAGCCAUCACCAGCGACUAUGUCGU |
| | | AGUGGGUAAAGCUCACAUGGGGAUCACCCAUGUGAGCAUCAAAG |
| 43658 | 317 | ACUGGCGCUUCUAUCUGAUUACUCUGAGCGCCAUCACCAGCGACUAUGUCGUAGUGGG |
| | | UAAAGCUCACAUGAGGAUCACCCAUGUGAGCAUCAGAG |
| 43659 | 318 | ACUGGCGCUUCUAUCUGAUUACUCUGAGCGCCAUCACCAGCGACUAUGUCGUAGUGGG |
| | | UAAAGCUGCACUAUGGGCGCAGCGUCAAUGACGCUGACGGUACAGGCCACAUGAGGAU |
| | | CACCCAUGUGGUAUAGUGCAGCAUCAGAG |
| 43660 | 319 | ACUGGCGCUUCUAUCUGAUUACUCUGAGCGCCAUCACCAGCGACUAUGUCGUAGUGGG |
| | | UAAAGCUGCACUAUGGGCGCAGCUCAUGAGGAUCACCCAUGAGCUGACGGUACAGGCC |
| | | ACAUGAGGAUCACCCAUGUGGUAUAGUGCAGCAUCAGAG |
| 43661 | 320 | ACUGGCGCUUCUAUCUGAUUACUCUGAGCGCCAUCACCAGCGACUAUGUCGUAGUGGG |
| | | UAAAGCUGCACUAUGGGCGCAGACAUGGCAGUCGUAACGACGCGGGUCUGACGGUACA |
| | | GGCCACAUGAGGAUCACCCAUGUGGUAUAGUGCAGCAUCAGAG |
| 44047 | 321 | ACUGGCGCUUUUAUCUGAUUACUUUGAGAGCCAUCACCAGCGACUAUGUCGU |
| | | AGUGGGUAAAGCUGCACUAUGGGGCCACAUGAGGAUCACCCAUGUGGUGUAC |
| | | AGCGCAGCGUCAAUGACGCUGACGAUAGUGCAGCAUCAAAG |

In some embodiments, a sgRNA variant comprises one or more additional changes to a sequence of SEQ ID NO:2238, SEQ ID NO:2239, SEQ ID NO:2240, SEQ ID NO:2241, SEQ ID NO:2243, SEQ ID NO: 2249, SEQ ID NO:2256, SEQ ID NO:2274, SEQ ID NO:2275, SEQ ID NO:2279, SEQ ID NO:2281, SEQ ID NO: 2285, SEQ ID NO: 43574, SEQ ID NO: 43577, or SEQ ID NO: 43593 of Table 3.

In some embodiments of the gRNA variants of the disclosure, the gRNA variant comprises at least one modification, wherein the at least one modification compared to the reference guide scaffold of SEQ ID NO:5 is selected from one or more of: (a) a C18G substitution in the triplex loop; (b) a G55 insertion in the stem bubble; (c) a U1 deletion; (d) a modification of the extended stem loop wherein (i) a 6 nt loop and 13 loop-proximal base pairs are replaced by a Uvsx hairpin; and (ii) a deletion of A99 and a substitution of G65U that results in a loop-distal base that is fully base-paired. In exemplary embodiments of the foregoing, the gRNA variant comprises the sequence of any one of SEQ ID NOS: 2238, 2241, 2244, 2248, 2249, 2256, 2259-2285, 43574 or 43577.

In some embodiments, a gRNA variant comprises an exogenous stem loop having a long non-coding RNA (lncRNA). As used herein, a lncRNA refers to a non-coding RNA that is longer than approximately 200 bp in length. In some embodiments, the 5′ and 3′ ends of the exogenous stem loop are base paired; i.e., interact to form a region of duplex RNA. In some embodiments, the 5′ and 3′ ends of the exogenous stem loop are base paired, and one or more regions between the 5′ and 3′ ends of the exogenous stem loop are not base paired.

In some embodiments, the disclosure provide gRNA variants with nucleotide modifications relative to reference gRNA having: (a) substitution of 1 to 15 consecutive or non-consecutive nucleotides in the gRNA variant in one or more regions; (b) a deletion of 1 to 10 consecutive or non-consecutive nucleotides in the gRNA variant in one or more regions; (c) an insertion of 1 to 10 consecutive or non-consecutive nucleotides in the gRNA variant in one or more regions; (d) a substitution of the scaffold stem loop or the extended stem loop with an RNA stem loop sequence from a heterologous RNA source with proximal 5′ and 3′ ends; or any combination of (a)-(d). Any of the substitutions, insertions and deletions described herein can be combined to generate a gNA variant of the disclosure. For example, a gNA variant can comprise at least one substitution and at least one deletion relative to a reference gRNA, at least one substitution and at least one insertion relative to a reference gRNA, at least one insertion and at least one deletion relative to a reference gRNA, or at least one substitution, one insertion and one deletion relative to a reference gRNA.

In some embodiments, a sgRNA variant of the disclosure comprises one or more additional changes to a previously generated variant, the previously generated variant itself serving as the sequence to be modified. In some embodiments, a sgRNA variant comprises one or more additional changes to a sequence of SEQ ID NO: 2238, SEQ ID NO: 2239, SEQ ID NO: 2240, SEQ ID NO: 2241, SEQ ID NO:2241, SEQ ID NO: 2249, SEQ ID NO:2274, SEQ ID NO:2275, SEQ ID NO: 2279, or SEQ ID NO: 2285, SEQ ID NO: 43574, SEQ ID NO: 43577, or SEQ ID NO: 43593.

In exemplary embodiments, a gRNA variant comprises one or more modification relative to gRNA scaffold variant 174 (SEQ ID NO:2238), wherein the resulting gRNA variant exhibits a functional improvement compared to the parent 174, when assessed in an in vitro or in vivo assay under comparable conditions.

In exemplary embodiments, a gRNA variant comprises one or more modification relative to gRNA scaffold variant 175 (SEQ ID NO:2239), wherein the resulting gRNA variant exhibits a functional improvement compared to the parent 174, when assessed in an in vitro or in vivo assay under comparable conditions.

In exemplary embodiments, a gRNA variant comprises one or more modification relative to gRNA scaffold variant 188 (SEQ ID NO:2249), wherein the resulting gRNA variant exhibits a functional improvement compared to the parent 215, when assessed in an in vitro or in vivo assay under comparable conditions.

In exemplary embodiments, a gRNA variant comprises one or more modification relative to gRNA scaffold variant 215 (SEQ ID NO:2275), wherein the resulting gRNA variant exhibits a functional improvement compared to the parent 215, when assessed in an in vitro or in vivo assay under comparable conditions.

In exemplary embodiments, a gRNA variant comprises one or more modification relative to gRNA scaffold variant 221 (SEQ ID NO: 2281), wherein the resulting gRNA variant exhibits a functional improvement compared to the parent 221, when assessed in an in vitro or in vivo assay under comparable conditions.

In exemplary embodiments, a gRNA variant comprises one or more modification relative to gRNA scaffold variant 225 (SEQ ID NO: 2285), wherein the resulting gRNA variant exhibits a functional improvement compared to the parent 225, when assessed in an in vitro or in vivo assay under comparable conditions.

In exemplary embodiments, a gRNA variant comprises one or more modification relative to gRNA scaffold variant 235 (SEQ ID NO: 43577), wherein the resulting gRNA variant exhibits a functional improvement compared to the parent 235, when assessed in an in vitro or in vivo assay under comparable conditions.

In exemplary embodiments, a gRNA variant comprises one or more modification relative to gRNA scaffold variant 251 (SEQ ID NO: 43593), wherein the resulting gRNA variant exhibits a functional improvement compared to the parent 251, when assessed in an in vitro or in vivo assay under comparable conditions.

In some embodiments, the gRNA variant comprises an exogenous extended stem loop, with such differences from a reference gRNA described as follows. In some embodiments, an exogenous extended stem loop has little or no identity to the reference stem loop regions disclosed herein (e.g., SEQ ID NO:15). In some embodiments, an exogenous stem loop is at least 10 bp, at least 20 bp, at least 30 bp, at least 40 bp, at least 50 bp, at least 60 bp, at least 70 bp, at least 80 bp, at least 90 bp, at least 100 bp, at least 200 bp, at least 300 bp, at least 400 bp, at least 500 bp, at least 600 bp, at least 700 bp, at least 800 bp, at least 900 bp, at least 1,000 bp, at least 2,000 bp, at least 3,000 bp, at least 4,000 bp, at least 5,000 bp, at least 6,000 bp, at least 7,000 bp, at least 8,000 bp, at least 9,000 bp, at least 10,000 bp, at least 12,000 bp, at least 15,000 bp or at least 20,000 bp. In some embodiments, the gRNA variant comprises an extended stem loop region comprising at least 10, at least 100, at least 500, at least 1000, or at least 10,000 nucleotides. In some embodiments, the heterologous stem loop increases the stability of the gRNA. In some embodiments, the heterologous RNA stem loop is capable of binding a protein, an RNA structure, a DNA sequence, or a small molecule. In some embodiments, an exogenous stem loop region replacing the stem loop comprises an RNA stem loop or hairpin in which the resulting gRNA has increased stability and, depending on the choice of loop, can interact with certain cellular proteins or RNA. Such exogenous extended stem loops can comprise, for example a thermostable RNA such as MS2 hairpin (ACAUGAGGAUCACCCAUGU (SEQ ID NO: 22)), Qβ hairpin (UGCAUGUCUAAGACAGCA (SEQ ID NO: 23)), U1 hairpin II (AAUCCAUUGCACUCCGGAUU (SEQ ID NO: 24)), Uvsx (CCUCUUCGGAGG (SEQ ID NO: 25)), PP7 hairpin (AGGAGUUUCUAUGGAAACCCU (SEQ ID NO: 26)), Phage replication loop (AGGUGGGACGACCUCUCGGUCGUCCUAUCU (SEQ ID NO: 27)), Kissing loop_a (UGCUCGCUCCGUUCGAGCA (SEQ ID NO: 28)), Kissing loop_b1 (UGCUCGACGCGUCCUCGAGCA (SEQ ID NO: 29)), Kissing loop_b2 (UGCUCGUUUGCGGCUACGAGCA (SEQ ID NO: 30)), G quadriplex M3q (AGGGAGGGAGGGAGAGG (SEQ ID NO: 31)), G quadriplex telomere basket (GGUUAGGGUUAGGGUUAGG (SEQ ID NO: 32)), Sarcin-ricin loop (CUGCUCAGUACGAGAGGAACCGCAG (SEQ ID NO: 33)) or Pseudoknots (UACACUGGGAUCGCUGAAUUAGAGAUCGGCGUCCUUUCAUUCUAUAUACUUUGG AGUUUUAAAAUGUCUCUAAGUACA (SEQ ID NO: 34)). In other embodiments, the stem loop comprises a “Rev response element” or “RRE”, capable of binding a retroviral Rev protein incorporated into an XDP fusion protein onto the gRNA. In some embodiments, one of the foregoing hairpin or RRE sequences is incorporated into the stem loop to help traffic the incorporation of the gRNA (and an associated CasX in an RNP complex) into a budding XDP (described more fully, below).

In the embodiments of the gRNA variants, the gRNA variant further comprises a spacer (or targeting sequence) region located at the 3′ end of the gRNA, capable of hybridizing with a target nucleic acid specific to a PTBP1 sequence described more fully, supra, which comprises at least 14 to about 35 nucleotides wherein the spacer is designed with a sequence that is complementary to a target DNA. In some embodiments, the encoded gRNA variant comprises a targeting sequence of at least 10 to 30 nucleotides complementary to a target DNA. In some embodiments, the targeting sequence has 14, 15, 16, 17, 18, 19, 20, 21, 22, 23, 24, 25, 26, 27, 28, 29, 30, 31, 32, 33, 34 or 35 nucleotides. In some embodiments, the encoded gRNA variant comprises a targeting sequence having 20 nucleotides. In some embodiments, the targeting sequence has 25 nucleotides. In some embodiments, the targeting sequence has 24 nucleotides. In some embodiments, the targeting sequence has 23 nucleotides. In some embodiments, the targeting sequence has 22 nucleotides. In some embodiments, the targeting sequence has 21 nucleotides. In some embodiments, the targeting sequence has 20 nucleotides. In some embodiments, the targeting sequence has 19 nucleotides. In some embodiments, the targeting sequence has 18 nucleotides. In some embodiments, the targeting sequence has 17 nucleotides. In some embodiments, the targeting sequence has 16 nucleotides. In some embodiments, the targeting sequence has 15 nucleotides. In some embodiments, the targeting sequence has 14 nucleotides.

### k. Complex Formation with CasX Protein

In some embodiments, upon expression, the gRNA variant is complexed as an RNP with a CasX variant protein comprising any one of the sequences of Table 4 (SEQ ID NOS: 36-99, 101-148 or 43662-43907), or a sequence having at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% identity thereto. In some embodiments, upon expression, the gRNA variant is complexed as an RNP with a CasX variant protein comprising any one of SEQ ID NOS: 59, 72-99, 101-148, or 43662-43907, or a sequence having at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% identity thereto. In some embodiments, upon expression, the gRNA variant is complexed as an RNP with a CasX variant protein comprising any one of SEQ ID NOS: 132-148 or 43662-43907, or a sequence having at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% identity thereto.

In some embodiments, a gRNA variant has an improved ability to form a complex with a CasX protein (such as a reference CasX or a CasX variant protein) when compared to a reference gRNA. In some embodiments, a gRNA variant has an improved affinity for a CasX protein (such as a reference or variant protein) when compared to a reference gRNA, thereby improving its ability to form a ribonucleoprotein (RNP) complex with the CasX protein, as described in the Examples. Improving ribonucleoprotein complex formation may, in some embodiments, improve the efficiency with which functional RNPs are assembled. In some embodiments, greater than 90%, greater than 93%, greater than 95%, greater than 96%, greater than 97%, greater than 98% or greater than 99% of RNPs comprising a gRNA variant and its spacer are competent for gene editing of a target nucleic acid.

Exemplary nucleotide changes that can improve the ability of gRNA variants to form a complex with CasX protein may, in some embodiments, include replacing the scaffold stem with a thermostable stem loop. Without wishing to be bound by any theory, replacing the scaffold stem with a thermostable stem loop could increase the overall binding stability of the gRNA variant with the CasX protein. Alternatively, or in addition, removing a large section of the stem loop could change the gRNA variant folding kinetics and make a functional folded gRNA easier and quicker to structurally-assemble, for example by lessening the degree to which the gRNA variant can get “tangled” in itself. In some embodiments, choice of scaffold stem loop sequence could change with different spacers that are utilized for the gRNA. In some embodiments, scaffold sequence can be tailored to the spacer and therefore the target sequence. Biochemical assays can be used to evaluate the binding affinity of CasX protein for the gRNA variant to form the RNP, including the assays of the Examples. For example, a person of ordinary skill can measure changes in the amount of a fluorescently tagged gRNA that is bound to an immobilized CasX protein, as a response to increasing concentrations of an additional unlabeled “cold competitor” gRNA. Alternatively, or in addition, fluorescence signal can be monitored to see how it changes as different amounts of fluorescently labeled gRNA are flowed over immobilized CasX protein. Alternatively, the ability to form an RNP can be assessed using in vitro cleavage assays against a defined target nucleic acid sequence, wherein the cleavage rate of the RNP comprising a gRNA variant is improved compared to an RNP comprising a reference gRNA when assessed in an in vitro assay.

### II. Proteins for Modifying a Target Nucleic Acid

The present disclosure provides systems comprising a CRISPR nuclease that have utility in genome editing of the PTBP1 gene in eukaryotic cells. In some embodiments, the CRISPR nuclease employed in the genome editing systems is a Class 2, Type V nuclease. Although members of Class 2, Type V CRISPR-Cas systems have differences, they share some common characteristics that distinguish them from the Cas9 systems. Firstly, the Class 2, Type V nucleases possess a single RNA-guided RuvC domain-containing effector but no HNH domain, and they recognize T-rich PAM 5′ upstream to the target region on the non-targeted strand, which is different from Cas9 systems which rely on G-rich PAM at 3′ side of target sequences. Type V nucleases generate staggered double-stranded breaks distal to the PAM sequence, unlike Cas9, which generates a blunt end in the proximal site close to the PAM. In addition, Type V nucleases degrade ssDNA in trans when activated by target dsDNA or ssDNA binding in cis. In some embodiments, the Type V nucleases of the embodiments recognize a 5′-TC PAM motif and produce staggered ends cleaved solely by the RuvC domain. In some embodiments, the Type V nuclease is selected from the group consisting of Cas12a, Cas12b, Cas12c, Cas12d (CasY), Cas12j, Cas12k, C2c4, C2c8, C2c5, C2c10, C2c9, CasZ and CasX. In some embodiments, the present disclosure provides systems comprising a CasX protein and one or more gRNA acids (CasX:gRNA system) that are specifically designed to modify a target nucleic acid sequence in eukaryotic cells.

The term “CasX protein”, as used herein, refers to a family of proteins, and encompasses all naturally-occurring CasX proteins (“reference CasX”), as well as CasX variants that share at least 50% to about 99% identity to naturally occurring CasX proteins and that possess one or more improved characteristics relative to a reference CasX protein, described more fully, below.

CasX proteins of the disclosure comprise at least one of the following domains: a non-target strand binding (NTSB) domain, a target strand loading (TSL) domain, a helical I domain, a helical II domain, an oligonucleotide binding domain (OBD), and a RuvC DNA cleavage domain.

In some embodiments, a CasX protein can bind and/or modify (e.g., nick, catalyze a double strand break, methylate, demethylate, etc.) a target nucleic acid at a specific sequence targeted by an associated gRNA, which hybridizes to a sequence within the target nucleic acid sequence.

### a. Reference CasX Proteins

The disclosure provides naturally-occurring CasX proteins (referred to herein as a “reference CasX protein”), which were subsequently modified to create the CasX variants of the disclosure. For example, reference CasX proteins can be isolated from naturally occurring prokaryotes, such as Deltaproteobacteria, Planctomycetes, or Candidatus Sungbacteria species. A reference CasX protein (interchangeably referred to herein as a reference CasX polypeptide) is a type II CRISPR/Cas endonuclease belonging to the CasX (interchangeably referred to as Cas12e) family of proteins that interacts with a guide RNA to form a ribonucleoprotein (RNP) complex.

In some cases, a reference CasX protein is isolated or derived from Deltaproteobacter having a sequence of:

| (SEQ ID NO: 1) | |
|---|---|
| 1 | MEKRINKIRK KLSADNATKP VSRSGPMKTL LVRVMTDDLK KRLEKRRKKP EVMPQVISNN |
| 61 | AANNLRMLLD DYTKMKEAIL QVYWQEFKDD HVGLMCKFAQ PASKKIDQNK LKPEMDEKGN |
| 121 | LTTAGFACSQ CGQPLFVYKL EQVSEKGKAY TNYFGRCNVA EHEKLILLAQ LKPEKDSDEA |
| 181 | VTYSLGKFGQ RALDFYSIHV TKESTHPVKP LAQIAGNRYA SGPVGKALSD ACMGTIASFL |
| 241 | SKYQDIIIEH QKVVKGNQKR LESLRELAGK ENLEYPSVTL PPQPHTKEGV DAYNEVIARV |
| 301 | RMWVNLNLWQ KLKLSRDDAK PLLRLKGFPS FPVVERRENE VDWWNTINEV KKLIDAKRDM |
| 361 | GRVFWSGVTA EKRNTILEGY NYLPNENDHK KREGSLENPK KPAKRQFGDL LLYLEKKYAG |
| 421 | DWGKVFDEAW ERIDKKIAGL TSHIEREEAR NAEDAQSKAV LTDWLRAKAS FVLERLKEMD |
| 481 | EKEFYACEIQ LQKWYGDLRG NPFAVEAENR VVDISGFSIG SDGHSIQYRN LLAWKYLENG |
| 541 | KREFYLLMNY GKKGRIRFTD GTDIKKSGKW QGLLYGGGKA KVIDLTFDPD DEQLIILPLA |
| 601 | FGTRQGREFI WNDLLSLETG LIKLANGRVI EKTIYNKKIG RDEPALFVAL TFERREVVDP |
| 661 | SNIKPVNLIG VDRGENIPAV IALTDPEGCP LPEFKDSSGG PTDILRIGEG YKEKQRAIQA |
| 721 | AKEVEQRRAG GYSRKFASKS RNLADDMVRN SARDLFYHAV THDAVLVFEN LSRGFGRQGK |
| 781 | RTFMTERQYT KMEDWLTAKL AYEGLTSKTY LSKTLAQYTS KTCSNCGFTI TTADYDGMLV |
| 841 | RLKKTSDGWA TTLNNKELKA EGQITYYNRY KRQTVEKELS AELDRLSEES GNNDISKWTK |
| 901 | GRRDEALFLL KKRFSHRPVQ EQFVCLDCGH EVHADEQAAL NIARSWLELN SNSTEFKSYK |
| 961 | SGKQPFVGAW QAFYKRRLKE VWKPNA. |

In some cases, a reference CasX protein is isolated or derived from Planctomycetes having a sequence of.

| (SEQ ID NO: 2) | |
|---|---|
| 1 | MQEIKRINKI RRRLVKDSNT KKAGKTGPMK TLLVRVMTPD LRERLENLRK KPENIPQPIS |
| 61 | NTSRANLNKL LTDYTEMKKA ILHVYWEEFQ KDPVGLMSRV AQPAPKNIDQ RKLIPVKDGN |
| 121 | ERLTSSGFAC SQCCQPLYVY KLEQVNDKGK PHTNYFGRCN VSEHERLILL SPHKPEANDE |
| 181 | LVTYSLGKFG QRALDFYSIH VTRESNHPVK PLEQIGGNSC ASGPVGKALS DACMGAVASF |
| 241 | LTKYQDIILE HQKVIKKNEK RLANLKDIAS ANGLAFPKIT LPPQPHTKEG IEAYNNVVAQ |
| 301 | IVIWVNLNLW QKLKIGRDEA KPLQRLKGFP SFPLVERQAN EVDWWDMVCN VKKLINEKKE |
| 361 | DGKVFWQNLA GYKRQEALLP YLSSEEDRKK GKKFARYQFG DLLLHLEKKH GEDWGKVYDE |
| 421 | AWERIDKKVE GLSKHIKLEE ERRSEDAQSK AALTDWLRAK ASFVIEGLKE ADKDEFCRCE |
| 481 | LKLQKWYGDL RGKPFAIEAE NSILDISGES KQYNCAFIWQ KDGVKKLNLY LIINYFKGGK |
| 541 | LRFKKIKPEA FEANRFYTVI NKKSGEIVPM EVNENFDDPN LIILPLAFGK RQGREFIWND |
| 601 | LLSLETGSLK LANGRVIEKT LYNRRTRQDE PALFVALTFE RREVLDSSNI KPMNLIGIDR |
| 661 | GENIPAVIAL TDPEGCPLSR FKDSLGNPTH ILRIGESYKE KQRTIQAAKE VEQRRAGGYS |
| 721 | RKYASKAKNL ADDMVRNTAR DLLYYAVTQD AMLIFENLSR GFGRQGKRTF MAERQYTRME |
| 781 | DWLTAKLAYE GLPSKTYLSK TLAQYTSKTC SNCGFTITSA DYDRVLEKLK KTATGWMTTI |
| 841 | NGKELKVEGQ ITYYNRYKRQ NVVKDLSVEL DRLSEESVNN DISSWTKGRS GEALSLLKKR |
| 901 | FSHRPVQEKF VCLNCGFETH ADEQAALNIA RSWLFLRSQE YKKYQTNKTT GNTDKRAFVE |
| 961 | TWQSFYRKKL KEVWKPAV. |

In some cases, a reference CasX protein is isolated or derived from Candidatus Sungbacteria having a sequence of

| (SEQ ID NO: 3) | |
|---|---|
| 1 | MDNANKPSTK SLVNTTRISD HFGVTPGQVT RVFSFGIIPT KRQYAIIERW FAAVEAARER |
| 61 | LYGMLYAHFQ ENPPAYLKEK FSYETFFKGR PVLNGLRDID PTIMTSAVFT ALRHKAEGAM |
| 121 | AAFHTNHRRL FEEARKKMRE YAECLKANEA LLRGAADIDW DKIVNALRTR LNTCLAPEYD |
| 181 | AVIADFGALC AFRALIAETN ALKGAYNHAL NQMLPALVKV DEPEEAEESP RLRFENGRIN |
| 241 | DLPKFPVAER ETPPDTETII RQLEDMARVI PDTAEILGYI HRIRHKAARR KPGSAVPLPQ |
| 301 | RVALYCAIRM ERNPEEDPST VAGHELGEID RVCEKRRQGL VRTPEDSQIR ARYMDIISER |
| 361 | ATLAHPDRWT EIQFLRSNAA SRRVRAETIS APFEGFSWTS NRTNPAPQYG MALAKDANAP |
| 421 | ADAPELCICL SPSSAAFSVR EKGGDLIYMR PTGGRRGKDN PGKEITWVPG SFDEYPASGV |
| 481 | ALKLRLYFGR SQARRMLINK TWGLLSDNPR VFAANAELVG KKRNPQDRWK LFFHMVISGP |
| 541 | PPVEYLDFSS DVRSRARTVI GINRGEVNPL AYAVVSVEDG QVLEEGLLGK KEYIDQLIET |
| 601 | RRRISEYQSR EQTPPRDLRQ RVRHLQDTVL GSARAKIHSL IAFWKGILAI ERLDDQFHGR |
| 661 | EQKIIPKKTY LANKTGFMNA LSFSGAVRVD KKGNPWGGMI EIYPGGISRT CTQCGTVWLA |
| 721 | RRPKNPGHRD AMVVIPDIVD DAAATGFDNV DCDAGTVDYG ELFTLSREWV RLTPRYSRVM |
| 781 | RGTLGDLERA IRQGDDRKSR QMLELALEPQ PQWGQFFCHR CGENGQSDVL AATNLARRAI |
| 841 | SLIRRLPDTD TPPTP. |

### b. CasX Variant Proteins

The present disclosure provides variants of a reference CasX protein (interchangeably referred to herein as “CasX variant” or “CasX variant protein”), wherein the CasX variants comprise at least one modification in at least one domain of the reference CasX protein, including the sequences of SEQ ID NOS:1-3. Any change in amino acid sequence of a reference CasX protein that leads to an improved characteristic of the CasX protein is considered a CasX variant protein of the disclosure. For example, CasX variants can comprise one or more amino acid substitutions, insertions, deletions, or swapped domains, or any combinations thereof, relative to a reference CasX protein sequence.

The CasX variants of the disclosure have one or more improved characteristics compared to reference CasX proteins, for example a reference protein of SEQ ID NO:1, SEQ ID NO:2 or SEQ ID NO:3, or the variant from which it was derived; e.g. CasX 491 or CasX 515. Exemplary improved characteristics of the CasX variant embodiments include, but are not limited to improved binding affinity to the gRNA, improved binding affinity to the target nucleic acid, improved ability to utilize a greater spectrum of PAM sequences in the editing and/or binding of target DNA, improved unwinding of the target DNA, increased editing activity, improved editing efficiency, improved editing specificity, increased percentage of a eukaryotic genome that can be efficiently edited, increased activity of the nuclease, increased target strand loading for double strand cleavage, decreased target strand loading for single strand nicking, decreased off-target cleavage, improved binding of the non-target strand of DNA, improved protein:gRNA (RNP) complex stability, improved protein:gRNA (RNP) complex solubility, and improved fusion characteristics, as described more fully, below. Exemplary improved characteristics are described in WO2020247882A1 and PCT/US20/36505, incorporated by reference herein. In the foregoing embodiments, the one or more of the improved characteristics of the CasX variant is at least about 1.1 to about 100,000-fold improved relative to the reference CasX protein of SEQ ID NO: 1, SEQ ID NO: 2, or SEQ ID NO: 3, when assayed in a comparable fashion. In other embodiments, the improvement is at least about 1.1-fold, at least about 2-fold, at least about 5-fold, at least about 10-fold, at least about 50-fold, at least about 100-fold, at least about 500-fold, at least about 1000-fold, at least about 5000-fold, at least about 10,000-fold, or at least about 100,000-fold compared to the reference CasX protein of SEQ ID NO: 1, SEQ ID NO: 2, or SEQ ID NO: 3, when assayed in a comparable fashion. In other cases, the one or more improved characteristics of an RNP of the CasX variant and the gRNA variant are at least about 1.1, at least about 10, at least about 100, at least about 1000, at least about 10,000, at least about 100,000-fold or more improved relative to an RNP of the reference CasX protein of SEQ ID NO:1, SEQ ID NO:2, or SEQ ID NO:3 and the gRNA of Table 2 or Table 3. In other cases, the one or more of the improved characteristics of an RNP of the CasX variant and the gRNA variant are about 1.1 to 100,00-fold, about 1.1 to 10,00-fold, about 1.1 to 1,000-fold, about 1.1 to 500-fold, about 1.1 to 100-fold, about 1.1 to 50-fold, about 1.1 to 20-fold, about 10 to 100,00-fold, about 10 to 10,00-fold, about 10 to 1,000-fold, about 10 to 500-fold, about 10 to 100-fold, about 10 to 50-fold, about 10 to 20-fold, about 2 to 70-fold, about 2 to 50-fold, about 2 to 30-fold, about 2 to 20-fold, about 2 to 10-fold, about 5 to 50-fold, about 5 to 30-fold, about 5 to 10-fold, about 100 to 100,00-fold, about 100 to 10,00-fold, about 100 to 1,000-fold, about 100 to 500-fold, about 500 to 100,00-fold, about 500 to 10,00-fold, about 500 to 1,000-fold, about 500 to 750-fold, about 1,000 to 100,00-fold, about 10,000 to 100,00-fold, about 20 to 500-fold, about 20 to 250-fold, about 20 to 200-fold, about 20 to 100-fold, about 20 to 50-fold, about 50 to 10,000-fold, about 50 to 1,000-fold, about 50 to 500-fold, about 50 to 200-fold, or about 50 to 100-fold, improved relative to an RNP of the reference CasX protein of SEQ ID NO:1, SEQ ID NO:2, or SEQ ID NO:3 and the gRNA of Table 2 or Table 3, when assayed in a comparable fashion. In other cases, the one or more improved characteristics of an RNP of the CasX variant and the gRNA variant are about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 40-fold, 45-fold, 50-fold, 55-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 310-fold, 320-fold, 330-fold, 340-fold, 350-fold, 360-fold, 370-fold, 380-fold, 390-fold, 400-fold, 425-fold, 450-fold, 475-fold, or 500-fold improved relative to an RNP of the reference CasX protein of SEQ ID NO:1, SEQ ID NO:2, or SEQ ID NO:3 and the gRNA of Table 2 or Table 3, when assayed in a comparable fashion.

The term CasX variant is inclusive of variants that are fusion proteins; i.e. the CasX is “fused to” a heterologous sequence. This includes CasX variants comprising CasX variant sequences and N-terminal, C-terminal, or internal fusions of the CasX to a heterologous protein or domain thereof.

In some embodiments, the CasX variant comprises at least one modification in the NTSB domain. In some embodiments, the CasX variant comprises at least one modification in the TSL domain. In some embodiments, the CasX variant comprises at least one modification in the helical I domain. In some embodiments, the CasX variant comprises at least one modification in the helical II domain. In some embodiments, the CasX variant comprises at least one modification in the OBD domain. In some embodiments, the CasX variant comprises at least one modification in the RuvC DNA cleavage domain. In some embodiments, the at least one modification in the RuvC DNA cleavage domain comprises an amino acid substitution of one or more of amino acids K682, G695, A708, V711, D732, A739, D733, L742, V747, F755, M771, M779, W782, A788, G791, L792, P793, Y797, M799, Q804, S819, or Y857 or a deletion of amino acid P793 of SEQ ID NO:2.

In some embodiments, the CasX variant protein comprises at least one modification in at least 1 domain, in at least each of 2 domains, in at least each of 3 domains, in at least each of 4 domains or in at least each of 5 domains of the reference CasX protein, including the sequences of SEQ ID NOS: 1-3. In some embodiments, the CasX variant protein comprises two or more modifications in at least one domain of the reference CasX protein. In some embodiments, the CasX variant protein comprises at least two modifications in at least one domain of the reference CasX protein, at least three modifications in at least one domain of the reference CasX protein or at least four modifications in at least one domain of the reference CasX protein. In some embodiments, wherein the CasX variant comprises two or more modifications compared to a reference CasX protein, each modification is made in a domain independently selected from the group consisting of a NTSBD, TSLD, Helical I domain, Helical II domain, OBD, and RuvC DNA cleavage domain. In some embodiments, the at least one modification of the CasX variant protein comprises a deletion of at least a portion of one domain of the reference CasX protein of SEQ ID NOS: 1-3. In some embodiments, the deletion is in the NTSBD, TSLD, Helical I domain, Helical II domain, OBD, or RuvC DNA cleavage domain. In other embodiments, the disclosure provides CasX variants wherein the CasX variants comprise at least one modification relative to another CasX variant; e.g., CasX variant 515 is a variant of CasX variant 491. All variants that improve one or more functions or characteristics of the CasX variant protein when compared to a reference CasX protein (or the variant from which it was derived) described herein are envisaged as being within the scope of the disclosure.

In some embodiments, the modification of the CasX variant is a mutation in one or more amino acids of the reference CasX. In other embodiments, the modification is a substitution of one or more domains of the reference CasX with one or more domains from a different CasX. In some embodiments, insertion includes the insertion of a part or all of a domain from a different CasX protein. Mutations can occur in any one or more domains of the reference CasX protein, and may include, for example, deletion of part or all of one or more domains, or one or more amino acid substitutions, deletions, or insertions in any domain of the reference CasX protein. The domains of CasX proteins include the non-target strand binding (NTSB) domain, the target strand loading (TSL) domain, the helical I domain, the helical II domain, the oligonucleotide binding domain (OBD), and the RuvC DNA cleavage domain. Any change in amino acid sequence of a reference CasX protein that leads to an improved characteristic of the CasX protein is considered a CasX variant protein of the disclosure. For example, CasX variants can comprise one or more amino acid substitutions, insertions, deletions, or swapped domains, or any combinations thereof, relative to a reference CasX protein sequence.

Suitable mutagenesis methods for generating CasX variant proteins of the disclosure may include, for example, Deep Mutational Evolution (DME), deep mutational scanning (DMS), error prone PCR, cassette mutagenesis, random mutagenesis, staggered extension PCR, gene shuffling, or domain swapping. In some embodiments, the CasX variants are designed, for example by selecting one or more desired mutations in a reference CasX. In certain embodiments, the activity of a reference CasX protein is used as a benchmark against which the activity of one or more CasX variants are compared, thereby measuring improvements in function of the CasX variants.

In some embodiments of the CasX variants described herein, the at least one modification comprises: (a) a substitution of 1 to 100 consecutive or non-consecutive amino acids in the CasX variant compared to a reference CasX of SEQ ID NO:1, SEQ ID NO:2, SEQ ID NO:3, CasX variant 491 or CasX variant 515; (b) a deletion of 1 to 100 consecutive or non-consecutive amino acids in the CasX variant compared to a reference CasX or the variant from which it was derived; (c) an insertion of 1 to 100 consecutive or non-consecutive amino acids in the CasX compared to a reference CasX or the variant from which it was derived; or (d) any combination of (a)-(c). In some embodiments, the at least one modification comprises: (a) a substitution of 5-10 consecutive or non-consecutive amino acids in the CasX variant compared to a reference CasX of SEQ ID NO:1, SEQ ID NO:2, SEQ ID NO:3, CasX 491 or CasX 515; (b) a deletion of 1-5 consecutive or non-consecutive amino acids in the CasX variant compared to a reference CasX or the variant from which it was derived; (c) an insertion of 1-5 consecutive or non-consecutive amino acids in the CasX compared to a reference CasX or the variant from which it was derived; or (d) any combination of (a)-(c).

In some embodiments, the CasX variant protein comprises or consists of a sequence that has at least 1, at least 2, at least 3, at least 4, at least 5, at least 6, at least 7, at least 8, at least 9, at least 10, at least 20, at least 30, at least 40, at least 50, at least 60, at least 70, at lease 80, at least 90, or at least 100 alterations relative to the sequence of SEQ ID NO:1, SEQ ID NO:2, SEQ ID NO:3, CasX variant 491 (with reference to Table 4) or CasX variant 515 (with reference to Table 4). These alterations can be amino acid insertions, deletions, substitutions, or any combinations thereof. The alterations can be in one domain or in any domain or any combination of domains of the CasX variant. Any amino acid can be substituted for any other amino acid in the substitutions described herein. The substitution can be a conservative substitution (e.g., a basic amino acid is substituted for another basic amino acid). The substitution can be a non-conservative substitution (e.g., a basic amino acid is substituted for an acidic amino acid or vice versa). For example, a proline in a reference CasX protein can be substituted for any of arginine, histidine, lysine, aspartic acid, glutamic acid, serine, threonine, asparagine, glutamine, cysteine, glycine, alanine, isoleucine, leucine, methionine, phenylalanine, tryptophan, tyrosine or valine to generate a CasX variant protein of the disclosure.

Any permutation of the substitution, insertion and deletion embodiments described herein can be combined to generate a CasX variant protein of the disclosure. For example, a CasX variant protein can comprise at least one substitution and at least one deletion relative to a reference CasX protein sequence, at least one substitution and at least one insertion relative to a reference CasX protein sequence, at least one insertion and at least one deletion relative to a reference CasX protein sequence, or at least one substitution, one insertion and one deletion relative to a reference CasX protein sequence.

In some embodiments, the CasX variant comprises at least one modification compared to the reference CasX sequence of SEQ ID NO:2 is selected from one or more of: (a) an amino acid substitution of L379R; (b) an amino acid substitution of A708K; (c) an amino acid substitution of T620P; (d) an amino acid substitution of E385P; (e) an amino acid substitution of Y857R; (f) an amino acid substitution of I658V; (g) an amino acid substitution of F399L; (h) an amino acid substitution of Q252K; (i) an amino acid substitution of L404K; and (j) an amino acid deletion of P793.

In some embodiments, the CasX variant protein comprises between 400 and 2000 amino acids, between 500 and 1500 amino acids, between 700 and 1200 amino acids, between 800 and 1100 amino acids, or between 900 and 1000 amino acids.

In some embodiments, a CasX variant protein comprises or consists of a sequence of SEQ ID NOS: 36-99, 101-148 or 43662-43907 as set forth in Table 4. In other embodiments, a CasX variant protein comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical to a sequence of SEQ ID NOS: 36-99, 101-148 or 43662-43907 as set forth in Table 4. In some embodiments, a CasX variant protein comprises a sequence of SEQ ID NOS: 59, 72-99, 101-148, or 43662-43907. In some embodiments, a CasX variant protein consists of a sequence of SEQ ID NOS: 59, 72-99, 101-148, or 43662-43907. In other embodiments, a CasX variant protein comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical to a sequence of SEQ ID NOS: 59, 72-99, 101-148, and 43662-43907. In some embodiments, a CasX variant protein comprises or consists of a sequence of SEQ ID NOS: 132-148 or 43662-43907. In other embodiments, a CasX variant protein comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical to a sequence of SEQ ID NOS: 132-148 or 43662-43907.

TABLE 4
CasX Variant Sequences

| SEQ ID | Variant | |
|---|---|---|
| NO | No. | Description of Variant |
| 36 | ND | TSL, Helical I, Helical II, OBD and RuvC domains from SEQ ID NO: 2 and an NTSB |
| | | domain from SEQ ID NO: 1 |
| 37 | ND | NTSB, Helical I, Helical II, OBD and RuvC domains from SEQ ID NO: 2 and a TSL |
| | | domain from SEQ ID NO: 1. |
| 38 | ND | TSL, Helical I, Helical II, OBD and RuvC domains from SEQ ID NO: 1 and an NTSB |
| | | domain from SEQ ID NO: 2 |
| 39 | ND | NTSB, Helical I, Helical II, OBD and RuvC domains from SEQ ID NO: 1 and an TSL |
| | | domain from SEQ ID NO: 2. |
| 40 | ND | NTSB, TSL, Helical I, Helical II and OBD domains SEQ ID NO: 2 and an exogenous |
| | | RuvC domain or a portion thereof from a second CasX protein. |
| 41 | ND | ND |
| 42 | ND | NTSB, TSL, Helical II, OBD and RuvC domains from SEQ ID NO: 2 and a Helical I |
| | | domain from SEQ ID NO: 1 |
| 43 | ND | NTSB, TSL, Helical I, OBD and RuvC domains from SEQ ID NO: 2 and a Helical II |
| | | domain from SEQ ID NO: 1 |
| 44 | ND | NTSB, TSL, Helical I, Helical II and RuvC domains from a first CasX protein and an |
| | | exogenous OBD or a part thereof from a second CasX protein |
| 45 | ND | ND |
| 46 | ND | ND |
| 47 | ND | substitution of L379R, a substitution of C477K, a substitution of A708K, a deletion of P |
| | | at position 793 and a substitution of T620P of SEQ ID NO: 2 |
| 48 | ND | substitution of M771A of SEQ ID NO: 2. |
| 49 | ND | substitution of L379R, a substitution of A708K, a deletion of P at position 793 and a |
| | | substitution of D732N of SEQ ID NO: 2. |
| 50 | ND | substitution of W782Q of SEQ ID NO: 2. |
| 51 | ND | substitution of M771Q of SEQ ID NO: 2 |
| 52 | ND | substitution of R458I and a substitution of A739V of SEQ ID NO: 2. |
| 53 | ND | L379R, a substitution of A708K, a deletion of P at position 793 and a substitution of |
| | | M771N of SEQ ID NO: 2 |
| 54 | ND | substitution of L379R, a substitution of A708K, a deletion of P at position 793 and a |
| | | substitution of A739T of SEQ ID NO: 2 |
| 55 | ND | substitution of L379R, a substitution of C477K, a substitution of A708K, a deletion of P |
| | | at position 793 and a substitution of D489S of SEQ ID NO: 2. |
| 56 | ND | substitution of L379R, a substitution of C477K, a substitution of A708K, a deletion of P |
| | | at position 793 and a substitution of D732N of SEQ ID NO: 2. |
| 57 | ND | substitution of V711K of SEQ ID NO: 2. |
| 58 | ND | substitution of L379R, a substitution of C477K, a substitution of A708K, a deletion of P |
| | | at position 793 and a substitution of Y797L of SEQ ID NO: 2. |
| 60 | ND | substitution of L379R, a substitution of C477K, a substitution of A708K, a deletion of P |
| | | at position 793 and a substitution of M771N of SEQ ID NO: 2. |
| 61 | ND | substitution of A708K, a deletion of P at position 793 and a substitution of E386S of |
| | | SEQ ID NO: 2. |
| 62 | ND | substitution of L379R, a substitution of C477K, a substitution of A708K and a deletion |
| | | of P at position 793 of SEQ ID NO: 2. |
| 63 | ND | substitution of L792D of SEQ ID NO: 2. |
| 64 | ND | substitution of G791F of SEQ ID NO: 2. |
| 65 | ND | substitution of A708K, a deletion of P at position 793 and a substitution of A739V of |
| | | SEQ ID NO: 2. |
| 66 | ND | substitution of L379R, a substitution of A708K, a deletion of P at position 793 and a |
| | | substitution of A739V of SEQ ID NO: 2. |
| 67 | ND | substitution of C477K, a substitution of A708K and a deletion of P at position 793 of |
| | | SEQ ID NO: 2. |
| 68 | ND | substitution of L249I and a substitution of M771N of SEQ ID NO: 2. |
| 69 | ND | substitution of V747K of SEQ ID NO: 2. |
| 70 | ND | substitution of L379R, a substitution of C477K, a substitution of A708K, a deletion of P |
| | | at position 793 and a substitution of M779N of SEQ ID NO: 2. |
| 71 | ND | L379R, F755M |
| 59 | 119 | ND |
| 72 | 429 | ND |
| 73 | 430 | ND |
| 74 | 431 | ND |
| 75 | 432 | ND |
| 76 | 433 | ND |
| 77 | 434 | ND |
| 78 | 435 | ND |
| 79 | 436 | ND |
| 80 | 437 | ND |
| 81 | 438 | ND |
| 82 | 439 | ND |
| 83 | 440 | ND |
| 84 | 441 | ND |
| 85 | 442 | ND |
| 86 | 443 | ND |
| 87 | 444 | ND |
| 88 | 445 | ND |
| 89 | 446 | ND |
| 90 | 447 | ND |
| 91 | 448 | ND |
| 92 | 449 | ND |
| 93 | 450 | ND |
| 94 | 451 | ND |
| 95 | 452 | ND |
| 96 | 453 | ND |
| 97 | 454 | ND |
| 98 | 455 | ND |
| 99 | 456 | ND |
| 101 | 457 | ND |
| 102 | 458 | ND |
| 103 | 459 | ND |
| 104 | 460 | ND |
| 105 | 278 | ND |
| 106 | 279 | ND |
| 107 | 280 | ND |
| 108 | 285 | ND |
| 109 | 286 | ND |
| 110 | 287 | ND |
| 111 | 288 | ND |
| 112 | 290 | ND |
| 113 | 291 | ND |
| 114 | 293 | ND |
| 115 | 300 | ND |
| 116 | 492 | ND |
| 117 | 493 | ND |
| 118 | 387 | ND |
| 119 | 395 | ND |
| 120 | 485 | ND |
| 121 | 486 | ND |
| 122 | 487 | ND |
| 123 | 488 | ND |
| 124 | 489 | ND |
| 125 | 490 | ND |
| 126 | 491 | ND |
| 127 | 494 | ND |
| 128 | 328 | ND |
| 129 | 388 | ND |
| 130 | 389 | ND |
| 131 | 390 | ND |
| 132 | 514 | ND |
| 133 | 515 | ND |
| 134 | 516 | ND |
| 135 | 517 | ND |
| 136 | 518 | ND |
| 137 | 519 | ND |
| 138 | 520 | ND |
| 139 | 522 | ND |
| 140 | 523 | ND |
| 141 | 524 | ND |
| 142 | 525 | ND |
| 143 | 526 | ND |
| 144 | 527 | ND |
| 145 | 528 | ND |
| 146 | 529 | ND |
| 147 | 530 | ND |
| 148 | 531 | ND |
| 43662 | 532 | ND |
| 43663 | 533 | ND |
| 43664 | 534 | ND |
| 43665 | 535 | ND |
| 43666 | 536 | ND |
| 43667 | 537 | ND |
| 43668 | 538 | ND |
| 43669 | 539 | ND |
| 43670 | 540 | ND |
| 43671 | 541 | ND |
| 43672 | 542 | ND |
| 43673 | 543 | ND |
| 43674 | 544 | ND |
| 43675 | 545 | ND |
| 43676 | 546 | ND |
| 43677 | 547 | ND |
| 43678 | 548 | ND |
| 43679 | 550 | ND |
| 43680 | 551 | ND |
| 43681 | 552 | ND |
| 43682 | 553 | ND |
| 43683 | 554 | ND |
| 43684 | 555 | ND |
| 43685 | 556 | ND |
| 43686 | 557 | ND |
| 43687 | 558 | ND |
| 43688 | 559 | ND |
| 43689 | 560 | ND |
| 43690 | 561 | ND |
| 43691 | 562 | ND |
| 43692 | 563 | ND |
| 43693 | 564 | ND |
| 43694 | 565 | ND |
| 43695 | 566 | ND |
| 43696 | 567 | ND |
| 43697 | 568 | ND |
| 43698 | 569 | ND |
| 43699 | 570 | ND |
| 43700 | 571 | ND |
| 43701 | 572 | ND |
| 43702 | 573 | ND |
| 43703 | 574 | ND |
| 43704 | 575 | ND |
| 43705 | 576 | ND |
| 43706 | 577 | ND |
| 43707 | 578 | ND |
| 43708 | 579 | ND |
| 43709 | 580 | ND |
| 43710 | 581 | ND |
| 43711 | 582 | ND |
| 43712 | 583 | ND |
| 43713 | 584 | ND |
| 43714 | 585 | ND |
| 43715 | 586 | ND |
| 43716 | 587 | ND |
| 43717 | 588 | ND |
| 43718 | 589 | ND |
| 43719 | 590 | ND |
| 43720 | 591 | ND |
| 43721 | 592 | ND |
| 43722 | 593 | ND |
| 43723 | 594 | ND |
| 43724 | 595 | ND |
| 43725 | 596 | ND |
| 43726 | 597 | ND |
| 43727 | 598 | ND |
| 43728 | 599 | ND |
| 43729 | 600 | ND |
| 43730 | 601 | ND |
| 43731 | 602 | ND |
| 43732 | 603 | ND |
| 43733 | 604 | ND |
| 43734 | 605 | ND |
| 43735 | 606 | ND |
| 43736 | 607 | ND |
| 43737 | 608 | ND |
| 43738 | 609 | ND |
| 43739 | 610 | ND |
| 43740 | 611 | ND |
| 43741 | 612 | ND |
| 43742 | 613 | ND |
| 43743 | 614 | ND |
| 43744 | 615 | ND |
| 43745 | 616 | ND |
| 43746 | 617 | ND |
| 43747 | 618 | ND |
| 43748 | 619 | ND |
| 43749 | 620 | ND |
| 43750 | 621 | ND |
| 43751 | 622 | ND |
| 43752 | 623 | ND |
| 43753 | 624 | ND |
| 43754 | 625 | ND |
| 43755 | 626 | ND |
| 43756 | 627 | ND |
| 43757 | 628 | ND |
| 43758 | 629 | ND |
| 43759 | 630 | ND |
| 43760 | 631 | ND |
| 43761 | 632 | ND |
| 43762 | 633 | ND |
| 43763 | 634 | ND |
| 43764 | 635 | ND |
| 43765 | 636 | ND |
| 43766 | 637 | ND |
| 43767 | 638 | ND |
| 43768 | 639 | ND |
| 43769 | 640 | ND |
| 43770 | 641 | ND |
| 43771 | 642 | ND |
| 43772 | 643 | ND |
| 43773 | 644 | ND |
| 43774 | 645 | ND |
| 43775 | 646 | ND |
| 43776 | 647 | ND |
| 43777 | 648 | ND |
| 43778 | 649 | ND |
| 43779 | 650 | ND |
| 43780 | 651 | ND |
| 43781 | 652 | ND |
| 43782 | 653 | ND |
| 43783 | 654 | ND |
| 43784 | 655 | ND |
| 43785 | 656 | ND |
| 43786 | 657 | ND |
| 43787 | 658 | ND |
| 43788 | 659 | ND |
| 43789 | 660 | ND |
| 43790 | 661 | ND |
| 43791 | 662 | ND |
| 43792 | 663 | ND |
| 43793 | 664 | ND |
| 43794 | 665 | ND |
| 43795 | 666 | ND |
| 43796 | 667 | ND |
| 43797 | 668 | ND |
| 43798 | 669 | ND |
| 43799 | 671 | ND |
| 43800 | 672 | ND |
| 43801 | 673 | ND |
| 43802 | 674 | ND |
| 43803 | 675 | ND |
| 43804 | 676 | ND |
| 43805 | 677 | ND |
| 43806 | 678 | ND |
| 43807 | 679 | ND |
| 43808 | 680 | ND |
| 43809 | 681 | ND |
| 43810 | 682 | ND |
| 43811 | 683 | ND |
| 43812 | 684 | ND |
| 43813 | 685 | ND |
| 43814 | 686 | ND |
| 43815 | 687 | ND |
| 43816 | 688 | ND |
| 43817 | 689 | ND |
| 43818 | 690 | ND |
| 43819 | 691 | ND |
| 43820 | 692 | ND |
| 43821 | 693 | ND |
| 43822 | 694 | ND |
| 43823 | 701 | ND |
| 43824 | 702 | ND |
| 43825 | 703 | ND |
| 43826 | 704 | ND |
| 43827 | 705 | ND |
| 43828 | 706 | ND |
| 43829 | 707 | ND |
| 43830 | 708 | ND |
| 43831 | 709 | ND |
| 43832 | 710 | ND |
| 43833 | 711 | ND |
| 43834 | 712 | ND |
| 43835 | 713 | ND |
| 43836 | 714 | ND |
| 43837 | 715 | ND |
| 43838 | 716 | ND |
| 43839 | 717 | ND |
| 43840 | 718 | ND |
| 43841 | 719 | ND |
| 43842 | 720 | ND |
| 43843 | 721 | ND |
| 43844 | 722 | ND |
| 43845 | 723 | ND |
| 43846 | 724 | ND |
| 43847 | 725 | ND |
| 43848 | 726 | ND |
| 43849 | 727 | ND |
| 43850 | 728 | ND |
| 43851 | 729 | ND |
| 43852 | 730 | ND |
| 43853 | 731 | ND |
| 43854 | 732 | ND |
| 43855 | 733 | ND |
| 43856 | 734 | ND |
| 43857 | 735 | ND |
| 43858 | 736 | ND |
| 43859 | 737 | ND |
| 43860 | 738 | ND |
| 43861 | 739 | ND |
| 43862 | 740 | ND |
| 43863 | 741 | ND |
| 43864 | 742 | ND |
| 43865 | 743 | ND |
| 43866 | 744 | ND |
| 43867 | 745 | ND |
| 43868 | 746 | ND |
| 43869 | 747 | ND |
| 43870 | 748 | ND |
| 43871 | 749 | ND |
| 43872 | 750 | ND |
| 43873 | 751 | ND |
| 43874 | 752 | ND |
| 43875 | 753 | ND |
| 43876 | 754 | ND |
| 43877 | 755 | ND |
| 43878 | 756 | ND |
| 43879 | 757 | ND |
| 43880 | 758 | ND |
| 43881 | 759 | ND |
| 43882 | 760 | ND |
| 43883 | 761 | ND |
| 43884 | 762 | ND |
| 43885 | 763 | ND |
| 43886 | 764 | ND |
| 43887 | 765 | ND |
| 43888 | 766 | ND |
| 43889 | 767 | ND |
| 43890 | 768 | ND |
| 43891 | 769 | ND |
| 43892 | 770 | ND |
| 43893 | 777 | ND |
| 43894 | 778 | ND |
| 43895 | 779 | ND |
| 43896 | 780 | ND |
| 43897 | 781 | ND |
| 43898 | 782 | ND |
| 43899 | 783 | ND |
| 43900 | 784 | ND |
| 43901 | 785 | ND |
| 43902 | 786 | ND |
| 43903 | 787 | ND |
| 43904 | 788 | ND |
| 43905 | 789 | ND |
| 43906 | 790 | ND |
| 43907 | 791 | ND |

### c. CasX Variant Proteins with Domains from Multiple Source Proteins

In certain embodiments, the disclosure provides a chimeric CasX protein comprising protein domains from two or more different CasX proteins, such as two or more reference CasX proteins, or two or more CasX variant protein sequences as described herein. As used herein, a “chimeric CasX protein” refers to a CasX containing at least two domains isolated or derived from different sources, such as two naturally occurring proteins, which may, in some embodiments, be isolated from different species. For example, in some embodiments, a chimeric CasX protein comprises a first domain from a first CasX protein and a second domain from a second, different CasX protein. In some embodiments, the first domain can be selected from the group consisting of the NTSB, TSL, Helical I, Helical II, OBD and RuvC domains. In some embodiments, the second domain is selected from the group consisting of the NTSB, TSL, Helical I, Helical II, OBD and RuvC domains with the second domain being different from the foregoing first domain. For example, a chimeric CasX protein may comprise an NTSB, TSL, Helical I, Helical II, OBD domains from a CasX protein of SEQ ID NO: 2, and a RuvC domain from a CasX protein of SEQ ID NO: 1, or vice versa. As a further example, a chimeric CasX protein may comprise an NTSB, TSL, Helical II, OBD and RuvC domain from CasX protein of SEQ ID NO: 2, and a Helical I domain from a CasX protein of SEQ ID NO: 1, or vice versa. Thus, in certain embodiments, a chimeric CasX protein may comprise an NTSB, TSL, Helical II, OBD and RuvC domain from a first CasX protein, and a Helical I domain from a second CasX protein. In some embodiments of the chimeric CasX proteins, the domains of the first CasX protein are derived from the sequences of SEQ ID NO: 1, SEQ ID NO: 2 or SEQ ID NO: 3, and the domains of the second CasX protein are derived from the sequences of SEQ ID NO: 1, SEQ ID NO: 2 or SEQ ID NO: 3, and the first and second CasX proteins are not the same. In some embodiments, domains of the first CasX protein comprise sequences derived from SEQ ID NO: 1 and domains of the second CasX protein comprise sequences derived from SEQ ID NO: 2. In some embodiments, domains of the first CasX protein comprise sequences derived from SEQ ID NO: 1 and domains of the second CasX protein comprise sequences derived from SEQ ID NO: 3. In some embodiments, domains of the first CasX protein comprise sequences derived from SEQ ID NO: 2 and domains of the second CasX protein comprise sequences derived from SEQ ID NO: 3. In some embodiments, a CasX protein comprises a first domain from a first CasX protein and a second domain from a second CasX protein, and at least one chimeric domain comprising at least two parts isolated from different CasX proteins using the approach of the embodiments described in this paragraph. In some embodiments, the at least one chimeric domain comprises a chimeric RuvC domain. As an example of the foregoing, the chimeric RuvC domain comprises amino acids 660 to 823 of SEQ ID NO: 1 and amino acids 921 to 978 of SEQ ID NO: 2. As an alternative example of the foregoing, a chimeric RuvC domain comprises amino acids 647 to 810 of SEQ ID NO: 2 and amino acids 934 to 986 of SEQ ID NO: 1. In a particular embodiment, the CasX variants of 514-791 have a NTSB and helical 1B domain of SEQ ID NO: 1, while the other domains are derived from SEQ ID NO: 2, it being understood that the variants have additional amino acid changes at select locations. In another particular embodiment, the CasX variant of 494 has a NTSB domain of SEQ DI NO: 1, while the other domains are derived from SEQ ID NO: 2.

In the case of split or non-contiguous domains such as helical I, RuvC and OBD, a portion of the non-contiguous domain can be replaced with the corresponding portion from any other source. For example, the helical I-I domain (sometimes referred to as helical I-a) in SEQ ID NO: 2 can be replaced with the corresponding helical I-I sequence from SEQ ID NO: 1, and the like. Domain sequences from reference CasX proteins, and their coordinates, are shown in Table 5. Representative examples of chimeric CasX proteins include the variants of CasX 472-483, 485-491 and 515, the sequences of which are set forth in Table 4.

TABLE 5
Domain coordinates in Reference CasX proteins

| Domain | Coordinates in | Coordinates in |
|---|---|---|
| Name | SEQ ID NO: 1 | SEQ ID NO: 2 |
| OBD a | 1-55 | 1-57 |
| helical I a | 56-99 | 58-101 |
| NTSB | 100-190 | 102-191 |
| helical I b | 191-331 | 192-332 |
| helical II | 332-508 | 333-500 |
| OBD b | 509-659 | 501-646 |
| RuvC a | 660-823 | 647-810 |
| TSL | 824-933 | 811-920 |
| RuvC b | 934-986 | 921-978 |

*OBD a and b, helical I a and b, and RuvC a and b are also referred to herein as OBD I and II, helical I-I and I-II, and RuvC I and II.

### d. Protein Affinity for the gRNA

In some embodiments, a CasX variant protein has improved affinity for the gRNA relative to a reference CasX protein, leading to the formation of the ribonucleoprotein complex. Increased affinity of the CasX variant protein for the gRNA may, for example, result in a lower K_{d} for the generation of a RNP complex, which can, in some cases, result in a more stable ribonucleoprotein complex formation. In some embodiments, increased affinity of the CasX variant protein for the gRNA results in increased stability of the ribonucleoprotein complex when delivered to human cells. This increased stability can affect the function and utility of the complex in the cells of a subject, as well as result in improved pharmacokinetic properties in blood, when delivered to a subject. In some embodiments, increased affinity of the CasX variant protein, and the resulting increased stability of the ribonucleoprotein complex, allows for a lower dose of the CasX variant protein to be delivered to the subject or cells while still having the desired activity, for example in vivo or in vitro gene editing. In some embodiments, a higher affinity (tighter binding) of a CasX variant protein to a gRNA allows for a greater amount of editing events when both the CasX variant protein and the gRNA remain in an RNP complex. Increased editing events can be assessed using editing assays such as the tdTom editing assays described herein. In some embodiments, the K_{d} of a CasX variant protein for a gRNA is increased relative to a reference CasX protein by a factor of at least about 1.1, at least about 1.2, at least about 1.3, at least about 1.4, at least about 1.5, at least about 1.6, at least about 1.7, at least about 1.8, at least about 1.9, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100. In some embodiments, the CasX variant has about 1.1 to about 10-fold increased binding affinity to the gRNA compared to the reference CasX protein of SEQ ID NO: 2.

In some embodiments, a higher affinity (tighter binding) of a CasX variant protein to a gRNA allows for a greater amount of editing events when both the CasX variant protein and the gRNA remain in an RNP complex. Increased editing events can be assessed using editing assays such as the EGFP disruption assay described herein.

Without wishing to be bound by theory, in some embodiments amino acid changes in the Helical I domain can increase the binding affinity of the CasX variant protein with the gRNA targeting sequence, while changes in the Helical II domain can increase the binding affinity of the CasX variant protein with the gRNA scaffold stem loop, and changes in the oligonucleotide binding domain (OBD) increase the binding affinity of the CasX variant protein with the gRNA triplex.

Methods of measuring CasX protein binding affinity for a CasX gRNA include in vitro methods using purified CasX protein and gRNA. The binding affinity for reference CasX and variant proteins can be measured by fluorescence polarization if the gRNA or CasX protein is tagged with a fluorophore. Alternatively, or in addition, binding affinity can be measured by biolayer interferometry, electrophoretic mobility shift assays (EMSAs), or filter binding. Additional standard techniques to quantify absolute affinities of RNA binding proteins such as the reference CasX and variant proteins of the disclosure for specific gRNAs such as reference gRNAs and variants thereof include, but are not limited to, isothermal calorimetry (ITC), and surface plasmon resonance (SPR), as well as the methods of the Examples.

### e. Affinity for Target Nucleic Acid

In some embodiments, a CasX variant protein has improved binding affinity for a target nucleic acid relative to the affinity of a reference CasX protein for a target nucleic acid, when complexed as an RNP, relative to the affinity of a reference CasX protein for a target nucleic acid sequence. In some embodiments, affinity of a CasX variant protein of the disclosure for a target nucleic acid molecule is increased relative to a reference CasX protein by a factor of at least about 1.1, at least about 1.2, at least about 1.3, at least about 1.4, at least about 1.5, at least about 1.6, at least about 1.7, at least about 1.8, at least about 1.9, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100.

CasX variants with higher affinity for their target nucleic acid may, in some embodiments, cleave the target nucleic acid sequence more rapidly than a reference CasX protein that does not have increased affinity for the target nucleic acid. In some embodiments, the improved affinity for the target nucleic acid sequence comprises improved affinity for the target nucleic acid sequence, improved binding affinity to a wider spectrum of PAM sequences, an improved ability to search DNA for the target nucleic acid sequence, or any combinations thereof, resulting in an increased ability to modify the target nucleic acid. In some embodiments, a CasX variant protein with improved target nucleic acid affinity has increased affinity for specific PAM sequences other than the canonical TTC PAM recognized by the reference CasX protein of SEQ ID NO: 2, including binding affinity for PAM sequences selected from the group consisting of TTC, ATC, GTC, and CTC. A higher overall affinity for DNA also, in some embodiments, can increase the frequency at which a CasX protein can effectively start and finish a binding and unwinding step, thereby facilitating target strand invasion and R-loop formation, and ultimately the cleavage of a target nucleic acid sequence.

In some embodiments, a CasX variant protein has improved binding affinity for the non-target strand of the target nucleic acid. As used herein, the term “non-target strand” refers to the strand of the DNA target nucleic acid sequence that does not form Watson and Crick base pairs with the targeting sequence in the gRNA and is complementary to the target DNA strand. In some embodiments, the CasX variant protein has about 1.1 to about 100-fold increased binding affinity to the non-target stand of the target nucleic acid compared to the reference protein of SEQ ID NO: 1, SEQ ID NO: 2, or SEQ ID NO: 3.

Methods of measuring CasX variant protein affinity for a target nucleic acid molecule may include electrophoretic mobility shift assays (EMSAs), filter binding, isothermal calorimetry (ITC), and surface plasmon resonance (SPR), fluorescence polarization and biolayer interferometry (BLI). Further methods of measuring CasX protein affinity for a target include in vitro biochemical assays that measure DNA cleavage events over time; e.g., determination of the k_{cleave} rate, as described in the Examples.

### f. Improved Specificity for a Target Site

In some embodiments, a CasX variant protein has improved specificity for a target nucleic acid sequence relative to a reference CasX protein of SEQ ID NOS: 1-3. As used herein, “specificity,” interchangeably referred to as “target specificity,” refers to the degree to which a CRISPR/Cas system ribonucleoprotein complex cleaves off-target sequences that are similar, but not identical to the target nucleic acid sequence; e.g., a CasX variant RNP with a higher degree of specificity would exhibit reduced off-target cleavage of sequences relative to a reference CasX protein. The specificity, and the reduction of potentially deleterious off-target effects, of CRISPR/Cas system proteins can be vitally important in order to achieve an acceptable therapeutic index for use in mammalian subjects.

In some embodiments, a CasX variant protein has improved specificity for a target site within the target sequence that is complementary to the targeting sequence of the gRNA relative to a reference CasX protein of SEQ ID NOS: 1-3. Without wishing to be bound by theory, it is possible that amino acid changes in the helical I and II domains that increase the specificity of the CasX variant protein for the target nucleic acid strand can increase the specificity of the CasX variant protein for the target nucleic acid overall. In some embodiments, amino acid changes that increase specificity of CasX variant proteins for target nucleic acid may also result in decreased affinity of CasX variant proteins for DNA.

Methods of testing CasX protein (such as variant or reference) target specificity may include guide and Circularization for In vitro Reporting of Cleavage Effects by Sequencing (CIRCLE-seq), or similar methods. In brief, in CIRCLE-seq techniques, genomic DNA is sheared and circularized by ligation of stem-loop adapters, which are nicked in the stem-loop regions to expose 4 nucleotide palindromic overhangs. This is followed by intramolecular ligation and degradation of remaining linear DNA. Circular DNA molecules containing a CasX cleavage site are subsequently linearized with CasX, and adapter adapters are ligated to the exposed ends followed by high-throughput sequencing to generate paired end reads that contain information about the off-target site. Additional assays that can be used to detect off-target events, and therefore CasX protein specificity include assays used to detect and quantify indels (insertions and deletions) formed at those selected off-target sites such as mismatch-detection nuclease assays and next generation sequencing (NGS). Exemplary mismatch-detection assays include nuclease assays, in which genomic DNA from cells treated with CasX and sgRNA is PCR amplified, denatured and rehybridized to form hetero-duplex DNA, containing one wild type strand and one strand with an indel. Mismatches are recognized and cleaved by mismatch detection nucleases, such as Surveyor nuclease or T7 endonuclease I.

### g. Protospacer and PAM Sequences

Herein, the protospacer is defined as the DNA sequence complementary to the targeting sequence of the guide RNA and the DNA complementary to that sequence, referred to as the target strand and non-target strand, respectively. As used herein, the PAM is a nucleotide sequence located is located 1 nucleotide 5′ of the sequence in the non-target strand that is complementary to the target nucleic acid sequence in the target strand of the target nucleic acid that, in conjunction with the targeting sequence of the gRNA, helps the orientation and positioning of the CasX for the potential cleavage of the protospacer strand(s).

PAM sequences may be degenerate, and specific RNP constructs may have different preferred and tolerated PAM sequences that support different efficiencies of cleavage. Following convention, unless stated otherwise, the disclosure refers to both the PAM and the protospacer sequence and their directionality according to the orientation of the non-target strand. This does not imply that the PAM sequence of the non-target strand, rather than the target strand, is determinative of cleavage or mechanistically involved in target recognition. For example, when reference is to a TTC PAM, it may in fact be the complementary GAA sequence that is required for target cleavage, or it may be some combination of nucleotides from both strands. In the case of the CasX proteins disclosed herein, the PAM is located 5′ of the protospacer with a single nucleotide separating the PAM from the first nucleotide of the protospacer. Thus, in the case of reference CasX, a TTC PAM should be understood to mean a sequence following the formula 5′- . . . NNTTCN(protospacer)NNNNNN . . . 3′ where ‘N’ is any DNA nucleotide and ‘(protospacer)’ is a DNA sequence having identity with the targeting sequence of the guide RNA. In the case of a CasX variant with expanded PAM recognition, a TTC, CTC, GTC, or ATC PAM should be understood to mean a sequence following the formulae:

5′- . . . NNTTCN(protospacer)NNNNNN . . . 3′;

5′- . . . NNCTCN(protospacer)NNNNNN . . . 3′;

5′- . . . NNGTCN(protospacer)NNNNNN . . . 3′; or

5′- . . . NNATCN(protospacer)NNNNNN . . . 3′. Alternatively, a TC PAM should be understood to mean a sequence following the formula.

5′- . . . NNNTCN(protospacer)NNNNNN . . . 3′.

Additionally, the CasX variant proteins of the disclosure have an enhanced ability to efficiently edit and/or bind target DNA, when complexed with a gRNA as an RNP, utilizing a PAM TC motif, including PAM sequences selected from TTC, ATC, GTC, or CTC, (in a 5′ to 3′ orientation), compared to an RNP of a reference CasX protein and reference gRNA. In the foregoing, the PAM sequence is located at least 1 nucleotide 5′ to the non-target strand of the protospacer having identity with the targeting sequence of the gRNA in an assay system compared to the editing efficiency and/or binding of an RNP comprising a reference CasX protein and reference gRNA in a comparable assay system. In one embodiment, an RNP of a CasX variant and gRNA variant exhibits greater editing efficiency and/or binding of a target sequence in the target DNA compared to an RNP comprising a reference CasX protein and a reference gRNA in a comparable assay system, wherein the PAM sequence of the target DNA is TTC. In another embodiment, an RNP of a CasX variant and gRNA variant exhibits greater editing efficiency and/or binding of a target sequence in the target DNA compared to an RNP comprising a reference CasX protein and a reference gRNA in a comparable assay system, wherein the PAM sequence of the target DNA is ATC. In another embodiment, an RNP of a CasX variant and gRNA variant exhibits greater editing efficiency and/or binding of a target sequence in the target DNA compared to an RNP comprising a reference CasX protein and a reference gRNA in a comparable assay system, wherein the PAM sequence of the target DNA is CTC. In another embodiment, an RNP of a CasX variant and gRNA variant exhibits greater editing efficiency and/or binding of a target sequence in the target DNA compared to an RNP comprising a reference CasX protein and a reference gRNA in a comparable assay system, wherein the PAM sequence of the target DNA is GTC. In the foregoing embodiments, the increased editing efficiency and/or binding affinity for the one or more PAM sequences is at least 1.5-fold greater, at least 5-fold greater, at least 10-fold greater, or at least 100-fold greater or more compared to the editing efficiency and/or binding affinity of an RNP of any one of the CasX proteins of SEQ ID NOS:1-3 and the gRNA of Table 2 or Table 3 for the PAM sequences. In some embodiments, an RNP comprising a CasX variant and a gRNA variant of the embodiments described herein exhibit higher percent editing of the target nucleic acid in a timed in vitro assay that is at least about 5-fold, at least about 10-fold, at least about 20-fold, or at least about 100-fold higher compared to an RNP of any one of the reference CasX proteins of SEQ ID NOS: 1-3 and the gRNA of SEQ ID NOs: 4-16 in a comparable assay. Exemplary assays demonstrating the improved editing are described herein, in the Examples.

### h. Unwinding of DNA

In some embodiments, a CasX variant protein has improved ability of unwinding DNA relative to a reference CasX protein. Poor dsDNA unwinding has been shown previously to impair or prevent the ability of CRISPR/Cas system proteins AnaCas9 or Cas14s to cleave DNA. Therefore, without wishing to be bound by any theory, it is likely that increased DNA cleavage activity by some CasX variant proteins of the disclosure is due, at least in part, to an increased ability to find and unwind the dsDNA at a target site. Methods of measuring the ability of CasX proteins (such as variant or reference) to unwind DNA include, but are not limited to, in vitro assays that observe increased on rates of dsDNA targets in fluorescence polarization or biolayer interferometry.

Without wishing to be bound by theory, it is thought that amino acid changes in the NTSB domain may produce CasX variant proteins with increased DNA unwinding characteristics. Alternatively, or in addition, amino acid changes in the OBD or the helical domain regions that interact with the PAM may also produce CasX variant proteins with increased DNA unwinding characteristics.

Methods of measuring the ability of CasX proteins (such as variant or reference) to unwind DNA include, but are not limited to, in vitro assays that observe increased on rates of dsDNA targets in fluorescence polarization or biolayer interferometry.

### i. Catalytic Activity

The ribonucleoprotein complex of the CasX:gRNA systems disclosed herein comprise a variant thereof that bind to a target nucleic acid and cleaves the target nucleic acid. In some embodiments, a CasX variant protein has improved catalytic activity relative to a reference CasX protein. Without wishing to be bound by theory, it is thought that in some cases cleavage of the target strand can be a limiting factor for Cas12-like molecules in creating a dsDNA break. In some embodiments, CasX variant proteins improve bending of the target strand of DNA and cleavage of this strand, resulting in an improvement in the overall efficiency of dsDNA cleavage by the CasX ribonucleoprotein complex.

In some embodiments, a CasX variant protein has increased nuclease activity compared to a reference CasX protein. Variants with increased nuclease activity can be generated, for example, through amino acid changes in the RuvC nuclease domain. In some embodiments, the CasX variant comprises a nuclease domain having nickase activity. In the foregoing, the CasX nickase of a CasX:gRNA system generates a single-stranded break within 10-18 nucleotides 3′ of a PAM site in the non-target strand. In other embodiments, the CasX variant comprises a nuclease domain having double-stranded cleavage activity. In the foregoing, the CasX of the CasX:gRNA system generates a double-stranded break within 18-26 nucleotides 5′ of a PAM site on the target strand and 10-18 nucleotides 3′ on the non-target strand. Nuclease activity can be assayed by a variety of methods, including those of the Examples. In some embodiments, a CasX variant has a K_{cleave} constant that is at least 2-fold, or at least 3-fold, or at least 4-fold, or at least 5-fold, or at least 6-fold, or at least 7-fold, or at least 8-fold, or at least 9-fold, or at least 10-fold greater compared to a reference CasX.

In some embodiments, a CasX variant protein has the improved characteristic of forming RNP with gRNA that result in a higher percentage of cleavage-competent RNP compared to an RNP of a reference CasX protein of SEQ ID NO: 1, SEQ ID NO: 2, or SEQ ID NO: 3 and the gRNA, as described in the Examples. By cleavage competent, it is meant that the RNP that is formed has the ability to cleave the target nucleic acid. In some embodiments, the CasX variant and the gRNA of the disclosure are able to form RNP exhibiting at least a 2% to at least 40%, or at least a 5% to at least a 20%, or at least a 10% to at least a 15% higher percentage of cleavage-competent conformations compared to an RNP of the reference CasX of SEQ ID NO: 1, SEQ ID NO: 2, or SEQ ID NO: 3 and the gRNA of Table 2. In the foregoing embodiment, the improved cleavage competency can be demonstrated in an in vitro assay, such as described in the Examples.

In some embodiments, a CasX variant protein has increased target strand loading for double strand cleavage compared to a reference CasX. Variants with increased target strand loading activity can be generated, for example, through amino acid changes in the TLS domain.

Without wishing to be bound by theory, amino acid changes in the TSL domain may result in CasX variant proteins with improved catalytic activity. Alternatively, or in addition, amino acid changes around the binding channel for the RNA:DNA duplex may also improve catalytic activity of the CasX variant protein.

In some embodiments, a CasX variant protein has increased collateral cleavage activity compared to a reference CasX protein. As used herein, “collateral cleavage activity” refers to additional, non-targeted cleavage of nucleic acids following recognition and cleavage of a target nucleic acid. In some embodiments, a CasX variant protein has decreased collateral cleavage activity compared to a reference CasX protein.

In some embodiments, for example those embodiments encompassing applications where cleavage of the target nucleic acid is not a desired outcome, improving the catalytic activity of a CasX variant protein comprises altering, reducing, or abolishing the catalytic activity of the CasX variant protein. In some embodiments, a ribonucleoprotein complex comprising a dCasX variant protein binds to a target nucleic acid and does not cleave the target nucleic acid.

In some embodiments, the CasX ribonucleoprotein complex comprising a CasX variant protein binds a target DNA but generates a single stranded nick in the target DNA. In some embodiments, particularly those embodiments wherein the CasX protein is a nickase, a CasX variant protein has decreased target strand loading for single strand nicking. Variants with decreased target strand loading may be generated, for example, through amino acid changes in the TSL domain.

Exemplary methods for characterizing the catalytic activity of CasX proteins may include, but are not limited to, in vitro cleavage assays, including those of the Examples, below. In some embodiments, electrophoresis of DNA products on agarose gels can interrogate the kinetics of strand cleavage.

### J. Casx Fusion Proteins

In some embodiments, the disclosure provides CasX proteins comprising a heterologous protein fused to the CasX variant of any of the embodiments described herein.

In some embodiments, the CasX variant protein comprises any one of SEQ ID NOS: 59, 72-99, 101-148, and 43662-43907, of the sequences of Table 4 fused to one or more proteins or domains thereof that has a different activity of interest, resulting in a fusion protein. For example, in some embodiments, the CasX variant protein is fused to a protein (or domain thereof) that inhibits transcription, modifies a target nucleic acid, or modifies a polypeptide associated with a nucleic acid (e.g., histone modification).

In some embodiments, a heterologous polypeptide (or heterologous amino acid such as a cysteine residue or a non-natural amino acid) can be inserted at one or more positions within a CasX protein to generate a CasX fusion protein. In other embodiments, a cysteine residue can be inserted at one or more positions within a CasX protein followed by conjugation of a heterologous polypeptide described below. In some alternative embodiments, a heterologous polypeptide or heterologous amino acid can be added at the N- or C-terminus of the CasX variant protein. In other embodiments, a heterologous polypeptide or heterologous amino acid can be inserted internally within the sequence of the CasX protein.

In some embodiments, the CasX variant fusion protein retains RNA-guided sequence specific target nucleic acid binding and cleavage activity. In some cases, the CasX variant fusion protein has (retains) 50% or more of the activity (e.g., cleavage and/or binding activity) of the corresponding CasX variant protein that does not have the insertion of the heterologous protein. In some cases, the CasX variant fusion protein retains at least about 60%, or at least about 70% or more, at least about 80%, or at least about 90%, or at least about 92%, or at least about 95%, or at least about 98%, or at least about 100% of the activity (e.g., cleavage and/or binding activity) of the corresponding CasX protein that does not have the insertion of the heterologous protein.

In some cases, the CasX variant fusion protein retains (has) target nucleic acid binding activity relative to the activity of the CasX protein without the inserted heterologous amino acid or heterologous polypeptide. In some cases, the CasX variant fusion protein retains at least about 60%, or at least about 70% or more, at least about 80%, or at least about 90%, or at least about 92%, or at least about 95%, or at least about 98%, or at least about 100% of the binding activity of the corresponding CasX protein that does not have the insertion of the heterologous protein.

In some cases, the CasX variant fusion protein retains (has) target nucleic acid binding and/or cleavage activity relative to the activity of the parent CasX protein without the inserted heterologous amino acid or heterologous polypeptide. For example, in some cases, the CasX variant fusion protein has (retains) 50% or more of the binding and/or cleavage activity of the corresponding parent CasX protein (the CasX protein that does not have the insertion). For example, in some cases, the CasX variant fusion protein has (retains) 60% or more (70% or more, 80% or more, 90% or more, 92% or more, 95% or more, 98% or more, or 100%) of the binding and/or cleavage activity of the corresponding CasX parent protein (the CasX protein that does not have the insertion). Methods of measuring cleaving and/or binding activity of a CasX protein and/or a CasX fusion protein will be known to one of ordinary skill in the art and any convenient method can be used.

A variety of heterologous polypeptides are suitable for inclusion in a CasX variant fusion protein of the disclosure. In some cases, the fusion partner can modulate transcription (e.g., inhibit transcription, increase transcription) of a target DNA. For example, in some cases the fusion partner is a protein (or a domain from a protein) that inhibits transcription (e.g., a transcriptional repressor, a protein that functions via recruitment of transcription inhibitor proteins, modification of target DNA such as methylation, recruitment of a DNA modifier, modulation of histones associated with target DNA, recruitment of a histone modifier such as those that modify acetylation and/or methylation of histones, and the like). In some cases the fusion partner is a protein (or a domain from a protein) that increases transcription (e.g., a transcription activator, a protein that acts via recruitment of transcription activator proteins, modification of target DNA such as demethylation, recruitment of a DNA modifier, modulation of histones associated with target DNA, recruitment of a histone modifier such as those that modify acetylation and/or methylation of histones, and the like).

In some cases, a fusion partner has enzymatic activity that modifies a target nucleic acid; e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity.

In some cases, a fusion partner has enzymatic activity that modifies a polypeptide (e.g., a histone) associated with a target nucleic acid (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

Examples of proteins (or fragments thereof) that can be used as a fusion partner to increase transcription include but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET domain containing 1A, histone lysine methyltransferase (SET1A), SET domain containing 1B, histone lysine methyltransferase (SET1B), lysine methyltransferase (MLL1 to 5), ASCL1 (ASH1) achaete-scute family bHLH transcription factor 1 (ASH1), SET and MYND domain containing 2 (SYMD2), nuclear receptor binding SET domain protein 1 (NSD1), and the like; histone lysine demethylases such as lysine demethylase 3A (JHDM2a)/Lysine-specific demethylase 3B (JHDM2b), lysine demethylase 6A (UTX), lysine demethylase 6B (JMJD3), and the like; histone acetyltransferases such as lysine acetyltransferase 1 (GCN5), lysine acetyltransferase 2 (PCAF), CREB binding protein (CBP), E1A binding protein p300 (p300), TATA-box binding protein associated factor 1 (TAF1), lysine acetyltransferase 5 (TIP60/PLIP), lysine acetyltransferase 6A (MOZ/MYST3), lysine acetyltransferase 6B (MORF/MYST4), SRC proto-oncogene, non-receptor tyrosine kinase (SRC1), nuclear receptor coactivator 3 (ACTR), MYB binding protein 1a (P160), clock circadian regulator (CLOCK), and the like; and DNA demethylases such as Ten-Eleven Translocation (TET) dioxygenase 1 (TET1CD), tet methylcytosine dioxygenase 1 (TET1), demeter (DME), demeter-like 1 (DML1), demeter-like 2 (DML2), protein ROS1 (ROS1), and the like.

Examples of proteins (or fragments thereof) that can be used as a fusion partner to decrease transcription include but are not limited to: transcriptional repressors such as the Kruppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as PR/SET domain containing protein (Pr-SET7/8), lysine methyltransferase 5B (SUV4-20H1), PR/SET domain 2 (RIZ1), and the like; histone lysine demethylases such as lysine demethylase 4A (JMJD2A/JHDM3A), lysine demethylase 4B (JMJD2B), lysine demethylase 4C (JMJD2C/GASC1), lysine demethylase 4D (JMJD2D), lysine demethylase 5A (JARID1A/RBP2), lysine demethylase 5B (JARID1B/PLU-1), lysine demethylase 5C (JARID 1C/SMCX), lysine demethylase 5D (JARID1D/SMCY), and the like; histone lysine deacetylases such as histone deacetylase 1 (HDAC1), HDAC2, HDAC3, HDAC8, HDAC4, HDAC5, HDAC7, HDAC9, sirtuin 1 (SIRT1), SIRT2, HDAC11, and the like; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), methyltransferase 1 (MET1), S-adenosyl-L-methionine-dependent methyltransferases superfamily protein (DRM3) (plants), DNA cytosine methyltransferase MET2a (ZMET2), chromomethylase 1 (CMT1), chromomethylase 2 (CMT2) (plants), and the like; and periphery recruitment elements such as Lamin A, Lamin B, and the like.

In some cases, the fusion partner to a CasX variant has enzymatic activity that modifies the target nucleic acid (e.g., ssRNA, dsRNA, ssDNA, dsDNA). Examples of enzymatic activity that can be provided by the fusion partner include but are not limited to: nuclease activity such as that provided by a restriction enzyme (e.g., FokI nuclease), methyltransferase activity such as that provided by a methyltransferase (e.g., Hhal DNA m5c-methyltransferase (M.Hhal), DNA methyltransferase 1 (DNMT1), DNA methyltransferase 3a (DNMT3a), DNA methyltransferase 3b (DNMT3b), MET1, DRM3 (plants), ZMET2, CMT1, CMT2 (plants), and the like); demethylase activity such as that provided by a demethylase (e.g., Ten-Eleven Translocation (TET) dioxygenase 1 (TET 1 CD), TET1, DME, DML1, DML2, ROS1, and the like), DNA repair activity, DNA damage activity, deamination activity such as that provided by a deaminase (e.g., a cytosine deaminase enzyme; e.g., an APOBEC protein such as rat apolipoprotein B mRNA editing enzyme, catalytic polypeptide 1 {APOBEC1}), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity).

In some cases, a CasX variant protein of the present disclosure is fused to a polypeptide selected from a domain for increasing transcription (e.g., a VP16 domain, a VP64 domain), a domain for decreasing transcription (e.g., a KRAB domain; e.g., from the Kox1 protein), a core catalytic domain of a histone acetyltransferase (e.g., histone acetyltransferase p300), a protein/domain that provides a detectable signal (e.g., a fluorescent protein such as GFP), a nuclease domain (e.g., a Fok1 nuclease), or a base editor (e.g., cytidine deaminase such as APOBEC1).

In some cases, a CasX variant comprises any one of SEQ ID NOS: 36-99, 101-148 or 43662-43907, or a sequence of Table 4 and a fusion partner having enzymatic activity that modifies a protein associated with the target nucleic acid (e.g., ssRNA, dsRNA, ssDNA, dsDNA) (e.g., a histone, an RNA binding protein, a DNA binding protein, and the like). Examples of enzymatic activity (that modifies a protein associated with a target nucleic acid) that can be provided by the fusion partner include but are not limited to: methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB 1, and the like, SET1A, SET1B, MLL1 to 5, ASH1, SYMD2, NSD1, DOT1L, Pr-SET7/8, SUV4-20H1, EZH2, RIZ1), demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3, and the like), acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HB01/MYST2, HMOF/MYST1, SRC1, ACTR, P160, CLOCK, and the like), deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11, and the like), kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

Additional examples of suitable fusion partners are (i) a dihydrofolate reductase (DHFR) destabilization domain (e.g., to generate a chemically controllable subject RNA-guided polypeptide or a conditionally active RNA-guided polypeptide), and (ii) a chloroplast transit peptide. In some cases, a CasX variant comprises any one of SEQ ID NOS: 59, 72-99, 101-148, or 43662-43907 and a fusion partner having enzymatic activity that modifies a protein associated with the target nucleic acid. In some cases, a CasX variant comprises any one of SEQ ID NOS: 132-148 or 43662-43907 and a fusion partner having enzymatic activity that modifies a protein associated with the target nucleic acid.

In some embodiments, a CasX variant comprises any one of SEQ ID NOS: 36-99, 101-148 or 43662-43907, or a sequence of Table 4, and a chloroplast transit peptide including, but are not limited to:

(SEQ ID NO: 478)
MASMISSSAVTTVSRASRGQSAAMAPFGGLKSMTGFPVRKVNTDITSITS
NGGRVKCMQVWPPIGKKKFETLSYLPPLTRDSRA;
(SEQ ID NO: 479)
MASMISSSAVTTVSRASRGQSAAMAPFGGLKSMTGFPVRKVNTDITSITS
NGGRVKS;
(SEQ ID NO: 480)
MASSMLSSATMVASPAQATMVAPFNGLKSSAAFPATRKANNDITSITSNG
GRVNCMQVWPPIEKKKFETLSYLPDLTDSGGRVNC;
(SEQ ID NO: 481)
MAQVSRICNGVQNPSLISNLSKSSQRKSPLSVSLKTQQHPRAYPISSSWG
LKKSGMTLIGSELRPLKVMSSVSTAC;
(SEQ ID NO: 482)
MAQVSRICNGVWNPSLISNLSKSSQRKSPLSVSLKTQQHPRAYPISSSWG
LKKSGMTLIGSELRPLKVMSSVSTAC;
(SEQ ID NO: 483)
MAQINNMAQGIQTLNPNSNFHKPQVPKSSSFLVFGSKKLKNSANSMLVLK
KDSIFMQLFCSFRISASVATAC;
(SEQ ID NO: 484)
MAALVTSQLATSGTVLSVTDRFRRPGFQGLRPRNPADAALGMRTVGASAA
PKQSRKPHRFDRRCLSMVV;
(SEQ ID NO: 485)
MAALTTSQLATSATGFGIADRSAPSSLLRHGFQGLKPRSPAGGDATSLSV
TTSARATPKQQRSVQRGSRRFPSVVVC;
(SEQ ID NO: 486)
MASSVLSSAAVATRSNVAQANMVAPFTGLKSAASFPVSRKQNLDITSIAS
NGGRVQC;
(SEQ ID NO: 487)
MESLAATSVFAPSRVAVPAARALVRAGTVVPTRRTSSTSGTSGVKCSAAV
TPQASPVISRSAAAA;
and
(SEQ ID NO: 488)
MGAAATSMQSLKFSNRLVPPSRRLSPVPNNVTCNNLPKSAAPVRTVKCCA
SSWNSTINGAAATTNGASAASS.

In some cases, a CasX variant comprises any one of SEQ ID NOS: 59, 72-99, 101-148, or 43662-43907 and a chloroplast transit peptide. In some cases, a CasX variant comprises any one of SEQ ID NOS: 132-148 or 43662-43907 and a chloroplast transit peptide.

In some cases, a CasX variant protein of the present disclosure can include an endosomal escape peptide. In some cases, an endosomal escape polypeptide comprises the amino acid sequence GLFXALLXLLXSLWXLLLXA (SEQ ID NO: 489), wherein each X is independently selected from lysine, histidine, and arginine. In some cases, an endosomal escape polypeptide comprises the amino acid sequence GLFHALLHLLHSLWHLLLHA (SEQ ID NO: 490), or HHHHHHHHH (SEQ ID NO: 491).

Non-limiting examples of fusion partners for use when targeting ssRNA target nucleic acids include (but are not limited to): splicing factors (e.g., RS domains); protein translation components (e.g., translation initiation, elongation, and/or release factors; e.g., eIF4G); RNA methylases; RNA editing enzymes (e.g., RNA deaminases; e.g., adenosine deaminase acting on RNA (ADAR), including A to I and/or C to U editing enzymes); helicases; RNA-binding proteins; and the like. It is understood that a heterologous polypeptide can include the entire protein or, in some cases, can include a fragment of the protein (e.g., a functional domain).

A fusion partner for a CasX variant can be any domain capable of interacting with ssRNA (which, for the purposes of this disclosure, includes intramolecular and/or intermolecular secondary structures; e.g., double-stranded RNA duplexes such as hairpins, stem-loops, etc.), whether transiently or irreversibly, directly or indirectly, including but not limited to an effector domain selected from the group comprising; endonucleases (for example RNase III, the CRR22 DYW domain, Dicer, and PIN (PilT N-terminus) domains from proteins such as SMG5 and SMG6); proteins and protein domains responsible for stimulating RNA cleavage (for example cleavage and polyadenylation specific factor {CPSF}, cleavage stimulation factor {CstF}, CFIm and CFIIm); exonucleases (for example chromatin-binding exonuclease XRN1 (XRN-1) or Exonuclease T); deadenylases (for example DNA 5′-adenosine monophosphate hydrolase {HNT3}); proteins and protein domains responsible for nonsense mediated RNA decay (for example UPF1 RNA helicase and ATPase {UPF1}, UPF2, UPF3, UPF3b, RNP SI, RNA binding motif protein 8A {Y14}, DEK proto-oncogene {DEK}, RNA-processing protein REF2 {REF2}, and Serine-arginine repetitive matrix 1 {SRm160}); proteins and protein domains responsible for stabilizing RNA (for example poly(A) binding protein cytoplasmic 1 {PABP}); proteins and protein domains responsible for repressing translation (for example argonaute RISC catalytic component 2 {Ago2} and Ago4); proteins and protein domains responsible for stimulating translation (for example Staufen); proteins and protein domains responsible for (e.g., capable of) modulating translation (e.g., translation factors such as initiation factors, elongation factors, release factors, etc.; e.g., eIF4G); proteins and protein domains responsible for polyadenylation of RNA (for example poly(A) polymerase (PAP1), PAP-associated domain-containing protein; Poly(A) RNA polymerase gld-2 {GLD-2}, and Star-PAP); proteins and protein domains responsible for polyuridinylation of RNA (for example Terminal uridylyltransferase {CID1} and terminal uridylate transferase); proteins and protein domains responsible for RNA localization (for example from insulin like growth factor 2 mRNA binding protein 1 {IMP1}, Z-DNA binding protein 1 {ZBP1}, She2p, She3p, and Bicaudal-D); proteins and protein domains responsible for nuclear retention of RNA (for example Rrp6); proteins and protein domains responsible for nuclear export of RNA (for example nuclear RNA export factor 1 {TAP}, nuclear RNA export factor 1 {NXF1}, THO Complex {THO}, TREX, REF, and Aly/REF export factor {Aly}); proteins and protein domains responsible for repression of RNA splicing (for example polypyrimidine tract binding protein 1 {PTB}, KH RNA binding domain containing, signal transduction associated 1 Sam68}, and heterogeneous nuclear ribonucleoprotein A1 {hnRNP A1}); proteins and protein domains responsible for stimulation of RNA splicing (for example serine/arginine-rich (SR) domains); proteins and protein domains responsible for reducing the efficiency of transcription (for example FUS RNA binding protein {FUS (TLS)}); and proteins and protein domains responsible for stimulating transcription (for example cyclin dependent kinase 7 {CDK7} and HIV Tat). Alternatively, the effector domain may be selected from the group comprising endonucleases; proteins and protein domains capable of stimulating RNA cleavage; exonucleases; deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc.; e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable heterologous polypeptide is a PUF RNA-binding domain, which is described in more detail in WO2012068627, which is hereby incorporated by reference in its entirety.

Some RNA splicing factors that can be used (in whole or as fragments thereof) as a fusion partner have modular organization, with separate sequence-specific RNA binding modules and splicing effector domains. For example, members of the serine/arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP A1 binds to exonic splicing silencers (ESSs) through its RRM domains and inhibits exon inclusion through a C-terminal glycine-rich domain. Some splicing factors can regulate alternative use of splice site (ss) by binding to regulatory sequences between the two alternative sites. For example, ASF/SF2 can recognize ESEs and promote the use of intron proximal sites, whereas hnRNP A1 can bind to ESSs and shift splicing towards the use of intron distal sites. One application for such factors is to generate ESFs that modulate alternative splicing of endogenous genes, particularly disease associated genes. For example, BCL2 like 1 (Bcl-x) pre-mRNA produces two splicing isoforms with two alternative 5′ splice sites to encode proteins of opposite functions. The long splicing isoform Bcl-xL is a potent apoptosis inhibitor expressed in long-lived post mitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple cc-elements that are located in either the core exon region or the exon extension region (i.e., between the two alternative 5′ splice sites). For more examples, see WO2010075303, which is hereby incorporated by reference in its entirety.

Further suitable fusion partners for use with a CasX variant include, but are not limited to proteins (or fragments thereof) that are boundary elements (e.g., CTCF), proteins and fragments thereof that provide periphery recruitment (e.g., Lamin A, Lamin B, etc.), and protein docking elements (e.g., FKBP/FRB, Pill/Abyl, etc.).

In some cases, a heterologous polypeptide (a fusion partner) for use with a CasX variant provides for subcellular localization, i.e., the heterologous polypeptide contains a subcellular localization sequence (e.g., a nuclear localization signal (NLS) for targeting to the nucleus, a sequence to keep the fusion protein out of the nucleus; e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like). In some embodiments, a subject RNA-guided polypeptide or a conditionally active RNA-guided polypeptide and/or subject CasX fusion protein does not include a NLS so that the protein is not targeted to the nucleus (which can be advantageous; e.g., when the target nucleic acid is an RNA that is present in the cytosol). In some embodiments, a fusion partner can provide a tag (i.e., the heterologous polypeptide is a detectable label) for ease of tracking and/or purification (e.g., a fluorescent protein; e.g., green fluorescent protein (GFP), yellow fluorescent protein (YFP), red fluorescent protein (RFP), cyan fluorescent protein (CFP), mCherry, tdTomato, and the like; a histidine tag; e.g., a 6XHis tag; a hemagglutinin (HA) tag; a FLAG tag; a Myc tag; and the like).

In some cases, a CasX variant protein includes (is fused to) a nuclear localization signal (NLS). In some cases, a CasX variant protein is fused to 2 or more, 3 or more, 4 or more, or 5 or more 6 or more, 7 or more, 8 or more NLSs. In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (2 or more, 3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus. In some cases, a CasX variant protein includes (is fused to) between 1 and 10 NLSs (e.g., 1-9, 1-8, 1-7, 1-6, 1-5, 2-10, 2-9, 2-8, 2-7, 2-6, or 2-5 NLSs). In some cases, a CasX variant protein includes (is fused to) between 2 and 5 NLSs (e.g., 2-4, or 2-3 NLSs).

In some cases, non-limiting examples of NLSs suitable for use with a CasX variant include sequences having at least about 80%, at least about 90%, or at least about 95% identity or are identical to sequences derived from: the NLS of the SV40 virus large T-antigen, having the amino acid sequence PKKKRKV (SEQ ID NO: 149); the NLS from nucleoplasmin (e.g., the nucleoplasmin bipartite NLS with the sequence KRPAATKKAGQAKKKK (SEQ ID NO: 150); the c-myc NLS having the amino acid sequence PAAKRVKLD (SEQ ID NO: 151) or RQRRNELKRSP (SEQ ID NO: 152); the hRNPA1 M9 NLS having the sequence NQSSNFGPMKGGNFGGRSSGPYGGGGQYFAKPRNQGGY (SEQ ID NO: 153); the sequence RMRIZFKNKGKDTAELRRRRVEVSVELRKAKKDEQILKRRNV (SEQ ID NO: 154) of the IBB domain from importin-alpha; the sequences VSRKRPRP (SEQ ID NO: 155) and PPKKARED (SEQ ID NO: 156) of the myoma T protein; the sequence PQPKKKPL (SEQ ID NO: 185) of human p53; the sequence SALIKKKKKMAP (SEQ ID NO: 157) of mouse c-abl IV; the sequences DRLRR (SEQ ID NO: 158) and PKQKKRK (SEQ ID NO: 159) of the influenza virus NS1; the sequence RKLKKKIKKL (SEQ ID NO: 160) of the Hepatitis virus delta antigen; the sequence REKKKFLKRR (SEQ ID NO: 161) of the mouse Mx1 protein; the sequence KRKGDEVDGVDEVAKKKSKK (SEQ ID NO: 162) of the human poly(ADP-ribose) polymerase; the sequence RKCLQAGMNLEARKTKK (SEQ ID NO: 163) of the steroid hormone receptors (human) glucocorticoid; the sequence PRPRKIPR (SEQ ID NO: 164) of Borna disease virus P protein (BDV-P1); the sequence PPRKKRTVV (SEQ ID NO: 165) of hepatitis C virus nonstructural protein (HCV-NS5A); the sequence NLSKKKKRKREK (SEQ ID NO: 166) of LEF1; the sequence RRPSRPFRKP (SEQ ID NO: 167) of ORF57 simirae; the sequence KRPRSPSS (SEQ ID NO: 168) of EBV LANA; the sequence KRGINDRNFWRGENERKTR (SEQ ID NO: 169) of Influenza A protein; the sequence PRPPKMARYDN (SEQ ID NO: 170) of human RNA helicase A (RHA); the sequence KRSFSKAF (SEQ ID NO: 186) of nucleolar RNA helicase II; the sequence KLKIKRPVK (SEQ ID NO: 171) of TUS-protein; the sequence PKKKRKVPPPPAAKRVKLD (SEQ ID NO: 172) associated with importin-alpha; the sequence PKTRRRPRRSQRKRPPT (SEQ ID NO: 173) from the Rex protein in HTLV-1; the sequence SRRRKANPTKLSENAKKLAKEVEN (SEQ ID NO: 43908) from the EGL-13 protein of Caenorhabditis Elegans; and the sequences KTRRRPRRSQRKRPPT (SEQ ID NO: 175), RRKKRRPRRKKRR (SEQ ID NO: 176), PKKKSRKPKKKSRK (SEQ ID NO: 177), HKKKHPDASVNFSEFSK (SEQ ID NO: 178), QRPGPYDRPQRPGPYDRP (SEQ ID NO: 179), LSPSLSPLLSPSLSPL (SEQ ID NO: 180), RGKGGKGLGKGGAKRHRK (SEQ ID NO: 181), PKRGRGRPKRGRGR (SEQ ID NO: 182), PKKKRKVPPPPAAKRVKLD (SEQ ID NO: 183), PKKKRKVPPPPKKKRKV (SEQ ID NO: 184), PAKRARRGYKC (SEQ ID NO: 43909), KLGPRKATGRW (SEQ ID NO: 43910), PRRKREE (SEQ ID NO: 43911), PYRGRKE (SEQ ID NO: 43912), PLRKRPRR (SEQ ID NO: 43913), PLRKRPRRGSPLRKRPRR (SEQ ID NO: 43914), PAAKRVKLDGGKRTADGSEFESPKKKRKV (SEQ ID NO: 43915), PAAKRVKLDGGKRTADGSEFESPKKKRKVGIHGVPAA (SEQ ID NO: 43916), PAAKRVKLDGGKRTADGSEFESPKKKRKVAEAAAKEAAAKEAAAKA (SEQ ID NO: 43917), PAAKRVKLDGGKRTADGSEFESPKKKRKVPG (SEQ ID NO: 43918), KRKGSPERGERKRHW (SEQ ID NO: 43919), KRTADSQHSTPPKTKRKVEFEPKKKRKV (SEQ ID NO: 43920), and PKKKRKVGGSKRTADSQHSTPPKTKRKVEFEPKKKRKV (SEQ ID NO: 43921). In some embodiments, the one or more NLS are linked to the CRISPR protein or to adjacent NLS with a linker peptide wherein the linker peptide is selected from the group consisting of RS, (G)n (SEQ ID NO: 43922), (GS)n (SEQ ID NO: 43923), (GSGGS)n (SEQ ID NO: 43924), (GGSGGS)n (SEQ ID NO: 43925), (GGGS)n (SEQ ID NO: 43926), GGSG (SEQ ID NO: 203), GGSGG (SEQ ID NO: 204), GSGSG (SEQ ID NO: 205), GSGGG (SEQ ID NO: 366), GGGSG (SEQ ID NO: 367), GSSSG (SEQ ID NO: 368), GPGP (SEQ ID NO: 369), GGP, PPP, PPAPPA (SEQ ID NO: 370), PPPG (SEQ ID NO: 43926), PPPGPPP (SEQ ID NO: 371), PPP(GGGS)n (SEQ ID NO: 43927), (GGGS)nPPP (SEQ ID NO: 43928), AEAAAKEAAAKEAAAKA (SEQ ID NO: 43929), and TPPKTKRKVEFE (SEQ ID NO: 43930), where n is 1 to 5. In general, NLS (or multiple NLSs) are of sufficient strength to drive accumulation of a CasX variant fusion protein in the nucleus of a eukaryotic cell. Detection of accumulation in the nucleus may be performed by any suitable technique. For example, a detectable marker may be fused to a CasX variant fusion protein such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

In some cases, a CasX variant fusion protein includes a “Protein Transduction Domain” or PTD (also known as a CPP—cell penetrating peptide), which refers to a protein, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from an extracellular space to an intracellular space, or from the cytosol to within an organelle. In some embodiments, a PTD is covalently linked to the amino terminus of a CasX variant fusion protein. In some embodiments, a PTD is covalently linked to the carboxyl terminus of a CasX variant fusion protein. In some cases, the PTD is inserted internally in the sequence of a CasX variant fusion protein at a suitable insertion site. In some cases, a CasX variant fusion protein includes (is conjugated to, is fused to) one or more PTDs (e.g., two or more, three or more, four or more PTDs). In some cases, a PTD includes one or more nuclear localization signals (NLS). Examples of PTDs include but are not limited to peptide transduction domain of HIV TAT comprising YGRKKRRQRRR (SEQ ID NO: 191), RKKRRQRR (SEQ ID NO: 192); YARAAARQARA (SEQ ID NO: 193); THRLPRRRRRR (SEQ ID NO: 194); and GGRRARRRRRR (SEQ ID NO: 195); a polyarginine sequence comprising a number of arginines sufficient to direct entry into a cell (e.g., 3, 4, 5, 6, 7, 8, 9, 10, or 10-50 arginines (SEQ ID NO: 43570)); a VP22 domain (Zender et al. (2002) Cancer Gene Ther. 9(6):489-96); a Drosophila Antennapedia protein transduction domain (Noguchi et al. (2003) Diabetes 52(7): 1732-1737); a truncated human calcitonin peptide (Trehin et al. (2004) Pharm. Research 21:1248-1256); polylysine (Wender et al. (2000) Proc. Natl. Acad. Sci. USA 97: 13003-13008); RRQRRTSKLMKR (SEQ ID NO: 196); Transportan GWTLNSAGYLLGKINLKALAALAKKIL (SEQ ID NO: 197); KALAWEAKLAKALAKALAKHLAKALAKALKCEA (SEQ ID NO: 198); and RQIKIWFQNRRMKWKK (SEQ ID NO: 199). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

In some embodiments, a CasX variant fusion protein can include a CasX protein that is linked to an internally inserted heterologous amino acid or heterologous polypeptide (a heterologous amino acid sequence) via a linker polypeptide (e.g., one or more linker polypeptides). In some embodiments, a CasX variant fusion protein can be linked at the C-terminal and/or N-terminal end to a heterologous polypeptide (fusion partner) via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers are generally produced by using synthetic, linker-encoding oligonucleotides to couple the proteins. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use. Exemplary linker polypeptides include peptides selected from the group consisting of RS, (G)n, (GS)n, (GSGGS)n (SEQ ID NO: 200), (GGSGGS)n (SEQ ID NO: 201), and (GGGS)n (SEQ ID NO: 202), where n is an integer of at least one), glycine-alanine polymers, alanine-serine polymers, glycine-proline polymers, proline polymers and proline-alanine polymers. Example linkers can comprise amino acid sequences including, but not limited to, GGSG (SEQ ID NO: 203), GGSGG (SEQ ID NO: 204), GSGSG (SEQ ID NO: 205), GSGGG (SEQ ID NO: 366), GGGSG (SEQ ID NO: 367), GSSSG (SEQ ID NO: 368), GPGP (SEQ ID NO: 369), GGP, PPP, PPAPPA (SEQ ID NO: 370), PPPGPPP (SEQ ID NO: 371), PPP(GGGS)n (SEQ ID NO: 43927), (GGGS)nPPP (SEQ ID NO: 43928), AEAAAKEAAAKEAAAKA (SEQ ID NO: 43929), and TPPKTKRKVEFE (SEQ ID NO: 43930), where n is 1 to 5. and the like. The ordinarily skilled artisan will recognize that design of a peptide conjugated to any elements described above can include linkers that are all or partially flexible, such that the linker can include a flexible linker as well as one or more portions that confer less flexible structure.

### III. Systems and Methods for Modification of PTBP1 Genes

The CRISPR proteins, guide nucleic acids, and variants thereof provided herein are useful for various applications, including as therapeutics, diagnostics, and for research. To effect the methods of the disclosure for gene editing, resulting in modification of the gene, provided herein are programmable Class 2, Type V CRISPR systems. The programmable nature of the systems provided herein allows for the precise targeting to achieve the desired effect (nicking, cleaving, etc.) at one or more regions of predetermined interest in the PTBP1 gene target nucleic acid. A variety of strategies and methods can be employed to modify the target nucleic acid sequence in a cell using the systems provided herein. As used herein “modifying” includes, but is not limited to, cleaving, nicking, editing, deleting, knocking out, knocking down, mutating, correcting, exon-skipping and the like. Depending on the system components utilized, the editing event may be a cleavage event followed by introducing random insertions or deletions (indels) or other mutations (e.g., a substitution, duplication, or inversion of one or more nucleotides), for example by utilizing the imprecise non-homologous DNA end joining (NHEJ) repair pathway, which may generate, for example, a frame shift mutation. Alternatively, the editing event may be a cleavage event followed by homology-directed repair (HDR), homology-independent targeted integration (HITI), micro-homology mediated end joining (MMEJ), single strand annealing (SSA) or base excision repair (BER), resulting in modification of the target nucleic acid sequence.

In some embodiments, the disclosure provides methods of modifying a PTBP1 target nucleic acid in a cell, the method comprising introducing into the cell a Class 2, Type V CRISPR system. In some embodiments, the disclosure provides methods of modifying a PTBP1 target nucleic acid in a cell, the method comprising introducing into the cell: i) a CasX:gRNA system comprising a CasX and a gRNA of any one of the embodiments described herein; ii) a CasX:gRNA system comprising a CasX, a gRNA, and a donor template of any one of the embodiments described herein; iii) a nucleic acid encoding the CasX and the gRNA, and optionally comprising the donor template; iv) a vector comprising the nucleic acid of (iii), above; v) a XDP comprising the CasX:gRNA system of any one of the embodiments described herein; or vi) combinations of two or more of (i) to (v), wherein the target nucleic acid sequence of the cell is modified by the CasX protein and, optionally, the donor template. In some embodiments, the disclosure provides CasX:gRNA systems for use in the methods of modifying the PTBP1 gene in a cell, wherein the system comprises a CasX variant comprising a sequence selected from the group consisting of the sequences of SEQ ID NOS: 36-99, 101-148 and 43662-43907 as set forth in Table 4, a CasX variant comprising a sequence selected from the group consisting of the sequences of SEQ ID NOS: 59, 72-99, 101-148, and 43662-43907, a CasX variant comprising a sequence selected from the group consisting of the sequences of SEQ ID NOS: 132-148 and 43662-43907, or a sequence having at least about 50%, at least about 60%, at least about 70%, at least about 80%, at least about 90%, or at least about 95%, or at least about 95%, or at least about 96%, or at least about 97%, or at least about 98%, or at least about 99% sequence identity thereto, the gRNA scaffold comprises a sequence selected from the group consisting of the sequences of SEQ ID NOS: 2101-2285, 43571-43661, 44045, and 44047 as set forth in Table 3, the gRNA scaffold comprises a sequence selected from the group consisting of the sequences of SEQ ID NOS: 2238-2285, 43571-43661, 44045 and 44047, the gRNA scaffold comprises a sequence selected from the group consisting of the sequences of SEQ ID NOS: 2281-2285, 43571-43661, 44045, and 44047 or a sequence at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical thereto, and the gRNA comprises a targeting sequence selected from the group consisting of the sequence of SEQ ID NOS: 492-2100 and 2286-43569 or a sequence at least 65% identical, at least 70% identical, at least 75% identical, at least 80% identical, at least 85% identical, at least 90% identical, or at least 95% identical thereto and having between 15 and 30 amino acids. In a particular embodiment, the targeting sequence of the gRNA of the CasX:gRNA system is selected from the group consisting of the sequence of SEQ ID NOS: 37971-37979, 38027, 38136, 38137, 38152-38158, 38160, 38162-38174, 38176, 38177, 38181, 38195, 38196, 38198, 38199, 38253-38256, 38259-38267, 38306 and 38311-38353. In a further particular embodiment, the CasX variant comprises a sequence selected from the group consisting of the sequences of SEQ ID NOS: 132-148 and 43662-43907, the gRNA scaffold comprises a sequence selected from the group consisting of the sequences of SEQ ID NOS: 2281-2285, 43571-43661 and 44045, and the targeting sequence of the gRNA of the CasX:gRNA system is selected from the group consisting of the sequence of SEQ ID NOS: 37971-37979, 38027, 38136, 38137, 38152-38158, 38160, 38162-38174, 38176, 38177, 38181, 38195, 38196, 38198, 38199, 38253-38256, 38259-38267, 38306 and 38311-38353.

In those cases where the CasX is delivered to the cell in the protein form and the gRNA is delivered in the RNA form, the CasX and gRNA can be pre-complexed and delivered as an RNP. Upon hybridization with the target nucleic acid by the CasX and the gRNA, the CasX introduces one or more single-strand breaks or double-strand breaks within or near the PTBP1 gene that result in a modification of the target nucleic acid such as a permanent indel (deletion or insertion) or other mutation (a base change, inversion or rearrangement with respect to the genomic sequence) in the target nucleic acid, as described herein, with a corresponding modulation of expression or alteration in the function of the PTBP1 gene product, thereby creating an edited cell. In other embodiments, the method comprises contacting the target nucleic acid sequence with a plurality of gRNAs targeted to different or overlapping portions of the PTBP1 gene wherein the CasX protein introduces multiple breaks in the target nucleic acid sequence that result in a permanent indel (deletion or insertion) or other mutation in the target nucleic acid, as described herein, with a corresponding modulation of expression or alteration in the function of the PTBP1 gene product, thereby creating an edited cell.

In some cases, the CasX:gRNA system for use in the methods of modifying the PTBP1 gene further comprises a donor template nucleic acid of any of the embodiments disclosed herein, wherein the donor template can be inserted by the homology-directed repair (HDR) or homology-independent targeted integration (HITI) repair mechanisms of the host cell. The donor template can be a short single-stranded or double-stranded oligonucleotide, or a long single-stranded or double-stranded oligonucleotide. The donor template may contain one or more single base changes, insertions, deletions, inversions or rearrangements with respect to the genomic sequence, provided that there is sufficient homology with the target nucleic acid sequence to support its integration into the target nucleic acid, which can result in a frame-shift or other mutation such that the PTBP1 protein is not expressed or is expressed at a lower level. In some embodiments, the donor template sequence comprises a non-homologous sequence flanked by two regions of homology to the break sites of the target nucleic acid, facilitating insertion of the non-homologous sequence at the target region which can be mediated by HDR or HITI. The exogenous donor template inserted by HITI can be any length, for example, a relatively short sequence of between 10 and 50 nucleotides in length, or a longer sequence of about 50-1000 nucleotides in length. The lack of homology can be, for example, having no more than 20-50% sequence identity and/or lacking in specific hybridization at low stringency. In other cases, the lack of homology can further include a criterion of having no more than 5, 6, 7, 8, or 9 bp identity. The donor template sequence inserted by HDR comprises a sequence flanked by two regions of homology (“homologous arms”) to the 5′ and 3′ sides of the break site(s) such that the repair mechanisms between the target DNA region and the two flanking sequences results in insertion of the donor template at the target region. In some embodiments, the donor template polynucleotide comprises at least about 10, at least about 50, at least about 100, or at least about 200, or at least about 300, or at least about 400, or at least about 500, or at least about 600, or at least about 700, or at least about 800, or at least about 900, or at least about 1000, or at least about 10,000, or at least about 15,000 nucleotides. In other embodiments, the donor template comprises at least about 10 to about 15,000 nucleotides, or at least about 100 to about 10,000 nucleotides, or at least about 400 to about 8,000 nucleotides, or at least about 600 to about 5000 nucleotides, or at least about 1000 to about 2000 nucleotides. The donor template sequence may comprise certain sequence differences as compared to the genomic sequence; e.g., restriction sites, nucleotide polymorphisms, selectable markers (e.g., drug resistance genes, fluorescent proteins, enzymes etc.), etc., which may be used to assess for successful insertion of the donor nucleic acid at the cleavage site or in some cases may be used for other purposes (e.g., to signify expression at the targeted genomic locus). Alternatively, these sequence differences may include flanking recombination sequences such as FLPs, loxP sequences, or the like, that can be activated at a later time for removal of the marker sequence.

In some embodiments, the method of the disclosure provides CasX protein and gRNA pairs that generate site-specific double strand breaks (DSBs) or single strand breaks (SSBs) (e.g., when the CasX protein is a nickase that can cleave only one strand of a target nucleic acid) within double-stranded DNA (dsDNA) target nucleic acids, which can then be repaired either by non-homologous end joining (NHEJ), homology-directed repair (HDR), homology-independent targeted integration (HITI), micro-homology mediated end joining (MMEJ), single strand annealing (SSA) or base excision repair (BER). In some cases, contacting a PTBP1 gene with a gene editing pair occurs under conditions that are permissive for non-homologous end joining or homology-directed repair. Thus, in some cases, the methods provided herein include contacting the PTBP1 gene with a donor template by introducing the donor template (either in vitro outside of a cell, in vitro inside a cell, in vivo inside a cell, or ex vivo), wherein the donor template, a portion of the donor template, a copy of the donor template, or a portion of a copy of the donor template integrates into the PTBP1 gene to replace a portion of the PTBP1 gene.

In some embodiments of the method of modifying a PTBP1 target nucleic acid of a cell in vitro or ex vivo, to induce cleavage or any desired modification to a target nucleic acid, the gRNA and/or the CasX protein of the present disclosure and, optionally, the donor template sequence, whether they be introduced as nucleic acids or polypeptides, vectors or XDPXDP, are provided to the cells for about 30 minutes to about 24 hours, or at least about 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days; e.g., every 1.5 days, every 2 days, every 3 days, or any other frequency from about every day to about every four days. The agent(s) may be provided to the subject cells one or more times; e.g., one time, twice, three times, or more than three times, and the cells allowed to incubate with the agent(s) for some amount of time following each contacting event e.g., 30 minutes to about 24 hours. In the case of in vitro-based methods, after the incubation period with the CasX and gRNA (and optionally the donor template), the media is replaced with fresh media and the cells are cultured further.

In some embodiments of the method of modifying a PTBP1 target nucleic acid in a cell, the method further comprises contacting the target nucleic acid sequence of the cell with: a) an additional CRISPR nuclease and a gRNA targeting a different or overlapping portion of the PTBP1 target nucleic acid compared to the first gRNA; b) a polynucleotide encoding the additional CRISPR nuclease and the gRNA of (a); c) a vector comprising the polynucleotide of (b); or d) a XDP comprising the additional CRISPR nuclease and the gRNA of (a), wherein the contacting results in modification of the PTBP1 target nucleic acid at a different location in the sequence compared to the first gRNA. In some cases, the additional CRISPR nuclease is a CasX protein having a sequence different from the CasX protein of any of the preceding claims. In other cases, the additional CRISPR nuclease is not a CasX protein and the additional CRISPR nuclease is selected from the group consisting of Cas9, Cas12a, Cas12b, Cas12c, Cas12d (CasY), Cas12j, Cas12k, Cas13a, Cas13b, Cas13c, Cas13d, CasY, Cas14, Cpf1, C2c1, Csn2, C2c4, C2c8, C2c5, C2c10, C2c9, Cas Phi, and sequence variants thereof.

In those cases where the modification results in a knock-down of the PTBP1 gene, expression of the PTBP1 protein is reduced by at least about 10%, at least about 20%, at least about 30%, at least about 40%, at least about 50%, at least about 60%, at least about 70%, at least about 80%, or at least about 90% in comparison to cells that have not been modified. In other cases, wherein the modification results in a knock-out of the PTBP1 gene, the target nucleic acid of the cells of the population is modified such that at least about 10%, at least about 20%, at least about 30%, at least about 40%, at least about 50%, at least about 60%, at least about 70%, at least about 80%, or at least about 90% of the cells do not express a detectable level of PTBP1 protein. Expression of a PTBP1 protein can be measured by flow cytometry, ELISA, cell-based assays, Western blot or other methods know in the art (Cho, C., et al. PTBP1-mediated regulation of AXL mRNA stability plays a role in lung tumorigenesis. Scientific Reports 9:16922 (2019)), or as described in the Examples.

In other embodiments of the method of modifying a target nucleic acid sequence, modifying the PTBP1 gene comprises binding of a CasX to the target nucleic acid sequence without cleavage. In some embodiments, the CasX is a catalytically inactive CasX (dCasX) protein that retains the ability to bind to the gRNA and to the PTBP1 target nucleic acid sequence but lacks the ability to cleave the nucleic acid sequence, thereby interfering with transcription of the PTBP1 allele. In some embodiments, the dCasX comprises a mutation at residues D672, E769, and/or D935 corresponding to the CasX protein of SEQ ID NO:1 or D659, E756 and/or D922 corresponding to the CasX protein of SEQ ID NO: 2. In some embodiments, the mutation is a substitution of alanine or glycine for the residue.

In some embodiments, the disclosure provides methods of modifying a PTBP1 target nucleic acid in a population of cells in vivo in a subject. In some embodiments of the method, the modifying comprises contacting the cells of the subject with a therapeutically effective dose of i) the system comprising a CasX variant and gRNA variant and, optionally, a donor template nucleic acid of any of the embodiments described herein; ii) nucleic acid encoding the system of (i); a vector comprising the nucleic acid of (ii); iii) an XDP comprising RNPs of the system of (i); or combinations of two or more of (i)-(iii), wherein the PTBP1 gene of the cells targeted by the gRNA is modified by the CasX variant protein. In some embodiments of the method, the modified cells of the population are eukaryotic, which can include rodent cells, mouse cells, rat cells, primate cells, non-human primate cells, human cells, central nervous system (CNS) cells, and peripheral nervous system (PNS) cells. In the case of cells of the CNS or PNS to be modified by the methods of the embodiments, the cells can be astrocytes, oligodendrocytes, glial cells, microglial cells, or fibroblasts. In some embodiments of the method, the modification of the PTBP1 target nucleic acid sequence and the down-regulation of PTBP1 results in reprogramming or conversion of the eukaryotic cells into functional neurons that then express nPTB (also known as PTBP2). In some embodiments of the method, the reprogramming of the eukaryotic cells into functional neurons results in the prevention or amelioration of the neurologic disease of the subject.

In other cases, the cells to be modified are cancer cells, which can include cells of a tumor, wherein the in vivo modification of the PTBP1 target nucleic acid sequence prevents or reduces tumorigenesis of the cells, or results in stasis of an existing tumor in a subject. In some embodiments, the disclosure provides methods of modifying a PTBP1 target nucleic acid in a population of tumor cells in vivo in a subject comprising contacting the tumor cells of the subject with a therapeutically effective dose of i) the system comprising a CasX variant and gRNA variant and, optionally, a donor template nucleic acid of any of the embodiments described herein; ii) nucleic acid encoding the system of (i); a vector comprising the nucleic acid of (ii); iii) an XDP comprising RNPs of the system of (i); or combinations of two or more of (i)-(iii), wherein the PTBP1 gene of the cells targeted by the gRNA is modified by the CasX variant protein. In the foregoing embodiment, the method results in stasis of an existing tumor for at least about 1 month, at least about 2 months, at least about 3 months, at least about 4 months, at least about 6 months, at least about 7 months, at least about 8 months, at least about 9 months, at least about 10 months, at least about 11 months, at least about 12 months, at least about 18 months, at least about 2 years, at least about 3 years, at least about 4 years, or at least about 5 years. In another embodiment, the method results in stasis of an existing tumor for at least about 1 to about 5 years, at least about 6 months to about 4 years, or at least about 1 year to about 3 years.

Introducing recombinant expression vectors comprising the components or the nucleic acids encoding the components of the system embodiments into a target cell can be carried out in vivo, in vitro or ex vivo. In some embodiments of the method, vectors may be provided directly to a target host cell. Methods of introducing a nucleic acid (e.g., a nucleic acid comprising a donor polynucleotide sequence, one or more nucleic acids (DNA or RNA) encoding a CasX protein and/or gRNA, or a vector comprising same) into a cell are known in the art, and any convenient method can be used to introduce a nucleic acid (e.g., an expression construct) into a cell. Suitable methods include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, nucleofection, electroporation, direct addition by cell penetrating CasX proteins that are fused to or recruit donor DNA, cell squeezing, calcium phosphate precipitation, direct microinjection, nanoparticle-mediated nucleic acid delivery, and the like. Nucleic acids may be introduced into the cells using well-developed commercially-available transfection techniques such as use of TransMessenger® reagents from Qiagen, Stemfect™ RNA Transfection Kit from Stemgent, and TransIT®-mRNA Transfection Kit from Mirus Bio LLC, Lonza nucleofection, Maxagen electroporation and the like. Introducing recombinant expression vectors comprising sequences encoding the CasX:gRNA systems (and, optionally, the donor sequences) of the disclosure into cells under in vitro conditions can occur in any suitable culture media and under any suitable culture conditions that promote the survival of the cells. For example, cells may be contacted with vectors comprising the subject nucleic acids (e.g., recombinant expression vectors having the donor template sequence and nucleic acid encoding the CasX and gRNA) such that the vectors are taken up by the cells. Vectors used for providing the nucleic acids encoding gRNAs and/or CasX proteins to a target host cell can include suitable promoters for driving the expression, that is, transcriptional activation of the nucleic acid of interest. In some cases, the encoding nucleic acid of interest will be operably linked to a promoter. This may include ubiquitously acting promoters, for example, the CMV-beta-actin promoter, or inducible promoters, such as promoters that are active in particular cell populations or that respond to the presence of drugs such as tetracycline or kanamycin. By transcriptional activation, it is intended that transcription will be increased above basal levels in the target host cell comprising the vector by at least about 10-fold, by at least about 100-fold, more usually by at least about 1000-fold. In addition, vectors used for providing a nucleic acid encoding a gRNA and/or a CasX protein to a cell may include nucleic acid sequences that encode for selectable markers in the target cells, so as to identify cells that have taken up the CasX protein and/or the gRNA.

For viral vector delivery, cells can be contacted with viral particles comprising the subject viral expression vectors and the nucleic acid encoding the CasX and gRNA and, optionally, the donor template. In some embodiments, the vector is an Adeno-Associated Viral (AAV) vector, wherein the AAV is selected from AAV1, AAV2, AAV3, AAV4, AAV5, AAV6, AAV7, AAV8, AAV9, AAV10, AAV11, AAV12, AAV-Rh74, AAVRh10, or a hybrid, a derivative or variant thereof. In other embodiments, the vector is a retroviral vector, described more fully, below. In other embodiments, the vector is a lentiviral vector. Retroviruses, for example, lentiviruses, may be suitable for use in methods of the present disclosure. Commonly used retroviral vectors are “defective”; e.g., are unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, and this envelope protein determines the specificity or tropisms of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Nucleic acids can also be introduced by direct micro-injection (e.g., injection of RNA).

### IV. Polynucleotides and Vectors

In some embodiments, the present disclosure provides polynucleotides encoding the Class 2, Type V CRISPR proteins and the polynucleotides of the gRNA that have utility in the editing of the PTBP1 gene. In other embodiments, the present disclosure provides polynucleotides encoding the CasX proteins and the polynucleotides of the gRNAs (e.g., the gDNAs and gRNAs) described herein. In an additional embodiment, the disclosure provides donor template polynucleotides encoding portions or all of an PTBP1 gene. In some cases, the donor template comprises a mutation or a heterologous sequence for knocking down or knocking out the PTBP1 gene upon its insertion in the target nucleic acid. In yet further embodiments, the disclosure relates to vectors comprising polynucleotides encoding the CasX proteins and the gRNAs described herein. In yet further embodiments, the disclosure provides vectors comprising the donor templates described herein.

In some embodiments, the disclosure provides polynucleotide sequences encoding the CasX variants of any of the embodiments described herein, including the CasX protein variants of SEQ ID NOS: 36-99, 101-148 or 43662-43907 as described in Table 4, or sequences having at least about 50%, at least about 60%, at least about 70%, at least about 80%, or at least about 90%, or at least about 95%, or at least about 96%, or at least about 97%, or at least about 98%, or at least about 99% sequence identity thereto. In some embodiments, the disclosure provides polynucleotide sequences encoding the CasX variants of any of the embodiments described herein, including the CasX protein variants of SEQ ID NOS: 59, 72-99, 101-148, or 43662-43907, or sequences having at least about 50%, at least about 60%, at least about 70%, at least about 80%, or at least about 90%, or at least about 95%, or at least about 96%, or at least about 97%, or at least about 98%, or at least about 99% sequence identity thereto. In some embodiments, the disclosure provides polynucleotide sequences encoding the CasX variants of any of the embodiments described herein, including the CasX protein variants of SEQ ID NOS: 132-148 or 43662-43907, or sequences having at least about 50%, at least about 60%, at least about 70%, at least about 80%, or at least about 90%, or at least about 95%, or at least about 96%, or at least about 97%, or at least about 98%, or at least about 99% sequence identity thereto. In some embodiments, the disclosure provides an isolated polynucleotide sequence encoding a gRNA scaffold sequence of any of the embodiments described herein, including the sequences of SEQ ID NOS: 4-16, 2101-2285, 43571-43661 or 44045, together with sequences encoding the targeting sequences selected from the group consisting of SEQ ID NOS: 492-2100 and 2286-43569. In some embodiments, the disclosure provides an isolated polynucleotide sequence encoding a gRNA scaffold sequence of any of SEQ ID NOS: 2238-2285, 43571-43661, 44045 and 44047, together with sequences encoding the targeting sequences selected from the group consisting of SEQ ID NOS: 492-2100 and 2286-43569. In some embodiments, the disclosure provides an isolated polynucleotide sequence encoding a gRNA scaffold sequence of any of SEQ ID NOS: 2281-2285, 43571-43661 or 44045, together with sequences encoding the targeting sequences selected from the group consisting of SEQ ID NOS: 492-2100 and 2286-43569. In a particular embodiment of the foregoing, the encoded targeting sequence is selected from the group consisting of the sequence of SEQ ID NOS: 37971-37979, 38027, 38136, 38137, 38152-38158, 38160, 38162-38174, 38176, 38177, 38181, 38195, 38196, 38198, 38199, 38253-38256, 38259-38267, 38306 and 38311-38353. In some embodiments, the sequences encoding the CasX protein are codon optimized for expression in a eukaryotic cell.

In some embodiments, the polynucleotide encodes a gRNA scaffold sequence of SEQ ID NOS: 4-16, 2101-2285, 43571-43661, 44045, or 44047 as set forth in Table 2 or Table 3, or a sequence having at least about 50%, at least about 60%, at least about 70%, at least about 80%, at least about 90%, at least about 95%, at least about 96%, at least about 97%, at least about 98%, at least about 99% sequence identity thereto. In some embodiments, the polynucleotide encodes a gRNA scaffold sequence of SEQ ID NOS: 2238-2285, 43571-43661, 44045 and 44047, or a sequence having at least about 50%, at least about 60%, at least about 70%, at least about 80%, at least about 90%, at least about 95%, at least about 96%, at least about 97%, at least about 98%, at least about 99% sequence identity thereto. In some embodiments, the polynucleotide encodes a gRNA scaffold sequence of SEQ ID NOS: 2281-2285, 43571-43661 or 44045, or a sequence having at least about 50%, at least about 60%, at least about 70%, at least about 80%, at least about 90%, at least about 95%, at least about 96%, at least about 97%, at least about 98%, at least about 99% sequence identity thereto. In other embodiments, the disclosure provides a polynucleotide sequence encoding a targeting sequence selected from the group consisting of SEQ ID NOS: 492-2100 and 2286-43569, or a sequence having at least about 65%, at least about 75%, at least about 85%, or at least about 95% identity to a sequence selected from the group consisting of SEQ ID NOS: 492-2100 and 2286-43569. In a particular embodiment of the foregoing, the encoded targeting sequence is selected from the group consisting of the sequence of SEQ ID NOS: 37971-37979, 38027, 38136, 38137, 38152-38158, 38160, 38162-38174, 38176, 38177, 38181, 38195, 38196, 38198, 38199, 38253-38256, 38259-38267, 38306 and 38311-38353. In some embodiments, the targeting sequence polynucleotide is, in turn, linked to the gRNA scaffold sequence; either as a sgRNA or a dgRNA. In other embodiments, the disclosure provides gRNAs comprising targeting sequence polynucleotides having one or more single nucleotide polymorphisms (SNP) relative to a sequence selected from the group consisting of SEQ ID NOS: 492-2100 and 2286-43569. In other embodiments, the disclosure provides gRNAs comprising targeting sequence polynucleotides having one or more single nucleotide polymorphisms (SNP) relative to a sequence selected from the group consisting of SEQ ID NOS: 37971-37979, 38027, 38136, 38137, 38152-38158, 38160, 38162-38174, 38176, 38177, 38181, 38195, 38196, 38198, 38199, 38253-38256, 38259-38267, 38306 and 38311-38353.

In other embodiments, the disclosure provides an isolated polynucleotide sequence encoding a gRNA comprising a targeting sequence that is complementary to, and therefore hybridizes with the PTBP1 gene. In other embodiments, the polynucleotide sequence encodes a gRNA comprising a targeting sequence that hybridizes with a PTBP1 exon; e.g., any one of exons 1-16. In other embodiments, the polynucleotide sequence encodes a gRNA comprising a targeting sequence that hybridizes with a PTBP1 intron. In other embodiments, the polynucleotide sequence encodes a gRNA comprising a targeting sequence that hybridizes with a PTBP1 intron-exon junction. In other embodiments, the polynucleotide sequence encodes a gRNA comprising a targeting sequence that hybridizes with an intergenic region of the PTBP1 gene. In other embodiments, the polynucleotide sequence encodes a gRNA comprising a targeting sequence that hybridizes with a PTBP1 regulatory element. In some cases, the PTBP1 regulatory element is 5′ of the PTBP1 gene. In other cases, the PTBP1 regulatory element is 3′ of the PTBP1 gene. In some cases, the PTBP1 regulatory element is in an intron of the PTBP1 gene. In other cases, the PTBP1 regulatory element comprises the 5′ UTR of the PTBP1 gene. In still other cases, the PTBP1 regulatory element comprises the 3′UTR of the PTBP1 gene. In some cases of the foregoing embodiments, the PTBP1 sequence is a wild-type sequence.

In other embodiments, the disclosure provides donor template nucleic acids, wherein the donor template comprises a nucleotide sequence having homology to a PTBP1 target nucleic acid sequence. In some embodiments, the PTBP1 donor template is intended for gene editing and comprises at least a portion of a PTBP1 gene. In some embodiments, the PTBP1 donor template comprises a sequence that hybridizes with the PTBP1 gene. In other embodiments, the PTBP1 donor sequence comprises a sequence that encodes at least a portion of a PTBP1 exon selected from the group consisting of PTBP1 exon 1, PTBP1 exon 2, PTBP1 exon 3, PTBP1 exon 4, PTBP1 exon 5, PTBP1 exon 6, PTBP1 exon 7, PTBP1 exon 8, PTBP1 exon 9, PTBP1 exon 10, PTBP1 exon 11, PTBP1 exon 12, PTBP1 exon 13, PTBP1 exon 14, PTBP1 exon 15, and PTBP1 exon 16. In other embodiments, the PTBP1 donor sequence has a sequence that encodes at least a portion of a PTBP1 intron. In other embodiments, the PTBP1 donor sequence has a sequence that encodes at least a portion of with a PTBP1 intron-exon junction. In other embodiments, the PTBP1 donor sequence has a sequence that encodes at least a portion of an intergenic region of the PTBP1 gene. In other embodiments, the PTBP1 donor sequence has a sequence that encodes at least a portion of a PTBP1 regulatory element. In some cases of the foregoing donor template embodiments, the PTBP1 sequence comprises one or more mutations relative to a wild-type PTBP1 gene such that upon its insertion into the PTBP1 gene, the gene is knocked down or knocked out, with a resulting loss of expression of the PTBP1 protein. In the foregoing embodiments, the donor template is at least 10 nucleotides, at least 100 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1,000 nucleotides, at least 2,000 nucleotides, at least 3,000 nucleotides, at least 4,000 nucleotides, at least 5,000 nucleotides, at least 6,000 nucleotides, at least 7,000 nucleotides, at least 8,000 nucleotides, at least 9,000 nucleotides, at least 10,000 nucleotides, at least 12,000 nucleotides, or at least 15,000 nucleotides. In some embodiments, the donor template comprises at least about 10 to about 15,000 nucleotides. In some embodiments, the donor template is a single-stranded DNA template. In other embodiments, the donor template is a single stranded RNA template. In other embodiments, the donor template is a double-stranded DNA template. In some embodiments, the donor template can be provided as naked nucleic acid in the systems to edit the gene and does not need to be incorporated into a vector. In other embodiments, the donor template can be incorporated into a vector to facilitate its delivery to a cell; e.g., in a viral vector.

In other aspects, the disclosure relates to methods to produce polynucleotide sequences encoding the CasX variants, or the gRNA of any of the embodiments described herein, or sequences complementary to the polynucleotide sequences, including homologous variants thereof, as well as methods to express the proteins expressed or RNA transcribed by the polynucleotide sequences. In general, the methods include producing a polynucleotide sequence coding for the reference CasX, the CasX variants, or the gRNA of any of the embodiments described herein and incorporating the encoding gene into an expression vector appropriate for a host cell. For production of the encoded reference CasX, the CasX variants, or the gRNA of any of the embodiments described herein, the methods include transforming an appropriate host cell with an expression vector comprising the encoding polynucleotide, and culturing the host cell under conditions causing or permitting the resulting reference CasX, the CasX variants, or the gRNA of any of the embodiments described herein to be expressed or transcribed in the transformed host cell, thereby producing the reference CasX, the CasX variants, or the gRNA, which are recovered by methods described herein or by standard purification methods known in the art or as described in the Examples. Standard recombinant techniques in molecular biology are used to make the polynucleotides and expression vectors of the present disclosure or as described in the Examples.

In accordance with the disclosure, nucleic acid sequences that encode the reference CasX, the CasX variants, or the gRNA of any of the embodiments described herein (or their complement) are used to generate recombinant DNA molecules that direct the expression in appropriate host cells. Several cloning strategies are suitable for performing the present disclosure, many of which are used to generate a construct that comprises a gene coding for a composition of the present disclosure. In some embodiments, the cloning strategy is used to create a gene that encodes a construct that comprises nucleotides encoding the reference CasX, the CasX variants, or the gRNA that is used to transform a host cell for expression of the composition.

In some approaches, a construct is first prepared containing the DNA sequence encoding a CasX variant, or a gRNA. Exemplary methods for the preparation of such constructs are described in the Examples. The construct is then used to create an expression vector suitable for transforming a host cell, such as a prokaryotic or eukaryotic host cell for the expression and recovery of the protein construct, in the case of the CasX, or the gRNA. Where desired, the host cell is an E. coli. In other embodiments, the host cell is a eukaryotic cell. The eukaryotic host cell can be selected from Baby Hamster Kidney fibroblast (BHK) cells, human embryonic kidney 293 (HEK293), human embryonic kidney 293T (HEK293T), NS0 cells, SP2/0 cells, YO myeloma cells, P3X63 mouse myeloma cells, PER cells, PER.C6 cells, hybridoma cells, NIH3T3 cells, CV-1 (simian) in Origin with SV40 genetic material (COS), HeLa, Chinese hamster ovary (CHO), or yeast cells, or other eukaryotic cells known in the art suitable for the production of recombinant products. Exemplary methods for the creation of expression vectors, the transformation of host cells and the expression and recovery of the CasX variants and the gRNA are described in the Examples.

The gene encoding the CasX variants or the gRNA constructs can be made in one or more steps, either fully synthetically or by synthesis combined with enzymatic processes, such as restriction enzyme-mediated cloning, PCR and overlap extension, including methods more fully described in the Examples. The methods disclosed herein can be used, for example, to ligate sequences of polynucleotides encoding the various components (e.g., CasX and gRNA) genes of a desired sequence. Genes encoding polypeptide compositions are assembled from oligonucleotides using standard techniques of gene synthesis.

In some embodiments, the nucleotide sequence encoding a CasX protein is codon optimized for the intended host cell. This type of optimization can entail a mutation of an encoding nucleotide sequence to mimic the codon preferences of the intended host organism or cell while encoding the same CasX protein. Thus, the codons can be changed, but the encoded protein or gRNA remains unchanged. For example, if the intended target cell of the CasX protein was a human cell, a human codon-optimized CasX-encoding nucleotide sequence could be used. As another non-limiting example, if the intended host cell were a mouse cell, then a mouse codon-optimized CasX-encoding nucleotide sequence could be generated. The gene design can be performed using algorithms that optimize codon usage and amino acid composition appropriate for the host cell utilized in the production of the reference CasX or the CasX variants. In one method of the disclosure, a library of polynucleotides encoding the components of the constructs is created and then assembled, as described above. The resulting genes are then assembled and the resulting genes used to transform a host cell and produce and recover the CasX variants, or the gRNA compositions for evaluation of its properties, as described herein.

The disclosure provides for the use of plasmid expression vectors containing replication and control sequences that are compatible with and recognized by the host cell and are operably linked to the gene encoding the polypeptide for controlled expression of the polypeptide or transcription of the RNA. Such vector sequences are well known for a variety of bacteria, yeast, and viruses. Useful expression vectors that can be used include, for example, segments of chromosomal, non-chromosomal and synthetic DNA sequences. “Expression vector” refers to a DNA construct containing a DNA sequence that is operably linked to a suitable control sequence capable of effecting the expression of the DNA encoding the polypeptide in a suitable host. The requirements are that the vectors are replicable and viable in the host cell of choice. Low- or high-copy number vectors may be used as desired. The control sequences of the vector include a promoter to effect transcription, an optional operator sequence to control such transcription, a sequence encoding suitable mRNA ribosome binding sites, and sequences that control termination of transcription and translation. In some embodiments, a nucleotide sequence encoding a gRNA is operably linked to a control element, e.g., a transcriptional control element, such as a promoter. In some embodiments, a nucleotide sequence encoding a CasX protein is operably linked to a control element, e.g., a transcriptional control element, such as a promoter. In other cases, the nucleotide encoding the CasX and gRNA are linked and are operably linked to a single control element. The promoter may be any DNA sequence, which shows transcriptional activity in the host cell of choice and may be derived from genes encoding proteins either homologous or heterologous to the host cell. Exemplary regulatory elements include a transcription promoter, a transcription enhancer element, a transcription termination signal, internal ribosome entry site (IRES) or P2A peptide to permit translation of multiple genes from a single transcript, polyadenylation sequences to promote downstream transcriptional termination, sequences for optimization of initiation of translation, and translation termination sequences. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. For example, in some cases, the transcriptional control element can be functional in eukaryotic cells, e.g., packaging cells for viral or XDP vectors, hematopoietic stem cells (HSC), hematopoietic progenitor cells (HPC), CD34+ cells, mesenchymal stem cells (MSC), embryonic stem (ES) cells, induced pluripotent stem cells (iPSC), common myeloid progenitor cells, proerythroblast cells, and erythroblast cells.

Non-limiting examples of pol II promoters include, but are not limited to EF-1alpha, EF-1alpha core promoter, Jens Tornoe (JeT), promoters from cytomegalovirus (CMV), CMV immediate early (CMVIE), CMV enhancer, herpes simplex virus (HSV) thymidine kinase, early and late simian virus 40 (SV40), the SV40 enhancer, long terminal repeats (LTRs) from retrovirus, mouse metallothionein-I, adenovirus major late promoter (Ad MLP), CMV promoter full-length promoter, the minimal CMV promoter, the chicken CE≤-actin promoter (CBA), CBA hybrid (CBh), chicken CE≤-actin promoter with cytomegalovirus enhancer (CB7), chicken beta-Actin promoter and rabbit beta-Globin splice acceptor site fusion (CAG), the rous sarcoma virus (RSV) promoter, the HIV-Ltr promoter, the hPGK promoter, the HSV TK promoter, a 7SK promoter, the Mini-TK promoter, the human synapsin I (SYN) promoter which confers neuron-specific expression, beta-actin promoter, super core promoter 1 (SCP1), the Mecp2 promoter for selective expression in neurons, the minimal IL-2 promoter, the Rous sarcoma virus enhancer/promoter (single), the spleen focus-forming virus long terminal repeat (LTR) promoter, the TBG promoter, promoter from the human thyroxine-binding globulin gene (Liver specific), the PGK promoter, the human ubiquitin C promoter (UBC), the UCOE promoter (Promoter of HNRPA2B1-CBX3), the synthetic CAG promoter, the Histone H2 promoter, the Histone H3 promoter, the U1a1 small nuclear RNA promoter (226 nt), the U1a1 small nuclear RNA promoter (226 nt), the U1b2 small nuclear RNA promoter (246 nt) 26, the GUSB promoter, the CBh promoter, rhodopsin (Rho) promoter, silencing-prone spleen focus forming virus (SFFV) promoter, a human H1 promoter (H1), a POL1 promoter, the TTR minimal enhancer/promoter, the b-kinesin promoter, mouse mammary tumor virus long terminal repeat (LTR) promoter, the human eukaryotic initiation factor 4A (EIF4A1) promoter, the ROSA26 promoter, the glyceraldehyde 3-phosphate dehydrogenase (GAPDH) promoter, tRNA promoters, and truncated versions and sequence variants of the foregoing. In a particular embodiment, the pol II promoter is EF-1alpha, wherein the promoter enhances transfection efficiency, the transgene transcription or expression of the CRISPR nuclease, the proportion of expression-positive clones and the copy number of the episomal vector in long-term culture.

Non-limiting examples of pol III promoters include, but are not limited to U6, mini U6, U6 truncated promoters, 7SK, and H1 variants, BiHi (Bidrectional H1 promoter), BiU6, Bi7SK, BiH1 (Bidirectional U6, 7SK, and H1 promoters), gorilla U6, rhesus U6, human 7SK, human H1 promoters, and sequence variants thereof. In the foregoing embodiment, the pol III promoter enhances the transcription of the gRNA.

Selection of the appropriate vector and promoter is well within the level of ordinary skill in the art, as it related to controlling expression, e.g., for modifying a PTBP1 gene. The expression vector may also contain a ribosome binding site for translation initiation and a transcription terminator. The expression vector may also include appropriate sequences for amplifying expression. The expression vector may also include nucleotide sequences encoding protein tags (e.g., 6xHis tag, hemagglutinin tag, fluorescent protein, etc.) that can be fused to the CasX protein, thus resulting in a chimeric CasX protein that are used for purification or detection.

Recombinant expression vectors of the disclosure can also comprise elements that facilitate robust expression of CasX proteins and the gRNAs of the disclosure. For example, recombinant expression vectors can include one or more of a polyadenylation signal (poly(A)), an intronic sequence or a post-transcriptional regulatory element such as a woodchuck hepatitis post-transcriptional regulatory element (WPRE). Exemplary poly(A) sequences include hGH poly(A) signal (short), HSV TK poly(A) signal, synthetic polyadenylation signals, SV40 poly(A) signal, β-globin poly(A) signal and the like. A person of ordinary skill in the art will be able to select suitable elements to include in the recombinant expression vectors described herein.

In some embodiments, provided herein are one or more recombinant expression vectors comprising one or more of: (i) a nucleotide sequence of a donor template nucleic acid where the donor template comprises a nucleotide sequence having homology to a sequence of the target PTBP1 locus of the target nucleic acid (e.g., a target genome); (ii) a nucleotide sequence that encodes a gRNA that hybridizes to a target sequence of the PTBP1 locus of the targeted genome (e.g., configured as a single or dual guide RNA) operably linked to a promoter that is operable in a target cell such as a eukaryotic cell; and (iii) a nucleotide sequence encoding a CasX protein operably linked to a promoter that is operable in a target cell such as a eukaryotic cell. In some embodiments, the sequences encoding the donor template, the gRNA and the CasX protein are in different recombinant expression vectors, and in other embodiments one or more polynucleotide sequences (for the donor template, CasX, and the gRNA) are in the same recombinant expression vector. In other cases, the CasX and gRNA are delivered to the target cell as an RNP (e.g., by electroporation or chemical means) and the donor template is delivered by a vector.

The polynucleotide sequence(s) are inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature. Various vectors are publicly available. The vector may, for example, be in the form of a plasmid, cosmid, viral particle, or phage that may conveniently be subjected to recombinant DNA procedures, and the choice of vector will often depend on the host cell into which it is to be introduced. Thus, the vector may be an autonomously replicating vector, i.e., a vector, which exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated. Once introduced into a suitable host cell, expression of the protein involved in antigen processing, antigen presentation, antigen recognition, and/or antigen response can be determined using any nucleic acid or protein assay known in the art. For example, the presence of transcribed mRNA of reference CasX or the CasX variants can be detected and/or quantified by conventional hybridization assays (e.g., Northern blot analysis), amplification procedures (e.g. RT-PCR), SAGE (U.S. Pat. No. 5,695,937), and array-based technologies (see e.g., U.S. Pat. Nos. 5,405,783, 5,412,087 and 5,445,934), using probes complementary to any region of the polynucleotide.

The polynucleotides and recombinant expression vectors can be delivered to the target host cells by a variety of methods. Such methods include, but are not limited to, viral infection, transfection, lipofection, electroporation, calcium phosphate precipitation, polyethyleneimine (PEI)-mediated transfection, DEAE-dextran mediated transfection, microinjection, liposome-mediated transfection, particle gun technology, nucleofection, direct addition by cell penetrating CasX proteins that are fused to or recruit donor DNA, cell squeezing, calcium phosphate precipitation, direct microinjection, nanoparticle-mediated nucleic acid delivery, and using the commercially available TransMessenger® reagents from Qiagen, Stemfect™ RNA Transfection Kit from Stemgent, and TransIT®-mRNA Transfection Kit from Mirus Bio LLC, Lonza nucleofection, Maxagen electroporation and the like.

A recombinant expression vector sequence can be packaged into a virus or virus-like particle (also referred to herein as a “particle” or “virion”) for subsequent infection and transformation of a cell, ex vivo, in vitro or in vivo. Such particles or virions will typically include proteins that encapsidate or package the vector genome. Suitable expression vectors may include viral expression vectors based on vaccinia virus; poliovirus; adenovirus; a retroviral vector (e.g., Murine Leukemia Virus), spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus; and the like. In some embodiments, a recombinant expression vector of the present disclosure is a recombinant adeno-associated virus (AAV) vector. In some embodiments, a recombinant expression vector of the present disclosure is a recombinant lentivirus vector. In some embodiments, a recombinant expression vector of the present disclosure is a recombinant retroviral vector.

In some embodiments, a recombinant expression vector of the present disclosure is a recombinant adeno-associated virus (AAV) vector. In some embodiments, a recombinant expression vector of the present disclosure is a recombinant lentivirus vector. In some embodiments, a recombinant expression vector of the present disclosure is a recombinant retroviral vector.

AAV is a small (20 nm), nonpathogenic virus that is useful in treating human diseases in situations that employ a viral vector for delivery to a cell such as a eukaryotic cell, either in vivo or ex vivo for cells to be prepared for administering to a subject. A construct is generated, for example a construct encoding any of the CasX proteins and/or CasX gRNA embodiments as described herein, and is flanked with AAV inverted terminal repeat (ITR) sequences, thereby enabling packaging of the AAV vector into an AAV viral particle.

An “AAV” vector may refer to the naturally occurring wild-type virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on capsid protein reactivity with defined antisera, e.g., there are many known serotypes of primate AAVs. In some embodiments, the AAV vector is selected from AAV1, AAV2, AAV3, AAV4, AAV5, AAV6, AAV7, AAV8, AAV9, AAV10, AAV11, AAV12, AAV 44.9, AAV-Rh74 (Rhesus macaque-derived AAV), and AAVRh10, and modified capsids of these serotypes. For example, serotype AAV-2 is used to refer to an AAV which contains capsid proteins encoded from the cap gene of AAV-2 and a genome containing 5′ and 3′ ITR sequences from the same AAV-2 serotype. Pseudotyped AAV refers to an AAV that contains capsid proteins from one serotype and a viral genome including 5′-3′ ITRs of a second serotype. Pseudotyped rAAV would be expected to have cell surface binding properties of the capsid serotype and genetic properties consistent with the ITR serotype. Pseudotyped recombinant AAV (rAAV) are produced using standard techniques described in the art. As used herein, for example, rAAV1 may be used to refer an AAV having both capsid proteins and 5′-3′ ITRs from the same serotype or it may refer to an AAV having capsid proteins from serotype 1 and 5′-3′ ITRs from a different AAV serotype, e.g., AAV serotype 2. For each example illustrated herein the description of the vector design and production describes the serotype of the capsid and 5′-3′ ITR sequences.

An “AAV virus” or “AAV viral particle” refers to a viral particle composed of at least one AAV capsid protein (preferably by all of the capsid proteins of a wild-type AAV) and an encapsidated polynucleotide. If the particle additionally comprises a heterologous polynucleotide (i.e., a polynucleotide other than a wild-type AAV genome to be delivered to a mammalian cell), it is typically referred to as “rAAV”. An exemplary heterologous polynucleotide is a polynucleotide comprising a CasX protein and/or sgRNA and, optionally, a donor template of any of the embodiments described herein.

By “adeno-associated virus inverted terminal repeats” or “AAV ITRs” is meant the art recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome.

The nucleotide sequences of AAV ITR regions are known. See, for example Kotin, R. M. (1994) Human Gene Therapy 5:793-801; Berns, K. I. “Parvoviridae and their Replication” in Fundamental Virology, 2^{nd} Edition, (B. N. Fields and D. M. Knipe, eds.). As used herein, an AAV ITR need not have the wild-type nucleotide sequence depicted, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV6, AAV7, AAV8, AAV9, AAV10, AAV-Rh74, and AAVRh10, and modified capsids of these serotypes. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Use of AAV serotypes for integration of heterologous sequences into a host cell is known in the art (see, e.g., WO2018195555A1 and US20180258424A1, incorporated by reference herein.)

By “AAV rep coding region” is meant the region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. By “AAV cap coding region” is meant the region of the AAV genome which encodes the capsid proteins VP1, VP2, and VP3, or functional homologues thereof. These Cap expression products supply the packaging functions which are collectively required for packaging the viral genome.

In some embodiments, AAV capsids utilized for delivery of the encoding sequences for the CasX and gRNA, and, optionally, the DMPK donor template nucleotides to a host cell can be derived from any of several AAV serotypes, including without limitation, AAV1, AAV2, AAV3, AAV4, AAV5, AAV6, AAV7, AAV8, AAV9, AAV10, AAV 11, AAV12, AAV 44.9, AAV-Rh74 (Rhesus macaque-derived AAV), and AAVRh10, and the AAV ITRs are derived from AAV serotype 2. In a particular embodiment, AAV1, AAV7, AAV6, AAV8, or AAV9 are utilized for delivery of the CasX, gRNA, and, optionally, donor template nucleotides, to a host muscle cell.

In order to produce rAAV viral particles, an AAV expression vector is introduced into a suitable host cell using known techniques, such as by transfection. Packaging cells are typically used to form virus particles; such cells include HEK293 cells (and other cells known in the art), which package adenovirus. A number of transfection techniques are generally known in the art; see, e.g., Sambrook et al. (1989) Molecular Cloning, a laboratory manual, Cold Spring Harbor Laboratories, New York. Particularly suitable transfection methods include calcium phosphate co-precipitation, direct microinjection into cultured cells, electroporation, liposome mediated gene transfer, lipid-mediated transduction, and nucleic acid delivery using high-velocity microprojectiles.

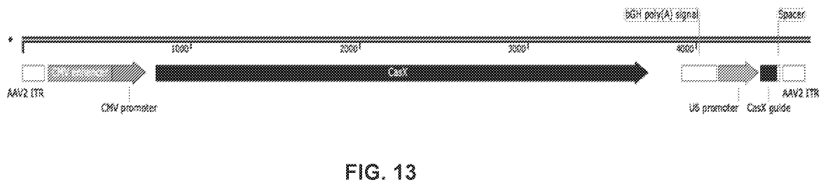

In an advantage of rAAV constructs of the present disclosure, the smaller size of the CRISPR Type V nucleases; e.g., the CasX of the embodiments, permits the inclusion of all the necessary editing and ancillary expression components into the transgene such that a single rAAV particle can deliver and transduce these components into a target cell in a form that results in the expression of the CRISPR nuclease and gRNA that are capable of effectively modifying the target nucleic acid of the target cell. A representative schematic of such a construct is presented in FIG. 13. This stands in marked contrast to other CRISPR systems, such as Cas9, where typically a two-particle system is employed to deliver the necessary editing components to a target cell. Thus, in some embodiments of the rAAV systems, the disclosure provides; i) a first plasmid comprising the ITRs, sequences encoding the CasX variant, sequences encoding one or more gRNA, a first promoter operably linked to the CasX and a second promoter operably linked to the gRNA, and, optionally, one or more enhancer elements; ii) a second plasmid comprising the rep and cap genes; and iii) a third plasmid comprising helper genes, wherein upon transfection of an appropriate packaging cell, the cell is capable of producing an rAAV having the ability to deliver to a target cell, in a single particle, sequences capable of expressing the CasX nuclease and gRNA having the ability to edit the target nucleic acid of the target cell. In some embodiments of the rAAV systems, the sequence encoding the CRISPR protein and the sequence encoding the at least first gRNA are less than about 3100, less than about 3090, less than about 3080, less than about 3070, less than about 3060, less than about 3050, or less than about 3040 nucleotides in length, such that the sequences encoding the first and second promoter and, optionally, one or more enhance elements can have at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In some embodiments of the rAAV systems, the sequence encoding the first promoter and the at least one accessory element have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In some embodiments of the rAAV systems, the sequence encoding the first and second promoters and the at least one accessory element have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length.

In some embodiments, host cells transfected with the above-described AAV expression vectors are rendered capable of providing AAV helper functions in order to replicate and encapsidate the nucleotide sequences flanked by the AAV ITRs to produce rAAV viral particles. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs (open reading frames), encoding the rep and cap coding regions, or functional homologues thereof. Accessory functions can be introduced into and then expressed in host cells using methods known to those of skill in the art. Commonly, accessory functions are provided by infection of the host cells with an unrelated helper virus. In some embodiments, accessory functions are provided using an accessory function vector. Depending on the host/vector system utilized, any of a number of suitable transcription and translation control elements, including constitutive and inducible promoters, transcription enhancer elements, transcription terminators, etc., may be used in the expression vector. In some embodiments, the disclosure provides host cells comprising the AAV vectors of the embodiments disclosed herein.

In other embodiments, suitable vectors may include virus-like particles (VLP). Virus-like particles (VLPs) are particles that closely resemble viruses, but do not contain viral genetic material and are therefore non-infectious. In some embodiments, VLPs comprise a polynucleotide encoding a transgene of interest, for example any of the CasX protein and/or a gRNA embodiments, and, optionally, donor template polynucleotides described herein, packaged with one or more viral structural proteins.

In other embodiments, the disclosure provides XDPs produced in vitro that comprise a CasX:gRNA RNP complex and, optionally, a donor template. Combinations of structural proteins from different viruses can be used to create XDPs, including components from virus families including Parvoviridae (e.g., adeno-associated virus), Retroviridae (e.g., alpharetrovirus, a betaretrovirus, a gammaretrovirus, a deltaretrovirus, a epsilonretrovirus, or a lentivirus), Flaviviridae (e.g., Hepatitis C virus), Paramyxoviridae (e.g., Nipah) and bacteriophages (e.g., Qβ, AP205). In some embodiments, the disclosure provides XDP systems designed using components of retrovirus, including lentiviruses (such as HIV) and alpharetrovirus, betaretrovirus, gammaretrovirus, deltaretrovirus, epsilonretrovirus, in which individual plasmids comprising polynucleotides encoding the various components are introduced into a packaging cell that, in turn, produce the XDP. In some embodiments, the disclosure provides XDP comprising one or more components of i) protease, ii) a protease cleavage site, iii) one or more components of a gag polyprotein selected from a matrix protein (MA), a nucleocapsid protein (NC), a capsid protein (CA), a p1 peptide, a p6 peptide, a P2A peptide, a P2B peptide, a P10 peptide, a p12 peptide, a PP21/24 peptide, a P12/P3/P8 peptide, and a P20 peptide; v) CasX; vi) gRNA, and vi) targeting glycoproteins or antibody fragments wherein the resulting XDP particle encapsidates a CasX:gRNA RNP. The polynucleotides encoding the Gag, CasX and gRNA can further comprise paired components designed to assist the trafficking of the components out of the nucleus of the host cell and into the budding XDP. Non-limiting examples of such trafficking components include hairpin RNA such as MS2 hairpin, PP7 hairpin, Qβ hairpin, and U1 hairpin II that have binding affinity for MS2 coat protein, PP7 coat protein, Qβ coat protein, and U1A signal recognition particle, respectively. In other embodiments, the gRNA can comprise Rev response element (RRE) or portions thereof that have binding affinity to Rev, which can be linked to the Gag polyprotein. The RRE can be selected from the group consisting of Stem IIB of Rev response element (RRE), Stem II-V of RRE, Stem II of RRE, Rev-binding element (RBE) of Stem IIB, and full-length RRE. In the foregoing embodiment, the components include sequences of UGGGCGCAGCGUCAAUGACGCUGACGGUACA (Stem JIB; SEQ ID NO: 43931), GCACUAUGGGCGCAGCGUCAAUGACGCUGACGGUACAGGCCAGACAAUUAUUGU CUGGUAUAGUGC (Stem II; SEQ ID NO: 43932), GCUGACGGUACAGGC (RBE, SEQ ID NO: 44046), CAGGAAGCACUAUGGGCGCAGCGUCAAUGACGCUGACGGUACAGGCCAGACAAU UAUUGUCUGGUAUAGUGCAGCAGCAGAACAAUUUGCUGAGGGCUAUUGAGGCGC AACAGCAUCUGUUGCAACUCACAGUCUGGGGCAUCAAGCAGCUCCAGGCAAGAA UCCUG (Stem II-V; SEQ ID NO: 43933), and AGGAGCUUUGUUCCUUGGGUUCUUGGGAGCAGCAGGAAGCACUAUGGGCGCAGC GUCAAUGACGCUGACGGUACAGGCCAGACAAUUAUUGUCUGGUAUAGUGCAGCA GCAGAACAAUUUGCUGAGGGCUAUUGAGGCGCAACAGCAUCUGUUGCAACUCAC AGUCUGGGGCAUCAAGCAGCUCCAGGCAAGAAUCCUGGCUGUGGAAAGAUACCU AAAGGAUCAACAGCUCCU (full-length RRE; SEQ ID NO: 43934). In other embodiments, the gRNA can comprise one or more RRE and one or more MS2 hairpin sequences. In a particular embodiment, the gRNA comprises an MS2 hairpin variant that is optimized to increase the binding affinity to the MS2 coat protein, thereby enhancing the incorporation of the gRNA and associated CasX into the budding XDP. gRNA variants comprising MS2 hairpin variants include gRNA variants 275-315 and 317-320 (SEQ ID NOS: 43617-43661 as shown in Table 3).

The targeting glycoproteins or antibody fragments on the surface that provides tropism of the XDP to the target cell, wherein upon administration and entry into the target cell, the RNP molecule is free to be transported into the nucleus of the cell. The envelope glycoprotein can be derived from any enveloped viruses known in the art to confer tropism to XDP, including but not limited to the group consisting of Argentine hemorrhagic fever virus, Australian bat virus, Autographa californica multiple nucleopolyhedrovirus, Avian leukosis virus, baboon endogenous virus, Bolivian hemorrhagic fever virus, Borna disease virus, Breda virus, Bunyamwera virus, Chandipura virus, Chikungunya virus, Crimean-Congo hemorrhagic fever virus, Dengue fever virus, Duvenhage virus, Eastern equine encephalitis virus, Ebola hemorrhagic fever virus, Ebola Zaire virus, enteric adenovirus, Ephemerovirus, Epstein-Bar virus (EBV), European bat virus 1, European bat virus 2, Fug Synthetic gP Fusion, Gibbon ape leukemia virus, Hantavirus, Hendra virus, hepatitis A virus, hepatitis B virus, hepatitis C virus, hepatitis D virus, hepatitis E virus, hepatitis G Virus (GB virus C), herpes simplex virus type 1, herpes simplex virus type 2, human cytomegalovirus (HHV5), human foamy virus, human herpesvirus (HHV), human Herpesvirus 7, human herpesvirus type 6, human herpesvirus type 8, human immunodeficiency virus 1 (HIV-1), human metapneumovirus, human T-lymphotropic virus 1, influenza A, influenza B, influenza C virus, Japanese encephalitis virus, Kaposi's sarcoma-associated herpesvirus (HHV8), Kaysanur Forest disease virus, La Crosse virus, Lagos bat virus, Lassa fever virus, lymphocytic choriomeningitis virus (LCMV), Machupo virus, Marburg hemorrhagic fever virus, measles virus, Middle eastern respiratory syndrome-related coronavirus, Mokola virus, Moloney murine leukemia virus, monkey pox, mouse mammary tumor virus, mumps virus, murine gammaherpesvirus, Newcastle disease virus, Nipah virus, Nipah virus, Norwalk virus, Omsk hemorrhagic fever virus, papilloma virus, parvovirus, pseudorabies virus, Quaranfil virus, rabies virus, RD 114 Endogenous Feline Retrovirus, respiratory syncytial virus (RSV), Rift Valley fever virus, Ross River virus, rRotavirus, Rous sarcoma virus, rubella virus, Sabia-associated hemorrhagic fever virus, SARS-associated coronavirus (SARS-CoV), Sendai virus, Tacaribe virus, Thogotovirus, tick-borne encephalitis causing virus, varicella zoster virus (HHV3), varicella zoster virus (HHV3), variola major virus, variola minor virus, Venezuelan equine encephalitis virus, Venezuelan hemorrhagic fever virus, vesicular stomatitis virus (VSV), VSV-G, Vesiculovirus, West Nile virus, western equine encephalitis virus, and Zika Virus.

In other embodiments, the disclosure provides XDP of the foregoing and further comprises one or more components of a pol polyprotein (e.g., a protease), and, optionally, a second CasX or a donor template. The disclosure contemplates multiple configurations of the arrangement of the encoded components, including duplicates of some of the encoded components. The foregoing offers advantages over other vectors in the art in that viral transduction to dividing and non-dividing cells is efficient and that the XDP delivers potent and short-lived RNP that escape a subject's immune surveillance mechanisms that would otherwise detect a foreign protein. Non-limiting, exemplary XDP systems are described in PCT/US20/63488 and WO2021113772A1, incorporated by reference herein. In some embodiments, the disclosure provides host cells comprising polynucleotides or vectors encoding any of the foregoing XDP embodiments.

Upon production and recovery of the XDP comprising the CasX:gRNA RNP of any of the embodiments described herein, the XDP can be used in methods to edit target cells of subjects by the administering of such XDP, as described more fully, below.

### V. Cells

In still another aspect, provided herein are populations of cells comprising a PTBP1 gene modified by any of the systems or method embodiments described herein. In some embodiments, the cells of the subject are modified in vivo by any of the systems or method embodiments described herein; e.g., to treat a subject having a neurologic disease or injury such as Parkinson's disease, Huntington's disease, Alzheimer's, amyotrophic lateral sclerosis (ALS), traumatic brain injury, traumatic spinal cord injury, amongst others, or cancer, described more fully below.

In some embodiments, the population of cells are modified by a Type V Cas nuclease and one or more guides targeted to the PTBP1 target nucleic acid. In some embodiments, the disclosure provides methods and populations of cells modified by introducing into each cell of the population: i) a CasX:gRNA system comprising a CasX and a gRNA of any one of the embodiments described herein; ii) a CasX:gRNA system comprising a CasX, a gRNA, and a donor template of any one of the embodiments described herein; iii) a nucleic acid encoding the CasX and the gRNA, and optionally comprising the donor template; iv) a vector comprising the nucleic acid of (iii), above, which can be an AAV of any of the embodiments described herein; v) a XDP comprising the CasX:gRNA system of any one of the embodiments described herein; or vi) combinations of two or more of (i) to (v), wherein the PTBP1 target nucleic acid sequence of the cells targeted by the gRNA is modified by the CasX protein and, optionally, the donor template. In some embodiments, the disclosure provides a population of cells wherein the cells have been modified such that at least 60%, at least 70%, at least 75%, at least 80%, at least 85%, at least 90%, or at least 95% of the modified cells do not express a detectable level of PTBP1 protein. In other embodiments, the disclosure provides a population of cells wherein the cells have been modified such that the expression of PTBP1 protein is reduced by at least 70%, at least 75%, at least 80%, at least 85%, at least 90%, or at least 95% compared to cells that have not been modified. In still other embodiments, the disclosure provides a population of cells wherein expression of the PTBP1 protein cannot be detected in the modified cells of the population. In the foregoing, the cells of the population modified by the methods of the disclosure can be fibroblasts, glial cells, microglial cells, oligodendrocytes or astrocytes, wherein the modification of the PTBP1 target nucleic acid sequence and the down-regulation of PTBP1 results in reprogramming or conversion of the cells into functional neurons that then express nPTB (also known as PTBP2), which can result in the prevention or amelioration of the neurologic disease of the subject. In some embodiments, the disclosure provides a population of cells wherein the cells have been modified such that the expression of nPTB in the modified cells is increased by at least about 10%, at least about 20%, at least about 30%, at least about 40%, at least about 50%, at least about 60%, at least about 70%, at least about 80%, or at least about 90% in comparison to cells in which the PTBP1 gene has not been modified. In other embodiments, the disclosure provides a population of cells wherein the cells have been modified such that at least about 10%, at least about 20%, at least about 30%, at least about 40%, at least about 50%, at least about 60%, at least about 70%, at least about 80%, or at least about 90% of the modified cells express a detectable level of nPTB protein. In some embodiments, the population of modified cells are animal cells; for example, a rodent, rat, mouse, rabbit or dog cell. In some embodiments, the cell is a human cell. In some embodiments, the cell is a non-human primate cell; e.g., a cynomolgus monkey cell. The effects of the modification can be assessed by flow cytometry, ELISA, cell-based assays, Western blot or other methods know in the art (Cho, C., et al. PTBP1-mediated regulation of AXL mRNA stability plays a role in lung tumorigenesis. Scientific Reports 9:16922 (2019)), the assays of the examples, or conventional assays known in the art.

### VI. Therapeutic Methods

In another aspect, the present disclosure relates to methods of treating a PTBP1-related disease in a subject in need thereof, including but not limited to neurologic diseases or cancers in which PTBP1 is implicated in the disease process or its amelioration. It will be understood that in some cases, PTBP1 protein is not an underlying cause of the disease, but the modification of the PTBP1 gene to reduce or eliminate the expression of the PTBP1 protein contributes to the prevention or amelioration of the disease and/or its signs and symptoms. Thus, use of the phrase “PTBP1-related disease” is intended to collectively encompass those diseases in which PTBP1 protein has either a causal role or its reduction in expression results in an improved therapeutic outcome. Non-limiting examples of neurologic diseases or injuries contemplated by the methods of treatment of the disclosure include, but are not limited to Parkinson's disease, Huntington's disease, Alzheimer's, amyotrophic lateral sclerosis (ALS), traumatic brain injury, traumatic spinal cord injury, amongst others. In those cases where the cells of the subject to be modified by the methods of the disclosure is a cell of the central nervous system (CNS) or the peripheral nervous system (PNS), the cell can be a fibroblast, a glial cell, a microglial cell, an oligodendrocyte or an astrocyte that, by the method of knocking-down or knocking-out of the PTBP1 gene, results in the reprogramming or conversion of the cells into functional neurons that then express nPTB (also known as PTBP2), which can result in the prevention or amelioration of the neurologic disease of the subject. In those cases where the cells of the subject to be modified by the methods of the disclosure is a cancer, the method results in the knocking-down or knocking-out of the PTBP1 gene in cells in which the PTBP1 protein is overexpressed, resulting in reduced tumorigenesis of the cells or stasis of an existing tumor in a subject. Non-limiting examples of cancers contemplated by the methods of treatment of the disclosure include, but are not limited to ovarian tumors, glioblastomas, bladder cancer, colon cancer and breast cancer.

In some embodiments, the allele related to the PTBP1-related disease of the subject to be modified is a wild-type sequence. A number of therapeutic strategies have been used to design the compositions for use in the methods of treatment of a subject with a PTBP1-related disease. In some embodiments, the disclosure provides methods of treating a PTBP1-related disease in a subject in need thereof in which repression or elimination of expression of the PTBP1 protein by modifying the PTBP1 gene in target cells of the subject ameliorates the signs, symptoms, or effects of the disease. In such embodiments, the method comprises administering to the subject a therapeutically effective dose of a Class 2, Type V CRISPR system embodiment disclosed herein.

In some embodiments, the method of treatment comprises administering to the subject a therapeutically effective dose of: i) the CasX:gRNA system comprising a first CasX protein and a first gRNA with a targeting sequence complementary to the target nucleic acid; ii) the CasX:gRNA system comprising a first CasX protein and a first gRNA with a targeting sequence complementary to the target nucleic acid and a donor template; iii) a nucleic acid encoding the CasX:gRNA system of (i) or (ii); iv) a vector comprising the nucleic acid of (iii), which can be an AAV of any of the embodiments described herein; v) a XDP comprising the CasX:gRNA system of (i) or (ii); or vi) combinations of two or more of (i)-(v), wherein said administering results in 1) modification of the PTBP1 target nucleic acid sequence by the CasX protein and, optionally, the donor template; and 2) decreased or elimination of expression of the PTBP1 protein in the modified cells of the subject, wherein the treatment prevents or ameliorates the signs, symptoms, or effects of the disease. The methods are described more fully, below.

In some embodiments of the method of treatment, the targeting sequence of the gRNA of the CasX:gRNA system used to target the specific sequence of the PTBP1 gene of the cells is selected from a sequence selected from the group consisting of SEQ ID NOS: 492-2100 and 2286-43569, or a sequence having at least about 65%, at least about 75%, at least about 85%, or at least about 95% identity thereto. In a particular embodiment, the targeting sequence of the gRNA of the CasX:gRNA system used to target the specific sequence of the PTBP1 gene of the cells is selected from a sequence selected from the group consisting of SEQ ID NOS: 37971-37979, 38027, 38136, 38137, 38152-38158, 38160, 38162-38174, 38176, 38177, 38181, 38195, 38196, 38198, 38199, 38253-38256, 38259-38267, 38306 and 38311-38353. In some embodiments, the targeting sequence of the gRNA has a sequence that hybridizes with a PTBP1 exon. In some embodiments, the targeting sequence of the gRNA has a sequence that hybridizes with a PTBP1 intron. In some embodiments, the targeting sequence of the gRNA has a sequence that hybridizes with a PTBP1 intron-exon junction. In some embodiments, the targeting sequence of the gRNA has a sequence that hybridizes with an intergenic region of the PTBP1 gene. In some embodiments, the targeting sequence of the gRNA has a sequence that hybridizes with a PTBP1 regulatory element. In some embodiments, the targeting sequence of the gRNA has a sequence complementary to the PTBP1 regulatory element, wherein the PTBP1 regulatory element is 5′ of the PTBP1 gene. In some embodiments, the targeting sequence of the gRNA has a sequence complementary to the PTBP1 regulatory element, wherein the PTBP1 regulatory element comprises the 5′ untranslated region (UTR) of the PTBP1 gene. In some embodiments, the targeting sequence of the gRNA has a sequence complementary to the PTBP1 regulatory element, wherein the PTBP1 regulatory element is 3′ of the PTBP1 gene. In some embodiments, the targeting sequence of the gRNA has a sequence complementary to the PTBP1 regulatory element, wherein the PTBP1 regulatory element comprises the 3′UTR of the PTBP1 gene. In some embodiments, the targeting sequence of the gRNA has a sequence complementary to the PTBP1 regulatory element, wherein the PTBP1 regulatory element comprises a promoter. In some embodiments, the targeting sequence of the gRNA has a sequence complementary to the PTBP1 regulatory element, wherein the PTBP1 regulatory element comprises an enhancer. In some embodiments, the CasX proteins and the gRNA scaffolds utilized in the methods of treating a PTBP1-related disease described herein comprise a CasX sequence selected from the sequences of SEQ ID NOS: 36-99, 101-148 and 43662-43907 as set forth in Table 4, a CasX sequence selected from the sequences of SEQ ID NOS: 59, 72-99, 101-148, and 43662-43907, a CasX sequence selected from the sequences of SEQ ID NOS: 132-148 and 43662-43907, or sequences having at least about 50%, at least about 60%, at least about 70%, at least about 80%, or at least about 90%, or at least about 95%, or at least about 99% sequence identity thereto, and the gRNA scaffold comprises any one of the sequences of SEQ ID NOS: 2101-2285, 43571-43661, 44045, or 44047 as set forth in in Table 3, the gRNA scaffold comprises any one of the sequences of SEQ ID NOS: 2238-2285, 43571-43661,44045, or 44047 the gRNA scaffold comprises any one of the sequences of SEQ ID NOS: 2281-2285, 43571-43661, 44045, or 44047, or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto. In some embodiments of the method of treatment, the CasX of the CasX:gRNA system consists of a sequence of SEQ ID NOS: 36-99, 101-148 or 43662-43907 as set forth in Table 4, the gRNA scaffold consists of a sequence of SEQ ID NOS: 2101-2285, 43571-43661 or 44045 as set forth in Table 3, and the targeting sequence of the gRNA consists of a sequence selected from the group consisting of SEQ ID NOS: 492-2100 and 2286-43569. In some embodiments of the method of treatment, the CasX of the CasX:gRNA system consists of a sequence of SEQ ID NOS: 59, 72-99, 101-148, or 43662-43907, the gRNA scaffold consists of a sequence of SEQ ID NOS: 2238-2285, 43571-43661, 44045, or 44047, and the targeting sequence of the gRNA consists of a sequence selected from the group consisting of SEQ ID NOS: 492-2100 and 2286-43569. In some embodiments of the method of treatment, the CasX of the CasX:gRNA system consists of a sequence of SEQ ID NOS: 132-148 or 43662-43907, the gRNA scaffold consists of a sequence of SEQ ID NOS: 2281-2285, 43571-43661, 44045, or 44047, and the targeting sequence of the gRNA consists of a sequence selected from the group consisting of SEQ ID NOS: 492-2100 and 2286-43569.

Upon hybridization with the target nucleic acid by the CasX and the gRNA, the CasX introduces one or more single-strand breaks or double-strand breaks within or near the PTBP1 gene that results in a modification of the target nucleic acid. In those cases where the method of treatment is intended to knock-down or knock-out the PTBP1 gene in the cells of the subject, the CasX:gRNA system is designed to modify the target nucleic acid by introducing a permanent indel (deletion or insertion) or other mutation in the target nucleic acid that, together with the host cell repair mechanisms, results in reduced expression of the PTBP1 protein. In some cases, the expression of the PTBP1 protein is reduced by at least about 10%, at least about 20%, at least about 30%, at least about 40%, at least about 50%, at least about 60%, at least about 70%, at least about 80%, or at least about 90% in comparison to cells that have not been modified. In other cases, the PTBP1 target nucleic acid of the cells of the subject are modified such that expression of the PTBP1 protein cannot be detected.

In other embodiments, the target nucleic acid of the cells of the subject is modified using a CasX and a plurality of gRNAs (e.g., two, three, four or more) targeted to different or overlapping portions of the PTBP1 gene wherein the CasX protein introduces multiple breaks in the target nucleic acid sequence. Similarly, as described supra, in those cases where the method of treatment is intended to knock-down or knock-out the PTBP1 gene in the cells of the subject, the CasX:gRNA system is designed to modify the target nucleic acid by introducing one or more permanent indels (deletion or insertion) or mutations in the target nucleic acid that, together with the host cell repair mechanisms, results in reduced expression of the PTBP1 protein.

In some embodiments, the disclosure provides methods of treating a PTBP1-related disease in a subject in need thereof comprising modifying the PTBP1 gene with a CasX, one or more gRNA, and a donor template, wherein the donor template sequence is flanked by an upstream sequence and a downstream sequence with homology adjacent to the break sites in the target nucleic acid introduced by the CasX (i.e., homologous arms), facilitating insertion of the donor template sequence. In those cases where the method of treatment is intended to knock-down or knock-out the PTBP1 gene in the cells of the subject, the donor template sequence is typically not identical to the genomic sequence that it replaces and may contain one or more single base changes, insertions, deletions, inversions or rearrangements with respect to the genomic sequence, provided that there is sufficient homology with the target nucleic acid sequence to support homology-directed repair or insertion by HITI, which can result in a frame-shift or other mutation such that the PTBP1 protein is not expressed or the expression of the PTBP1 is reduced by at least about 10%, at least about 20%, at least about 30%, at least about 40%, at least about 50%, at least about 60%, at least about 70%, at least about 80%, at least about 90% in comparison to cells that have not been modified. The donor template inserted by HITI can be any length, for example, a relatively short sequence of between 1 and 50 nucleotides in length, or a longer sequence of about 50-1000 nucleotides in length. The donor template can be a short single-stranded or double-stranded oligonucleotide, or can be a long single-stranded or double-stranded oligonucleotide. The donor template of the embodiments can be designed to encode a PTBP1 exon, a PTBP1 intron, a PTBP1 intron-exon junction, a PTBP1 regulatory element, or an intergenic region. The donor template sequence may comprise certain sequence differences as compared to the genomic sequence; e.g., restriction sites, nucleotide polymorphisms, barcodes, selectable markers (e.g., drug resistance genes, fluorescent proteins, enzymes etc.), etc., which may be used to assess for successful insertion of the donor nucleic acid at the cleavage site or, in some cases, may be used for other purposes (e.g., to signify expression at the targeted genomic locus). Alternatively, these sequence differences may include flanking recombination sequences such as FLPs, loxP sequences, or the like, that can be activated at a later time for removal of the marker sequence.

In some embodiments of the method of treatment, the method comprises administration to the subject a therapeutically effective dose of a vector comprising polynucleotides encoding the CasX protein and the gRNA, wherein the contacting of the cells of the subject with the vector results in expression of the CasX and gRNA and modification of the target nucleic acid of the cells by the CasX:gRNA complex. The vectors disclosed herein may be delivered to a subject by multiple technologies including, but not limited to, DNA injection (also referred to as DNA vaccination) with and without in vivo electroporation, liposome mediated, nanoparticle facilitated, or use of recombinant vectors such as recombinant lentivirus, recombinant adenovirus, and recombinant adenovirus associated virus. In some embodiments, the method comprises administration of the vector comprising a polynucleotide encoding a CasX and a plurality of gRNAs targeted to the PTBP1 gene wherein the administration results in contacting the subject target nucleic acid within cells of the subject with the expression product(s) of the vectors wherein the PTBP1 gene is modified in the cell of the subject. In other embodiments of the method of treatment, the method comprises contacting the cell with a vector encoding the CasX protein and the gRNA and further comprising a donor template wherein said contacting results in modification of the target nucleic acid of the cell by cleavage by the CasX protein and insertion of the donor template into the target nucleic acid. In other embodiments of the method of treatment, the method comprises contacting the cell with a first vector encoding the CasX protein and the gRNA and a second vector comprising the donor template. In other embodiments, the method comprises administration of the vector comprising a polynucleotide encoding a CasX and a plurality of gRNAs targeted to the PTBP1 gene and a second vector comprising a donor template polynucleotide encoding at least a portion of or the entirety of a PTBP1 gene wherein the administration of the vectors results in contacting the subject target nucleic acid within a cell of the subject with the expression product(s) of the CasX and gRNA vectors and the donor template wherein the PTBP1 gene is modified in the cell of the subject. In those cases wherein the CasX:gRNA system is designed to knock-down/knock-out the PTBP1 gene, the donor template comprises one or more single base changes, insertions, deletions, inversions or rearrangements with respect to the genomic sequence in the PTBP1 gene portion, whereupon insertion of the donor template the gene is knocked-down or knocked-out. In some embodiments, the vector is a viral particle. In some embodiments, the vector is an AAV vector (described supra). The vectors of the embodiments are administered to the subject at a therapeutically effective dose. In some embodiments, the vector is administered to the subject at a dose of at least about 1×10⁵ vector genomes (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg. In other embodiments, the vector is administered to the subject at a dose of at least about 1×10⁵ vg/kg to at least about 1×10¹⁶ vg/kg, or at least about 1×10⁶ vg/kg to about 1×10¹⁵ vg/kg, or at least about 1×10⁷ vg/kg to about 1×10¹⁴ vg/kg, or at least about 1×10⁸ vg/kg to about 1×10¹⁴ vg/kg. In other embodiments of the method of treatment, the method comprises administration to the subject a therapeutically effective dose of a XDP comprising the CasX protein and the gRNA and, optionally, the donor template (described, supra), wherein the contacting of the cells of the subject with the XDP results in modification of the target nucleic acid of the cells by the CasX:gRNA complex. In some embodiments, the method comprises administration of the XDP comprising a CasX and a plurality of gRNAs targeted to different locations in the PTBP1 gene, wherein the contacting of the cells of the subject with the XDP results in modification of the target nucleic acid of the cells by the CasX:gRNA complexes. As previously described, the components can be designed to knock-down/knock-out the PTBP1 gene. The XDP of the embodiments are administered to the subject at a therapeutically effective dose. In some embodiments, the XDP is administered to the subject at a dose of at least about 1×10⁵ particles/kg, at least about 1×10⁶ particles/kg, at least about 1×10⁷ particles/kg at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, at least about 1×10¹⁶ particles/kg. In other embodiments, the XDP is administered to the subject at a dose of at least about 1×10⁵ particles/kg to at least about 1×10¹⁶ particles/kg, or at least about 1×10⁶ particles/kg to about 1×10¹⁵ particles/kg, or at least about 1×10⁷ particles/kg to about 1×10¹⁴ particles/kg, or at least about 1×10⁸ particles/kg to about 1×10¹⁴ particles/kg.

In other embodiments of the method of treatment, the method further comprises administration to the cells of a subject an additional CRISPR protein, or a polynucleotide (or a vector comprising the polynucleotide) encoding the additional CRISPR protein. In the foregoing embodiment, the additional CRISPR protein has a sequence different from the first CasX protein of the method. In some embodiments, the additional CRISPR protein is not a CasX protein; the additional CRISPR nuclease is selected from the group consisting of Cas9, Cas12a, Cas12b, Cas12c, Cas12d (CasY), Cas12j, Cas12k, Cas13a, Cas13b, Cas13c, Cas13d, CasX, CasY, Cas14, Cpf1, C2c1, Csn2, Cas Phi, and sequence variants thereof.

In some embodiments, the method comprises administering to the subject the compositions of the embodiments described herein (i.e., the CasX protein, the one or more gRNA, and, optionally the donor template, or the one or more polynucleotides encoding the CasX protein, the gRNA and the donor template, the vector or the XDP of the embodiments) at a therapeutically effective dose via an administration route selected from the group consisting of intraparenchymal, intravenous, intra-arterial, intracerebroventricular, intracisternal, intrathecal, intracranial, intra-striatal, lumbar, and intraperitoneal routes. It will be appreciated that for some neurologic diseases, the location of administration in the CNS or PNS may be more specific; e.g., regions such as the cortex, the corpus striatum, the spinal cord, or, in the case of treatment of Parkinson's disease, the substantia nigra. In some embodiments of the methods of treating a PTBP1-related disease or injury in a subject, the subject is selected from the group consisting of mouse, rat, non-human primate, and human. In a particular embodiment, the subject is a human. In the case of the treatment of cancer, the vector or XDP may be delivered parenterally or may be delivered directly into or proximally to the tumor. In the embodiments of the method of treatment, the vector or XDP may be administered according to a treatment regimen comprising one or more consecutive doses using a therapeutically effective dose of the vector or XDP. In some embodiments of the treatment regimen, the therapeutically effective dose of the vector or XDP is administered as a single dose. In other embodiments of the treatment regimen, the therapeutically effective dose of the vector or XDP is administered to the subject as two or more doses over a period of at least every two weeks, at least every month, at least every two months, at least every three months, at least every four months, at least every five months, at least every six months, or on an annual basis, or once every 2 or 3 years.

In some embodiments of the disclosure, the methods of treatment can prevent, treat and/or ameliorate a PTBP1-related disease of a subject by the administration to the subject of a therapeutically effective amount of a population of cells modified in vitro or ex vivo by CasX:gRNA system composition(s) of the embodiments described herein. In some cases, the CasX and gRNA is delivered to the cells of the population as an RNP (embodiments of which are described herein, supra), and, optionally, the donor template, wherein the target nucleic acid is modified such that the PTBP1 protein is not expressed or is expressed at a reduced level. In other cases, the CasX and gRNA is delivered to the cells of the population in a vector (embodiments of which are described herein, supra), wherein the target nucleic acid is modified such that the PTBP1 protein is not expressed or is expressed at a reduced level. In some embodiments, the cells of the population to be modified by the administration of the compositions are cells of the central nervous system (CNS) or the peripheral nervous system (PNS). In some embodiments, the cell is a glial cell, a microglial cell, an oligodendrocyte, an astrocyte, or a fibroblast, wherein the cell is reprogrammed and transformed into a functional neuron by the method. In some cases, the cells have been modified such that expression of the PTBP1 protein is decreased by at least about 50%, at least about 60%, at least about 70%, at least about 80%, at least about 90%, or at least about 95% in comparison to a cell that has not been modified. In other cases, the cells have been modified such that at least about 1%, at least about 5%, at least about 10%, at least about 20%, at least about 30%, at least about 40%, at least about 50%, at least about 60%, at least about 70%, at least about 80%, at least about 90%, or at least about 95% of the cells do not express a detectable level of the PTBP1 protein. In other embodiments of the method, the modification of the cells of the subject results in an increase in expression of nPTB in the modified cells by at least about 10%, at least about 20%, at least about 30%, at least about 40%, at least about 50%, at least about 60%, at least about 70%, at least about 80%, or at least about 90% in comparison to cells in which the PTBP1 gene has not been modified. In other embodiments of the method, the cells of the subject are modified such that at least about 10%, at least about 20%, at least about 30%, at least about 40%, at least about 50%, at least about 60%, at least about 70%, at least about 80%, or at least about 90% of the cells express a detectable level of nPTB protein.

In some embodiments, the method of treatment further comprises administering a chemotherapeutic agent, such as an immunosuppressive agent or carbidopa-levodopa for a neurologic disease, or a cytotoxic, alkylating agent, or monoclonal antibody for a cancer.

In some embodiments of the method of treating a subject having a neurologic PTBP1-disease, administering to the subject of a therapeutically effective amount of a vector, a XDP, a CasX-gRNA composition, or a plurality of modified cells of any one of the embodiments described herein can produce a beneficial effect in helping to prevent, to treat (e.g., reduce the severity) or prevent the progression of the disease, or result in an improvement in one or more clinical parameters or endpoints associated with the disease in the subject, notwithstanding that the subject may still be afflicted with the underlying disease. It will be understood that the clinical parameter or endpoint is dependent on the underlying disease of the subject. In the case of Parkinson's disease, the clinical parameter or endpoint is selected from one or any combination of the group consisting of disease progression, Unified Parkinson's Disease Rating Scale (UPDRS), Unified Dyskinesia Rating Scale (UDysRS), Parkinson's Disease Quality of Life Questionnaire (PDQ-39) score, Movement Disorder Society-Sponsored Unified Parkinson's Disease Rating Scale (MDS-UPDRS), changes from baseline of motor score as measured by Inertial Measurement Unit (IMU) on Finger taping (FT) and Pronation-supination movement of the hands (PSH), delay in time to clinically meaningful worsening of motor progression, levodopa's duration of effect (“on time”), Clinical Global Impression—Improvement (CGI-I), change from baseline in Zarit Burden Interview score (ZBI), EQ-5D summary index, total disease duration, patient cognitive status (MMSE), and change from baseline in fatigue. In the case of Huntington's disease, the clinical parameter or endpoint is selected from one or any combination of the group consisting of Unified Huntington's Disease Rating Scale (UHDRS), cognitive decline, psychiatric abnormalities, motor impairment, changes in baseline in striatal volume, Stroop word test, total motor score (TMS), bradykinesia, dystonia, Symbol Digit Modalities Test, University of Pennsylvania Smell Identification Test, emotion recognition, speeded tapping, paced tapping, the Trail Making Test, intracranial-corrected volumes (ICV), and the Everyday Cognition Rating Scale (ECOG). In the case of ALS, the clinically-relevant endpoint is selected from one or any combination of the group consisting of ALS Functional Rating Scale (ALSFRS-(R)), combined assessment of function and survival, time to death, time to tracheostomy or persistent assisted ventilation (DTP), forced vital capacity (% FVC), manual muscle test, maximum voluntary isometric contraction, duration of response, progression-free survival, time to progression of disease, and time-to-treatment failure. In the embodiments of the foregoing, the subject is a mammal selected from rodent, mouse, rat, non-human primate, and human. In the case of Alzheimer's disease, the clinical parameter or endpoint is selected from one or any combination of the group consisting of change in Alzheimer's Disease Assessment Scale-Cognitive subscale (ADAS-Cog₁₄) score, change in the Cohen-Mansfield Agitation Inventory (CMAI) score, change in the Alzheimer's Disease Cooperative Study-Instrumental Activities of Daily Living (ADCS-iADL) score, Clinical Dementia Rating Scale-Sum of Boxes (CDR-SB) score, DIAN Multivariate Cognitive Endpoint, Preclinical Alzheimer Cognitive Composite 5 (PACC5) score, Mini-Mental State Exam (MMSE) score, cognitive impairment, functional impairment, brain amyloid levels measured by amyloid positron emission tomography (PET), brain tau levels measured by PET, spinal fluid amyloid-β levels, and spinal fluid tau levels.

In some embodiments, the disclosure provides a method of treating a subject having a cancer in which PTBP1 protein in overexpressed. Non-limiting examples of such cancers include ovarian cancer, glioblastoma, bladder cancer, colon cancer and breast cancer. In some cases, the method of treating a subject having a cancer comprising modification of the PTBP1 gene in cells of a tumor by the administration of one or more therapeutically effective doses of a vector or XDP of an embodiment described herein, wherein the in vivo modification of the PTBP1 target nucleic acid sequence prevents or reduces tumorigenesis of the cells or results in stasis of an existing tumor in a subject. In the foregoing embodiment, the method results in stasis of an existing tumor for at least about 1 month, at least about 2 months, at least about 3 months, at least about 4 months, at least about 6 months, at least about 7 months, at least about 8 months, at least about 9 months, at least about 10 months, at least about 11 months, at least about 12 months, at least about 18 months, at least about 2 years, at least about 3 years, at least about 4 years, or at least about 5 years. In another embodiment, the method results in stasis of an existing tumor for at least about 1 month to about 5 years, at least about 6 months to about 4 years, or at least about 1 year to about 3 years. In other embodiments of the method of treating a subject having a cancer, the administering to the subject of a therapeutically effective amount of a vector or a XDP encoding or comprising a CasX-gRNA composition of any one of the embodiments described herein can produce a beneficial effect in helping to prevent, to treat (e.g., reduce the severity) or prevent the progression of the cancer or result in an improvement in one or more clinical parameters or endpoints associated with the disease in the subject selected from tumor shrinkage as a complete, partial or incomplete response; time-to-progression; time to treatment failure; biomarker response; progression-free survival; disease free-survival; time to recurrence; time to metastasis; time of overall survival; improvement of quality of life; and improvement of symptoms. In the embodiments of the foregoing, the subject is a mammal selected from rodent, mouse, rat, non-human primate, and human.

In some embodiments, the disclosure provides compositions comprising CasX and gRNA gene editing pairs, for use as a medicament for the treatment of a subject having a PTBP1-related disease. In the foregoing, the CasX can be a CasX variant comprising a sequence of SEQ ID NOS: 59, 72-99, 101-148, or 43662-43907, and the gRNA can be a gRNA variant comprising SEQ ID NOS: 2101-2285, 43571-43661 or 44045 having a targeting sequence complementary to a target nucleic acid sequence within the PTBP1 gene or that comprises a sequence selected from the group consisting of SEQ ID NOS: 492-2100 and 2286-43569. In the foregoing, the CasX can be a CasX variant comprising a sequence of SEQ ID NOS: 59, 72-99, 101-148, and 43662-43907 and the gRNA can be a gRNA variant comprising SEQ ID NOS: 2238-2285, 43571-43661 or 44045 having a targeting sequence complementary to a target nucleic acid sequence within the PTBP1 gene or that comprises a sequence selected from the group consisting of SEQ ID NOS: 492-2100 and 2286-43569. In the foregoing, the CasX can be a CasX variant comprising a sequence of SEQ ID NOS: 132-148 or 43662-43907, and the gRNA can be a gRNA variant comprising SEQ ID NOS: 2281-2285, 43571-43661,44045, or 44047 having a targeting sequence complementary to a target nucleic acid sequence within the PTBP1 gene or that comprises a sequence selected from the group consisting of SEQ ID NOS: 492-2100 and 2286-43569. In other embodiments, the disclosure provides compositions of vectors comprising or encoding the gene editing pairs of CasX and gRNA for use as a medicament for the treatment of a subject having a PTBP1 related disease.

### VII. Kits and Articles of Manufacture

In another aspect, provided herein are kits comprising the compositions of the embodiments described herein. In some embodiments, the kit comprises a CasX variant protein and one or a plurality of gRNA variants of any of the embodiments of the disclosure comprising a targeting sequence region specific for a PTBP1 gene, optionally a donor template, and a suitable container (for example a tube, vial or plate). In other embodiments, the kit comprises a nucleic acid encoding a CasX protein and one or a plurality of gRNA of any of the embodiments of the disclosure comprising a targeting sequence region specific for a PTBP1 gene, optionally a donor template, and a suitable container. In other embodiments, the kit comprises a vector comprising a nucleic acid encoding a CasX protein and one or a plurality of gRNA of any of the embodiments of the disclosure comprising a targeting sequence region specific for a PTBP1 gene, and a suitable container. In still other embodiments, the kit comprises a XDP comprising a CasX protein and one or a plurality of gRNA of any of the embodiments of the disclosure comprising a targeting sequence region specific for a PTBP1 gene, optionally a donor template, and a suitable container. In still other embodiments, the kit comprises a composition comprising a plurality of cells edited using the CasX systems described herein.

In some embodiments, the kit further comprises a buffer, a nuclease inhibitor, a protease inhibitor, a liposome, a therapeutic agent, a label, a label visualization reagent, or any combination of the foregoing. In some embodiments, the kit further comprises a pharmaceutically acceptable carrier, diluent or excipient.

In some embodiments, the kit comprises appropriate control compositions for gene modifying applications, and instructions for use.

The present description sets forth numerous exemplary configurations, methods, parameters, and the like. It should be recognized, however, that such description is not intended as a limitation on the scope of the present disclosure, but is instead provided as a description of exemplary embodiments.

### ENUMERATED EMBODIMENTS

The invention may be defined by reference to the following enumerated, illustrative embodiments.

### Set I

Embodiment 1. A composition comprising a Class 2 Type V CRISPR protein and a first guide nucleic acid (gNA), wherein the gNA comprises a targeting sequence complementary to a polypyrimidine tract-binding protein 1 (PTBP1) gene target nucleic acid sequence.

Embodiment 2. The composition of embodiment 1, wherein the gNA comprises a targeting sequence complementary to a target nucleic acid sequence selected from the group consisting of:
a. a PTBP1 intron;
b. a PTBP1 exon;
c. a PTBP1 intron-exon junction;
d. a PTBP1 regulatory element; and
e. an intergenic region.

Embodiment 3. The composition of embodiment 1, wherein the PTBP1 gene comprises a wild-type sequence.

Embodiment 4. The composition of any one of embodiments 1-3, wherein the gNA is a guide RNA (gRNA).

Embodiment 5. The composition of any one of embodiments 1-3, wherein the gNA is a guide DNA (gDNA).

Embodiment 6. The composition of any one of embodiments 1-3, wherein the gNA is a chimera comprising DNA and RNA.

Embodiment 7. The composition of any one of embodiments 1-6, wherein the gNA is a single-molecule gNA (sgNA).

Embodiment 8. The composition of any one of embodiments 1-6, wherein the gNA is a dual-molecule gNA (dgNA).

Embodiment 9. The composition of any one of embodiments 1-8, wherein the targeting sequence of the gNA comprises a sequence selected from the group consisting of the sequences of SEQ ID NOS: 415-457, 492-2100 and 2286-43569, or a sequence having at least about 65%, at least about 75%, at least about 85%, or at least about 95% identity thereto.

Embodiment 10. The composition of any one of embodiments 1-8, wherein the targeting sequence of the gNA comprises a sequence selected from the group consisting of the sequences of SEQ ID NOS: 415-457, 492-2100 and 2286-43569.

Embodiment 11. The composition of any one of embodiments 1-8, wherein the targeting sequence of the gNA comprises a sequence of SEQ ID NOS: 415-457, 492-2100 and 2286-43569 with a single nucleotide removed from the 3′ end of the sequence.

Embodiment 12. The composition of any one of embodiments 1-8, wherein the targeting sequence of the gNA comprises a sequence of SEQ ID NOS: 415-457, 492-2100 and 2286-43569 with two nucleotides removed from the 3′ end of the sequence.

Embodiment 13. The composition of any one of embodiments 1-8, wherein the targeting sequence of the gNA comprises a sequence of SEQ ID NOS: 415-457, 492-2100 and 2286-43569 with three nucleotides removed from the 3′ end of the sequence.

Embodiment 14. The composition of any one of embodiments 1-8, wherein the targeting sequence of the gNA comprises a sequence of SEQ ID NOS: 415-457, 492-2100 and 2286-43569 with four nucleotides removed from the 3′ end of the sequence.

Embodiment 15. The composition of any one of embodiments 1-8, wherein the targeting sequence of the gNA comprises a sequence of SEQ ID NOS: 415-457, 492-2100 and 2286-43569 with five nucleotides removed from the 3′ end of the sequence.

Embodiment 16. The composition of any one of embodiments 1-15, wherein the targeting sequence of the gNA is complementary to a sequence of a PTBP1 exon.

Embodiment 17. The composition of embodiment 16, wherein the targeting sequence of the gNA is complementary to a sequence selected from the group consisting of PTBP1 exon 1, PTBP1 exon 2, PTBP1 exon 3, PTBP1 exon 4, PTBP1 exon 5, PTBP1 exon 6, PTBP1 exon 7, PTBP1 exon 8, PTBP1 exon 9, PTBP1 exon 10, PTBP1 exon 11, PTBP1 exon 12, PTBP1 exon 13, PTBP1 exon 14, PTBP1 exon 15, and PTBP1 exon 16.

Embodiment 18. The composition of embodiment 17, wherein the targeting sequence of the gNA is complementary to a sequence selected from the group consisting of PTBP1 exon 1, PTBP1 exon 2, and PTBP1 exon 3.

Embodiment 19. The composition of any one of embodiments 1-18, further comprising a second gNA, wherein the second gNA has a targeting sequence complementary to a different or overlapping portion of the PTBP1 target nucleic acid compared to the targeting sequence of the gNA of the first gNA.

Embodiment 20. The composition of embodiment 19, wherein the second gNA has a targeting sequence complementary to the same exon targeted by the first gNA.

Embodiment 21. The composition of any one of embodiments 1-20, wherein the first or second gNA has a scaffold comprising a sequence having at least about 50%, at least about 60%, at least about 70%, at least about 80%, at least about 90%, at least about 95%, at least about 96%, at least about 97%, at least about 98%, at least about 99%, or 100% sequence identity to a sequence selected from the group consisting of SEQ ID NO: 4-16 and 2101-2285.

Embodiment 22. The composition of any one of embodiments 1-20, wherein the first or second gNA has a scaffold comprising a sequence selected from the group consisting of SEQ ID NOs:2101-2285.

Embodiment 23. The composition of any one of embodiments 1-20, wherein the first or second gNA has a scaffold consisting of a sequence selected from the group consisting of SEQ ID NOs:2101-2285.

Embodiment 24. The composition of any one of embodiments 1-20, wherein the first or second gNA scaffold comprises a sequence having at least one modification relative to a reference gNA sequence selected from the group consisting of SEQ ID NOS: 4-16.

Embodiment 25. The composition of embodiment 24, wherein the at least one modification of the reference gNA comprises at least one substitution, deletion, or substitution of a nucleotide of the reference gNA sequence.

Embodiment 26. The composition of any one of embodiments 1-25, wherein the first or second gNA is chemically modified.

Embodiment 27. The composition of any one of embodiments 1-26, wherein the Class 2 Type V CRISPR protein is a reference CasX protein having a sequence of any one of SEQ ID NOS: 1-3, a CasX variant protein having a sequence of Table 4, or a sequence having at least about 50%, at least about 60%, at least about 70%, at least about 80%, at least about 90%, or at least about 95%, or at least about 95%, or at least about 96%, or at least about 97%, or at least about 98%, or at least about 99% sequence identity thereto.

Embodiment 28. The composition of embodiment 27, wherein the Class 2 Type V CRISPR protein is a CasX variant protein comprising a sequence of SEQ ID NOS: 36-99, 101-148, 188, 190, 208, 210, 212, 214, 216-229, 240, 242, 244, 246, 248, 250, 252, 254, 256 or 258.

Embodiment 29. The composition of embodiment 27, wherein the CasX variant protein consists of a sequence of SEQ ID NOS: 36-99, 101-148, 188, 190, 208, 210, 212, 214, 216-229, 240, 242, 244, 246, 248, 250, 252, 254, 256 or 258.

Embodiment 30. The composition of embodiment 27, wherein the CasX variant protein comprises at least one modification relative to a reference CasX protein having a sequence selected from SEQ ID NOS:1-3.

Embodiment 31. The composition of embodiment 30, wherein the at least one modification comprises at least one amino acid substitution, deletion, or substitution in a domain of the CasX variant protein relative to the reference CasX protein.

Embodiment 32. The composition of embodiment 31, wherein the domain is selected from the group consisting of a non-target strand binding (NTSB) domain, a target strand loading (TSL) domain, a helical I domain, a helical II domain, an oligonucleotide binding domain (OBD), and a RuvC DNA cleavage domain.

Embodiment 33. The composition of any one of embodiments 27-32, wherein the CasX protein further comprises one or more nuclear localization signals (NLS).

Embodiment 34. The composition of embodiment 33, wherein the one or more NLS are selected from the group of sequences consisting of PKKKRKV (SEQ ID NO: 149), KRPAATKKAGQAKKKK (SEQ ID NO: 150), PAAKRVKLD (SEQ ID NO: 151), RQRRNELKRSP (SEQ ID NO: 152), NQSSNFGPMKGGNFGGRSSGPYGGGGQYFAKPRNQGGY (SEQ ID NO: 153), RMRIZFKNKGKDTAELRRRRVEVSVELRKAKKDEQILKRRNV (SEQ ID NO: 154), VSRKRPRP (SEQ ID NO: 155), PPKKARED (SEQ ID NO: 156), PQPKKKPL (SEQ ID NO: 185), SALIKKKKKMAP (SEQ ID NO: 157), DRLRR (SEQ ID NO: 158), PKQKKRK (SEQ ID NO: 159), RKLKKKIKKL (SEQ ID NO: 160), REKKKFLKRR (SEQ ID NO: 161), KRKGDEVDGVDEVAKKKSKK (SEQ ID NO: 162), RKCLQAGMNLEARKTKK (SEQ ID NO: 163), PRPRKIPR (SEQ ID NO: 164), PPRKKRTVV (SEQ ID NO: 165), NLSKKKKRKREK (SEQ ID NO: 166), RRPSRPFRKP (SEQ ID NO: 167), KRPRSPSS (SEQ ID NO: 168), KRGINDRNFWRGENERKTR (SEQ ID NO: 169), PRPPKMARYDN (SEQ ID NO: 170), KRSFSKAF (SEQ ID NO: 186), KLKIKRPVK (SEQ ID NO: 171), PKTRRRPRRSQRKRPPT (SEQ ID NO: 173), RRKKRRPRRKKRR (SEQ ID NO: 176), PKKKSRKPKKKSRK (SEQ ID NO: 177), HKKKHPDASVNFSEFSK (SEQ ID NO: 178), QRPGPYDRPQRPGPYDRP (SEQ ID NO: 179), LSPSLSPLLSPSLSPL (SEQ ID NO: 180), RGKGGKGLGKGGAKRHRK (SEQ ID NO: 181), PKRGRGRPKRGRGR (SEQ ID NO: 182), MSRRRKANPTKLSENAKKLAKEVEN (SEQ ID NO: 174), PKKKRKVPPPPAAKRVKLD (SEQ ID NO: 172), and PKKKRKVPPPPKKKRKV (SEQ ID NO: 184).

Embodiment 35. The composition of embodiment 33 or embodiment 34, wherein the one or more NLS are expressed at or near the C-terminus of the CasX protein.

Embodiment 36. The composition of embodiment 33 or embodiment 34, wherein the one or more NLS are expressed at or near the N-terminus of the CasX protein.

Embodiment 37. The composition of embodiment 33 or embodiment 34, comprising one or more NLS located at or near the N-terminus and at or near the C-terminus of the CasX protein.

Embodiment 38. The composition of any one of embodiments 27-37, wherein the CasX variant is capable of forming a ribonuclear protein complex (RNP) with a guide nucleic acid (gNA).

Embodiment 39. The composition of embodiment 39, wherein an RNP of the CasX variant protein and the gNA variant exhibit at least one or more improved characteristics as compared to an RNP of a reference CasX protein of SEQ ID NO: 1, SEQ ID NO: 2, or SEQ ID NO: 3 and a gNA comprising a sequence of SEQ ID NOs: 4-16.

Embodiment 40. The composition of embodiment 39, wherein the improved characteristic is selected from one or more of the group consisting of improved folding of the CasX variant; improved binding affinity to a guide nucleic acid (gNA); improved binding affinity to a target DNA; improved ability to utilize a greater spectrum of one or more PAM sequences, including ATC, CTC, GTC, or TTC, in the editing of target DNA; improved unwinding of the target DNA; increased editing activity; improved editing efficiency; improved editing specificity; increased nuclease activity; increased target strand loading for double strand cleavage; decreased target strand loading for single strand nicking; decreased off-target cleavage; improved binding of non-target DNA strand; improved protein stability; improved protein solubility; improved protein:gNA complex (RNP) stability; improved protein:gNA complex solubility; improved protein yield; improved protein expression; and improved fusion characteristics.

Embodiment 41. The composition of embodiment 39 or embodiment 40, wherein the improved characteristic of the RNP of the CasX variant protein and the gNA variant is at least about 1.1 to about 100-fold or more improved relative to the RNP of the reference CasX protein of SEQ ID NO: 1, SEQ ID NO: 2, or SEQ ID NO: 3 and the gNA comprising a sequence of SEQ ID NOs: 4-16.

Embodiment 42. The composition of embodiment 39 or embodiment 40, wherein the improved characteristic of the CasX variant protein is at least about 1.1, at least about 2, at least about 10, at least about 100-fold or more improved relative to the reference CasX protein of SEQ ID NO: 1, SEQ ID NO: 2, or SEQ ID NO: 3 and the gNA comprising a sequence of SEQ ID NOs: 4-16.

Embodiment 43. The composition of any one of embodiments 39-42, wherein the improved characteristic comprises editing efficiency, and the RNP of the CasX variant protein and the gNA variant comprises a 1.1 to 100-fold improvement in editing efficiency compared to the RNP of the reference CasX protein of SEQ ID NO: 2 and the gNA of SEQ ID NOs: 4-16.

Embodiment 44. The composition of any one of embodiments 38-43, wherein the RNP comprising the CasX variant and the gNA variant exhibits greater editing efficiency and/or binding of a target sequence in the target DNA when any one of the PAM sequences TTC, ATC, GTC, or CTC is located 1 nucleotide 5′ to the non-target strand of the protospacer having identity with the targeting sequence of the gNA in a cellular assay system compared to the editing efficiency and/or binding of an RNP comprising a reference CasX protein and a reference gNA in a comparable assay system.

Embodiment 45. The composition of embodiment 44, wherein the PAM sequence is TTC.

Embodiment The composition of embodiment 44, wherein the PAM sequence is ATC.

Embodiment The composition of embodiment 44, wherein the PAM sequence is CTC.

Embodiment 48. The composition of embodiment 44, wherein the PAM sequence is GTC.

Embodiment 49. The composition of any one of embodiments 44-48, wherein the increased binding affinity for the one or more PAM sequences is at least 1.5-fold greater compared to the binding affinity of any one of the CasX proteins of SEQ ID NOS: 1-3 for the PAM sequences.

Embodiment 50. The composition of any one of embodiments 38-49, wherein the RNP has at least a 5%, at least a 10%, at least a 15%, or at least a 20% higher percentage of cleavage-competent RNP compared to an RNP of the reference CasX and the gNA of SEQ ID NOs: 4-16.

Embodiment 51. The composition of any one of embodiments 27-50, wherein the CasX variant protein comprises a RuvC DNA cleavage domain having nickase activity.

Embodiment 52. The composition of any one of embodiments 27-50, wherein the CasX variant protein comprises a RuvC DNA cleavage domain having double-stranded cleavage activity.

Embodiment 53. The composition of any one of embodiments 1-38, wherein the CasX protein is a catalytically inactive CasX (dCasX) protein, and wherein the dCasX and the gNA retain the ability to bind to the PTBP1 target nucleic acid.

Embodiment The composition of embodiment 53, wherein the dCasX comprises a mutation at residues:
a. D672, E769, and/or D935 corresponding to the CasX protein of SEQ ID NO: 1; or
b. D659, E756 and/or D922 corresponding to the CasX protein of SEQ ID NO: 2.

Embodiment 55. The composition of embodiment 54, wherein the mutation is a substitution of alanine for the residue.

Embodiment 56. The composition of any one of embodiments 1-52, further comprising a donor template nucleic acid.

Embodiment 57. The composition of embodiment 56, wherein the donor template comprises a nucleic acid comprising at least a portion of a PTBP1 gene selected from the group consisting of a PTBP1 exon, a PTBP1 intron, a PTBP1 intron-exon junction, and a PTBP1 regulatory element.

Embodiment 58. The composition of embodiment 57, wherein the donor template sequence comprises one or more mutations relative to a corresponding portion of a wild-type PTBP1 gene.

Embodiment 59. The composition of embodiment 57 or embodiment 58, wherein the donor template comprises a nucleic acid comprising at least a portion of a PTBP1 exon selected from the group consisting of PTBP1 exon 1, PTBP1 exon 2, PTBP1 exon 3, PTBP1 exon 4, PTBP1 exon 5, PTBP1 exon 6, PTBP1 exon 7, PTBP1 exon 8, PTBP1 exon 9, PTBP1 exon 10, PTBP1 exon 11, PTBP1 exon 12, PTBP1 exon 13, PTBP1 exon 14, PTBP1 exon 15, and PTBP1 exon 16.

Embodiment 60. The composition of embodiment 59, wherein the donor template comprises a nucleic acid comprising at least a portion of a PTBP1 exon selected from the group consisting of PTBP1 exon 1, PTBP1 exon 2, and PTBP1 exon 3.

Embodiment 61. The composition of any one of embodiments 56-60, wherein the donor template ranges in size from 10-15,000 nucleotides.

Embodiment 62. The composition of any one of embodiments 56-61, wherein the donor template is a single-stranded DNA template or a single stranded RNA template.

Embodiment 63. The composition of any one of embodiments 56-61, wherein the donor template is a double-stranded DNA template.

Embodiment 64. The composition of any one of embodiments 56-63, wherein the donor template comprises homologous arms at or near the 5′ and 3′ ends of the donor template that are complementary to sequences flanking cleavage sites in the PTBP1 target nucleic acid introduced by the Class 2 Type V CRISPR protein.

Embodiment 65. A nucleic acid comprising the donor template of any one of embodiments 56-64.

Embodiment 66. A nucleic acid comprising a sequence that encodes the CasX of any one of embodiments 27-55.

Embodiment 67. A nucleic acid comprising a sequence that encodes the gNA of any one of embodiments 1-26.

Embodiment 68. The nucleic acid of embodiment 66, wherein the sequence that encodes the CasX protein is codon optimized for expression in a eukaryotic cell.

Embodiment 69. A vector comprising the gNA of any one of embodiments 1-26, the CasX protein of any one of embodiments 27-55, or the nucleic acid of any one of embodiments 65-68.

Embodiment 70. The vector of embodiment 69, wherein the vector further comprises a promoter.

Embodiment 71. The vector of embodiment 69 or embodiment 70, wherein the vector is selected from the group consisting of a retroviral vector, a lentiviral vector, an adenoviral vector, an adeno-associated viral (AAV) vector, a herpes simplex virus (HSV) vector, a virus-like particle (VLP), a plasmid, a minicircle, a nanoplasmid, a DNA vector, and an RNA vector.

Embodiment 72. The vector of embodiment 71, wherein the vector is an AAV vector.

Embodiment 73. The vector of embodiment 72, wherein the AAV vector is selected from AAV1, AAV2, AAV3, AAV4, AAV5, AAV6, AAV7, AAV8, AAV9, AAV10, AAV11, AAV12, AAV-Rh74, or AAVRh10.

Embodiment 74. The vector of embodiment 71, wherein the vector is a retroviral vector.

Embodiment 75. The vector of embodiment 71, wherein the vector is a VLP comprising one or more components of a gag polyprotein.

Embodiment 76. The vector of embodiment 75, wherein the one or more components of the gag polyprotein are selected from the group consisting of matrix protein (MA), nucleocapsid protein (NC), capsid protein (CA), and p1-p6 protein.

Embodiment 77. The vector of embodiment 75 or embodiment 76, comprising the CasX protein and the gNA.

Embodiment 78. The vector of embodiment 77, wherein the CasX protein and the gNA are associated together in an RNP.

Embodiment 79. The vector of any one of embodiments 76-78, further comprising the donor template.

Embodiment 80. A host cell comprising the vector of any one of embodiments 69-79.

Embodiment 81. The host cell of embodiment 80, wherein the host cell is selected from the group consisting of BHK, HEK293, HEK293T, NS0, SP2/0, YO myeloma cells, P3X63 mouse myeloma cells, PER, PER.C6, NIH3T3, COS, HeLa, CHO, and yeast cells.

Embodiment 82. A method of modifying a PTBP1 target nucleic acid sequence in a population of cells, the method comprising introducing into cells of the population:
a. the composition of any one of embodiments 1-64;
b. the nucleic acid of any one of embodiments 65-68;
c. the vector as in any one of embodiments 69-74;
d. the VLP of any one of embodiments 76-79; or
e. combinations of two or more of (a)-(d),
wherein the PTBP1 gene target nucleic acid sequence of the cells targeted by the first gNA is modified by the CasX protein.

Embodiment 83. The method of embodiment 82, wherein the modifying comprises introducing a single-stranded break in the PTBP1 gene target nucleic acid sequence of the cells of the population.

Embodiment 84. The method of embodiment 82, wherein the modifying comprises introducing a double-stranded break in the PTBP1 gene target nucleic acid sequence of the cells of the population.

Embodiment 85. The method of any one of embodiments 82-84, further comprising introducing into the cells of the population a second gNA or a nucleic acid encoding the second gNA, wherein the second gNA has a targeting sequence complementary to a different or overlapping portion of the PTBP1 gene target nucleic acid compared to the first gNA, and wherein introducing the second gNA results in an additional break in the PTBP1 target nucleic acid of the cells of the population.

Embodiment 86. The method of any one of embodiments 82-85, wherein the modifying comprises introducing an insertion, deletion, substitution, duplication, or inversion of one or more nucleotides in the PTBP1 gene of the cells of the population.

Embodiment 87. The method of any one of embodiments 82-86, wherein the method comprises insertion of the donor template into the break site(s) of the PTBP1 gene target nucleic acid sequence of the cells of the population.

Embodiment 88. The method of embodiment 87, wherein the insertion of the donor template is mediated by homology-directed repair (HDR) or homology-independent targeted integration (HITI).

Embodiment 89. The method of embodiment 87 or embodiment 88, wherein insertion of the donor template results in a knock-down or knock-out of the PTBP1 gene in the cells of the population.

Embodiment 90. The method of any one of embodiments 82-89, wherein the PTBP1 gene of the cells of the population is modified such that expression of the PTBP1 protein is reduced by at least about 10%, at least about 20%, at least about 30%, at least about 40%, at least about 50%, at least about 60%, at least about 70%, at least about 80%, or at least about 90% in comparison to cells in which the PTBP1 gene has not been modified.

Embodiment 91. The method of any one of embodiments 82-89, wherein the PTBP1 gene of the cells of the population is modified such that at least about 10%, at least about 20%, at least about 30%, at least about 40%, at least about 50%, at least about 60%, at least about 70%, at least about 80%, or at least about 90% of the cells do not express a detectable level of PTBP1 protein.

Embodiment 92. The method of any one of embodiments 82-91, wherein the cells are eukaryotic.

Embodiment 93. The method of embodiment 92, wherein the eukaryotic cells are selected from the group consisting of rodent cells, mouse cells, rat cells, and non-human primate cells.

Embodiment 94. The method of embodiment 92, wherein the eukaryotic cells are human cells.

Embodiment 95. The method of any one of embodiments 92-94, wherein the eukaryotic cells are selected from the group consisting of microglial cells, astrocytes, oligodendrocytes, and fibroblasts.

Embodiment 96. The method of embodiment 95, wherein the modification of the PTBP1 target nucleic acid sequence results in reprogramming of the eukaryotic cells into neurons.

Embodiment 97. The method of any one of embodiment 82-96, wherein the modification of the PTBP1 gene target nucleic acid sequence of the population of cells occurs in vitro or ex vivo.

Embodiment 98. The method of any one of embodiment 82-96, wherein the modification of the PTBP1 gene target nucleic acid sequence of the population of cells occurs in vivo in a subject.

Embodiment 99. The method of embodiment 98, wherein the subject is selected from the group consisting of a rodent, a mouse, a rat, and a non-human primate.

Embodiment 100. The method of embodiment 98, wherein the subject is a human.

Embodiment 101. The method of any one of embodiments 98-100, wherein the method comprises administering a therapeutically effective dose of an AAV vector to the subject.

Embodiment 102. The method of embodiment 101, wherein the AAV vector is administered to the subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

Embodiment 103. The method of embodiment 101, wherein the AAV vector is administered to the subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

Embodiment 104. The method of any one of embodiments 98-100, wherein the method comprises administering a therapeutically effective dose of a VLP to the subject.

Embodiment 105. The method of embodiment 104, wherein the VLP is administered to the subject at a dose of at least about 1×10⁵ particles/kg, at least about 1×10⁶ particles/kg, at least about 1×10⁷ particles/kg at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, at least about 1×10¹⁶ particles/kg.

Embodiment 106. The method of embodiment 104, wherein the VLP is administered to the subject at a dose of at least about 1×10⁵ particles/kg to about 1×10¹⁶ particles/kg, or at least about 1×10⁶ particles/kg to about 1×10¹⁵ particles/kg, or at least about 1×10⁷ particles/kg to about 1×10¹⁴ particles/kg.

Embodiment 107. The method of any one of embodiments 99-106, wherein the vector or VLP is administered to the subject by a route of administration selected from intraparenchymal, intravenous, intra-arterial, intracerebroventricular, intracisternal, intrathecal, intracranial, lumbar, intraperitoneal, or combinations thereof.

Embodiment 108. The method of any one of embodiments 82-107, further comprising contacting the PTBP1 gene target nucleic acid sequence of the population of cells with:
a. an additional CRISPR nuclease and a gNA targeting a different or overlapping portion of the PTBP1 target nucleic acid compared to the first gNA;
b. a polynucleotide encoding the additional CRISPR nuclease and the gNA of (a);
c. a vector comprising the polynucleotide of (b); or
d. a VLP comprising the additional CRISPR nuclease and the gNA of (a),
wherein the contacting results in modification of the PTBP1 gene at a different location in the sequence compared to the sequence targeted by the first gNA.

Embodiment 109. The method of embodiment 108, wherein the additional CRISPR nuclease is a CasX protein having a sequence different from the CasX protein of any of the preceding embodiments.

Embodiment 110. The method of embodiment 108, wherein the additional CRISPR nuclease is not a CasX protein.

Embodiment 111. The method of embodiment 110, wherein the additional CRISPR nuclease is selected from the group consisting of Cas9, Cas12a, Cas12b, Cas12c, Cas12d (CasY), Cas12J, Cas13a, Cas13b, Cas13c, Cas13d, CasX, CasY, Cas14, Cpf1, C2c1, Csn2, and sequence variants thereof.

Embodiment 112. A population of cells modified by the method of any one of embodiments 82-111, wherein the cells have been modified such that at least 70%, at least 75%, at least 80%, at least 85%, at least 90%, or at least 95% of the modified cells do not express a detectable level of PTBP1 protein.

Embodiment 113. A population of cells modified by the method of any one of embodiments 82-111, wherein the cells have been modified such that the expression of PTBP1 protein is reduced by at least 70%, at least 75%, at least 80%, at least 85%, at least 90%, or at least 95% compared to cells where the PTBP1 gene has not been modified.

Embodiment 114. A method of treating a PTBP1-related disease in a subject in need thereof, the method comprising administering to the subject a therapeutically effective amount of the cells of embodiment 112 or embodiment 113.

Embodiment 115. The method of embodiment 114, wherein the PTBP1-related disease is a neurologic disease or neurologic injury.

Embodiment 116. The method of embodiment 115, wherein the neurologic disease or neurologic injury is selected from the group consisting of Parkinson's disease, Huntington's disease, Alzheimer's, amyotrophic lateral sclerosis (ALS), traumatic brain injury, and traumatic spinal cord injury.

Embodiment 117. The method of any one of embodiments 114-116, wherein the cells are autologous with respect to the subject to be administered the cells.

Embodiment 118. The method of any one of embodiments 114-116, wherein the cells are allogeneic with respect to the subject to be administered the cells.

Embodiment 119. The method of any one of embodiments 114-118, wherein the cells or their progeny persist in the subject for at least one month, two month, three months, four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the modified cells to the subject.

Embodiment 120. The method of any one of embodiments 114-119, wherein the method further comprises administering a chemotherapeutic agent.

Embodiment 121. The method of any one of embodiments 114-120, wherein the subject is selected from the group consisting of a rodent, a mouse, a rat, and a non-human primate.

Embodiment 122. The method of any one of embodiments 114-120, wherein the subject is a human.

Embodiment 123. A method of treating a PTBP1-related disease in a subject in need thereof, comprising modifying a PTBP1 gene in cells of the subject, the modifying comprising contacting said cells with a therapeutically effective dose of:
a. the composition of any one of embodiments 1-64;
b. the nucleic acid of any one of embodiments 65-68;
c. the vector as in any one of embodiments 69-74;
d. the VLP of any one of embodiments 75-79; or
e. combinations of two or more of (a)-(d),
wherein the PTBP1 gene of the cells targeted by the first gNA is modified by the CasX protein.

Embodiment 124. The method of embodiment 123, wherein the modifying comprises introducing a single-stranded break in the PTBP1 gene of the cells.

Embodiment 125. The method of embodiment 123, wherein the modifying comprises introducing a double-stranded break in the PTBP1 gene of the cells.

Embodiment 126. The method of any one of embodiments 123-125, further comprising introducing into the cells of the subject a second gNA or a nucleic acid encoding the second gNA, wherein the second gNA has a targeting sequence complementary to a different or overlapping portion of the target nucleic acid compared to the first gNA, resulting in an additional break in the PTBP1 target nucleic acid of the cells of the subject.

Embodiment 127. The method of any one of embodiments 123-126, wherein the modifying comprises introducing an insertion, deletion, substitution, duplication, or inversion of one or more nucleotides in the PTBP1 gene of the cells.

Embodiment 128. The method of embodiment 127, wherein the modifying results in a knock-down or knock-out of the PTBP1 gene in the modified cells of the subject.

Embodiment 129. The method of any one of embodiments 123-126, wherein the method comprises insertion of the donor template into the break site(s) of the PTBP1 gene target nucleic acid sequence of the cells.

Embodiment 130. The method of embodiment 129, wherein the insertion of the donor template is mediated by homology-directed repair (HDR) or homology-independent targeted integration (HITI).

Embodiment 131. The method of embodiment 129 or embodiment 130, wherein insertion of the donor template results in a knock-down or knock-out of the PTBP1 gene in the modified cells of the subject.

Embodiment 132. The method of any one of embodiments 123-131, wherein the PTBP1 gene of the cells are modified such that expression of the PTBP1 protein by the modified cells is reduced by at least about 10%, at least about 20%, at least about 30%, at least about 40%, at least about 50%, at least about 60%, at least about 70%, at least about 80%, or at least about 90% in comparison to cells that have not been modified.

Embodiment 133. The method of any one of embodiments 123-131, wherein the PTBP1 gene of the cells of the subject are modified such that at least 70%, at least 75%, at least 80%, at least 85%, at least 90%, or at least 95% of the modified cells do not express a detectable level of PTBP1 protein.

Embodiment 134. The method of any one of embodiments 123-133, wherein the PTBP1-related disease is a neurologic disease or neurologic injury.

Embodiment 135. The method of embodiment 134, wherein the neurologic disease or neurologic injury is selected from the group consisting of Parkinson's disease, Huntington's disease, Alzheimer's, amyotrophic lateral sclerosis (ALS), traumatic brain injury, and traumatic spinal cord injury.

Embodiment 136. The method of any one of embodiments 123-135, wherein the cells modified by the method are selected from the group consisting of microglial cells, astrocytes, oligodendrocytes, and fibroblasts.

Embodiment 137. The method of embodiment 136, wherein the cells are reprogrammed into functional neurons.

Embodiment 138. The method of any one of embodiments 123-133, wherein the PTBP1-related disease is a cancer.

Embodiment 139. The method of embodiment 138, wherein the cancer is selected from the group consisting of ovarian cancer, glioblastoma, bladder cancer, colon cancer and breast cancer.

Embodiment 140. The method of embodiment 138 or embodiment 139, wherein the modification of the PTBP1 gene results in prevention or reduction of tumorigenesis of the cells.

Embodiment The method of embodiment 138 or embodiment 139, wherein the modification of the PTBP1 target nucleic acid sequence results in stasis of an existing tumor in a subject.

Embodiment 142. The method of any one of embodiments 123-141, wherein the subject is selected from the group consisting of rodent, mouse, rat, and non-human primate.

Embodiment 143. The method of any one of embodiments 123-141, wherein the subject is a human.

Embodiment 144. The method of any one of embodiments 123-143, wherein the vector is AAV and is administered to the subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

Embodiment 145. The method of any one of embodiments 123-143, wherein the vector is AAV and is administered to the subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

Embodiment 146. The method of any one of embodiments 123-143, wherein the VLP is administered to the subject at a dose of at least about 1×10⁵ particles/kg, at least about 1×10⁶ particles/kg, at least about 1×10⁷ particles/kg at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, at least about 1×10¹⁶ particles/kg.

Embodiment 147. The method of any one of embodiments 123-143, wherein the VLP is administered to the subject at a dose of at least about 1×10⁵ particles/kg to about 1×10¹⁶ particles/kg, or at least about 1×10⁶ particles/kg to about 1×10¹⁵ particles/kg, or at least about 1×10⁷ particles/kg to about 1×10¹⁴ particles/kg.

Embodiment 148. The method of any one of embodiments 123-147, wherein the vector or VLP is administered to the subject by a route of administration selected from intraparenchymal, intravenous, intra-arterial, intracerebroventricular, intracisternal, intrathecal, intracranial, lumbar, intraperitoneal, or combinations thereof.

Embodiment 149. The method of any one of embodiments 121-148, wherein the method results in improvement in at least one clinically-relevant endpoint in the subject.

150. The method of embodiment 149, wherein the disease is Parkinson's disease and the clinically-relevant endpoint is selected from the group consisting of disease progression, Unified Parkinson's Disease Rating Scale (UPDRS), Unified Dyskinesia Rating Scale (UDysRS), Parkinson's Disease Quality of Life Questionnaire (PDQ-39) score, Movement Disorder Society-Sponsored Unified Parkinson's Disease Rating Scale (MDS-UPDRS), changes from baseline of motor score as measured by Inertial Measurement Unit (IMU) on Finger taping (FT) and Pronation-supination movement of the hands (PSH), delay in time to clinically meaningful worsening of motor progression, levodopa's duration of effect (“on time”), Clinical Global Impression—Improvement (CGI-I), change from baseline in Zarit Burden Interview score (ZBI), EQ-5D summary index, total disease duration, patient cognitive status (MMSE), and change from baseline in fatigue.

Embodiment 151. The method of embodiment 149, wherein the disease is Huntington's disease and the clinically-relevant endpoint is selected from the group consisting of Unified Huntington's Disease Rating Scale (UHDRS), cognitive decline, psychiatric abnormalities, motor impairment, changes in baseline in striatal volume, Stroop word test, total motor score (TMS), bradykinesia, dystonia, Symbol Digit Modalities Test, University of Pennsylvania Smell Identification Test, emotion recognition, speeded tapping, paced tapping, the Trail Making Test, intracranial-corrected volumes (ICV), and the Everyday Cognition Rating Scale (ECOG).

Embodiment 152. The method of embodiment 149, wherein the disease is ALS and the clinically-relevant endpoint is selected from the group consisting of ALS Functional Rating Scale (ALSFRS-(R)), combined assessment of function and survival, time to death, time to tracheostomy, time to persistent assisted ventilation (DTP), forced vital capacity (% FVC), manual muscle test, maximum voluntary isometric contraction, duration of response, progression-free survival, time to progression of disease, and time-to-treatment failure.

Embodiment 153. The method of embodiment 149, wherein the disease is Alzheimer's disease and the clinically-relevant endpoint is selected from the group consisting of change in Alzheimer's Disease Assessment Scale-Cognitive subscale (ADAS-Cog14) score, change in the Cohen-Mansfield Agitation Inventory (CMAI) score, change in the Alzheimer's Disease Cooperative Study-Instrumental Activities of Daily Living (ADCS-iADL) score, Clinical Dementia Rating Scale-Sum of Boxes (CDR-SB) score, DIAN Multivariate Cognitive Endpoint, Preclinical Alzheimer Cognitive Composite 5 (PACC5) score, Mini-Mental State Exam (MMSE) score, cognitive impairment, functional impairment, brain amyloid levels measured by amyloid positron emission tomography (PET), brain tau levels measured by PET, spinal fluid amyloid-β levels, and spinal fluid tau levels.

Embodiment 154. The method of embodiment 149, wherein the disease is cancer and the clinically-relevant endpoint is selected from the group consisting of tumor shrinkage as a complete, partial or incomplete response; time-to-progression; time to treatment failure; biomarker response; progression-free survival; disease free-survival; time to recurrence; time to metastasis; time of overall survival; improvement of quality of life; and improvement of symptoms.

Embodiment 155. The composition of any one of embodiments 1-64, the nucleic acid of any one of embodiments 65-68, the vector of any one of 69-74, the VLP of any one of embodiments 75-79, the host cell of embodiment 80 or embodiment 81, or the population of cells of embodiment 112-113, for use as a medicament for the treatment of a PTBP1 related disease.

Embodiment 156. The composition of embodiment 1, wherein the target nucleic acid sequence is complementary to a non-target strand sequence located 1 nucleotide 3′ of a protospacer adjacent motif (PAM) sequence.

Embodiment 157. The composition of embodiment 156, wherein the PAM sequence comprises a TC motif.

Embodiment 158. The composition of embodiment 157, wherein the PAM sequence comprises ATC, GTC, CTC or TTC.

Embodiment 159. The composition of any one of embodiments 156-158, wherein the Class 2 Type V CRISPR protein comprises a RuvC domain.

Embodiment 160. The composition of embodiment 159, wherein the RuvC domain generates a staggered double-stranded break in the target nucleic acid sequence.

Embodiment 161. The composition of any one of embodiments 156-160, wherein the Class 2 Type V CRISPR protein does not comprise an HNH nuclease domain.

### Set II

Embodiment 1. A system comprising a Class 2, Type V CRISPR protein and a first guide ribonucleic acid (gRNA), wherein the gRNA comprises a targeting sequence complementary to a polypyrimidine tract-binding protein 1 (PTBP1) gene target nucleic acid sequence.

Embodiment 2. The system of embodiment 1, wherein the gRNA comprises a targeting sequence complementary to a target nucleic acid sequence selected from the group consisting of:
a. a PTBP1 intron;
b. a PTBP1 exon;
c. a PTBP1 intron-exon junction;
d. a PTBP1 regulatory element; and
e. an intergenic region.

Embodiment 3. The system of embodiment 1 or embodiment 2, wherein the PTBP1 gene comprises a wild-type sequence.

Embodiment 4. The system of any one of embodiments 1-3, wherein the gRNA is a guide RNA (gRNA).

Embodiment 5. The system of any one of embodiments 1-3, wherein the gRNA is a chimera comprising DNA and RNA.

Embodiment 6. The system of any one of embodiments 1-5, wherein the gRNA is a single-molecule gRNA (sgRNA).

Embodiment 7. The system of any one of embodiments 1-5, wherein the gRNA is a dual-molecule gRNA (dgRNA).

Embodiment 8. The system of any one of embodiments 1-7, wherein the targeting sequence of the gRNA comprises a sequence selected from the group consisting of the sequences of SEQ ID NOS: 492-2100 and 2286-43569, or a sequence having at least about 65%, at least about 75%, at least about 85%, or at least about 95% identity thereto.

Embodiment 9. The system of any one of embodiments 1-7, wherein the targeting sequence of the gRNA comprises a sequence selected from the group consisting of the sequences of SEQ ID NOS: 492-2100 and 2286-43569.

Embodiment 10. The system of any one of embodiments 1-7, wherein the targeting sequence of the gRNA comprises a sequence of SEQ ID NOS: 492-2100 and 2286-43569 with a single nucleotide removed from the 3′ end of the sequence.

Embodiment 11. The system of any one of embodiments 1-7, wherein the targeting sequence of the gRNA comprises a sequence of SEQ ID NOS: 492-2100 and 2286-43569 with two nucleotides removed from the 3′ end of the sequence.

Embodiment 12. The system of any one of embodiments 1-7, wherein the targeting sequence of the gRNA comprises a sequence of SEQ ID NOS: 492-2100 and 2286-43569 with three nucleotides removed from the 3′ end of the sequence.

Embodiment 13. The system of any one of embodiments 1-7, wherein the targeting sequence of the gRNA comprises a sequence of SEQ ID NOS: 492-2100 and 2286-43569 with four nucleotides removed from the 3′ end of the sequence.

Embodiment 14. The system of any one of embodiments 1-7, wherein the targeting sequence of the gRNA comprises a sequence of SEQ ID NOS: 492-2100 and 2286-43569 with five nucleotides removed from the 3′ end of the sequence.

Embodiment 15. The system of any one of embodiments 1-7, wherein the targeting sequence of the gRNA comprises a sequence of SEQ ID NOS: 37971-37979, 38027, 38136, 38137, 38152-38158, 38160, 38162-38174, 38176, 38177, 38181, 38195, 38196, 38198, 38199, 38253-38256, 38259-38267, 38306 and 38311-38353.

Embodiment 16. The system of embodiment 15, wherein the targeting sequence of the gRNA is complementary to a sequence selected from the group consisting of PTBP1 exon 1, PTBP1 exon 2, PTBP1 exon 3, PTBP1 exon 4, PTBP1 exon 5, PTBP1 exon 6, PTBP1 exon 7, PTBP1 exon 8, PTBP1 exon 9, PTBP1 exon 10, PTBP1 exon 11, PTBP1 exon 12, PTBP1 exon 13, PTBP1 exon 14, PTBP1 exon 15, and PTBP1 exon 16.

Embodiment 17. The system of embodiment 16, wherein the targeting sequence of the gRNA is complementary to a sequence selected from the group consisting of PTBP1 exon 1, PTBP1 exon 2, and PTBP1 exon 3.

Embodiment 18. The system of any one of embodiments 1-17, further comprising a second gRNA, wherein the second gRNA has a targeting sequence complementary to a different or overlapping portion of the PTBP1 target nucleic acid compared to the targeting sequence of the gRNA of the first gRNA.

Embodiment 19. The system of embodiment 18, wherein the second gRNA has a targeting sequence complementary to the same exon targeted by the first gRNA.

Embodiment 20. The system of embodiment 18 or embodiment 19, wherein the first or second gRNA scaffold comprises a sequence having at least one modification relative to a reference gRNA sequence selected from the group consisting of SEQ ID NOS: 4-16.

Embodiment 21. The system of embodiment 20, wherein the at least one modification of the reference gRNA comprises;
a. at least one nucleotide substitution in a region of the gRNA variant;
b. at least one nucleotide deletion in a region of the gRNA variant;
c. at least one nucleotide insertion in a region of the gRNA variant;
d. a substitution of all or a portion of a region of the gRNA variant;
e. a deletion of all or a portion of a region of the gRNA variant; or
f. any combination of (a)-(e).

Embodiment 22. The system of embodiment 21, wherein the modified region of the gRNA variant is selected from the group consisting of extended stem loop, scaffold stem loop, triplex, and pseudoknot.

Embodiment 23. The gRNA variant of embodiment 22, wherein the scaffold stem further comprises a bubble.

Embodiment 24. The gRNA variant of embodiment 22 or embodiment 23, wherein the triplex further comprises a loop region.

Embodiment 25. The gRNA variant of any one of embodiments 21-24, wherein the scaffold further comprises a 5′ unstructured region.

Embodiment 26. The gRNA variant of any one of embodiments 21-25, wherein the at least one modification comprises:
a. a substitution of 1 to 15 consecutive or non-consecutive nucleotides in the gRNA variant in one or more regions;
b. a deletion of 1 to 10 consecutive or non-consecutive nucleotides in the gRNA variant in one or more regions;
c. an insertion of 1 to 10 consecutive or non-consecutive nucleotides in the gRNA variant in one or more regions;
d. a substitution of the scaffold stem loop or the extended stem loop with an RNA stem loop sequence from a heterologous RNA source; or
e. any combination of (a)-(d).

Embodiment 27. The gRNA variant of any one of embodiments 21-26, wherein the heterologous extended stem loop region comprises at least 10, at least 20, at least 100, at least 500, at least 1000, or at least 10,000 nucleotides.

Embodiment 28. The gRNA variant of embodiment 27, wherein the heterologous extended stem loop sequence increases the stability of the gRNA.

Embodiment 29. The gRNA variant of embodiment 27 or embodiment 28, wherein the heterologous RNA stem loop sequence is selected from one or more of MS2 hairpin, Qβ hairpin, U1 hairpin II, Uvsx, PP7 stem loop, or Rev Response Element (RRE), or a sequence variant thereof.

Embodiment 30. The gRNA variant of embodiment 29, wherein the heterologous RNA stem loop is capable of binding a protein, an RNA structure, a DNA sequence, or a small molecule.

Embodiment 31. The system of any one of embodiments 1-30, wherein the first or second gRNA has a scaffold comprising a sequence having at least about 50%, at least about 60%, at least about 70%, at least about 80%, at least about 90%, at least about 95%, at least about 96%, at least about 97%, at least about 98%, at least about 99%, or 100% sequence identity to a sequence selected from the group consisting of SEQ ID NOS: 2238-2285, 43571-43661, 44045 and 44047.

Embodiment 32. The system of any one of embodiments 1-30, wherein the first or second gRNA has a scaffold comprising a sequence selected from the group consisting of SEQ ID NOs: 2238-2285, 43571-43661, 44045 and 44047.

Embodiment 33. The system of any one of embodiments 1-30, wherein the first or second gRNA has a scaffold consisting of a sequence selected from the group consisting of SEQ ID NOs: 2238-2285, 43571-43661, 44045 and 44047.

Embodiment 34. The system of any one of embodiments 1-33, wherein the Class 2, Type V CRISPR protein is a CasX variant protein having at least one modification relative to a reference CasX protein having a sequence selected from the group consisting of SEQ ID NOS: 1-3 wherein the CasX variant exhibits at least one improved characteristic as compared to the reference CasX protein.

Embodiment 35. The system of embodiment 34, wherein the at least one modification comprises at least one amino acid substitution, deletion, or substitution in a domain of the CasX variant protein relative to the reference CasX protein.

Embodiment 36. The system of embodiment 35, wherein the domain is selected from the group consisting of a non-target strand binding (NTSB) domain, a target strand loading (TSL) domain, a helical I domain, a helical II domain, an oligonucleotide binding domain (OBD), and a RuvC DNA cleavage domain.

Embodiment 37. The system of any one of embodiments 34-36, wherein the CasX variant comprises an NTSB domain derived from SEQ ID NO: 1 and TSL, helical I, helical II domain, OBD, and RuvC domains derived from SEQ ID NO: 2.

Embodiment 38. The system of embodiment 37, wherein the CasX variant comprises the sequence of SEQ ID NO: 127.

Embodiment 39. The system of embodiment 37, wherein the CasX variant comprises a helical 1B domain derived from SEQ ID NO: 1

Embodiment 40. The system of embodiment 39, wherein the CasX variant comprises the sequence of SEQ ID NOS: 132-148 or 43662-43907.

Embodiment 41. The system of any one of embodiments 34-36, wherein the Class 2, Type V CRISPR protein is a CasX variant protein comprising a sequence selected from the group consisting of SEQ ID NOS: 59, 72-99, 101-148, and 43662-43907, or a sequence having at least about 50%, at least about 60%, at least about 70%, at least about 80%, at least about 90%, or at least about 95%, or at least about 95%, or at least about 96%, or at least about 97%, or at least about 98%, or at least about 99% sequence identity thereto.

Embodiment 42. The system of any one of embodiments 34-36, wherein the Class 2, Type V CRISPR protein is a CasX variant protein comprising a sequence selected from the group consisting of SEQ ID NOS: 59, 72-99, 101-148, and 43662-43907.

Embodiment 43. The system of any one of embodiments 34-36, wherein the CasX variant protein consists of a sequence selected from the group consisting of SEQ ID NOS: 59, 72-99, 101-148, and 43662-43907.

Embodiment 44. The system of any one of embodiments 34-43, wherein the CasX variant protein further comprises one or more nuclear localization signals (NLS).

Embodiment 45. The system of embodiment 44, wherein the one or more NLS are selected from the group of sequences consisting of PKKKRKV (SEQ ID NO: 149), KRPAATKKAGQAKKKK (SEQ ID NO: 150), PAAKRVKLD (SEQ ID NO: 151), RQRRNELKRSP (SEQ ID NO: 152), NQSSNFGPMKGGNFGGRSSGPYGGGGQYFAKPRNQGGY (SEQ ID NO: 153), RMRIZFKNKGKDTAELRRRRVEVSVELRKAKKDEQILKRRNV (SEQ ID NO: 154), VSRKRPRP (SEQ ID NO: 155), PPKKARED (SEQ ID NO: 156), PQPKKKPL (SEQ ID NO: 185), SALIKKKKKMAP (SEQ ID NO: 157), DRLRR (SEQ ID NO: 158), PKQKKRK (SEQ ID NO: 159), RKLKKKIKKL (SEQ ID NO: 160), REKKKFLKRR (SEQ ID NO: 161), KRKGDEVDGVDEVAKKKSKK (SEQ ID NO: 162), RKCLQAGMNLEARKTKK (SEQ ID NO: 163), PRPRKIPR (SEQ ID NO: 164), PPRKKRTVV (SEQ ID NO: 165), NLSKKKKRKREK (SEQ ID NO: 166), RRPSRPFRKP (SEQ ID NO: 167), KRPRSPSS (SEQ ID NO: 168), KRGINDRNFWRGENERKTR (SEQ ID NO: 169), PRPPKMARYDN (SEQ ID NO: 170), KRSFSKAF (SEQ ID NO: 186), KLKIKRPVK (SEQ ID NO: 171), PKTRRRPRRSQRKRPPT (SEQ ID NO: 173), RRKKRRPRRKKRR (SEQ ID NO: 176), PKKKSRKPKKKSRK (SEQ ID NO: 177), HKKKHPDASVNFSEFSK (SEQ ID NO: 178), QRPGPYDRPQRPGPYDRP (SEQ ID NO: 179), LSPSLSPLLSPSLSPL (SEQ ID NO: 180), RGKGGKGLGKGGAKRHRK (SEQ ID NO: 181), PKRGRGRPKRGRGR (SEQ ID NO: 182), MSRRRKANPTKLSENAKKLAKEVEN (SEQ ID NO: 174), PKKKRKVPPPPAAKRVKLD (SEQ ID NO: 172), and PKKKRKVPPPPKKKRKV (SEQ ID NO: 184).

Embodiment 46. The system of embodiment 44 or embodiment 45, wherein the one or more NLS are located at or near the C-terminus of the CasX variant protein.

Embodiment 47. The system of embodiment 44 or embodiment 45, wherein the one or more NLS are located at or near the N-terminus of the CasX variant protein.

Embodiment 48. The system of embodiment 44 or embodiment 45, comprising one or more NLS located at or near the N-terminus and at or near the C-terminus of the CasX variant protein.

Embodiment 49. The system of any one of embodiments 34-48, wherein the CasX variant is capable of forming a ribonuclear protein complex (RNP) with a gRNA.

Embodiment 50. The system of embodiment 50, wherein an RNP of the CasX variant protein and the gRNA variant exhibit at least one or more improved characteristics as compared to an RNP of a reference CasX protein of SEQ ID NO: 1, SEQ ID NO: 2, or SEQ ID NO: 3 and a gRNA comprising a sequence of SEQ ID NOs: 4-16.

Embodiment 51. The system of embodiment 50, wherein the improved characteristic is selected from one or more of the group consisting of improved folding of the CasX variant; improved binding affinity to a guide ribonucleic acid (gRNA); improved binding affinity to a target DNA; improved ability to utilize a greater spectrum of one or more PAM sequences, including ATC, CTC, GTC, or TTC, in the editing of target DNA; improved unwinding of the target DNA; increased editing activity; improved editing efficiency; improved editing specificity; increased nuclease activity; increased target strand loading for double strand cleavage; decreased target strand loading for single strand nicking; decreased off-target cleavage; improved binding of non-target DNA strand; improved protein stability; improved protein solubility; improved protein:gRNA complex (RNP) stability; and improved fusion characteristics.

Embodiment 52. The system of embodiment 50 or embodiment 51, wherein the improved characteristic of the RNP of the CasX variant protein and the gRNA variant is at least about 1.1 to about 100-fold or more improved relative to the RNP of the reference CasX protein of SEQ ID NO: 1, SEQ ID NO: 2, or SEQ ID NO: 3 and the gRNA comprising a sequence of SEQ ID NOs: 4-16.

Embodiment 53. The system of embodiment 50 or embodiment 51, wherein the improved characteristic of the CasX variant protein is at least about 1.1, at least about 2, at least about 10, at least about 100-fold or more improved relative to the reference CasX protein of SEQ ID NO: 1, SEQ ID NO: 2, or SEQ ID NO: 3 and the gRNA comprising a sequence of SEQ ID NOs: 4-16.

Embodiment 54. The system of any one of embodiments 50-53, wherein the improved characteristic comprises editing efficiency, and the RNP of the CasX variant protein and the gRNA variant comprises a 1.1 to 100-fold improvement in editing efficiency compared to the RNP of the reference CasX protein of SEQ ID NO: 2 and the gRNA of SEQ ID NOs: 4-16.

Embodiment 55. The system of any one of embodiments 49-54, wherein the RNP comprising the CasX variant and the gRNA variant exhibits greater editing efficiency and/or binding of a target sequence in the target DNA when any one of the PAM sequences TTC, ATC, GTC, or CTC is located 1 nucleotide 5′ to the non-target strand of the protospacer having identity with the targeting sequence of the gRNA in a cellular assay system compared to the editing efficiency and/or binding of an RNP comprising a reference CasX protein and a reference gRNA in a comparable assay system.

Embodiment 56. The system of embodiment 55, wherein the PAM sequence is TTC.

Embodiment 57. The system of embodiment 55, wherein the PAM sequence is ATC.

Embodiment 58. The system of embodiment 55, wherein the PAM sequence is CTC.

Embodiment 59. The system of embodiment 55, wherein the PAM sequence is GTC.

Embodiment 60. The system of any one of embodiments 55-59, wherein the increased binding affinity for the one or more PAM sequences is at least 1.5-fold greater compared to the binding affinity of any one of the reference CasX proteins of SEQ ID NOS: 1-3 for the PAM sequences.

Embodiment 61. The system of any one of embodiments 49-60, wherein the CasX variant and the gRNA variant are able to form RNP having at least about a 5%, at least about a 10%, at least about a 15%, or at least about a 20% higher percentage of cleavage-competent conformation compared to an RNP of any one of the reference CasX proteins of SEQ ID NOS: 1-3 and the gRNA of SEQ ID NOs: 4-16.

Embodiment 62. The system of any one of embodiments 49-61, wherein the RNP comprising the CasX variant and the gRNA variant exhibit a cleavage rate for the target nucleic acid in a timed in vitro assay that is at least about 5-fold, at least about 10-fold, or at least about 20-fold higher compared to an RNP of any one of the reference CasX proteins of SEQ ID NOS: 1-3 and the gRNA of SEQ ID NOs: 4-16 in a comparable assay.

Embodiment 63. The system of any one of embodiments 49-62, wherein the RNP comprising the CasX variant and the gRNA variant exhibit higher percent editing of the target nucleic acid in a timed in vitro assay that is at least about 5-fold, at least about 10-fold, at least about 20-fold, or at least about 100-fold higher compared to an RNP of any one of the reference CasX proteins of SEQ ID NOS: 1-3 and the gRNA of SEQ ID NOs: 4-16 in a comparable assay.

Embodiment 64. The system of any one of embodiments 34-63, wherein the CasX variant protein comprises a RuvC DNA cleavage domain having nickase activity.

Embodiment 65. The system of any one of embodiments 34-63, wherein the CasX variant protein comprises a RuvC DNA cleavage domain having double-stranded cleavage activity.

Embodiment 66. The system of any one of embodiments 34-49, wherein the CasX variant protein is a catalytically inactive CasX variant protein (dCasX), and wherein the dCasX and the gRNA retain the ability to bind to the PTBP1 target nucleic acid.

Embodiment 67. The system of embodiment 66, wherein the dCasX comprises a mutation at residues:
a. D672, E769, and/or D935 corresponding to the CasX protein of SEQ ID NO: 1; or
b. D659, E756 and/or D922 corresponding to the CasX protein of SEQ ID NO: 2.

Embodiment 68. The system of embodiment 67, wherein the mutation is a substitution of alanine for the residue.

Embodiment 69. The system of any one of embodiments 1-65, further comprising a donor template nucleic acid.

Embodiment 70. The system of embodiment 69, wherein the donor template comprises a nucleic acid comprising at least a portion of a PTBP1 gene selected from the group consisting of a PTBP1 exon, a PTBP1 intron, a PTBP1 intron-exon junction, and a PTBP1 regulatory element.

Embodiment 71. The system of embodiment 70, wherein the donor template sequence comprises one or more mutations relative to a corresponding portion of a wild-type PTBP1 gene.

Embodiment 72. The system of embodiment 70 or embodiment 71, wherein the donor template comprises a nucleic acid comprising at least a portion of a PTBP1 exon selected from the group consisting of PTBP1 exon 1, PTBP1 exon 2, PTBP1 exon 3, PTBP1 exon 4, PTBP1 exon 5, PTBP1 exon 6, PTBP1 exon 7, PTBP1 exon 8, PTBP1 exon 9, PTBP1 exon 10, PTBP1 exon 11, PTBP1 exon 12, PTBP1 exon 13, PTBP1 exon 14, PTBP1 exon 15, and PTBP1 exon 16.

Embodiment 73. The system of embodiment 72, wherein the donor template comprises a nucleic acid comprising at least a portion of a PTBP1 exon selected from the group consisting of PTBP1 exon 1, PTBP1 exon 2, and PTBP1 exon 3.

Embodiment 74. The system of any one of embodiments 69-73, wherein the donor template ranges in size from 10-15,000 nucleotides.

Embodiment 75. The system of any one of embodiments 69-74, wherein the donor template is a single-stranded DNA template or a single stranded RNA template.

Embodiment 76. The system of any one of embodiments 69-74, wherein the donor template is a double-stranded DNA template.

Embodiment 77. The system of any one of embodiments 69-76, wherein the donor template comprises homologous arms at or near the 5′ and 3′ ends of the donor template that are complementary to sequences flanking cleavage sites in the PTBP1 target nucleic acid introduced by the Class 2, Type V CRISPR protein.

Embodiment 78. A nucleic acid comprising the donor template of any one of embodiments 69-77.

Embodiment 79. A nucleic acid comprising a sequence that encodes the CasX variant of any one of embodiments 34-68.

Embodiment 80. A nucleic acid comprising a sequence that encodes the gRNA of any one of embodiments 1-33.

Embodiment 81. The nucleic acid of embodiment 79, wherein the sequence that encodes the CasX variant protein is codon optimized for expression in a eukaryotic cell.

Embodiment 82. A vector comprising the gRNA of any one of embodiments 1-33, the CasX variant protein of any one of embodiments 34-68, or the nucleic acid of any one of embodiments 78-81.

Embodiment 83. The vector of embodiment 82, wherein the vector further comprises a promoter.

Embodiment 84. The vector of embodiment 82, wherein the vector is selected from the group consisting of a retroviral vector, a lentiviral vector, an adenoviral vector, an adeno-associated viral (AAV) vector, a herpes simplex virus (HSV) vector, a CasX delivery particle (XDP), a plasmid, a minicircle, a nanoplasmid, a DNA vector, and an RNA vector.

Embodiment 85. The vector of embodiment 84, wherein the vector is an AAV vector.

Embodiment 86. The vector of embodiment 85, wherein the AAV vector is selected from AAV1, AAV2, AAV3, AAV4, AAV5, AAV6, AAV7, AAV8, AAV9, AAV10, AAV11, AAV12, AAV-Rh74, or AAVRh10.

Embodiment 87. The vector of embodiment 84, wherein the vector is a retroviral vector.

Embodiment 88. The vector of embodiment 84, wherein the vector is a XDP comprising one or more components of a gag polyprotein.

Embodiment 89. The vector of embodiment 88, wherein the one or more components of the gag polyprotein are selected from the group consisting of matrix protein (MA), a nucleocapsid protein (NC), a capsid protein (CA), a p1 peptide, a p6 peptide, a P2A peptide, a P2B peptide, a P10 peptide, a p12 peptide, a PP21/24 peptide, a P12/P3/P8 peptide, a P20 peptide, a protease cleavage site.

Embodiment 90. The vector of embodiment 88 or embodiment 89, comprising the CasX variant protein and the gRNA.

Embodiment 91. The vector of embodiment 90, wherein the CasX variant protein and the gRNA are associated together in an RNP.

Embodiment 92. The vector of any one of embodiments 88-91, further comprising a glycoprotein tropism factor.

Embodiment 93. The vector of any one of embodiments 88-92, wherein the glycoprotein tropism factor has binding affinity for a cell surface marker of a target cell and facilitates entry of the XDP into the target cell.

Embodiment 94. The vector of any one of embodiments 82-93, further comprising the donor template.

Embodiment 95. A host cell comprising the vector of any one of embodiments 82-94.

Embodiment 96. The host cell of embodiment 95, wherein the host cell is selected from the group consisting of Baby Hamster Kidney fibroblast (BHK) cells, human embryonic kidney 293 (HEK293), human embryonic kidney 293T (HEK293T) cells, NS0 cells, SP2/0 cells, YO myeloma cells, P3X63 mouse myeloma cells, PER cells, PER.C6 cells, hybridoma cells, NIH3T3 cells, CV-1 (simian) in Origin with SV40 genetic material (COS) cells, HeLa cells, Chinese hamster ovary (CHO) cells, or yeast cells.

Embodiment 97. A method of modifying a PTBP1 target nucleic acid sequence in a population of cells, the method comprising introducing into cells of the population:
a. the system of any one of embodiments 1-77;
b. the nucleic acid of any one of embodiments 78-81;
c. the vector as in any one of embodiments 82-87;
d. the XDP of any one of embodiments 89-93; or
e. combinations of two or more of (a)-(d),
wherein the PTBP1 gene target nucleic acid sequence of the cells targeted by the first gRNA is modified by the CasX variant protein.

Embodiment 98. The method of embodiment 97, wherein the modifying comprises introducing a single-stranded break in the PTBP1 gene target nucleic acid sequence of the cells of the population.

Embodiment 99. The method of embodiment 97, wherein the modifying comprises introducing a double-stranded break in the PTBP1 gene target nucleic acid sequence of the cells of the population.

Embodiment 100. The method of any one of embodiments 97-99, further comprising introducing into the cells of the population a second gRNA or a nucleic acid encoding the second gRNA, wherein the second gRNA has a targeting sequence complementary to a different or overlapping portion of the PTBP1 gene target nucleic acid compared to the first gRNA, and wherein introducing the second gRNA results in an additional break in the PTBP1 target nucleic acid of the cells of the population.

Embodiment 101. The method of any one of embodiments 97-100, wherein the modifying comprises introducing an insertion, deletion, substitution, duplication, or inversion of one or more nucleotides in the PTBP1 gene of the cells of the population.

Embodiment 102. The method of any one of embodiments 97-101, wherein the modifying comprises insertion of the donor template into the break site(s) of the PTBP1 gene target nucleic acid sequence of the cells of the population.

Embodiment 103. The method of embodiment 102, wherein the insertion of the donor template is mediated by homology-directed repair (HDR) or homology-independent targeted integration (HITI).

Embodiment 104. The method of any one of embodiments 97-102, wherein the modifying results in at least about 10%, at least about 20%, at least about 30%, at least about 40%, at least about 50%, at least about 60%, at least about 70%, at least about 80%, or at least about 90% edits in the PTBP1 gene in the modified cells of the population.

Embodiment 105. The method of any one of embodiments 97-104, wherein the modifying results in a knock-down or knock-out of the PTBP1 gene in the cells of the population.

Embodiment 106. The method of any one of embodiments 97-105, wherein the PTBP1 gene of the cells of the population is modified such that expression of the PTBP1 protein is reduced by at least about 10%, at least about 20%, at least about 30%, at least about 40%, at least about 50%, at least about 60%, at least about 70%, at least about 80%, or at least about 90% in comparison to cells in which the PTBP1 gene has not been modified.

Embodiment 107. The method of any one of embodiments 97-105, wherein the PTBP1 gene of the cells of the population is modified such that at least about 10%, at least about 20%, at least about 30%, at least about 40%, at least about 50%, at least about 60%, at least about 70%, at least about 80%, or at least about 90% of the cells do not express a detectable level of PTBP1 protein.

Embodiment 108. The method of any one of embodiments 97-107, wherein the cells are eukaryotic.

Embodiment 109. The method of embodiment 108, wherein the eukaryotic cells are selected from the group consisting of rodent cells, mouse cells, rat cells, and non-human primate cells.

Embodiment 110. The method of embodiment 108, wherein the eukaryotic cells are human cells.

Embodiment 111. The method of any one of embodiments 108-110, wherein the eukaryotic cells are selected from the group consisting of microglial cells, astrocytes, oligodendrocytes, and fibroblasts.

Embodiment 112. The method of embodiment 111, wherein the modification of the PTBP1 target nucleic acid sequence results in reprogramming of the eukaryotic cells into neurons.

Embodiment 113. The method of embodiment 112, wherein the modification of the PTBP1 target nucleic acid sequence results in an increase in expression of nPTB in the modified cells by at least about 10%, at least about 20%, at least about 30%, at least about 40%, at least about 50%, at least about 60%, at least about 70%, at least about 80%, or at least about 90% in comparison to cells in which the PTBP1 gene has not been modified.

Embodiment 114. The method of embodiment 112 or embodiment 113, wherein the PTBP1 gene of the cells of the population is modified such that at least about 10%, at least about 20%, at least about 30%, at least about 40%, at least about 50%, at least about 60%, at least about 70%, at least about 80%, or at least about 90% of the cells express a detectable level of nPTB protein.

Embodiment 115. The method of any one of embodiment 97-114, wherein the modification of the PTBP1 gene target nucleic acid sequence of the population of cells occurs in vitro or ex vivo.

Embodiment 116. The method of any one of embodiment 97-114, wherein the modification of the PTBP1 gene target nucleic acid sequence of the population of cells occurs in vivo in a subject.

Embodiment 117. The method of embodiment 116, wherein the subject is selected from the group consisting of a rodent, a mouse, a rat, and a non-human primate.

Embodiment 118. The method of embodiment 116, wherein the subject is a human.

Embodiment 119. The method of any one of embodiments 116-118, wherein the method comprises administering a therapeutically effective dose of an AAV vector to the subject.

Embodiment 120. The method of embodiment 119, wherein the AAV vector is administered to the subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

Embodiment 121. The method of embodiment 119, wherein the AAV vector is administered to the subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

Embodiment 122. The method of any one of embodiments 116-118, wherein the method comprises administering a therapeutically effective dose of a CasX delivery particle (XDP) to the subject.

Embodiment 123. The method of embodiment 122, wherein the XDP is administered to the subject at a dose of at least about 1×10⁵ particles/kg, at least about 1×10⁶ particles/kg, at least about 1×10⁷ particles/kg at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, at least about 1×10¹⁶ particles/kg.

Embodiment 124. The method of embodiment 122, wherein the XDP is administered to the subject at a dose of at least about 1×10⁵ particles/kg to about 1×10¹⁶ particles/kg, or at least about 1×10⁶ particles/kg to about 1×10¹⁵ particles/kg, or at least about 1×10⁷ particles/kg to about 1×10¹⁴ particles/kg

Embodiment 125. The method of any one of embodiments 116-124, wherein the vector or XDP is administered to the subject by a route of administration selected from intraparenchymal, intravenous, intra-arterial, intracerebroventricular, intracisternal, intrathecal, intracranial, lumbar, intraperitoneal, or combinations thereof.

Embodiment 126. The method of any one of embodiments 97-125, further comprising contacting the PTBP1 gene target nucleic acid sequence of the population of cells with:
a. an additional CRISPR nuclease and a gRNA targeting a different or overlapping portion of the PTBP1 target nucleic acid compared to the first gRNA;
b. a polynucleotide encoding the additional CRISPR nuclease and the gRNA of (a);
c. a vector comprising the polynucleotide of (b); or
d. a XDP comprising the additional CRISPR nuclease and the gRNA of (a),
wherein the contacting results in modification of the PTBP1 gene at a different location in the sequence compared to the sequence targeted by the first gRNA.

Embodiment 127. The method of embodiment 126, wherein the additional CRISPR nuclease is a CasX variant protein having a sequence different from the CasX variant protein of any of the preceding embodiments.

Embodiment 128. The method of embodiment 126, wherein the additional CRISPR nuclease is not a CasX protein.

Embodiment 129. The method of embodiment 128, wherein the additional CRISPR nuclease is selected from the group consisting of Cas9, Cas12a, Cas12b, Cas12c, Cas12d (CasY), Cas12J, Cas12k, Cas13a, Cas13b, Cas13c, Cas13d, Cas12j, Cas12k, CasY, Cas14, Cpf1, C2c1, Csn2, and sequence variants thereof.

Embodiment 130. A population of cells modified by the method of any one of embodiments 97-129, wherein the cells have been modified such that at least 70%, at least 75%, at least 80%, at least 85%, at least 90%, or at least 95% of the modified cells do not express a detectable level of PTBP1 protein.

Embodiment 131. A population of cells modified by the method of any one of embodiments 97-129, wherein the cells have been modified such that the expression of PTBP1 protein is reduced by at least 70%, at least 75%, at least 80%, at least 85%, at least 90%, or at least 95% compared to cells where the PTBP1 gene has not been modified.

Embodiment 132. A population of cells modified by the method of any one of embodiments 97-129, wherein the cells have been modified such that the expression of nPTB protein is increased by at least about 10%, at least about 20%, at least about 30%, at least about 40%, at least about 50%, at least about 60%, at least about 70%, at least about 80%, or at least about 90% in comparison to cells in which the PTBP1 gene has not been modified.

Embodiment 133. A method of treating a PTBP1-related disease in a subject in need thereof, the method comprising administering to the subject a therapeutically effective amount of the cells of any one of embodiments 130-132.

Embodiment 134. The method of embodiment 133, wherein the PTBP1-related disease is a neurologic disease or neurologic injury.

Embodiment 135. The method of embodiment 134, wherein the neurologic disease or neurologic injury is selected from the group consisting of Parkinson's disease, Huntington's disease, Alzheimer's, amyotrophic lateral sclerosis (ALS), traumatic brain injury, and traumatic spinal cord injury.

Embodiment 136. The method of any one of embodiments 133-135, wherein the cells are autologous with respect to the subject to be administered the cells.

Embodiment 137. The method of any one of embodiments 133-135, wherein the cells are allogeneic with respect to the subject to be administered the cells.

Embodiment 138. The method of any one of embodiments 133-137, wherein the cells or their progeny persist in the subject for at least one month, two month, three months, four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the modified cells to the subject.

Embodiment 139. The method of any one of embodiments 133-138, wherein the method further comprises administering a chemotherapeutic agent.

Embodiment 140. The method of any one of embodiments 133-139, wherein the subject is selected from the group consisting of a rodent, a mouse, a rat, and a non-human primate.

Embodiment 141. The method of any one of embodiments 133-139, wherein the subject is a human.

Embodiment 142. A method of treating a PTBP1-related disease in a subject in need thereof, comprising modifying a PTBP1 gene in cells of the subject, the modifying comprising contacting said cells with a therapeutically effective dose of:
a. the system of any one of embodiments 1-77;
b. the nucleic acid of any one of embodiments 78-81;
c. the vector as in any one of embodiments 82-87;
d. the XDP of any one of embodiments 88-93; or
e. combinations of two or more of (a)-(d),
wherein the PTBP1 gene of the cells targeted by the first gRNA is modified by the CasX variant protein.

Embodiment 143. The method of embodiment 142, wherein the modifying comprises introducing a single-stranded break in the PTBP1 gene of the cells.

Embodiment 144. The method of embodiment 142, wherein the modifying comprises introducing a double-stranded break in the PTBP1 gene of the cells.

Embodiment 145. The method of any one of embodiments 142-144, further comprising introducing into the cells of the subject a second gRNA or a nucleic acid encoding the second gRNA, wherein the second gRNA has a targeting sequence complementary to a different or overlapping portion of the target nucleic acid compared to the first gRNA, resulting in an additional break in the PTBP1 target nucleic acid of the cells of the subject.

Embodiment 146. The method of any one of embodiments 142-145, wherein the modifying comprises introducing an insertion, deletion, substitution, duplication, or inversion of one or more nucleotides in the PTBP1 gene of the cells.

Embodiment 147. The method of any one of embodiments 142-145, wherein the modifying comprises insertion of the donor template into the break site(s) of the PTBP1 gene target nucleic acid sequence of the cells.

Embodiment 148. The method of embodiment 147, wherein the insertion of the donor template is mediated by homology-directed repair (HDR) or homology-independent targeted integration (HITI).

Embodiment 149. The method of any one of embodiments 142-148, wherein the modifying results in edits in the PTBP1 gene in at least about 10%, at least about 20%, at least about 30%, at least about 40%, at least about 50%, at least about 60%, at least about 70%, at least about 80%, or at least about 90% edits of the modified cells of the subject.

Embodiment 150. The method of any one of embodiments 142-149, wherein the modifying results in a knock-down or knock-out of the PTBP1 gene in the modified cells of the subject.

Embodiment 151. The method of any one of embodiments 142-149, wherein the PTBP1 gene of the cells of the subject are modified such that expression of the PTBP1 protein by the modified cells is reduced by at least about 10%, at least about 20%, at least about 30%, at least about 40%, at least about 50%, at least about 60%, at least about 70%, at least about 80%, or at least about 90% in comparison to cells that have not been modified.

Embodiment 152. The method of any one of embodiments 142-149, wherein the PTBP1 gene of the cells of the subject are modified such that at least 50%, at least 60%, at least 70%, at least 75%, at least 80%, at least 85%, at least 90%, or at least 95% of the modified cells do not express a detectable level of PTBP1 protein.

Embodiment 153. The method of any one of embodiments 142-152, wherein the cells modified by the method are selected from the group consisting of microglial cells, astrocytes, oligodendrocytes, and fibroblasts.

Embodiment 154. The method of embodiment 153, wherein the modification results in reprogramming of the modified cells into neurons.

Embodiment 155. The method of any one of embodiments 142-154, wherein the modification results in an increase in expression of nPTB in the modified cells by at least about 10%, at least about 20%, at least about 30%, at least about 40%, at least about 50%, at least about 60%, at least about 70%, at least about 80%, or at least about 90% in comparison to cells in which the PTBP1 gene has not been modified.

Embodiment 156. The method of any one of embodiments 142-155, wherein at least about 10%, at least about 20%, at least about 30%, at least about 40%, at least about 50%, at least about 60%, at least about 70%, at least about 80%, or at least about 90% of the modified cells express a detectable level of nPTB protein.

Embodiment 157. The method of any one of embodiments 142-156, wherein the PTBP1-related disease is a neurologic disease or neurologic injury.

Embodiment 158. The method of embodiment 157, wherein the neurologic disease or neurologic injury is selected from the group consisting of Parkinson's disease, Huntington's disease, Alzheimer's, amyotrophic lateral sclerosis (ALS), traumatic brain injury, and traumatic spinal cord injury.

Embodiment 159. The method of any one of embodiments 142-152, wherein the PTBP1-related disease is a cancer.

Embodiment 160. The method of embodiment 159, wherein the cancer is selected from the group consisting of ovarian cancer, glioblastoma, bladder cancer, colon cancer and breast cancer.

Embodiment 161. The method of embodiment 159 or embodiment 160, wherein the modification of the PTBP1 gene results in prevention or reduction of tumorigenesis of the cells.

Embodiment 162. The method of embodiment 159 or embodiment 160, wherein the modification of the PTBP1 target nucleic acid sequence results in stasis of an existing tumor in a subject.

Embodiment 163. The method of any one of embodiments 142-162, wherein the subject is selected from the group consisting of rodent, mouse, rat, and non-human primate.

Embodiment 164. The method of any one of embodiments 142-162, wherein the subject is a human.

Embodiment 165. The method of any one of embodiments 142-164, wherein the vector is AAV and is administered to the subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

Embodiment 166. The method of any one of embodiments 142-164, wherein the vector is AAV and is administered to the subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

Embodiment 167. The method of any one of embodiments 142-164, wherein the XDP is administered to the subject at a dose of at least about 1×10⁵ particles/kg, at least about 1×10⁶ particles/kg, at least about 1×10⁷ particles/kg at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, at least about 1×10¹⁶ particles/kg.

Embodiment 168. The method of any one of embodiments 142-164, wherein the XDP is administered to the subject at a dose of at least about 1×10⁵ particles/kg to about 1×10¹⁶ particles/kg, or at least about 1×10⁶ particles/kg to about 1×10¹⁵ particles/kg, or at least about 1×10⁷ particles/kg to about 1×10¹⁴ particles/kg

Embodiment 169. The method of any one of embodiments 142-168, wherein the vector or XDP is administered to the subject by a route of administration selected from intraparenchymal, intravenous, intra-arterial, intracerebroventricular, intracisternal, intrathecal, intracranial, lumbar, intraperitoneal, or combinations thereof.

Embodiment 170. The method of any one of embodiments 142-169, wherein the method results in improvement in at least one clinically-relevant endpoint in the subject.

Embodiment 171. The method of embodiment 170, wherein the disease is Parkinson's disease and the clinically-relevant endpoint is selected from the group consisting of disease progression, Unified Parkinson's Disease Rating Scale (UPDRS), Unified Dyskinesia Rating Scale (UDysRS), Parkinson's Disease Quality of Life Questionnaire (PDQ-39) score, Movement Disorder Society-Sponsored Unified Parkinson's Disease Rating Scale (MDS-UPDRS), changes from baseline of motor score as measured by Inertial Measurement Unit (IMU) on Finger taping (FT) and Pronation-supination movement of the hands (PSH), delay in time to clinically meaningful worsening of motor progression, levodopa's duration of effect (“on time”), Clinical Global Impression—Improvement (CGI-I), change from baseline in Zarit Burden Interview score (ZBI), EQ-5D summary index, total disease duration, patient cognitive status (MMSE), and change from baseline in fatigue.

Embodiment 172. The method of embodiment 170, wherein the disease is Huntington's disease and the clinically-relevant endpoint is selected from the group consisting of Unified Huntington's Disease Rating Scale (UHDRS), cognitive decline, psychiatric abnormalities, motor impairment, changes in baseline in striatal volume, Stroop word test, total motor score (TMS), bradykinesia, dystonia, Symbol Digit Modalities Test, University of Pennsylvania Smell Identification Test, emotion recognition, speeded tapping, paced tapping, the Trail Making Test, intracranial-corrected volumes (ICV), and the Everyday Cognition Rating Scale (ECOG).

Embodiment 173. The method of embodiment 170, wherein the disease is ALS and the clinically-relevant endpoint is selected from the group consisting of ALS Functional Rating Scale (ALSFRS-(R)), combined assessment of function and survival, time to death, time to tracheostomy, time to persistent assisted ventilation (DTP), forced vital capacity (% FVC), manual muscle test, maximum voluntary isometric contraction, duration of response, progression-free survival, time to progression of disease, and time-to-treatment failure.

Embodiment 174. The method of embodiment 170, wherein the disease is Alzheimer's disease and the clinically-relevant endpoint is selected from the group consisting of change in Alzheimer's Disease Assessment Scale-Cognitive subscale (ADAS-Cog14) score, change in the Cohen-Mansfield Agitation Inventory (CMAI) score, change in the Alzheimer's Disease Cooperative Study-Instrumental Activities of Daily Living (ADCS-iADL) score, Clinical Dementia Rating Scale-Sum of Boxes (CDR-SB) score, DIAN Multivariate Cognitive Endpoint, Preclinical Alzheimer Cognitive Composite 5 (PACC5) score, Mini-Mental State Exam (MMSE) score, cognitive impairment, functional impairment, brain amyloid levels measured by amyloid positron emission tomography (PET), brain tau levels measured by PET, spinal fluid amyloid-β levels, and spinal fluid tau levels.

Embodiment 175. The method of embodiment 170, wherein the disease is cancer and the clinically-relevant endpoint is selected from the group consisting of tumor shrinkage as a complete, partial or incomplete response; time-to-progression; time to treatment failure; biomarker response; progression-free survival; disease free-survival; time to recurrence; time to metastasis; time of overall survival; improvement of quality of life; and improvement of symptoms.

Embodiment 176. The system of any one of embodiments 1-77, the nucleic acid of any one of embodiments 78-81, the vector of any one of 82-87, the XDP of any one of embodiments 88-93, the host cell of embodiment 95 or embodiment 96, or the population of cells of any one of embodiments 130-132, for use as a medicament for the treatment of a PTBP1 related disease.

Embodiment 177. The system of any one of embodiments 1-77, wherein the target nucleic acid sequence is complementary to a non-target strand sequence located 1 nucleotide 3′ of a protospacer adjacent motif (PAM) sequence.

Embodiment 178. The system of embodiment 177, wherein the PAM sequence comprises a TC motif.

Embodiment 179. The system of embodiment 178, wherein the PAM sequence comprises ATC, GTC, CTC or TTC.

Embodiment 180. The system of any one of embodiments 177-179, wherein the Class 2 Type V CRISPR protein comprises a RuvC domain.

Embodiment 181. The system of embodiment 180, wherein the RuvC domain generates a staggered double-stranded break in the target nucleic acid sequence.

Embodiment 182. The system of any one of embodiments 177-181, wherein the Class 2 Type V CRISPR protein does not comprise an HNH nuclease domain.

Embodiment 183. A composition of the Class 2, type V CRISPR protein of any one of embodiments 34-65 and the gRNA of any one of embodiments 1-33 as gene editing pairs for use as a medicament for the treatment of a subject having a PTBP1-related disease.

### EXAMPLES

### Example 1: Generating CasX Variant Constructs

In order to generate the CasX 488 construct (sequences in Table 6), the codon-optimized CasX 119 construct (based on the CasX Stx2 construct, encoding Planctomycetes CasX SEQ ID NO: 2, with amino acid substitutions and deletions) was cloned into a destination plasmid (pStX) using standard cloning methods. In order to generate the CasX 491 construct (sequences in Table 6), the codon-optimized CasX 484 construct (based on the CasX Stx2 construct, encoding Planctomycetes CasX SEQ ID NO: 2, with substitutions and deletions of certain amino acids, with fused NLS, and linked guide and non-targeting sequences) was cloned into a destination plasmid (pStX) using standard cloning methods. Construct CasX 1 (CasX SEQ ID NO: 1) was cloned into a destination vector using standard cloning methods. To build CasX 488, the CasX 119 construct DNA was PCR amplified in two reactions using Q5 DNA polymerase according to the manufacturer's protocol, using universal appropriate primers. To build CasX 491, the codon optimized CasX 484 construct DNA was PCR amplified in two reactions using Q5 DNA polymerase according to the manufacturer's protocol, using appropriate primers. The CasX 1 construct was PCR amplified in two reactions using Q5 DNA polymerase according to the manufacturer's protocol, universal appropriate primers. Each of the PCR products were purified by gel extraction from a 1% agarose gel (Gold Bio Cat #A-201-500) using Zymoclean Gel DNA Recovery Kit according to the manufacturer's protocol. The corresponding fragments were then pieced together using Gibson assembly (New England BioLabs Cat #E2621S) following the manufacturer's protocol. Assembled products in pStx1 were transformed into chemically-competent Turbo Competent E. coli bacterial cells, plated on LB-Agar plates containing kanamycin. Individual colonies were picked and miniprepped using Qiagen spin Miniprep Kit following the manufacturer's protocol. The resultant plasmids were sequenced using Sanger sequencing to ensure correct assembly. The correct clones were then subcloned into the mammalian expression vector pStx34 using restriction enzyme cloning. The pStx34 backbone and the CasX 488 and 491 clones in pStx1 were digested with XbaI and BamHI respectively. The digested backbone and respective insert fragments were purified by gel extraction from a 1% agarose gel (Gold Bio Cat #A-201-500) using Zymoclean Gel DNA Recovery Kit according to the manufacturer's protocol. The clean backbone and insert were then ligated together using T4 Ligase (New England Biolabs Cat #M0202L) according to the manufacturer's protocol. The ligated products were transformed into chemically-competent Turbo Competent E. coli bacterial cells, plated on LB-Agar plates containing carbenicillin. Individual colonies were picked and miniprepped using Qiagen spin Miniprep Kit following the manufacturer's protocol. The resultant plasmids were sequenced using Sanger sequencing to ensure correct assembly.

To build CasX 515 (sequences in Table 6), the CasX 491 construct DNA was PCR amplified in two reactions using Q5 DNA polymerase according to the manufacturer's protocol, using appropriate primers. To build CasX 527 (sequences in Table 6), the CasX 491 construct DNA was PCR amplified in two reactions using Q5 DNA polymerase according to the manufacturer's protocol, using appropriate primers. The PCR products were purified by gel extraction from a 1% agarose gel using Zymoclean Gel DNA Recovery Kit according to the manufacturer's protocol. The pStX backbone was digested using XbaI and SpeI in order to remove the 2931 base pair fragment of DNA between the two sites in plasmid pStx56. The digested backbone fragment was purified by gel extraction from a 1% agarose gel using Zymoclean Gel DNA Recovery Kit according to the manufacturer's protocol. The insert and backbone fragments were then pieced together using Gibson assembly (New England BioLabs Cat #E2621S) following the manufacturer's protocol. Assembled products in the pStx56 were transformed into chemically-competent Turbo Competent E. coli bacterial cells, plated on LB-Agar plates containing kanamycin. Individual colonies were picked and miniprepped using Qiagen spin Miniprep Kit following the manufacturer's protocol. The resultant plasmids were sequenced using Sanger sequencing to ensure correct assembly. pStX34 includes an EF-lα promoter for the protein as well as a selection marker for both puromycin and carbenicillin. pStX56 includes an EF-lα promoter for the protein as well as a selection marker for both puromycin and kanamycin Sequences encoding the targeting sequences that target the gene of interest were designed based on CasX PAM locations. Targeting sequence DNA was ordered as single-stranded DNA (ssDNA) oligos (Integrated DNA Technologies) consisting of the targeting sequence and the reverse complement of this sequence. These two oligos were annealed together and cloned into pStX individually or in bulk by Golden Gate assembly using T4 DNA Ligase and an appropriate restriction enzyme for the plasmid. Golden Gate products were transformed into chemically or electro-competent cells such as NEB Turbo competent E. coli (NEB Cat #C2984I), plated on LB-Agar plates containing the appropriate antibiotic. Individual colonies were picked and miniprepped using Qiaprep spin Miniprep Kit and following the manufacturer's protocol. The resultant plasmids were sequenced using Sanger sequencing to ensure correct ligation.

To build CasX 535-537 (sequences in Table 6), the CasX 515 construct DNA was PCR amplified in two reactions for each construct using Q5 DNA polymerase according to the manufacturer's protocol. For CasX 535, appropriate primers were used for the amplification. For CasX 536 appropriate primers were used. For CasX 537, appropriate primers were used. The PCR products were purified by gel extraction from a 1% agarose gel using Zymoclean Gel DNA Recovery Kit according to the manufacturer's protocol. The pStX backbone was digested using XbaI and SpeI in order to remove the 2931 base pair fragment of DNA between the two sites in plasmid pStx56. The digested backbone fragment was purified by gel extraction from a 1% agarose gel using Zymoclean Gel DNA Recovery Kit according to the manufacturer's protocol. The insert and backbone fragments were then pieced together using Gibson assembly following the manufacturer's protocol. Assembled products in pStx56 were transformed into chemically-competent Turbo Competent E. coli bacterial cells, plated on LB-Agar plates containing kanamycin. Individual colonies were picked and miniprepped using Qiagen spin Miniprep Kit following the manufacturer's protocol. The resultant plasmids were sequenced using Sanger sequencing to ensure correct assembly. pStX34 includes an EF-lα promoter for the protein as well as a selection marker for both puromycin and carbenicillin. pStX56 includes an EF-lα promoter for the protein as well as a selection marker for both puromycin and kanamycin. Sequences encoding the targeting sequences that target the gene of interest were designed based on CasX PAM locations. Targeting sequence DNA was ordered as single-stranded DNA (ssDNA) oligos (Integrated DNA Technologies) consisting of the targeting sequence and the reverse complement of this sequence. These two oligos were annealed together and cloned into pStX individually or in bulk by Golden Gate assembly using T4 DNA Ligase and an appropriate restriction enzyme for the plasmid. Golden Gate products were transformed into chemically or electro-competent cells such as NEB Turbo competent E. coli, plated on LB-Agar plates containing the appropriate antibiotic. Individual colonies were picked and miniprepped using Qiaprep spin Miniprep Kit and following the manufacturer's protocol. The resultant plasmids were sequenced using Sanger sequencing to ensure correct ligation.

All subsequent CasX variants, such as CasX 544 and CasX 660-664, 668, 670, 672, 676, and 677 were cloned using the same methodology as described above using Gibson assembly with mutation-specific internal primers and universal forward and reverse primers (the differences between them were the mutation specific primers designed as well as which CasX base construct was used). SaCas9 and SpyCas9 control plasmids were prepared similarly to pStX plasmids described above, with the protein and guide regions of pStX exchanged for the respective protein and guide. Targeting sequences for SaCas9 and SpyCas9 were either obtained from the literature or were rationally designed according to established methods.

The expression and recovery of the CasX constructs was performed using standard methodologies and are summarized as follows:

### Purification:

Frozen samples were thawed overnight at 4° C. with magnetic stirring. The viscosity of the resulting lysate was reduced by sonication and lysis was completed by homogenization in two passes at 20 k PSI using a NanoDeBEE (BEE International). Lysate was clarified by centrifugation at 50,000×g, 4° C., for 30 minutes and the supernatant was collected. The clarified supernatant was applied to a Heparin 6 Fast Flow column (Cytiva) using an AKTA Pure FPLC (Cytiva). The column was washed with 5 CV of Heparin Buffer A (50 mM HEPES-NaOH, 250 mM NaCl, 5 mM MgCl2, 0.5 mM TCEP, 10% glycerol, pH 8), then with 3 CV of Heparin Buffer B (Buffer A with the NaCl concentration adjusted to 500 mM). Protein was eluted with 1.75 CV of Heparin Buffer C (Buffer A with the NaCl concentration adjusted to 1 M). The eluate was applied to a StrepTactin HP column (Cytiva) using the FPLC. The column was washed with 10 CV of Strep Buffer (50 mM HEPES-NaOH, 500 mM NaCl, 5 mM MgCl2, 0.5 mM TCEP, 10% glycerol, pH 8). Protein was eluted from the column using 1.65 CV of Strep Buffer with 2.5 mM Desthiobiotin added. CasX-containing fractions were pooled, concentrated at 4° C. using a 50 kDa cut-off spin concentrator (Amicon), and purified by size exclusion chromatography on a Superdex 200 pg column (Cytiva). The column was equilibrated with SEC Buffer (25 mM sodium phosphate, 300 mM NaCl, 1 mM TCEP, 10% glycerol, pH 7.25) and operated by FPLC. CasX-containing fractions that eluted at the appropriate molecular weight were pooled, concentrated at 4° C. using a 50 kDa cut-off spin concentrator, aliquoted, and snap-frozen in liquid nitrogen before being stored at −80° C.

CasX variant 488: The average yield was 2.7 mg of purified CasX protein per liter of culture at 98.8% purity, as evaluated by colloidal Coomassie staining.

CasX Variant 491: The average yield was 12.4 mg of purified CasX protein per liter of culture at 99.4% purity, as evaluated by colloidal Coomassie staining.

CasX variant 515: The average yield was 7.8 mg of purified CasX protein per liter of culture at 90% purity, as evaluated by colloidal Coomassie staining.

CasX variant 526: The average yield was 13.79 mg per liter of culture, at 93% purity. Purity was evaluated by colloidal Coomassie staining.

CasX variant 668: The average yield was 3.32 mg per liter of culture, at 93% purity. Purity was evaluated by colloidal Coomassie staining.

CasX variant 672: The average yield was 6.50 mg per liter of culture, at 88% purity. Purity was evaluated by colloidal Coomassie staining.

CasX variant 676: The average yield was 5.05 mg per liter of culture, at 92% purity. Purity was evaluated by colloidal Coomassie staining.

CasX variant 677: The average yield was 2.93 mg per liter of culture, at 81% purity. Purity was evaluated by colloidal Coomassie staining.

TABLE 6
CasX variant DNA and amino acid sequences

| | SEQ ID NO of DNA | |
|---|---|---|
| Construct | Sequence | SEQ ID NO of Amino Acid Sequence |
| CasX 488 | 43936 | 123 |
| CasX 491 | 43937 | 126 |
| CasX 515 | 43938 | 133 |
| CasX 527 | 43939 | 144 |
| CasX 535 | 43940 | 43665 |
| CasX 536 | 43941 | 43666 |
| CasX 537 | 43942 | 43667 |
| CasX 583 | 43943 | 43712 |
| CasX 660 | 43944 | 43789 |
| CasX 661 | 43945 | 43790 |
| CasX 662 | 43946 | 43791 |
| CasX 663 | 43947 | 43792 |
| CasX 664 | 43948 | 43793 |
| CasX 668 | 43949 | 43797 |
| CasX 670 | 43950 | 43935 |
| CasX 672 | 43951 | 43800 |
| CasX 676 | 43952 | 43804 |
| CasX 677 | 43953 | 43805 |

### Example 2: Generation of RNA Guides

For the generation of RNA single guides and targeting sequences, templates for in vitro transcription were generated by performing PCR with Q5 polymerase, template primers for each backbone, and amplification primers with the T7 promoter and the targeting sequence. The DNA primer sequences for the T7 promoter, guide and targeting sequence for guides and targeting sequences are presented in Table 7, below. The sg1, sg2, sg32, sg64, sg174, and sg235 guides correspond to SEQ ID NOS: 4, 5, 2104, 2106, 2238, and 43577, respectively, with the exception that sg2, sg32, and sg64 were modified with an additional 5′ G to increase transcription efficiency (compare sequences in Table 7 to Table 3). The 7.37 targeting sequence targets beta2-microglobulin (B2M). Following PCR amplification, templates were cleaned and isolated by phenol-chloroform-isoamyl alcohol extraction followed by ethanol precipitation.

In vitro transcriptions were carried out in buffer containing 50 mM Tris pH 8.0, 30 mM MgCl2, 0.01% Triton X-100, 2 mM spermidine, 20 mM DTT, 5 mM NTPs, 0.5 μM template, and 100 μg/mL T7 RNA polymerase. Reactions were incubated at 37° C. overnight. 20 units of DNase I (Promega #M6101)) were added per 1 mL of transcription volume and incubated for one hour. RNA products were purified via denaturing PAGE, ethanol precipitated, and resuspended in 1× phosphate buffered saline. To fold the sgRNAs, samples were heated to 70° C. for 5 min and then cooled to room temperature. The reactions were supplemented to 1 mM final MgCl2 concentration, heated to 50° C. for 5 min and then cooled to room temperature. Final RNA guide products were stored at −80° C.

TABLE 7
Sequences for generation of guide RNA

| | SEQ ID | | SEQ |
|---|---|---|---|
| Primer | NO: | RNA product | ID NO: |
| T7 promoter | 259 | Used for all | ND |
| primer | | | |
| sg2 backbone | 260 | GGUACUGGCGCUUUUAUCUCAUUACUUUGAGAGCCAUCAC | 272 |
| fwd | | CAGCGACUAUGUCGUAUGGGUAAAGCGCUUAUUUAUCGGA | |
| sg2 backbone | 261 | GAGAAAUCCGAUAAAUAAGAAGCAUCAAAGGGCCGAGAUG | |
| rev | | UCUCGCUCCG | |
| sg2.7.37 spacer | 262 | | |
| primer | | | |
| sg32 backbone | 263 | GGUACUGGCGCUUUUAUCUCAUUACUUUGAGAGCCAUCAC | 273 |
| fwd | | CAGCGACUAUGUCGUAUGGGUAAAGCGCCCUCUUCGGAGG | |
| sg32 backbone | 264 | GAAGCAUCAAAGGGCCGAGAUGUCUCG | |
| rev | | | |
| sg32.7.37 spacer | 265 | | |
| primer | | | |
| sg64 backbone | 266 | GGUACUGGCGCCUUUAUCUCAUUACUUUGAGAGCCAUCAC | 274 |
| fwd | | CAGCGACUAUGUCGUAUGGGUAAAGCGCUUACGGACUUCG | |
| sg64 backbone | 267 | GUCCGUAAGAAGCAUCAAAGGGCCGAGAUGUCUCGCUCCG | |
| rev | | | |
| sg64.7.37 spacer | 268 | | |
| primer | | | |
| sg174 backbone | 269 | ACUGGCGCUUUUAUCUgAUUACUUUGAGAGCCAUCACCAG | 275 |
| fwd | | CGACUAUGUCGUAgUGGGUAAAGCUCCCUCUUCGGAGGGA | |
| sg174 backbone | 270 | GCAUCAAAGGGCCGAGAUGUCUCGCUCCG | |
| rev | | | |
| sg174.7.37 | 271 | | |
| spacer primer | | | |
| sg235 backbone | ND | ACUGGCGCUUCUAUCUGAUUACUCUGAGCGCCAUCACCAG | 43957 |
| fwd | | CGACUAUGUCGUAGUGGGUAAAGCCGCUUACGGACUUCGG | |
| sg235 backbone | ND | UCCGUAAGAGGCAUCAGAG | |
| rev | | | |
| sg235.7.37 | ND | | |
| spacer primer | | | |

### Example 3: Assessing Binding Affinity to the Guide RNA

Purified wild-type and improved CasX will be incubated with synthetic single-guide RNA containing a 3′ Cy7.5 moiety in low-salt buffer containing magnesium chloride as well as heparin to prevent non-specific binding and aggregation. The sgRNA will be maintained at a concentration of 10 pM, while the protein will be titrated from 1 pM to 100 μM in separate binding reactions. After allowing the reaction to come to equilibrium, the samples will be run through a vacuum manifold filter-binding assay with a nitrocellulose membrane and a positively charged nylon membrane, which bind protein and nucleic acid, respectively. The membranes will be imaged to identify guide RNA, and the fraction of bound vs unbound RNA will be determined by the amount of fluorescence on the nitrocellulose vs nylon membrane for each protein concentration to calculate the dissociation constant of the protein-sgRNA complex. The experiment will also be carried out with improved variants of the sgRNA to determine if these mutations also affect the affinity of the guide for the wild-type and mutant proteins. We will also perform electromobility shift assays to qualitatively compare to the filter-binding assay and confirm that soluble binding, rather than aggregation, is the primary contributor to protein-RNA association.

### Example 4: Assessing Binding Affinity to the Target DNA

Purified wild-type and improved CasX will be complexed with single-guide RNA bearing a targeting sequence complementary to the target nucleic acid. The RNP complex will be incubated with double-stranded target DNA containing a PAM and the appropriate target nucleic acid sequence with a 5′ Cy7.5 label on the target strand in low-salt buffer containing magnesium chloride as well as heparin to prevent non-specific binding and aggregation. The target DNA will be maintained at a concentration of 1 nM, while the RNP will be titrated from 1 pM to 100 μM in separate binding reactions. After allowing the reaction to come to equilibrium, the samples will be run on a native 5% polyacrylamide gel to separate bound and unbound target DNA. The gel will be imaged to identify mobility shifts of the target DNA, and the fraction of bound vs unbound DNA will be calculated for each protein concentration to determine the dissociation constant of the RNP-target DNA ternary complex.

### Example 5: Assessing Differential PAM Recognition In Vitro

### 1. Comparison of Reference and CasX Variants

In vitro cleavage assays were performed using CasX2 (SEQ ID NO:2), CasX119, and CasX438 complexed with sg174.7.37, essentially as describe in Example 8. Fluorescently labeled dsDNA targets with a 7.37 spacer and either a TTC, CTC, GTC, or ATC PAM were used (sequences are in Table 8). Time points were taken at 0.25, 0.5, 1, 2, 5, 10, 30, and 60 minutes. Gels were imaged with an Cytiva Typhoon and quantified using the IQTL 8.2 software. Apparent first-order rate constants for non-target strand cleavage (k_{cleave}) were determined for each CasX:sgRNA complex on each target. Rate constants for targets with non-TTC PAM were compared to the TTC PAM target to determine whether the relative preference for each PAM was altered in a given protein variant.

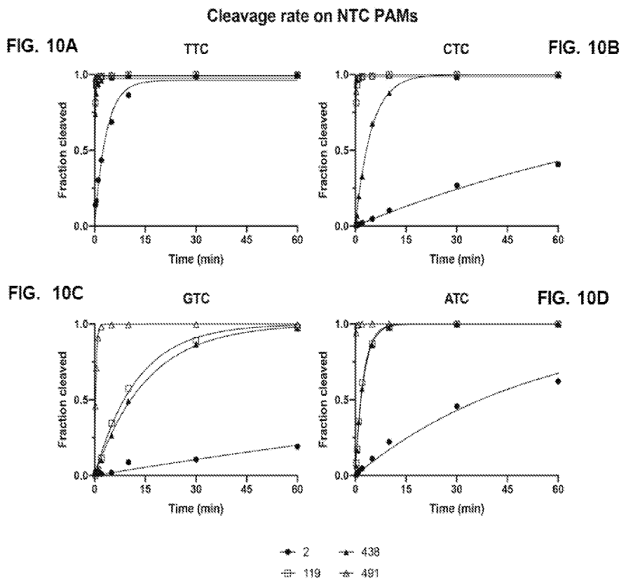

For all variants, the TTC target supported the highest cleavage rate, followed by the ATC, then the CTC, and finally the GTC target (FIGS. 10A-D, Table 9). For each combination of CasX variant and NTC PAM, the cleavage rate k_{cleave} is shown. For all non-NTC PAMs, the relative cleavage rate as compared to the TTC rate for that variant is shown in parentheses. All non-TTC PAMs exhibited substantially decreased cleavage rates (>10-fold for all). The ratio between the cleavage rate of a given non-TTC PAM and the TTC PAM for a specific variant remained generally consistent across all variants. The CTC target supported cleavage 3.5-4.3% as fast as the TTC target; the GTC target supported cleavage 1.0-1.4% as fast; and the ATC target supported cleavage 6.5-8.3% as fast. The exception is for 491, where the kinetics of cleavage at TTC PAMs are too fast to allow accurate measurement, which artificially decreases the apparent difference between TTC and non-TTC PAMs. Comparing the relative rates of 491 on GTC, CTC, and ATC PAMs, which fall within the measurable range, results in ratios comparable to those for other variants when comparing across non-TTC PAMs, consistent with the rates increasing in tandem. Overall, differences between the variants are not substantial enough to suggest that the relative preference for the various NTC PAMs have been altered. However, the higher basal cleavage rates of the variants allow targets with ATC or CTC PAMs to be cleaved nearly completely within 10 minutes, and the apparent k_{cleaves} are comparable to or greater than the k_{cleave} of CasX2 on a TTC PAM (Table 9). This increased cleavage rate may cross the threshold necessary for effective genome editing in a human cell, explaining the apparent increase in PAM flexibility for these variants. Experiments utilizing CasX variants 515-791 (SEQ ID NOS: 133-148 and 43662-43907) would be conducted using similar methodology, and would be expected to result in enhanced cleavage rates relative to reference CasX utilizing one or more PAM sequences in the target nucleic acid.

TABLE 8
Sequences of DNA substrates used in in vitro PAM cleavage assay

| Guide* | DNA Sequence | SEQ ID NO |
|---|---|---|
| 7.37 TTC PAM | AGCGCGAGCACAGCTAAGGCCACGGAGCGAGACATCTCGGCCCGAATGCTG | 43958 |
| TS | TCAGCTTCA | |
| 7.37 TTC PAM | TGAAGCTGACAGCATTCGGGCCGAGATGTCTCGCTCCGTGGCCTTAGCTGT | 43959 |
| NTS | GCTCGCGCT | |
| 7.37 CTC PAM | AGCGCGAGCACAGCTAAGGCCACGGAGCGAGACATCTCGGCCCGAGTGCTG | 43960 |
| TS | TCAGCTTCA | |
| 7.37 CTC PAM | TGAAGCTGACAGCACTCGGGCCGAGATGTCTCGCTCCGTGGCCTTAGCTGT | 43961 |
| NTS | GCTCGCGCT | |
| 7.37 GTC PAM | AGCGCGAGCACAGCTAAGGCCACGGAGCGAGACATCTCGGCCCGACTGCTG | 43962 |
| TS | TCAGCTTCA | |
| 7.37 GTC PAM | TGAAGCTGACAGCAGTCGGGCCGAGATGTCTCGCTCCGTGGCCTTAGCTGT | 43963 |
| NTS | GCTCGCGCT | |
| 7.37 ATC PAM | AGCGCGAGCACAGCTAAGGCCACGGAGCGAGACATCTCGGCCCGATTGCTG | 43964 |
| TS | TCAGCTTCA | |
| 7.37 ATC PAM | TGAAGCTGACAGCAATCGGGCCGAGATGTCTCGCTCCGTGGCCTTAGCTGT | 43965 |
| NTS | GCTCGCGCT | |

*The PAM sequences for each are bolded.
TS—target strand.
NTS—Non-target strand.

TABLE 9
Apparent cleavage rates of CasX variants against NTC PAMs

| Variant | TTC | CTC | GTC | ATC |
|---|---|---|---|---|
| 2 | 0.267 min⁻¹ | 9.29E-3 min⁻¹ | 3.75E-3 min⁻¹ | 1.87E-2 min⁻¹ |
| | | (0.035) | (0.014) | (0.070) |
| 119 | 8.33 min⁻¹ | 0.303 min⁻¹ | 8.64E-2 min⁻¹ | 0.540 min⁻¹ |
| | | (0.036) | (0.010) | (0.065) |
| 438 | 4.94 min⁻¹ | 0.212 min⁻¹ | 1.31E-2 min⁻¹ | 0.408 min⁻¹ |
| | | (0.043) | (0.013) | (0.083) |
| 491 | 16.42 min⁻¹ | 8.605 min⁻¹ | 2.447 min⁻¹ | 11.33 min⁻¹ |
| | | (0.524) | (0.149) | (0.690) |

### 2. Comparison of PAM Recognition Using Single CasX Variant

Materials and Methods: Fluorescently labeled dsDNA targets with a 7.37 spacer and either a TTC, CTC, GTC, ATC, TTT, CTT, GTT, or ATT PAM were used (sequences are in Table 10). Oligos were ordered with a 5′ amino modification and labeled with a Cy7.5 NHS ester for target strand oligos and a Cy5.5 NHS ester for non-target strand oligos. dsDNA targets were formed by mixing the oligos in a 1:1 ratio in 1×cleavage buffer (20 mM Tris HCl pH 7.5, 150 mM NaCl, 1 mM TCEP, 5% glycerol, 10 mM MgCl2), heating to 95° C. for 10 minutes, and allowing the solution to cool to room temperature.

CasX variant 491 was complexed with sg174.7.37. The guide was diluted in 1× cleavage buffer to a final concentration of 1.5 μM, and then protein was added to a final concentration of 1 μM. The RNP was incubated at 37° C. for 10 minutes and then put on ice.

Cleavage assays were carried out by diluting RNP in cleavage buffer to a final concentration of 200 nM and adding dsDNA target to a final concentration of 10 nM. Time points were taken at 0.25, 0.5, 1, 2, 5, and 10 minutes and quenched by adding to an equal volume of 95% formamide and 20 mM EDTA. Cleavage products were resolved by running on a 10% urea-PAGE gel. Gels were imaged with an Amersham Typhoon and quantified using the IQTL 8.2 software. Apparent first-order rate constants for non-target strand cleavage (kcleave) were determined for each target using GraphPad Prism.

### Results:

The relative cleavage rate of the 491.174 RNP on various PAMs was investigated. In addition to aiding in the prediction of cleavage efficiencies of targets and potential off-targets in cells, these data will also allow us to adjust the cleavage rate of synthetic targets. In the case of self-limiting AAV vectors, where new protospacers can be added within the vector to allow for self-targeting, we reasoned that the rate of episome cleavage could be adjusted up or down by changing the PAM. Experiments utilizing CasX variants 515-791 (SEQ ID NOS: 133-148 and 43662-43907) would be conducted using similar methodology, and would be expected to result in cleavage rates relative to reference CasX utilizing one or more PAM sequences in the target nucleic acid.

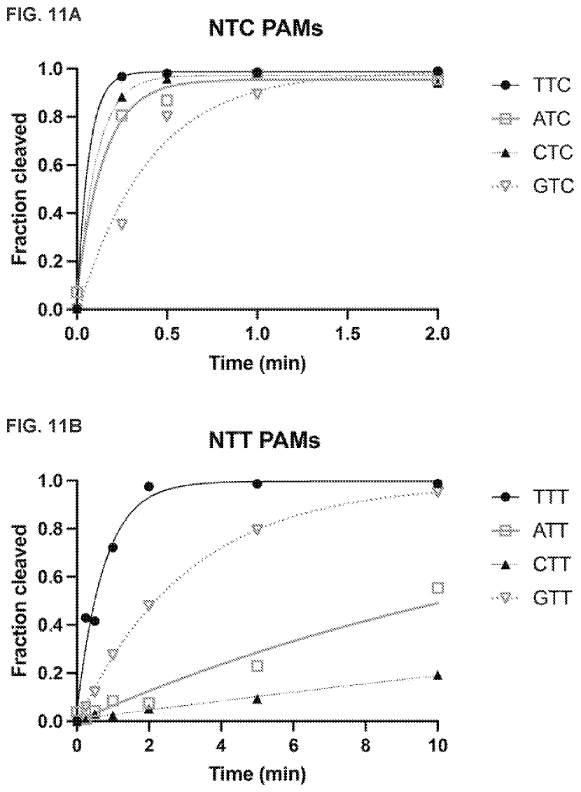

We tested the cleavage rate of the RNP against various dsDNA substrates that were identical in sequence aside from the PAM. This experimental setup should allow for the isolation of the effects of the PAM itself, rather than convoluting PAM recognition with effects resulting from spacer sequence and genomic context. All NTC and NTT PAMs were tested. As expected, the RNP cleaved the target with the TTC PAM most quickly, converting essentially all of it to product by the first time point (FIG. 11A). CTC was cleaved roughly half as quickly, though the rapid cleavage of TTC makes determining an accurate kcleave difficult under these assay conditions, which are optimized to capture a broader array of cleavage rates (FIG. 11A, Table 11). The GTC target was cleaved most slowly of the NTC PAMs, with a cleavage rate roughly six-fold slower than the TTC target. All NTT PAMs were cleaved more slowly than all NTC PAMs, with TTT cut most efficiently, followed by GTT (FIG. 11B, Table 11). The relative efficiency of GTT cleavage among all NTT PAMs, compared to the low rate of GTC cleavage compared to all NTC PAMs, demonstrates that recognition of individual PAM nucleotides is context-dependent, with nucleotide identity at one position in the PAM affecting sequence preference at the other positions.

The PAM sequences tested here yield cleavage rates spanning three orders of magnitude while still maintaining cleavage activity at the same spacer sequence. These data demonstrate that cleavage rates at a given synthetic target can be readily modified by changing the associated PAM, allowing for adjustment of self-cleavage activity to allow for efficient targeting of the genomic target prior to cleavage and elimination of the AAV episome.

TABLE 10
Sequences of DNA substrates used in in vitro PAM cleavage assay*

| PAM & Strand | Spacer and PAM Sequence | SEQ ID NO |
|---|---|---|
| 7.37 TTC PAM TS | AGCGCGAGCACAGCTAAGGCCACGGAGCGAGACATCTCGGCCCGAAT | 43958 |
| | GCTGTCAGCTTCA | |
| 7.37 TTC PAM | TGAAGCTGACAGCATTCGGGCCGAGATGTCTCGCTCCGTGGCCTTAG | 43959 |
| NTS | CTGTGCTCGCGCT | |
| 7.37 CTC PAM TS | AGCGCGAGCACAGCTAAGGCCACGGAGCGAGACATCTCGGCCCGAGT | 43960 |
| | GCTGTCAGCTTCA | |
| 7.37 CTC PAM | TGAAGCTGACAGCACTCGGGCCGAGATGTCTCGCTCCGTGGCCTTAG | 43961 |
| NTS | CTGTGCTCGCGCT | |
| 7.37 GTC PAM TS | AGCGCGAGCACAGCTAAGGCCACGGAGCGAGACATCTCGGCCCGACT | 43962 |
| | GCTGTCAGCTTCA | |
| 7.37 GTC PAM | TGAAGCTGACAGCAGTCGGGCCGAGATGTCTCGCTCCGTGGCCTTAG | 43963 |
| NTS | CTGTGCTCGCGCT | |
| 7.37 ATC PAM TS | AGCGCGAGCACAGCTAAGGCCACGGAGCGAGACATCTCGGCCCGATT | 43964 |
| | GCTGTCAGCTTCA | |
| 7.37 ATC PAM | TGAAGCTGACAGCAATCGGGCCGAGATGTCTCGCTCCGTGGCCTTAG | 43965 |
| NTS | CTGTGCTCGCGCT | |
| 7.37 TTT PAM TS | AGCGCGAGCACAGCTAAGGCCACGGAGCGAGACATCTCGGCCCAAAT | 43966 |
| | GCTGTCAGCTTCA | |
| 7.37 TTT PAM | TGAAGCTGACAGCATTTGGGCCGAGATGTCTCGCTCCGTGGCCTTAG | 43967 |
| NTS | CTGTGCTCGCGCT | |
| 7.37 CTT PAM TS | AGCGCGAGCACAGCTAAGGCCACGGAGCGAGACATCTCGGCCCTAGT | 43968 |
| | GCTGTCAGCTTCA | |
| 7.37 CTT PAM | TGAAGCTGACAGCACTTGGGCCGAGATGTCTCGCTCCGTGGCCTTAG | 43969 |
| NTS | CTGTGCTCGCGCT | |
| 7.37 GTT PAM TS | AGCGCGAGCACAGCTAAGGCCACGGAGCGAGACATCTCGGCCCTACT | 43970 |
| | GCTGTCAGCTTCA | |
| 7.37 GTT PAM | TGAAGCTGACAGCAGTTGGGCCGAGATGTCTCGCTCCGTGGCCTTAG | 43971 |
| NTS | CTGTGCTCGCGCT | |
| 7.37 ATT PAM TS | AGCGCGAGCACAGCTAAGGCCACGGAGCGAGACATCTCGGCCCTATT | 43972 |
| | GCTGTCAGCTTCA | |
| 7.37 ATT PAM | TGAAGCTGACAGCAATTGGGCCGAGATGTCTCGCTCCGTGGCCTTAG | 43973 |
| NTS | CTGTGCTCGCGCT | |

*The DNA sequences used to generate each dsDNA substrate are shown.
The PAM sequences for each are bolded.
TS—target strand.
NTS—Non-target strand.

TABLE 11
Apparent cleavage rates of CasX
491.174 against NTC and NTT PAMs

| PAM | TTC | ATC | CTC | GTC | TTT | ATT | CTT | GTT |
|---|---|---|---|---|---|---|---|---|
| k_{cleave} | 15.6* | 6.66 | 9.45 | 2.52 | 1.33 | 0.0675 | 0.0204 | 0.330 |
| (min⁻¹) | | | | | | | | |

*The rate of TTC cleavage exceeds the resolution of this assay, so the resulting k_{cleave} should be taken as a lower bound.

### Example 6: Assessing Nuclease Activity for Double-Strand Cleavage

Purified wild-type and engineered CasX variants will be complexed with single-guide RNA bearing a fixed HRS targeting sequence. The RNP complexes will be added to buffer containing MgCl2 at a final concentration of 100 nM and incubated with double-stranded target DNA with a 5′ Cy7.5 label on either the target or non-target strand at a concentration of 10 nM. Aliquots of the reactions will be taken at fixed time points and quenched by the addition of an equal volume of 50 mM EDTA and 95% formamide. The samples will be run on a denaturing polyacrylamide gel to separate cleaved and uncleaved DNA substrates. The results will be visualized and the cleavage rates of the target and non-target strands by the wild-type and engineered variants will be determined. To more clearly differentiate between changes to target binding vs the rate of catalysis of the nucleolytic reaction itself, the protein concentration will be titrated over a range from 10 nM to 1 uM and cleavage rates will be determined at each concentration to generate a pseudo-Michaelis-Menten fit and determine the kcat* and KM*. Changes to KM* are indicative of altered binding, while changes to kcat* are indicative of altered catalysis.

### Example 7: The PASS Assay Identifies CasX Protein Variants of Differing PAM Sequence Specificity

The purpose of the experiment was to identify the PAM sequence specificities of CasX proteins 2 (SEQ ID NO: 2), 491 (SEQ ID NO: 126), 515 (SEQ ID NO: 133), 533 (SEQ ID NO: 43663), 535 (SEQ ID NO: 43665), 668 (SEQ ID NO: 43797), and 672 (SEQ ID NO: 43800). To accomplish this, the HEK293 cell line PASS_V1.01 or PASS_V1.02 was treated with the above CasX proteins in at least two replicate experiments, and Next-generation sequencing (NGS) was performed to calculate the percent editing using a variety of spacers at their intended target sites.

Materials and Methods: A multiplexed pooled approach was taken to assay clonal protein variants using the PASS system. Briefly, two pooled HEK cell lines were generated and termed PASS_V1.01 and PASS_V1.02. Each cell within the pool contained a genome-integrated single-guide RNA (sgRNA), paired with a specific target site. After transfection of protein-expression constructs, editing at a specific target by a specific spacer could be quantified by NGS. Each guide-target pair was designed to provide data related to activity, specificity, and targetability of the CasX-guide RNP complex.

Paired spacer-target sequences were synthesized by Twist Biosciences and obtained as an equimolar pool of oligonucleotides. This pool was amplified by PCR and cloned by Golden Gate cloning to generate a final library of plasmids named p77. Each plasmid contained a sgRNA expression element and a target site, along with a GFP expression element. The sgRNA expression element consisted of a U6 promoter driving transcription of gRNA scaffold 174 (SEQ ID NO:2238), followed by a spacer sequence which would target the RNP of the guide and CasX variant to the intended target site. 250 possible unique, paired spacer-target synthetic sequences were designed and synthesized. A pool of lentivirus was then produced from this plasmid library using the LentiX production system (Takara Bio USA, Inc) according to the manufacturer's instructions. The resulting viral preparation was then quantified by qPCR and transduced into a standard HEK293 cell line at a low multiplicity of infection so as to generate single copy integrations. The resulting cell line was then purified by fluorescence-activated cell sorting (FACS) to complete the production of PASS_V1.01 or PASS_V1.02. A cell line was then seeded in six-well plate format and treated in duplicate with either water or was transfected with 2 μg of plasmid p67, delivered by Lipofectamine Transfection Reagent (ThermoFisher) according to the manufacturer's instructions. Plasmid p67 contains an EF-1alpha promoter driving expression of a CasX protein tagged with the SV40 Nuclear Localization Sequence. After two days, treated cells were collected, lysed, and genomic DNA was extracted using a genomic DNA isolation kit (Zymo Research). Genomic DNA was then PCR amplified with custom primers to generate amplicons compatible with Illumina NGS and sequenced on a NextSeq instrument. Sample reads were demultiplexed and filtered for quality. Editing outcome metrics (fraction of reads with indels) were then quantified for each spacer-target synthetic sequence across treated samples.

To assess the PAM sequence specificity for a CasX protein, editing outcome metrics for four different PAM sequences were categorized. For TTC PAM target sites, 48 different spacer-target pairs were quantified; for ATC, CTC, and GTC PAM target sites, 14, 22, and 11 individual target sites were quantified, respectively. For some CasX proteins, replicate experiments were repeated dozens of times over several months. For each of these experiments, the average editing efficiency was calculated for each of the above described spacers. The average editing efficiency across the four categories of PAM sequence was then calculated from all such experiments, along with the standard deviation of these measurements.

Results: Table 12 lists the average editing efficiency across PAM categories and across CasX protein variants, along with the standard deviation of these measurements. The number of measurements for each category is also indicated. These data indicate that the engineered CasX variants 491 and 515 are specific for the canonical PAM sequence TTC, while other engineered variants of CasX performed more or less efficiently at the PAM sequences tested. In particular, the average rank order of PAM preferences for CasX 491 is TTC>>ATC>CTC>GTC, or TTC>>ATC>GTC>CTC for CasX 515, while the wild-type CasX 2 exhibits an average rank order of TTC>>GTC>CTC>ATC. Note that for the lower editing PAM sequences the error of these average measurements is high. In contrast, CasX variants 535, 668, and 672 have considerably broader PAM recognition, with a rank order of TTC>CTC>ATC>GTC. Finally, CasX 533 exhibits a completely re-ordered ranking relative to the WT CasX, ATC>CTC>>GTC>TTC. These data can be used to engineer maximally-active therapeutic CasX molecules for a target DNA sequence of interest.

Under the conditions of the experiments, a set of CasX proteins was identified that are improved for double-stranded DNA cleavage in human cells at target DNA sequences associated with a PAM of sequence TTC, ATC, CTC, or GTC, supporting that CasX variants with an altered spectrum of PAM specificity, relative to wild-type CasX, can be generated.

TABLE 12
Average editing of selected CasX proteins at spacers associated with PAM sequences
of TTC, ATC, CTC, or GTC

| | PAM | Average Percent | Standard | Number of |
|---|---|---|---|---|
| CasX Name | Sequence | Editing | Deviation | Measurements |
| 2 | ATC | 0.40 | 1.35 | 336 |
| 2 | CTC | 0.46 | 2.29 | 528 |
| 2 | GTC | 0.69 | 6.27 | 264 |
| 2 | TTC | 5.28 | 7.34 | 1152 |
| 491 | ATC | 6.86 | 8.29 | 364 |
| 491 | CTC | 4.54 | 6.40 | 572 |
| 491 | GTC | 3.40 | 6.68 | 286 |
| 491 | TTC | 40.41 | 23.13 | 1248 |
| 515 | ATC | 4.47 | 5.49 | 252 |
| 515 | CTC | 3.36 | 4.80 | 396 |
| 515 | GTC | 3.65 | 10.75 | 198 |
| 515 | TTC | 36.75 | 24.89 | 864 |
| 533 | ATC | 47.50 | 15.86 | 96 |
| 533 | CTC | 25.90 | 14.74 | 28 |
| 533 | GTC | 6.34 | 8.36 | 44 |
| 533 | TTC | 0.87 | 3.05 | 22 |
| 535 | ATC | 9.70 | 10.20 | 56 |
| 535 | CTC | 11.77 | 13.59 | 88 |
| 535 | GTC | 7.62 | 15.04 | 44 |
| 535 | TTC | 29.29 | 18.78 | 192 |
| 668 | ATC | 44.69 | 24.40 | 56 |
| 668 | CTC | 46.14 | 26.57 | 88 |
| 668 | GTC | 30.48 | 24.06 | 44 |
| 668 | TTC | 55.34 | 28.59 | 192 |
| 672 | ATC | 25.51 | 20.85 | 56 |
| 672 | CTC | 30.05 | 22.95 | 88 |
| 672 | GTC | 14.21 | 13.38 | 44 |
| 672 | TTC | 52.36 | 27.64 | 192 |

### Example 8: CasX:gRNA In Vitro Cleavage Assays

### 1. Assembly of RNP

Purified wild-type and RNP of CasX and single guide RNA (sgRNA) were either prepared immediately before experiments or prepared and snap-frozen in liquid nitrogen and stored at −80° C. for later use. To prepare the RNP complexes, the CasX protein was incubated with sgRNA at 1:1.2 molar ratio. Briefly, sgRNA was added to Buffer #1 (25 mM NaPi, 150 mM NaCl, 200 mM trehalose, 1 mM MgCl2), then the CasX was added to the sgRNA solution, slowly with swirling, and incubated at 37° C. for 10 min to form RNP complexes. RNP complexes were filtered before use through a 0.22 m Costar 8160 filters that were pre-wet with 200 μl Buffer #1. If needed, the RNP sample was concentrated with a 0.5 ml Ultra 100-K_{d} cutoff filter, (Millipore part #UFC510096), until the desired volume was obtained. Formation of competent RNP was assessed as described below.

### 2. Determining Cleavage-Competent Fractions for Protein Variants Compared to Wild-Type Reference CasX

The ability of CasX variants to form active RNP compared to reference CasX was determined using an in vitro cleavage assay. The beta-2 microglobulin (B2M) 7.37 target for the cleavage assay was created as follows. DNA oligos with the sequence TGAAGCTGACAGCATTCGGGCCGAGATGTCTCGCTCCGTGGCCTTAGCTGTGCTCGC GCT (non-target strand, NTS (SEQ ID NO: 364)) and AGCGCGAGCACAGCTAAGGCCACGGAGCGAGACATCTCGGCCCGAATGCTGTCAGC TTCA (target strand, TS (SEQ ID NO: 365)) were purchased with 5′ fluorescent labels (LI-COR IRDye 700 and 800, respectively). dsDNA targets were formed by mixing the oligos in a 1:1 ratio in 1×cleavage buffer (20 mM Tris HCl pH 7.5, 150 mM NaCl, 1 mM TCEP, 5% glycerol, 10 mM MgCl2), heating to 95° C. for 10 minutes, and allowing the solution to cool to room temperature.

CasX RNPs were reconstituted with the indicated CasX and guides (see graphs) at a final concentration of 1 μM with 1.5-fold excess of the indicated guide unless otherwise specified in 1×cleavage buffer (20 mM Tris HCl pH 7.5, 150 mM NaCl, 1 mM TCEP, 5% glycerol, 10 mM MgCl2) at 37° C. for 10 min before being moved to ice until ready to use. The 7.37 target was used, along with sgRNAs having spacers complementary to the 7.37 target.

Cleavage reactions were prepared with final RNP concentrations of 100 nM and a final target concentration of 100 nM. Reactions were carried out at 37° C. and initiated by the addition of the 7.37 target DNA. Aliquots were taken at 5, 10, 30, 60, and 120 minutes and quenched by adding to 95% formamide, 20 mM EDTA. Samples were denatured by heating at 95° C. for 10 minutes and run on a 10% urea-PAGE gel. The gels were either imaged with a LI-COR Odyssey CLx and quantified using the LI-COR Image Studio software or imaged with a Cytiva Typhoon and quantified using the Cytiva IQTL software. The resulting data were plotted and analyzed using Prism. We assumed that CasX acts essentially as a single-turnover enzyme under the assayed conditions, as indicated by the observation that sub-stoichiometric amounts of enzyme fail to cleave a greater-than-stoichiometric amount of target even under extended time-scales and instead approach a plateau that scales with the amount of enzyme present. Thus, the fraction of target cleaved over long time-scales by an equimolar amount of RNP is indicative of what fraction of the RNP is properly formed and active for cleavage. The cleavage traces were fit with a biphasic rate model, as the cleavage reaction clearly deviates from monophasic under this concentration regime, and the plateau was determined for each of three independent replicates. The mean and standard deviation were calculated to determine the active fraction (Table 13).

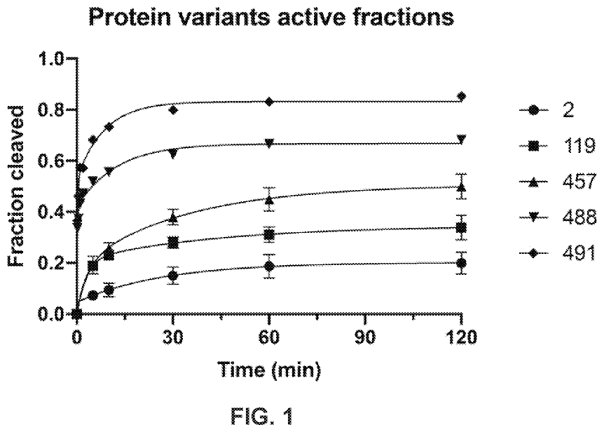

Apparent active (competent) fractions were determined for RNPs formed for CasX2+guide 174+7.37 spacer, CasX119+guide 174+7.37 spacer, CasX457+guide 174+7.37 spacer, CasX488+guide 174+7.37 spacer, and CasX491+guide 174+7.37 spacer as shown in FIG. 1. The determined active fractions are shown in Table 13. All CasX variants had higher active fractions than the wild-type CasX2, indicating that the engineered CasX variants form significantly more active and stable RNP with the identical guide under tested conditions compared to wild-type CasX. This may be due to an increased affinity for the sgRNA, increased stability or solubility in the presence of sgRNA, or greater stability of a cleavage-competent conformation of the engineered CasX:sgRNA complex. An increase in solubility of the RNP was indicated by a notable decrease in the observed precipitate formed when CasX457, CasX488, or CasX491 was added to the sgRNA compared to CasX2.

### 3. In Vitro Cleavage Assays—Determining Cleavage-Competent Fractions for Single Guide Variants Relative to Reference Single Guides

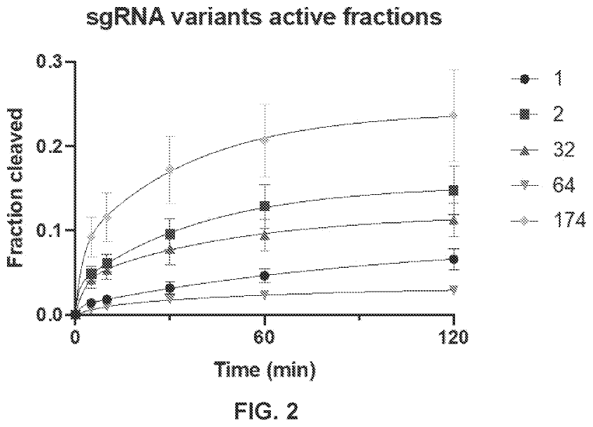

Cleavage-competent fractions were also determined using the same protocol for CasX2.2.7.37, CasX2.32.7.37, CasX2.64.7.37, and CasX2.174.7.37 to be 16±3%, 13±3%, 5±2%, and 22±5%, as shown in FIG. 2 and Table 11.

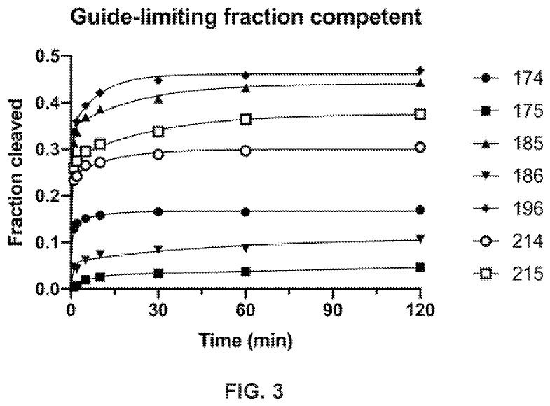

A second set of guides were tested under different conditions to better isolate the contribution of the guide to RNP formation. Guides 174, 175, 185, 186, 196, 214, and 215 with 7.37 spacer were mixed with CasX 491 at final concentrations of 1 μM for the guide and 1.5 μM for the protein, rather than with excess guide as before. Results are shown in FIG. 3 and Table 11. Many of these guides exhibited additional improvement over 174, with 185 and 196 achieving 91±4% and 91±1% competent fractions, respectively, compared with 80±9% for 174 under these guide-limiting conditions.

The data indicate that both CasX variants and sgRNA variants are able to form a higher degree of active RNP with guide RNA compare to wild-type CasX and wild-type sgRNA.

The apparent cleavage rates of CasX variants 119, 457, 488, and 491 compared to wild-type reference CasX were determined using an in vitro fluorescent assay for cleavage of the target 7.37.

### 4. In Vitro Cleavage Assays—Determining k_{cleave} for CasX Variants Compared to Wild-Type Reference CasX

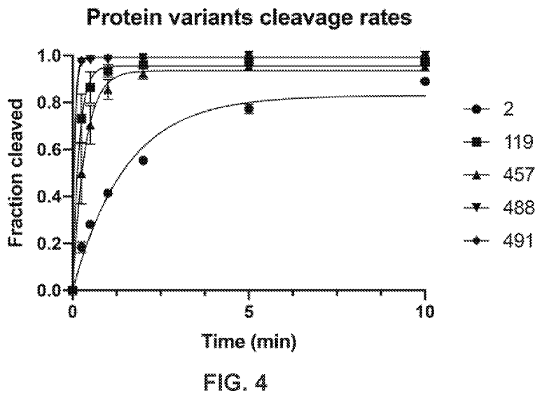

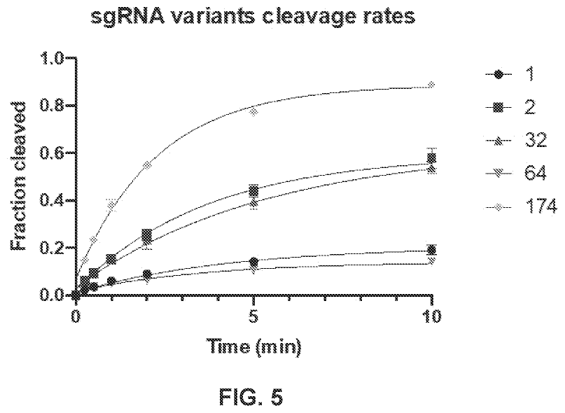

CasX RNPs were reconstituted with the indicated CasX (see FIG. 4) at a final concentration of 1 μM with 1.5-fold excess of the indicated guide in 1×cleavage buffer (20 mM Tris HCl pH 7.5, 150 mM NaCl, 1 mM TCEP, 5% glycerol, 10 mM MgCl2) at 37° C. for 10 min before being moved to ice until ready to use. Cleavage reactions were set up with a final RNP concentration of 200 nM and a final target concentration of 10 nM. Reactions were carried out at 37° C. except where otherwise noted and initiated by the addition of the target DNA. Aliquots were taken at 0.25, 0.5, 1, 2, 5, and 10 minutes and quenched by adding to 95% formamide, 20 mM EDTA. Samples were denatured by heating at 95° C. for 10 minutes and run on a 10% urea-PAGE gel. The gels were imaged with a LI-COR Odyssey CLx and quantified using the LI-COR Image Studio software or imaged with a Cytiva Typhoon and quantified using the Cytiva IQTL software. The resulting data were plotted and analyzed using Prism, and the apparent first-order rate constant of non-target strand cleavage (k_{cleave}) was determined for each CasX:sgRNA combination replicate individually. The mean and standard deviation of three replicates with independent fits are presented in Table 11, and the cleavage traces are shown in FIG. 5.

Apparent cleavage rate constants were determined for wild-type CasX2, and CasX variants 119, 457, 488, and 491 with guide 174 and spacer 7.37 utilized in each assay (see Table 11 and FIG. 4). All CasX variants had improved cleavage rates relative to the wild-type CasX2. CasX 457 cleaved more slowly than 119, despite having a higher competent fraction as determined above. CasX488 and CasX491 had the highest cleavage rates by a large margin; as the target was almost entirely cleaved in the first timepoint, the true cleavage rate exceeds the resolution of this assay, and the reported k_{cleave} should be taken as a lower bound.

The data indicate that the CasX variants have a higher level of activity, with k_{cleave} rates reaching at least 30-fold higher compared to wild-type CasX2.

### 5. In Vitro Cleavage Assays: Comparison of Guide Variants to Wild-Type Guides

Cleavage assays were also performed with wild-type reference CasX2 and reference guide 2 compared to guide variants 32, 64, and 174 to determine whether the variants improved cleavage. The experiments were performed as described above. As many of the resulting RNPs did not approach full cleavage of the target in the time tested, we determined initial reaction velocities (V₀) rather than first-order rate constants. The first two timepoints (15 and 30 seconds) were fit with a line for each CasX:sgRNA combination and replicate. The mean and standard deviation of the slope for three replicates were determined.

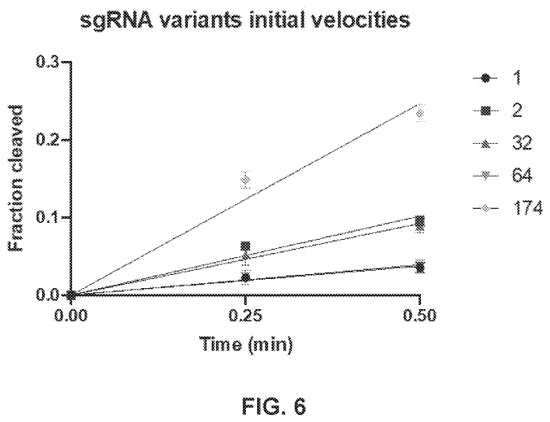

Under the assayed conditions, the V₀ for CasX2 with guides 2, 32, 64, and 174 were 20.4±1.4 nM/min, 18.4±2.4 nM/min, 7.8±1.8 nM/min, and 49.3±1.4 nM/min (see Table 13 and FIG. 5 and FIG. 6). Guide 174 showed substantial improvement in the cleavage rate of the resulting RNP (˜2.5-fold relative to 2, see FIG. 6), while guides 32 and 64 performed similar to or worse than guide 2. Notably, guide 64 supports a cleavage rate lower than that of guide 2 but performs much better in vivo (data not shown). Some of the sequence alterations to generate guide 64 likely improve in vivo transcription at the cost of a nucleotide involved in triplex formation. Improved expression of guide 64 likely explains its improved activity in vivo, while its reduced stability may lead to improper folding in vitro.

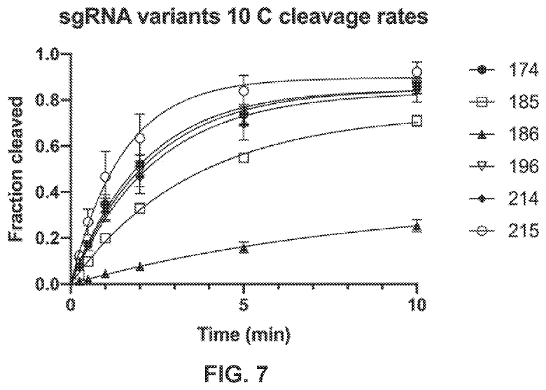

Additional experiments were carried out with guides 174, 175, 185, 186, 196, 214, and 215 with spacer 7.37 and CasX 491 to determine relative cleavage rates. To reduce cleavage kinetics to a range measurable with our assay, the cleavage reactions were incubated at 10° C. Results are in FIG. 7 and Table 13. Under these conditions, 215 was the only guide that supported a faster cleavage rate than 174. 196, which exhibited the highest active fraction of RNP under guide-limiting conditions, had kinetics essentially the same as 174, again highlighting that different variants result in improvements of distinct characteristics.

The data support that, under the conditions of the assay, use of the majority of the guide variants with CasX results in RNP with a higher level of activity than one with the wild-type guide, with improvements in initial cleavage velocity ranging from ˜2-fold to >6-fold. Numbers in Table 13 indicate, from left to right, CasX variant, sgRNA scaffold, and spacer sequence of the RNP construct. In the RNP construct names in the table below, CasX protein variant, guide scaffold and spacer are indicated from left to right.

### 6. In Vitro Cleavage Assays: Comparing Cleavage Rate and Competent Fraction of 515.174 and 526.174 Against Reference 2.2

We wished to compare engineered protein CasX variants 515 and 526 in complex with engineered single-guide variant 174 against the reference wild-type protein 2 (SEQ ID NO:2) and minimally-engineered guide variant 2 (SEQ ID NO:5). RNP complexes were assembled as described above, with 1.5-fold excess guide. Cleavage assays to determine k_{cleave} and competent fraction were performed as described above, with both performed at 37° C., and with different timepoints used to determine the competent fraction for the wild-type vs engineered RNPs due to the significantly different times needed for the reactions to near completion.

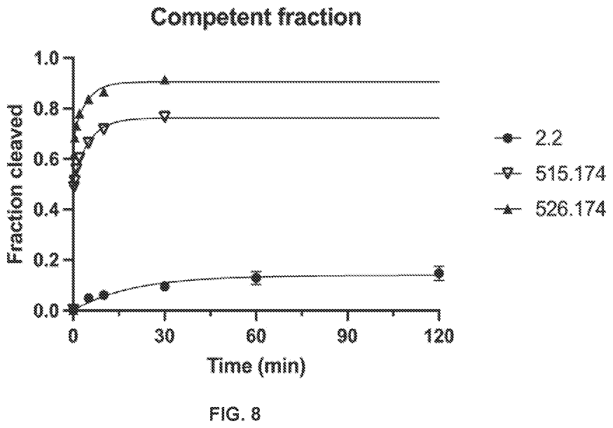

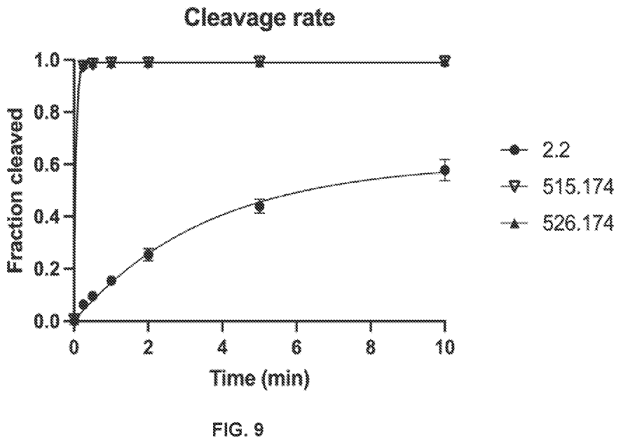

The resulting data clearly demonstrate the dramatic improvements made to RNP activity by engineering both protein and guide. RNPs of 515.174 and 526.174 had competent fractions of 76% and 91%, respectively, as compared to 16% for 2.2 (FIG. 8, Table 13). In the kinetic assay, both 515.174 and 526.174 cut essentially all of the target DNA by the first timepoint, exceeding the resolution of the assay and resulting in estimated cleavage rates of 17.10 and 19.87 min⁻¹, respectively (FIG. 9, Table 13). An RNP of 2.2, by contrast, cut on average less than 60% of the target DNA by the final 10-minute timepoint and has an estimated k_{cleave} nearly two orders of magnitude lower than the engineered RNPs. The modifications made to the protein and guide have resulted in RNPs that are more stable, more likely to form active particles, and cut DNA much more efficiently on a per-particle basis as well.

TABLE 13
Results of cleavage and RNP formation assays

| RNP | | Initial | Competent |
|---|---|---|---|
| Construct | k_{cleave}* | velocity* | fraction |
| 2.2.7.37 | | 20.4 + | 16 + 3% |
| | | 1.4 nM/min | |
| 2.32.7.37 | | 18.4 + | 13 + 3% |
| | | 2.4 nM/min | |
| 2.64.7.37 | | 7.8 + | 5 + 2% |
| | | 1.8 nM/min | |
| 2.174.7.37 | 0.51 + 0.01 min⁻¹ | 49.3 + | 22 + 5% |
| | | 1.4 nM/min | |
| 119.174.7.37 | 6.29 + 2.11 min⁻¹ | | 35 + 6% |
| 457.174.7.37 | 3.01 + 0.90 min⁻¹ | | 53 + 7% |
| 488.174.7.37 | 15.19 min⁻¹ | | 67% |
| 491.174.7.37 | 16.59 min⁻¹/0.293 | | 83% /17% |
| | min⁻¹ (10° C.) | | (guide-limited) |
| 491.175.7.37 | 0.089 min⁻¹ | | 5% |
| | (10° C.) | | (guide-limited) |
| 491.185.7.37 | 0.227 min⁻¹ | | 44% |
| | (10° C.) | | (guide-limited) |
| 491.186.7.37 | 0.099 min⁻¹ | | 11% |
| | (10° C.) | | (guide-limited) |
| 491.196.7.37 | 0.292 min⁻¹ | | 46% |
| | (10° C.) | | (guide-limited) |
| 491.214.7.37 | 0.284 min⁻¹ | | 30% |
| | (10° C.) | | (guide-limited) |
| 491.215.7.37 | 0.398 min⁻¹ | | 38% |
| | (10° C.) | | (guide-limited) |
| 515.174.7.37 | 17.10 min⁻¹* | | 76% |
| 526.174.7.37 | 19.87 min⁻¹* | | 91% |

*Mean and standard deviation
**Rate exceeds resolution of assay

### Example 9: Testing Effects of Spacer Length on In Vitro Cleavage Kinetics

Ribonuclear protein complexes (RNP) of two CasX variants and guide RNA with spacers of varying length were tested for in vitro cleavage activity to determine what spacer length supports the most efficient cleavage of a target nucleic acid and whether spacer length preference changes with the protein.

### Methods:

Ribonuclear protein complexes (RNP) of CasX and guide RNA with spacers of varying length were tested for in vitro cleavage activity to determine what spacer length supports the most efficient cleavage of a target nucleic acid.

CasX variant 515 and 526 were purified as described above. Guides with scaffold 174 (SEQ ID NO: 2238) were prepared by in vitro transcription (IVT). IVT templates were generated by PCR using Q5 polymerase (NEB M0491) according to the recommended protocol, template oligos for each scaffold backbone, and amplification primers with the T7 promoter and the 7.37 spacer (GGCCGAGATGTCTCGCTCCG; targeting tdTomato (SEQ ID NO: 316)) of 20 nucleotides or truncated from the 3′ end to 18 or 19 nucleotides. Spacer sequences as well as the oligonucleotides used to generate each template are shown in Table 14. The resulting templates were then used with T7 RNA polymerase to produce RNA guides according to standard protocols. The guides were purified using denaturing polyacrylamide gel electrophoresis and refolded prior to use.

CasX RNPs were reconstituted by diluting CasX to 1 μM in 1×cleavage buffer (20 mM Tris HCl pH 7.5, 150 mM NaCl, 1 mM TCEP, 5% glycerol, 10 mM MgCl2) and adding sgRNA to 1.2 μM and incubating at 37° C. for 10 min before being moved to ice until ready to use. Fluorescently-labeled 7.37 target DNA was purchased as individual oligonucleotides from Integrated DNA Technologies (full sequences in Table 14), and dsDNA target was prepared by heating an equimolar mix of the two complementary strands in 1×cleavage buffer and slow-cooling to room temperature.

RNPs were diluted in cleavage buffer to a final concentration of 200 nM and incubated at 10° C. without shaking. Cleavage reactions were initiated by the addition of 7.37 target DNA to a final concentration of 10 nM. Timepoints were taken at 0.25, 0.5, 1, 2, 5, 10, and 30 minutes. Timepoints were quenched by adding to an equal volume of 95% formamide, 20 mM EDTA. Samples were denatured by heating at 95° C. for 10 minutes and run on a 10% urea-PAGE gel. Gels were imaged with an Amersham Typhoon and analyzed with IQTL software. The resulting data were plotted and analyzed using Prism. The cleavage of the non-target strand was fit with a single exponential function to determine the apparent first-order rate constant (k_{cleave}).

### Results:

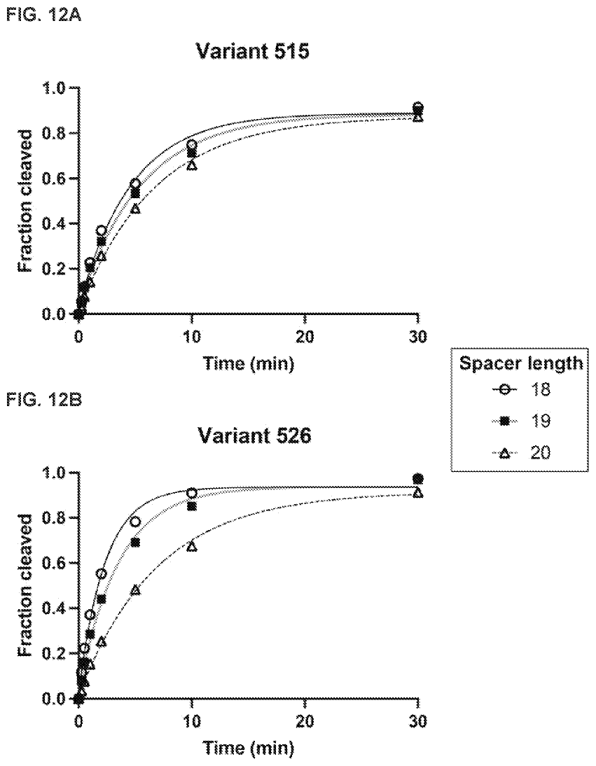

Cleavage rates were compared for CasX variants 515 and 526 in complex with sgRNAs with 18, 19, or 20 nucleotide spacers to determine which spacer length resulted in the most efficient cleavage for each protein variant. Consistent with other experiments performed with in vitro-transcribed sgRNA, the 18-nt spacer guide performed best for both protein variants (FIGS. 12A and B, Table 15). The 18-nt spacer was 1.4-fold faster than the 20-nt spacer for protein 515, and it was 3-fold faster than the 20-nt spacer for protein 526. The 19-nt spacer had intermediate activity for both proteins, though again the difference was more pronounced for variant 526. In general, spacers shorter than 20-nt have been observed to have increased activity across a range of proteins, spacers, and delivery methods, but the degree of improvement and the optimal spacer length have varied. These data show that two engineered proteins that are quite similar in sequence (different in only two residues) can have changes in activity as a result of spacer length that are similar in direction but substantially different in degree.

TABLE 14
Relevant sequences and oligonucleotides

| | | |
|---|---|---|
| 7.37 target | IR700- | SEQ ID NO: 364 |
| sequence non-target | TGAAGCTGACAGCATTCGGGCCGAGATG | |
| strand | TCTCGCTCCGTGGCCTTAGCTGTGCTCG | |
| | CGCT | |
| 7.37 target | IR800- | SEQ ID NO: 365 |
| sequence target | AGCGCGAGCACAGCTAAGGCCACGGAGC | |
| strand | GAGACATCTCGGCCCGAATGCTGTCAGC | |
| | TTCA | |
| 20-nt spacer | GGCCGAGATGTCTCGCTCCG | SEQ ID NO: 316 |
| sequence | | |
| 18-nt spacer | GGCCGAGATGTCTCGCTC | SEQ ID NO: 43974 |
| sequence | | |
| 19-nt spacer | GGCCGAGATGTCTCGCTCC | SEQ ID NO: 43975 |
| sequence | | |
| Scaffold 174 | GAAATTAATACGACTCACTATAACTGGC | SEQ ID NO: 269 |
| template fwd | GCTTTTATCTGATTACTTTGAGAGCCAT | |
| | CACCAGCGACTATGTCGTAGTGGGTAAA | |
| | GCT | |
| Scaffold 174 | CTTTGATGCTCCCTCCGAAGAGGGAGCT | SEQ ID NO: 270 |
| template rev | TTACCCACTACGACATAGTCGC | |
| T7 amplification | GAAATTAATACGACTCACTATA | SEQ ID NO: 259 |
| primer | | |
| Scaffold 174 20-nt | CGGAGCGAGACATCTCGGCCCTTTGATG | SEQ ID NO: 271 |
| spacer primer | CTCCCTCC | |
| Scaffold 174 18-nt | GAGCGAGACATCTCGGCCCTTTGATGCT | SEQ ID NO: 43976 |
| spacer primer | CCCTCC | |
| Scaffold 174 19-nt | GGAGCGAGACATCTCGGCCCTTTGATGC | SEQ ID NO: 43977 |
| spacer primer | TCCCTCC | |

TABLE 15
Cleavage rates of RNPs with truncated spacers

| | 515 k_{cleave} | 526 k_{cleave} |
|---|---|---|
| Spacer length | min⁻¹) | (min⁻¹) |
| 18 | 0.215 | 0.427 |
| 19 | 0.182 | 0.282 |
| 20 | 0.150 | 0.143 |

### Example 10: Assessing Binding Affinity to the Guide RNA

Purified wild-type and improved CasX will be incubated with synthetic single-guide RNA containing a 3′ Cy7.5 moiety in low-salt buffer containing magnesium chloride as well as heparin to prevent non-specific binding and aggregation. The sgRNA will be maintained at a concentration of 10 pM, while the protein will be titrated from 1 pM to 100 μM in separate binding reactions. After allowing the reaction to come to equilibrium, the samples will be run through a vacuum manifold filter-binding assay with a nitrocellulose membrane and a positively charged nylon membrane, which bind protein and nucleic acid, respectively. The membranes will be imaged to identify guide RNA, and the fraction of bound vs unbound RNA will be determined by the amount of fluorescence on the nitrocellulose vs nylon membrane for each protein concentration to calculate the dissociation constant of the protein-sgRNA complex. The experiment will also be carried out with improved variants of the sgRNA to determine if these mutations also affect the affinity of the guide for the wild-type and mutant proteins. We will also perform electromobility shift assays to qualitatively compare to the filter-binding assay and confirm that soluble binding, rather than aggregation, is the primary contributor to protein-RNA association.

### Example 11: Assessing Binding Affinity to the Target DNA

Purified wild-type and improved CasX will be complexed with single-guide RNA bearing a targeting sequence complementary to the target nucleic acid. The RNP complex will be incubated with double-stranded target DNA containing a PAM and the appropriate target nucleic acid sequence with a 5′ Cy7.5 label on the target strand in low-salt buffer containing magnesium chloride as well as heparin to prevent non-specific binding and aggregation. The target DNA will be maintained at a concentration of 1 nM, while the RNP will be titrated from 1 pM to 100 μM in separate binding reactions. After allowing the reaction to come to equilibrium, the samples will be run on a native 5% polyacrylamide gel to separate bound and unbound target DNA. The gel will be imaged to identify mobility shifts of the target DNA, and the fraction of bound vs unbound DNA will be calculated for each protein concentration to determine the dissociation constant of the RNP-target DNA ternary complex. The experiments are expected to demonstrate the improved binding affinity of the RNP comprising a CasX variant and gRNA variant compared to an RNP comprising a reference CasX and reference gRNA.

### Example 12: Improved Guide RNA Variants Demonstrate Enhanced On-Target Activity at Mouse and Human RHO Exon 1 Loci In Vitro

Experiments were conducted to identify novel engineered guide RNA variants with increased activity at different genomic targets, including the therapeutically-relevant mouse and human Rho exon 1. Previous assays identified many different “hotspot” regions (e.g., stem loop) within the scaffold sequences holding the potential to significantly increase editing efficiency as well as specificity (sequences in Table 16). Additionally, screens were conducted to identify scaffold variants that would increase the overall activity of our CRISPR system in an AAV vector across multiple different PAM-spacer combinations, without triggering off-target or non-specific editing. Achieving increased editing efficiency compared to current benchmark vectors would allow reduced viral vector doses to be used in in vivo studies, improving the safety of AAV-mediated CasX-guide systems.

### Materials and Methods:

New CasX variant sequences and gRNA scaffold variants were inserted into an AAV transgene construct for plasmid and viral vector validation. We conceptually broke up the AAV transgene between ITRs into different parts, which consisted of our therapeutic cargo (CasX and gRNA variants+spacer) and accessory elements (e.g., promoters, NLS, poly(A)) relevant to expression in mammalian cells. Each part in the AAV genome was separated by restriction enzyme sites to allow for modular cloning. Parts were ordered as gene fragments from Twist, were PCR amplified and digested with corresponding restriction enzymes, cleaned, then ligated into a vector digested with the same enzymes. New AAV constructs were then transformed into chemically competent E. coli (Turbos or Stbl3s), which were plated on Kanamycin LB-Agar plates following recovery at 37° C. for 1 hour. Single colonies were picked, mini-prepped, and Sanger-sequenced. Sequence-verified constructs were then cloned into a BbsI Golden-Gate assembly with spacer 12.7 (targeting tdTomato: CTGCATTCTAGTTGTGGTTT (SEQ ID NO: 43978)). Spacers were made by annealing two oligos and diluting in water. The transformation and miniprep protocols were then repeated and spacer-cloned vectors were sequence-verified again. Validated constructs were maxi-prepped. To assess the quality of maxi-preps, constructs were processed in two separate digests with XmaI (which cuts at several sites in each of the ITRs) and XhoI, which cuts once in the AAV genome. These digests and the uncut construct were then run on a 100 Agarose gel and imaged on a ChemiDoc. If the plasmid was >90% supercoiled, was the correct size, and the ITRs were intact, the construct moved on to be tested via nucleofection and subsequently used AAV vector production.

TABLE 16
Guide sequences cloned into p59.491.U6.X.Y. plasmids.
(X = guide; Y = spacer)

| Guide | Spacer | SEQ | | SEQ | | SEQ ID |
|---|---|---|---|---|---|---|
| Construct | Sequence | ID NO | Guide Sequence | ID NO | Guide + Spacer Sequence | NO |
| 174.12.7 | CTGCATT | 43978 | ACTGGCGCTTTT | 43983 | ACTGGCGCTTTTATCTGATTACT | 43993 |
| | CTAGTTG | | ATCTGATTACTT | | TTGAGAGCCATCACCAGCGACT | |
| | TGGTTT | | TGAGAGCCATCA | | ATGTCGTAGTGGGTAAAGCTCC | |
| | | | CCAGCGACTATG | | CTCTTCGGAGGGAGCATCAAAG | |
| | | | TCGTAGTGGGTA | | CTGCATTCTAGTTGTGGTTT | |
| | | | AAGCTCCCTCTT | | | |
| | | | CGGAGGGAGCA | | | |
| | | | TCAAAG | | | |
| 229.12.7 | CTGCATT | 43978 | ACTGGCACTTTT | 43984 | ACTGGCACTTTTATCTGATTACT | 43994 |
| | CTAGTTG | | ATCTGATTACTT | | TTGAGAGCCATCACCAGCGACT | |
| | TGGTT | | TGAGAGCCATCA | | ATGTCGTATGGGTAAAGCGCTT | |
| | | | CCAGCGACTATG | | ACGGACTTCGGTCCGTAAGAAG | |
| | | | TCGTATGGGTAA | | CATCAAAGCTGCATTCTAGTTG | |
| | | | AGCGCTTACGGA | | TGGTTT | |
| | | | CTTCGGTCCGTA | | | |
| | | | AGAAGCATCAA | | | |
| | | | AG | | | |
| 230.12.7 | CTGCATT | 43978 | ACTGGCACTTCT | 43985 | ACTGGCACTTCTATCTGATTAC | 43995 |
| | CTAGTTG | | ATCTGATTACTC | | TCTGAGAGCCATCACCAGCGAC | |
| | TGGTT | | TGAGAGCCATCA | | TATGTCGTATGGGTAAAGCGCT | |
| | | | CCAGCGACTATG | | TACGGACTTCGGTCCGTAAGAA | |
| | | | TCGTATGGGTAA | | GCATCAGAGCTGCATTCTAGTT | |
| | | | AGCGCTTACGGA | | GTGGTTT | |
| | | | CTTCGGTCCGTA | | | |
| | | | AGAAGCATCAG | | | |
| | | | A | | | |
| 231.12.7 | CTGCATT | 43978 | ACTGGCGCTTCT | 43986 | ACTGGCGCTTCTATCTGATTAC | 43996 |
| | CTAGTTG | | ATCTGATTACTC | | TCTGAGAGCCATCACCAGCGAC | |
| | TGGTT | | TGAGAGCCATCA | | TATGTCGTATGGGTAAAGCCGC | |
| | | | CCAGCGACTATG | | TTACGGACTTCGGTCCGTAAGA | |
| | | | TCGTATGGGTAA | | GGCATCAGAGCTGCATTCTAGT | |
| | | | AGCCGCTTACGG | | TGTGGTTT | |
| | | | ACTTCGGTCCGT | | | |
| | | | AAGAGGCATCA | | | |
| | | | GAG | | | |
| 232.12.7 | CTGCATT | 43978 | ACTGGCACTTCT | 43987 | ACTGGCACTTCTATCTGATTAC | 43997 |
| | CTAGTTG | | ATCTGATTACTC | | TCTGAGCGCCATCACCAGCGAC | |
| | TGGTT | | TGAGCGCCATCA | | TATGTCGTATGGGTAAAGCCGC | |
| | | | CCAGCGACTATG | | TTACGGACTTCGGTCCGTAAGA | |
| | | | TCGTATGGGTAA | | GGCATCAGAGCTGCATTCTAGT | |
| | | | AGCCGCTTACGG | | TGTGGTTT | |
| | | | ACTTCGGTCCGT | | | |
| | | | AAGAGGCATCA | | | |
| | | | GAG | | | |
| 233.12.7 | CTGCATT | 43978 | ACTGGCGCTTCT | 43988 | ACTGGCGCTTCTATCTGATTAC | 43998 |
| | CTAGTTG | | ATCTGATTACTC | | TCTGAGCGCCATCACCAGCGAC | |
| | TGGTT | | TGAGCGCCATCA | | TATGTCGTATGGGTAAAGCCGC | |
| | | | CCAGCGACTATG | | TTACGGACTTCGGTCCGTAAGA | |
| | | | TCGTATGGGTAA | | GGCATCAGAGCTGCATTCTAGT | |
| | | | AGCCGCTTACGG | | TGTGGTTT | |
| | | | ACTTCGGTCCGT | | | |
| | | | AAGAGGCATCA | | | |
| | | | GAG | | | |
| 234.12.7 | CTGCATT | 43978 | ACTGGCGCTTCT | 43989 | ACTGGCGCTTCTATCTGATTAC | 43999 |
| | CTAGTTG | | ATCTGATTACTC | | TCTGAGCGCCATCACCAGCGAC | |
| | TGGTT | | TGAGCGCCATCA | | TATGTCGTATGGGTAAAGCGCC | |
| | | | CCAGCGACTATG | | TTACGGACTTCGGTCCGTAAGG | |
| | | | TCGTATGGGTAA | | AGCATCAGAGCTGCATTCTAGT | |
| | | | AGCGCCTTACGG | | TGTGGTTT | |
| | | | ACTTCGGTCCGT | | | |
| | | | AAGGAGCATCA | | | |
| | | | GAG | | | |
| 235.12.7 | CTGCATT | 43978 | ACTGGCGCTTCT | 43990 | ACTGGCGCTTCTATCTGATTAC | 44000 |
| | CTAGTTG | | ATCTGATTACTC | | TCTGAGCGCCATCACCAGCGAC | |
| | TGGTT | | TGAGCGCCATCA | | TATGTCGTAGTGGGTAAAGCCG | |
| | | | CCAGCGACTATG | | CTTACGGACTTCGGTCCGTAAG | |
| | | | TCGTAGTGGGTA | | AGGCATCAGAGCTGCATTCTAG | |
| | | | AAGCCGCTTACG | | TTGTGGTT | |
| | | | GACTTCGGTCCG | | | |
| | | | TAAGAGGCATC | | | |
| | | | AGAG | | | |
| 236.12.7 | CTGCATT | 43978 | ACGGGACTTTCT | 43991 | ACGGGACTTTCTATCTGATTAC | 44001 |
| | CTAGTTG | | ATCTGATTACTC | | TCTGAAGTCCCTCACCAGCGAC | |
| | TGGTT | | TGAAGTCCCTCA | | TATGTCGTATGGGTAAAGCCGC | |
| | | | CCAGCGACTATG | | TTACGGACTTCGGTCCGTAAGA | |
| | | | TCGTATGGGTAA | | GGCATCAGAGCTGCATTCTAGT | |
| | | | AGCCGCTTACGG | | TGTGGTT | |
| | | | ACTTCGGTCCGT | | | |
| | | | AAGAGGCATCA | | | |
| | | | GAG | | | |
| 237.12.7 | CTGCATT | 43978 | ACCTGTAGTTCT | 43992 | ACCTGTAGTTCTATCTGATTACT | 44002 |
| | CTAGTTG | | ATCTGATTACTC | | CTGACTACAGTCACCAGCGACT | |
| | TGGTT | | TGACTACAGTCA | | ATGTCGTATGGGTAAAGCCGCT | |
| | | | CCAGCGACTATG | | TACGGACTTCGGTCCGTAAGAG | |
| | | | TCGTATGGGTAA | | GCATCAGAGCTGCATTCTAGTT | |
| | | | AGCCGCTTACGG | | GTGGTT | |
| | | | ACTTCGGTCCGT | | | |
| | | | AAGAGGCATCA | | | |
| | | | GAG | | | |
| 174.11.30 | AAGGG | 43979 | ACTGGCGCTTTT | 43983 | ACTGGCGCTTTTATCTGATTACT | 44003 |
| | GCTCCG | | ATCTGATTACTT | | TTGAGAGCCATCACCAGCGACT | |
| | CACCAC | | TGAGAGCCATCA | | ATGTCGTAGTGGGTAAAGCTCC | |
| | GCC | | CCAGCGACTATG | | CTCTTCGGAGGGAGCATCAAAG | |
| | | | TCGTAGTGGGTA | | AAGGGGCTCCGCACCACGCC | |
| | | | AAGCTCCCTCTT | | | |
| | | | CGGAGGGAGCA | | | |
| | | | TCAAAG | | | |
| 229.11.30 | AAGGG | 43979 | ACTGGCACTTTT | 43984 | ACTGGCACTTTTATCTGATTACT | 44004 |
| | GCTCCG | | ATCTGATTACTT | | TTGAGAGCCATCACCAGCGACT | |
| | CACCAC | | TGAGAGCCATCA | | ATGTCGTATGGGTAAAGCGCTT | |
| | GCC | | CCAGCGACTATG | | ACGGACTTCGGTCCGTAAGAAG | |
| | | | TCGTATGGGTAA | | CATCAAAGAAGGGGCTCCGCA | |
| | | | AGCGCTTACGGA | | CCACGCC | |
| | | | CTTCGGTCCGTA | | | |
| | | | AGAAGCATCAA | | | |
| | | | AG | | | |
| 230.11.30 | AAGGG | 43979 | ACTGGCACTTCT | 43985 | ACTGGCACTTCTATCTGATTAC | 44005 |
| | GCTCCG | | ATCTGATTACTC | | TCTGAGAGCCATCACCAGCGAC | |
| | CACCAC | | TGAGAGCCATCA | | TATGTCGTATGGGTAAAGCGCT | |
| | GCC | | CCAGCGACTATG | | TACGGACTTCGGTCCGTAAGAA | |
| | | | TCGTATGGGTAA | | GCATCAGAAAGGGGCTCCGCA | |
| | | | AGCGCTTACGGA | | CCACGCC | |
| | | | CTTCGGTCCGTA | | | |
| | | | AGAAGCATCAG | | | |
| | | | A | | | |
| 231.11.30 | AAGGG | 43979 | ACTGGCGCTTCT | 43986 | ACTGGCGCTTCTATCTGATTAC | 44006 |
| | GCTCCG | | ATCTGATTACTC | | TCTGAGAGCCATCACCAGCGAC | |
| | CACCAC | | TGAGAGCCATCA | | TATGTCGTATGGGTAAAGCCGC | |
| | GCC | | CCAGCGACTATG | | TTACGGACTTCGGTCCGTAAGA | |
| | | | TCGTATGGGTAA | | GGCATCAGAGAAGGGGCTCCG | |
| | | | AGCCGCTTACGG | | CACCACGCC | |
| | | | ACTTCGGTCCGT | | | |
| | | | AAGAGGCATCA | | | |
| | | | GAG | | | |
| 232.11.30 | AAGGG | 43979 | ACTGGCACTTCT | 43987 | ACTGGCACTTCTATCTGATTAC | 44007 |
| | GCTCCG | | ATCTGATTACTC | | TCTGAGCGCCATCACCAGCGAC | |
| | CACCAC | | TGAGCGCCATCA | | TATGTCGTATGGGTAAAGCCGC | |
| | GCC | | CCAGCGACTATG | | TTACGGACTTCGGTCCGTAAGA | |
| | | | TCGTATGGGTAA | | GGCATCAGAGAAGGGGCTCCG | |
| | | | AGCCGCTTACGG | | CACCACGCC | |
| | | | ACTTCGGTCCGT | | | |
| | | | AAGAGGCATCA | | | |
| | | | GAG | | | |
| 233.11.30 | AAGGG | 43979 | ACTGGCGCTTCT | 43988 | ACTGGCGCTTCTATCTGATTAC | 44008 |
| | GCTCCG | | ATCTGATTACTC | | TCTGAGCGCCATCACCAGCGAC | |
| | CACCAC | | TGAGCGCCATCA | | TATGTCGTATGGGTAAAGCCGC | |
| | GCC | | CCAGCGACTATG | | TTACGGACTTCGGTCCGTAAGA | |
| | | | TCGTATGGGTAA | | GGCATCAGAGAAGGGGCTCCG | |
| | | | AGCCGCTTACGG | | CACCACGCC | |
| | | | ACTTCGGTCCGT | | | |
| | | | AAGAGGCATCA | | | |
| | | | GAG | | | |
| 234.11.30 | AAGGG | 43979 | ACTGGCGCTTCT | 43989 | ACTGGCGCTTCTATCTGATTAC | 44009 |
| | GCTCCG | | ATCTGATTACTC | | TCTGAGCGCCATCACCAGCGAC | |
| | CACCAC | | TGAGCGCCATCA | | TATGTCGTATGGGTAAAGCGCC | |
| | GCC | | CCAGCGACTATG | | TTACGGACTTCGGTCCGTAAGG | |
| | | | TCGTATGGGTAA | | AGCATCAGAGAAGGGGCTCCG | |
| | | | AGCGCCTTACGG | | CACCACGCC | |
| | | | ACTTCGGTCCGT | | | |
| | | | AAGGAGCATCA | | | |
| | | | GAG | | | |
| 235.11.30 | AAGGG | 43979 | ACTGGCGCTTCT | 43990 | ACTGGCGCTTCTATCTGATTAC | 44010 |
| | GCTCCG | | ATCTGATTACTC | | TCTGAGCGCCATCACCAGCGAC | |
| | CACCAC | | TGAGCGCCATCA | | TATGTCGTAGTGGGTAAAGCCG | |
| | GCC | | CCAGCGACTATG | | CTTACGGACTTCGGTCCGTAAG | |
| | | | TCGTAGTGGGTA | | AGGCATCAGAGAAGGGGCTCC | |
| | | | AAGCCGCTTACG | | GCACCACGCC | |
| | | | GACTTCGGTCCG | | | |
| | | | TAAGAGGCATC | | | |
| | | | AGAG | | | |
| 236.11.30 | AAGGG | 43979 | ACGGGACTTTCT | 43991 | ACGGGACTTTCTATCTGATTAC | 44011 |
| | GCTCCG | | ATCTGATTACTC | | TCTGAAGTCCCTCACCAGCGAC | |
| | CACCAC | | TGAAGTCCCTCA | | TATGTCGTATGGGTAAAGCCGC | |
| | GCC | | CCAGCGACTATG | | TTACGGACTTCGGTCCGTAAGA | |
| | | | TCGTATGGGTAA | | GGCATCAGAGAAGGGGCTCCG | |
| | | | AGCCGCTTACGG | | CACCACGCC | |
| | | | ACTTCGGTCCGT | | | |
| | | | AAGAGGCATCA | | | |
| | | | GAG | | | |
| 237.11.30 | AAGGG | 43979 | ACCTGTAGTTCT | 43992 | ACCTGTAGTTCTATCTGATTACT | 44012 |
| | GCTCCG | | ATCTGATTACTC | | CTGACTACAGTCACCAGCGACT | |
| | CACCAC | | TGACTACAGTCA | | ATGTCGTATGGGTAAAGCCGCT | |
| | GCC | | CCAGCGACTATG | | TACGGACTTCGGTCCGTAAGAG | |
| | | | TCGTATGGGTAA | | GCATCAGAGAAGGGGCTCCGC | |
| | | | AGCCGCTTACGG | | ACCACGCC | |
| | | | ACTTCGGTCCGT | | | |
| | | | AAGAGGCATCA | | | |
| | | | GAG | | | |
| 174.11.31 | AAGTGG | 43980 | ACTGGCGCTTTT | 43983 | ACTGGCGCTTTTATCTGATTACT | 44013 |
| | CTCCGCA | | ATCTGATTACTT | | TTGAGAGCCATCACCAGCGACT | |
| | CCACGCC | | TGAGAGCCATCA | | ATGTCGTAgTGGGTAAAGCTCC | |
| | | | CCAGCGACTATG | | CTCTTCGGAGGGAGCATCAAAG | |
| | | | TCGTAgTGGGTA | | AAGTGGCTCCGCACCACGCC | |
| | | | AAGCTCCCTCTT | | | |
| | | | CGGAGGGAGCA | | | |
| | | | TCAAAG | | | |
| 235.11.31 | AAGTGG | 43980 | ACTGGCGCTTCT | 43990 | ACTGGCGCTTCTATCTGATTAC | 44014 |
| | CTCCGCA | | ATCTGATTACTC | | TCTGAGCGCCATCACCAGCGAC | |
| | CCACGCC | | TGAGCGCCATCA | | TATGTCGTAGTGGGTAAAGCCG | |
| | | | CCAGCGACTATG | | CTTACGGACTTCGGTCCGTAAG | |
| | | | TCGTAGTGGGTA | | AGGCATCAGAGAAGTGGCTCC | |
| | | | AAGCCGCTTACG | | GCACCACGCC | |
| | | | GACTTCGGTCCG | | | |
| | | | TAAGAGGCATC | | | |
| | | | AGAG | | | |
| 174.11.1 | AAGGGG | 43981 | ACTGGCGCTTTT | 43983 | ACTGGCGCTTTTATCTGATTACT | 44015 |
| | CTGCGTA | | ATCTGATTACTT | | TTGAGAGCCATCACCAGCGACT | |
| | CCACACC | | TGAGAGCCATCA | | ATGTCGTAGTGGGTAAAGCTCC | |
| | | | CCAGCGACTATG | | CTCTTCGGAGGGAGCATCAAAG | |
| | | | TCGTAGTGGGTA | | AAGGGGCTGCGTACCACACC | |
| | | | AAGCTCCCTCTT | | | |
| | | | CGGAGGGAGCA | | | |
| | | | TCAAAG | | | |
| 235.11.1 | AAGGGG | 43981 | ACTGGCGCTTCT | 43990 | ACTGGCGCTTCTATCTGATTAC | 44016 |
| | CTGCGTA | | ATCTGATTACTC | | TCTGAGCGCCATCACCAGCGAC | |
| | CCACACC | | TGAGCGCCATCA | | TATGTCGTAGTGGGTAAAGCCG | |
| | | | CCAGCGACTATG | | CTTACGGACTTCGGTCCGTAAG | |
| | | | TCGTAGTGGGTA | | AGGCATCAGAGAAGGGGCTG | |
| | | | AAGCCGCTTACG | | CGTACCACACC | |
| | | | GACTTCGGTCCG | | | |
| | | | TAAGAGGCATC | | | |
| | | | AGAG | | | |
| 235.NT | GGGTCT | 43982 | ACTGGCGCTTCT | 43990 | ACTGGCGCTTCTATCTGATTAC | 44017 |
| | TCGAGA | | ATCTGATTACTC | | TCTGAGCGCCATCACCAGCGAC | |
| | AGACCC | | TGAGCGCCATCA | | TATGTCGTAGTGGGTAAAGCCG | |
| | | | CCAGCGACTATG | | CTTACGGACTTCGGTCCGTAAG | |
| | | | TCGTAGTGGGTA | | AGGCATCAGAGGGGTCTTCG | |
| | | | AAGCCGCTTACG | | AGAAGACCC | |
| | | | GACTTCGGTCCG | | | |
| | | | TAAGAGGCATC | | | |
| | | | AGAG | | | |

### Reporter Cell Lines:

An immortalized neural progenitor cell line isolated from the Ai9-tdTomato was cultured in suspension in pre-equilibrated mNPC medium (DMEM/F12 with GlutaMax, 10 mM HEPES, 1× MEM Non-Essential Amino Acids, 1× Penicillin/Streptomycin, 1:1000 2-mercaptoethanol, 1× B-27 supplement, minus vitamin A, 1× N2 with supplemented growth factors bFGF and EGF). Prior to testing, cells were lifted using accutase, with gentle resuspension, monitoring for complete separation of the neurospheres. Cells were then quenched with media, spun down and resuspended in fresh media. Cells were counted and directly used for nucleofection or 10,000 cells were incubated in a 96-well plate coated with PLF (1× Poly-DL-ornithine hydrobromide, 10 mg/mL in sterile diH20, 1× Laminin, and 1× Fibronectin), 2 days prior to AAV transduction.

A HEK293T dual reporter cell line was generated by knocking into HEK293T cells two transgene cassettes that constitutively expressed exon 1 of the human RHO gene linked to GFP and exon 1 of the human P23H.RHO gene linked to mscarlet. The modified cells were expanded by serial passage every 3-5 days and maintained in Fibroblast (FB) medium, consisting of Dulbecco's Modified Eagle Medium (DMEM; Corning Cellgro, #10-013-CV) supplemented with 10% fetal bovine serum (FBS; Seradigm, #1500-500), and 100 Units/mL penicillin and 100 mg/mL streptomycin (100×-Pen-Strep; GIBCO #15140-122), and can additionally include sodium pyruvate (100×, ThermoFisher #11360070), non-essential amino acids (100× ThermoFisher #11140050), HEPES buffer (100× ThermoFisher #15630080), and 2-mercaptoethanol (1000× ThermoFisher #21985023). The cells were incubated at 37° C. and 5% C02. After 1-2 weeks, GFP+/mscarlet+ cells were bulk-sorted into FB medium. The reporter lines were expanded by serial passage every 3-5 days and maintained in FB medium in an incubator at 37° C. and 5% C02. Reporter clones were generated by a limiting dilution method. The clonal lines were characterized via flow cytometry, genomic sequencing, and functional modification of the RHO locus using a previously validated RHO targeting CasX molecule. The optimal reporter lines were identified as ones that i) had a single copies of WTRHO.GFP and mutRHO.mscarlet correctly integrated per cell, ii) maintained doubling times equivalent to unmodified cells, and iii) resulted in reduction in GFP and mscarlet fluorescence upon disruption of the RHO gene when assayed using the methods described below.

### Nucleofection:

AAV cis-plasmids driving expression of the CasX-scaffold-guide system were nucleofected in mNPCs using the Lonza P3 Primary Cell 96-well Nucleofector Kit. For the ARPE-19 line, the Lonza SF solution and supplement was used. Plasmids were diluted to concentrations of 200 ng/μl, 100 ng/μL. 5 μL of DNA per construct was added to the P3 or SF solution containing 200,000 tdTomato mNPCs or ARPE-19 cells respectively. The combined solution was nucleofected using a Lonza 4D Nucleofector System according to manufacturer's guidelines. Following nucleofection, the solution was quenched with appropriate culture media. The solution was then aliquoted in triplicate (approx. 67,000 cells per well) in a 96-well plate. 48 hours after transfection, treated cells were replenished with fresh mNPC media containing growth factors. 5 days after transfection, tdTomato mNPCs were lifted and activity was assessed by FACS.

### AAV Production:

Suspension HEK293T cells were adapted from parental HEK293T and grown in FreeStyle 293 media. For screening purposes, small scale cultures (20-30 mL cultured in 125 mL Erlenmeyer flasks and agitated at 110 rpm) were diluted to a density of 1.5e+6 cells/mL on the day of transfection. Endotoxin-free pAAV plasmids with the transgene flanked by ITR repeats were co-transfected with plasmids supplying the adenoviral helper genes for replication and AAV rep/cap genome using PEIMax (Polysciences) in serum-free OPTIMEM media. Cultures were supplemented with 10% CDM4HEK293 (HyClone) 3 hours post-transfection. Three days later, cultures were centrifuged at 1000 rpm for 10 minutes to separate the supernatant from the cell pellet. The supernatant was mixed with 40% PEG 2.5M NaCl (8% final concentration) and incubated on ice for at least 2 hours to precipitate AAV viral particles. The cell pellet, containing the majority of the AAV vectors, was resuspended in lysis media (0.15M NaCl, 50 mM Tris HCl, 0.05% Tween, pH 8.5), sonicated on ice (15 seconds, 30% amplitude) and treated with Benzonase (250 U/μL, Novagen) for 30 minutes at 37° C. Crude lysate and PEG-treated supernatant were then spin at 4000 rpm for 20 minutes at 4° C. to resuspend the PEG precipitated AAV (pellet) with cell debris-free crude lysate (supernatant). clarified further using 0.45 μm filter.

To determine the viral genome titer, 1 μL from crude lysate viruses was digested with DNase and ProtK, followed by quantitative PCR. 5 μL of digested virus was used in a 25 μL qPCR reaction composed of IDT primetime master mix and a set of primer and 6′FAM/Zen/IBFQ probe (IDT) designed to amplify the CMV promoter region. Ten-fold serial dilutions (5 μl each of 2e+9 to 2e+4 DNA copies/mL) of an AAV ITR plasmid was used as reference standards to calculate the titer (viral genome (vg)/mL) of viral samples. QPCR program was set up as: initial denaturation step at 95° C. for 5 minutes, followed by 40 cycles of denaturation at 95° C. for 1 min and annealing/extension at 60° C. for 1 min.

### AAV Transduction:

10,000 cells/well of mNPCs were seeded on PLF-coated wells in 96-well plates 48-hours before AAV transduction. All viral infection conditions were performed in triplicate, with normalized number of vg among experimental vectors, in a series of 3-fold dilution of multiplicity of infection (MOI) ranging from ˜1.0e+6 to 1.0e+4 vg/cell. Calculations were based on an estimated number of 20,000 cells per well at the time of transfection. Final volume of 50 μL of AAV vectors diluted in pre-equilibrated mNPC medium supplemented with bFGF/EGF growth factors (20 ng/ml final concentration) were applied to each well. 48 hours post-transfection, complete media change was performed with fresh media supplemented with growth factors. Editing activity (tdT+ cell quantification) was assessed by FACS 5 days post-transfection.

### Assessing Editing Activity by FACS:

5 days after transfection, treated tdTomato mNPCs or ARPE-19 cells in 96-well plates were washed with dPBS and treated with 50 μL TrypLE and Trypsin (0.25%) for 15 and 5 minutes respectively. Following cell dissociation, treated wells were quenched with media containing DMEM, 10% FBS and 1× Penicillin/Streptomycin. Resuspended cells were transferred to round-bottom 96-well plates and centrifuged for 5 min at 1000 g. Cell pellets were then resuspended with dPBS containing 1× DAPI, and plates were loaded into an Attune NxT Flow Cytometer Autosampler. The Attune NxT flow cytometer was run using the following gating parameters: FSC-A×SSC-A to select cells, FSC-H×FSC-A to select single cells, FSC-A×VL1-A to select DAPI-negative alive cells, and FSC-A×YL1-A to select tdTomato positive cells.

NGS Analysis of Indels at mRHO Exon 1 Locus:

5 days after transfection, treated tdTomato mNPCs in 96-well plates were washed with dPBS and treated with 50 μL TrypLE and Trypsin (0.25%) for 15 and 5 minutes respectively. Following cell dissociation, treated wells were quenched with media containing DMEM, 10% FBS and 1× Penicillin/Streptomycin. Cells were then spun down and resulting cell pellets washed with PBS prior to processing them for gDNA extraction using the Zymo mini DNA kit according to the manufacturer's instructions. For assessing editing levels occurring at the mouse RHO exon 1 locus, amplicons were amplified from 200 ng of gDNA with a set of primers, bead-purified (Beckman coulter, Agencourt Ampure XP) and then re-amplified to incorporate an Illumina adapter sequence and a 16 nt unique molecular identifier (UMI). Quality and quantification of the amplicon was assessed using a Fragment Analyzer DNA analyzer kit (Agilent, dsDNA 35-1500 bp). Amplicons were sequenced on the Illumina Miseq according to the manufacturer's instructions. Raw fastq files from sequencing were processed as follows: (1) the sequences were trimmed for quality and for adapter sequences using the program cutadapt (v. 2.1); (2) the sequences from read 1 and read 2 were merged into a single insert sequence using the program flash2 (v2.2.00); and (3) the consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). CasX activity was quantified as the total percent of reads that contain insertions, substitutions and/or deletions anywhere within this window.

### Results:

Different editing experiments were conducted to quantify on-target cleavage mediated by CasX 491 paired with novel gRNA scaffold variants (guide 174 & 229-237) with different spacers targeting multiple genomic loci of interest. Constructs were cloned into the AAV backbone p59, flanked by ITR2 sequences, driving expression of the protein Cas 491 under the control of a CMV promoter, as well the scaffold-spacer under the control of the human U6 promoter. The mNPC-tdT reporter cell lines was used to assess the dual-cut efficiency mediated by a single spacer at the tdTomato locus (spacer 12.7, TTCN PAM), as well as the single cut efficiency at the endogenous mouse RHO exon 1 locus (spacer 11.30, CTCN PAM). A dual reporter system integrated in an ARPE-19 derived cell line was also used to assess on-target editing at the exogenously expressed human WT Rho locus (spacers 11.1, CTCN PAM).

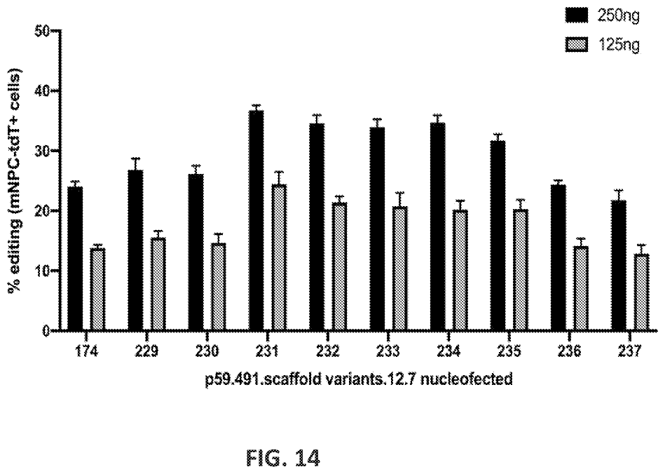

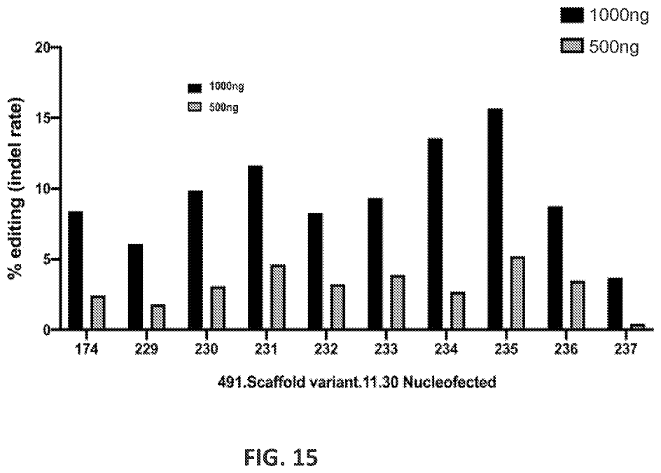

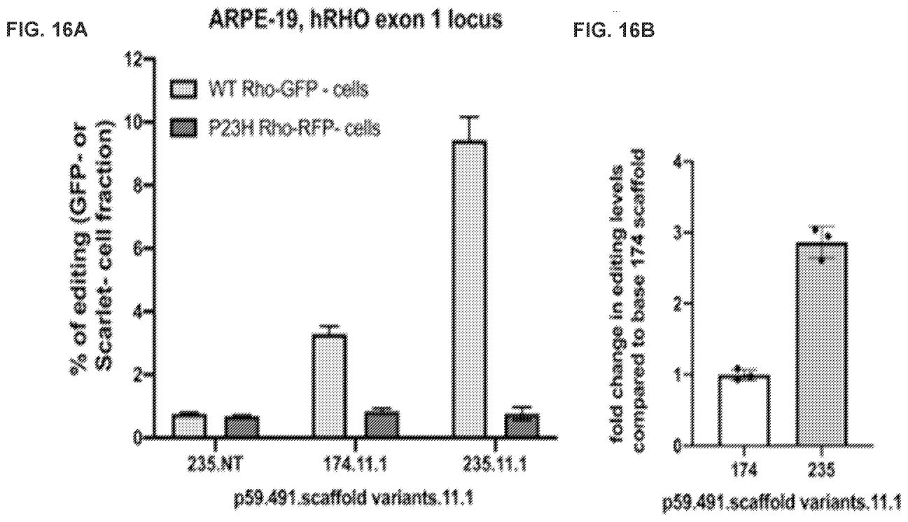

Scaffold variants with spacers 12.7 and 11.30 were tested via nucleofection in the mouse NPC cell line at two different doses indicated in FIG. 14 and FIG. 15, respectively. Constructs were compared to the current benchmark gRNA scaffold 174 activity. At both targeted loci, constructs of guide scaffold variants 231, 233, 234, and 235 performed at higher levels than the construct containing scaffold 174 (FIGS. 14 and 15). Scaffold 235 displayed a 2-fold increase in activity at mRHO exon 1 locus compared to scaffold 174. We further validated that scaffold 235 consistently improved activity without increased off-target cleavage by nucleofecting dual reporter ARPE-19 cell line with construct p59.491.174.11.1 and p59.491.235.11.1 as well as a non-target spacer control. Spacer 11.1 was targeting the exogenously expressed mRHO-GFP gene. Scaffold 235 displayed 3-fold increased activity compared to 174 (9% vs 3% of Rho-GFP-cells, respectively, FIGS. 16A & B). Allele-specificity was assessed by looking at percent of P23H—RHO-Scarlett-cell population, which sequence differs from WT by 1 bp.

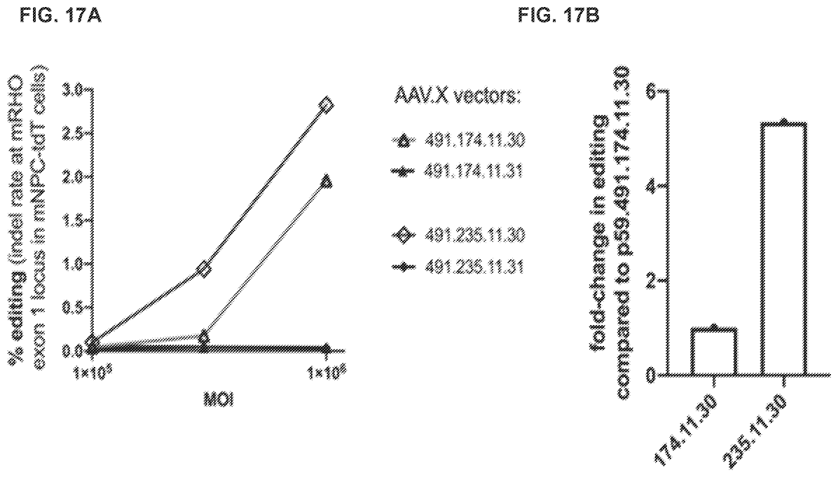

Finally, we sought to demonstrate that these scaffold variants packaged efficiently in AAV and remained potent when delivered virally. mNPC transduced with AAV vectors expressing guide scaffold variants 174 and 235 with spacer 11.30 (on target, mouse WT RHO) and 11.31 (off-target, mouse P23 RHO) showed increased activity of constructs containing the 235 scaffold variant compared to scaffold 174 at on-target locus (>5-fold increase, FIGS. 17A & B) at 3.0e+5 MOI while no off-target indels were detectable.

The results support that scaffold variants with novel structural mutations can be engineered with increased activity in dual reporter systems with therapeutically-relevant genomic targets such as mouse and human RHO exon 1 loci. Furthermore, while the newly-characterized scaffold displayed an overall >2-fold increase in activity, no off-target cleavage with 1-bp mismatch spacer region was detected. This is relevant for allele-specific therapeutic strategy such as adRP P23H Rho, which mutated allele differs from WT sequence by 1 nucleotide, targeted by spacer 11.31. This study further validates the use of guide scaffold 235 in AAV vectors designed for P23H RHO rescue and genotoxic studies, as well as for other therapeutic targets.

### Example 13: CasX:gRNA Constructs for Editing of PTBP1

This example describes the methods to make and test compositions capable of modifying a PTBP1 locus.

### A) Method to Design PTBP1-Modifying Spacers:

20 bp XTC PAM spacers are designed to target the following regions in the human genome:
(a) PTBP1 cis enhancer elements
(b) PTBP1 proximal non-coding genetic elements highly conserved across vertebrates (UCSC genome browser)
(c) PTBP1 genomic locus. The PTBP1 gene is defined as the sequence that spans chr19:797,075-812,327 of the human genome (GRCh38/hg38) (the notation refers to the chromosome 19 (chr19), starting at the 797,075 bp of that chromosome, and extending to the 812,327 bp of that chromosome).

PTBP1 targeting spacers may be similarly assembled from other genomes.

### B) Methods for Generating PTBP1 Targeting Constructs:

In order to generate PTBP1 targeting constructs, PTBP1 targeting spacers (representative examples listed below) are cloned into a base mammalian-expression plasmid construct (pStX) that is comprised of the following components: codon optimized CasX (construct CasX 491 (SEQ ID NO: 126) and guide 174 (SEQ ID NO: 2238)+NLS; and a mammalian selection marker, puromycin. Spacer sequence DNA will be ordered as single-stranded DNA (ssDNA) oligos from Integrated DNA Technologies (IDT) consisting of the spacer sequence and the reverse complement of this sequence. These two oligos are annealed together and cloned into pStX individually or in bulk by Golden Gate Assembly using T4 DNA Ligase (New England BioLabs Cat #M0202L) and an appropriate restriction enzyme for the plasmid. Assembled products are transformed into chemically- or electro-competent bacterial cells, plated on LB-Agar plates (LB: Teknova Cat #L9315, Agar: Quartzy Cat #214510) containing carbenicillin and incubated until colonies appeared. Individual colonies are picked and miniprepped using a Qiagen spin Miniprep Kit (Qiagen Cat #27104), following the manufacturer's protocol. The resultant plasmids will be sequenced using Sanger sequencing to ensure correct ligation. SaCas9 and SpyCas9 control plasmids, with spacers chosen based on Cas protein-specific PAMs prepared similarly to pStX plasmids described above.

### Example 14: Use of CasX:gNA Systems to Edit the Mouse PTBP1 Locus in Primary Mouse Astrocytes when Delivered Via CasX-Delivery Particles (XDPs) In Vitro

Experiments were performed to demonstrate that CasX and guide RNAs can edit at the mouse PTBP1 locus in cultured primary mouse astrocytes when delivered via CasX-delivery particles (XDPs).

### Materials and Methods:

### XDP Construct Cloning:

XDP plasmid constructs comprising sequences coding for CasX protein 491, guide scaffold variant 174, and a spacer targeting PTBP1 were transformed into chemically-competent E. coli cells, which were plated on kanamycin LB-agar plates following recovery at 37° C. for 1 hour. Single colonies were picked, mini-prepped, and Sanger-sequenced. Sequence-verified constructs were then cloned into an expression vector via Golden Gate assembly with the PTBP1-targeting spacers. PTBP1 spacers were randomly selected based on PAM availability; in this experiment, spacers 28.1 to 28.12 (see Table 17 for sequences) were tested in an initial screen to assess editing efficiency. Spacers were made by annealing two oligos and cloned via Golden Gate assembly with the appropriate restriction enzymes. Cloned spacers were subjected to transformation, mini-prepping, and Sanger-sequencing for verification as described.

TABLE 17
Spacers for targeting mouse PTBP1

| | | SEQ | | | SEQ |
|---|---|---|---|---|---|
| Spacer | Sequence | ID NO | Spacer | Sequence | ID NO |
| 28.1 | GUCUUCCGUCUUGACUGACG | 393 | 28.44 | CUAGACCAGACCAUGGCAGC | 436 |
| 28.2 | GUCUUGACUGACGGCCGUCC | 394 | 28.45 | UUUGGUGUGUCUCAGUACAG | 437 |
| 28.3 | AUUGGUCAAAAGGUUACUCC | 395 | 28.46 | AGGUGCGCCCGGCAUAAUGU | 438 |
| 28.4 | CCCUUGGAGUAACCUUUUGA | 396 | 28.47 | CUCCCACCUUUGCCAUCCCU | 439 |
| 28.5 | CGCUGCGGUCUGUGGGCGUG | 397 | 28.48 | CGCUCAUCCUGACCCCAGCG | 440 |
| 28.6 | GCUAUUCCUGCGCCUCCGCU | 398 | 28.49 | CCUCUCCCCAGAAGGAAGGG | 441 |
| 28.7 | UGCGCCUCCGCUCCGUUCCC | 399 | 28.5 | GGAGGCCCCUUCCCCUCUCC | 442 |
| 28.8 | CCGCGGGUCUCUUCCGUGUG | 400 | 28.51 | UUCUGGGGAGAGGGGAAGGG | 443 |
| 28.9 | GUGUGCCAUGGACGGGUAAG | 401 | 28.52 | GGGGAGAGGGGAAGGGGCCU | 444 |
| 28.10 | CAGCGGGGAUCCGACGAGCU | 402 | 28.53 | CUUCAAGCACCUCUGGCGAG | 445 |
| 28.11 | CCACGUGUGUCAGCAACGGC | 403 | 28.54 | AGCACCUCUGGCGAGGACGA | 446 |
| 28.12 | UCAUGAGCAGCUCUGCCUCA | 404 | 28.55 | AUCUACUAGGGACACUGCGU | 447 |
| 28.13 | CCCAGCUCGCCCUGUACCUG | 405 | 28.56 | UCCAUGCAUGCUCCGGCGGC | 448 |
| 28.14 | UGCCUCUGCCAGGUCCUUUC | 406 | 28.57 | UUUGCAGGCCUCUCUGUCCC | 449 |
| 28.15 | AUUGGCUGAAAAGGGAUAGG | 407 | 28.58 | CAGCACCUGCCAACCCUGGG | 450 |
| 28.16 | UGCUAUCGUUUCCAUUGGCU | 408 | 28.59 | GAUUGCUGACCAAAAGGACA | 451 |
| 28.17 | GCCAAUGGAAACGAUAGCAA | 409 | 28.6 | GUCCUUUUGGUCAGCAAUCU | 452 |
| 28.18 | AAGGUGACAACAGGAGCGCA | 410 | 28.61 | CCCGCUGCACAUCACCGUAG | 453 |
| 28.19 | GACAUGGAUGACUCUGGAAG | 411 | 28.62 | AAGGCGUCUACGGUGAUGUG | 454 |
| 28.2 | AGAGUCAUCCAUGUCAGAAA | 412 | 28.63 | UAUUGAACAGGAUCUUCACC | 455 |
| 28.21 | AAAGGGCAGCCCUAGGGAGA | 413 | 28.64 | CCUUCUUAUUGAACAGGAUC | 456 |
| 28.22 | GCAUGAGAAGGUUGGUAACC | 414 | 28.65 | AUAAGAAGGAGAACGCACUU | 457 |
| 28.23 | CCUUCAGCAUGAGAAGGUUG | 415 | 28.66 | AGCACAAGCCUGUGCCCUGU | 458 |
| 28.24 | UCCCCUUCAGCAUGAGAAGG | 416 | 28.67 | GGUGGCUCAUGGCUGGACAA | 459 |
| 28.25 | UCUCAAUGAAGGCCUGGUUC | 417 | 28.68 | ACCCUUUGUCCAGCCAUGAG | 460 |
| 28.26 | CAUGCUGAAGGGGAAGAACC | 418 | 28.69 | CGUGCAGCUUGUGCCCGUUC | 461 |
| 28.27 | UUGAGAUGAACACAGAGGAG | 419 | 28.7 | UGAAGCGGUGCAGCGGGGAG | 462 |
| 28.28 | UGGACAGCCCAUCUACAUCC | 420 | 28.71 | UGGAGCCUGGUUUCUUGAAG | 463 |
| 28.29 | CCAACCACAAAGAGCUCAAG | 421 | 28.72 | GGAAGUUCUUGGAGCCUGGU | 464 |
| 28.3 | UCCACAGCGUGCCCAGGCAG | 422 | 28.73 | AGAAACCAGGCUCCAAGAAC | 465 |
| 28.31 | AGACUGGACGGAGUUUACAG | 423 | 28.74 | AGAACAUCUUUCCACCCUCA | 466 |
| 28.32 | UGCAUCCACGGCAGCAGCGG | 424 | 28.75 | ACCCUCAGCUACCCUGCACC | 467 |
| 28.33 | ACAAUGAUCCUGAGCACUGG | 425 | 28.76 | UGGCCCACCCACAAACAAGA | 468 |
| 28.34 | ACCCAGUGACCCUGGACGUG | 426 | 28.77 | CCAGCAACGGUGGUGUGGUC | 469 |
| 28.35 | CACUUGGUCGGCCCCAGCUA | 427 | 28.78 | AGUUCUUCCAGUGAGUAAAG | 470 |
| 28.36 | CACAUCCUCUCUUGCAGAUC | 428 | 28.79 | UCCAGUGAGUAAAGCCCUGC | 471 |
| 28.37 | GGACGGUGCCAAACUUAGAG | 429 | 28.8 | AGUGAGUAAAGCCCUGCCUU | 472 |
| 28.38 | CUAAGUUUGGCACCGUCCUG | 430 | 28.81 | UCCUCCACCCUUAACCCUAC | 473 |
| 28.39 | UGGUGAACGUGAUGAUCUUC | 431 | 28.82 | UGCAGGGGUGGAGUGGGGGG | 474 |
| 28.4 | CCAAGAACAACCAGUUCCAG | 432 | 28.83 | AUCAGCGCCUGCACAGCCUC | 475 |
| 28.41 | AGGCGCUGCUGCAGUAUGCU | 433 | 28.84 | CGCCCAGGUCAUGGUUGUGC | 476 |
| 28.42 | GGCCAUCCAGGGACUGUGGG | 434 | 28.85 | AAGUCCACCAUCUAGGUGCC | 477 |
| 28.43 | CCAAGCUCACCAGUCUCAAU | 435 | | | |

### Xdp Production:

XDP containing ribonucleoproteins (RNPs) of CasX 491 protein and single guide RNA with scaffold 174 and a spacer targeting the PTBP1 locus were produced using either suspension-adapted or adherent HEK293T Lenti-X cells. The methods to produce XDPs are described in WO2021113772A1, incorporated by reference in its entirety.

Briefly, for XDPs produced in suspension, suspension-adapted Lenti-X cells, maintained in FreeStyle 293 Expression media, were seeded in 40 mL of media at 1.5E6 cells/mL just before transfection. The next day, Lenti-X cells were transfected with the following plasmids using PEI Max (Polypus): XDP structural plasmids (which encode the HIV-1 gag-pol structural components, as well as the CasX variant), a plasmid encoding a single guide RNA with scaffold 174 and a spacer targeting PTBP1, and a plasmid encoding VSV-G (glycoprotein 2 or GP2) for pseudotyping the XDP. Media was supplemented with CDM4HEK293 from Cytiva within 24 hours post-transfection. XDP-containing media was collected 72 hours post-transfection and filtered through a 0.45 m PES filter. The supernatant was concentrated and purified via centrifugation. XDPs were resuspended in 125 μL of freezing buffer (15% Trehalose, 150 mM NaCl in PBS).

For XDPs produced in adherent culture, HEK293T Lenti-X cells were maintained in 10% FBS supplemented DMEM with HEPES and Glutamax (Thermo Fisher). Cells were seeded in 15 cm dishes at 20×10E6 cells per dish in 20 mL of media. Cells were allowed to settle and grow for 24 hours before transfection. Once they reached 70-90% confluency, cells were transfected with the following plasmids using PEI Max (Polypus): XDP structural plasmids (also encoding CasX), a plasmid encoding a single guide with a spacer targeting PTBP1, and a plasmid encoding VSV-G (GP2). Media was aspirated from the plates 24 hours post-transfection and replaced with Opti-MEM (Thermo Fisher). XDP-containing media was collected 72 hours post-transfection and filtered through a 0.45 m PES filter. The supernatant was concentrated and purified via centrifugation. XDPs were resuspended in freezing buffer.

### XDP Transduction of Astrocytes In Vitro:

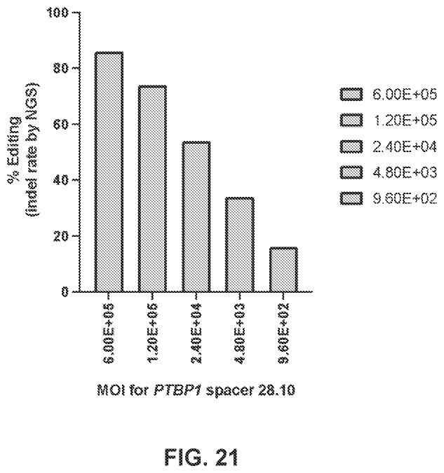

Mouse midbrain astrocytes were grown in BrainBits Nb Astro media supplemented with Pen/Strep using 10 cm plates coated with PLF (Poly-DL-ornithine hydrobromide, laminin, and fibronectin). Cells were trypsinized at 90% confluency and seeded on a 24-well tissue culture plate, also coated with PLF, at a density of 120,000 cells per well for the assay. Next day, XDP particles containing CasX RNPs with PTBP1-targeting spacers (28.1 to 28.12) were applied. Based on cell plating density and total volume of virion applied, the effective XDP MOI was ˜6E5; for the dose-dependency experiment depicted in FIG. 21, five-fold serial dilutions were tested, starting with the effective MOI of 6E5. Cells were fed with a 50% media exchange every other day. Five days post-transduction, cells were trypsinized for editing analysis by next-generation sequencing (NGS).

### NGS Processing and Analysis:

Genomic DNA (gDNA) from harvested cells were extracted using the Zymo Quick-DNA Miniprep Plus kit following the manufacturer's instructions. Target amplicons were formed by amplifying regions of interest from 200 ng of extracted gDNA with a set of primers specific to the mouse PTBP1 locus. These gene-specific primers contain an additional sequence at the 5′ end to introduce an Illumina adapter and a 16-nucleotide unique molecule identifier. Amplified DNA products were purified with the Ampure XP DNA cleanup kit. Quality and quantification of the amplicon were assessed using a Fragment Analyzer DNA Analysis kit (Agilent, dsDNA 35-1500 bp). Amplicons were sequenced on the Illumina Miseq according to the manufacturer's instructions. Raw fastq files from sequencing were quality-controlled and processed using cutadapt v2.1, flash2 v2.2.00, and CRISPResso2 v2.0.29. Each sequence was quantified for containing an insertion or deletion (indel) relative to the reference sequence, in a window around the 3′ end of the spacer (30 bp window centered at −3 bp from 3′ end of spacer). CasX activity was quantified as the total percent of reads that contain insertions, substitutions, and/or deletions anywhere within this window for each sample.

### Results:

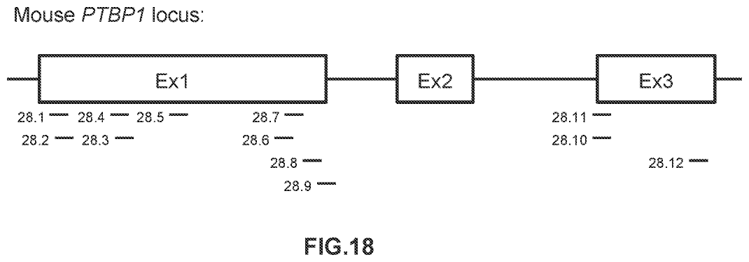

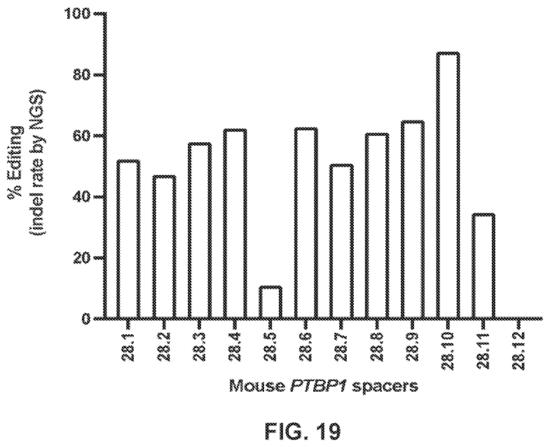

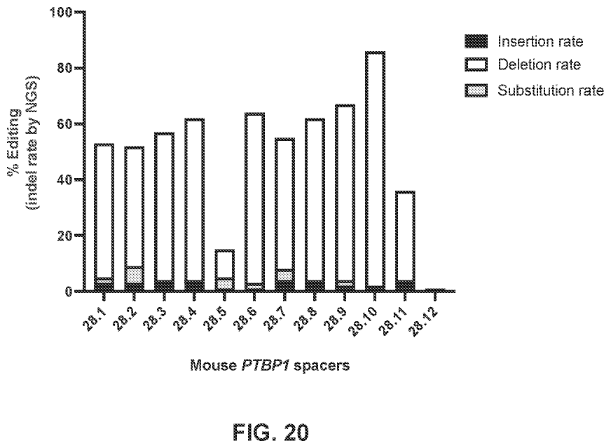

An initial screen testing 12 spacers targeting the mouse PTBP1 locus using CasX variant 491 and scaffold variant 174, which were delivered as RNPs via XDPs, was performed in primary mouse astrocytes. These 12 spacers targeted either exon 1 or 3 of the murine PTBP1 gene (see FIG. 18 for exon region targeted by spacers). FIG. 19 shows the editing results of the initial screen testing and demonstrated that 11 out of 12 spacers were able to edit the target locus, with spacer 28.10 achieving the highest editing efficiency of ˜88%. Additional analysis showed that most mutations generated by these PTBP1-targeting spacers were deletions (FIG. 20). Further characterization of top spacer 28.10 revealed that editing of primary mouse midbrain astrocytes was able to occur in a dose-dependent manner, with >80% editing rate achieved at the highest MOI of 6E5 (FIG. 21).

The experiments demonstrate that CasX variant 491 and guide scaffold 174 with spacers targeting the mouse PTBP1 locus were able to edit on-target efficiently in primary mouse astrocytes when packaged as RNPs and delivered in vitro via XDPs.

### Example 15: Use of CasX:gNA Systems to Edit the Mouse PTBP1 Locus in Mouse Astrocytes when Packaged and Delivered Via a Single Adeno-Associated Virus (AAV) Vector In Vitro

Experiments were performed to demonstrate the following: 1) CasX and guide RNAs, when delivered via AAVs, can edit at the PTBP1 locus in cultured murine midbrain astrocytes; 2) the ability to encode, package, and deliver CasX with a dual-guide system within a single AAV vector particle for targeted editing, which is not achievable with Cas9-based systems; and 3) the ability to mark edited astrocytes with tdTomato fluorescence to track their conversion into neurons mediated by PTBP1-editing using our engineered dual-guide vector system with CasX in cultured primary mouse astrocytes.

### Materials and Methods:

### AAV Construct Cloning:

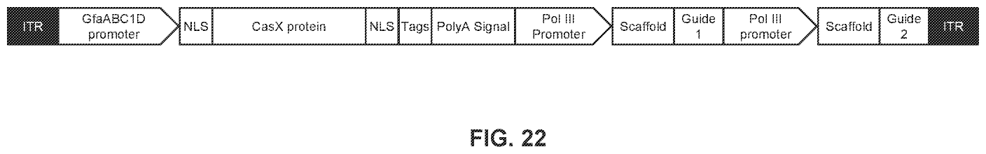

CasX variant 491 and guide scaffold variant 174 were used in these experiments. AAV constructs containing an astrocyte-specific promoter driving CasX expression, and two Pol III promoter-scaffold 174-spacer combinations (depicted in FIG. 22) were transformed into chemically-competent E. coli cells. Transformed cells were plated on kanamycin LB-agar plates following recovery at 37° C. for 1 hour, and single colonies were picked, mini-prepped, and Sanger-sequenced. Sequence-verified constructs were then assembled using Golden Gate cloning with the indicated combinations of spacers (see Table 18). Spacer 28.10 targeting PTBP1 was selected for this experiment because it demonstrated the highest editing activity, as described in Example 14. Spacers were made by annealing two oligos and cloned via Golden Gate assembly using the appropriate restriction enzymes. Sequence-validated constructs were maxi-prepped and subjected to quality assessment prior to transfection for AAV production.

TABLE 18
Sequences of AAV constructs with dual-guide system.
NT = non-targeting guide.

| Construct | Component | | SEQ |
|---|---|---|---|
| ID | Name | DNA Sequence | ID NO |
| 1 and 2 | 5' ITR | CCTGCAGGCAGCTGCGCGCTCGCTCGCTCACTGAGGCCGCCCGG | 44018 |
| | | GCGTCGGGCGACCTTTGGTCGCCCGGCCTCAGTGAGCGAGCGAG | |
| | | CGCGCAGAGAGGGAGTGGCCAACTCCATCACTAGGGGTTCCT | |
| | buffer sequence | GCGGCCTCTAGACTCGAG | 44019 |
| | GfaABCID | AACATATCCTGGTGTGGAGTAGGGGACGCTGCTCTGACAGAGGC | 44020 |
| | promoter | TCGGGGGCCTGAGCTGGCTCTGTGAGCTGGGGAGGAGGCAGACA | |
| | | GCCAGGCCTTGTCTGCAAGCAGACCTGGCAGCATTGGGCTGGCC | |
| | | GCCCCCCAGGGCCTCCTCTTCATGCCCAGTGAATGACTCACCTT | |
| | | GGCACAGACACAATGTTCGGGGTGGGCACAGTGCCTGCTTCCCG | |
| | | CCGCACCCCAGCCCCCCTCAAATGCCTTCCGAGAAGCCCATTGA | |
| | | GCAGGGGGCTTGCATTGCACCCCAGCCTGACAGCCTGGCATCTT | |
| | | GGGATAAAAGCAGCACAGCCCCCTAGGGGCTGCCCTTGCTGTGT | |
| | | GGCGCCACCGGCGGTGGAGAACAAGGCTCTATTCAGCCTGTGCC | |
| | | CAGGAAAGGGGATCAGGGGATGCCCAGGCATGGACAGTGGGTGG | |
| | | CAGGGGGGGAGAGGAGGGCTGTCTGCTTCCCAGAAGTCCAAGGA | |
| | | CACAAATGGGTGAGGGGAGAGCTCTCCCCATAGCTGGGCTGCGG | |
| | | CCCAACCCCACCCCCTCAGGCTATGCCAGGGGGTGTTGCCAGGG | |
| | | GCACCCGGGCATCGCCAGTCTAGCCCACTCCTTCATAAAGCCCT | |
| | | CGCATCCCAGGAGCGAGCAGAGCCAGAGCAGGTTGGAGAGGAGA | |
| | | CGCATCACCTCCGCTGCTCGCGGGGTCT | |
| | buffer sequence | ACCGGT | 44021 |
| | Kozak | GCCACC | 44022 |
| | Start codon | ATGGCC | 44023 |
| | SV40 NLS | CCAAAGAAGAAGCGGAAGGTC | 230 |
| | linker | TCTAGA | 44024 |
| | CasX 491 | CAAGAGATCAAGAGAATCAACAAGATCAGAAGGAGACTGGTCAA | 215 |
| | | GGACAGCAACACAAAGAAGGCCGGCAAGACAGGCCCCATGAAAA | |
| | | CCCTGCTCGTCAGAGTGATGACCCCTGACCTGAGAGAGCGGCTG | |
| | | GAAAACCTGAGAAAGAAGCCCGAGAACATCCCTCAGCCTATCAG | |
| | | CAACACCAGCAGGGCCAACCTGAACAAGCTGCTGACCGACTACA | |
| | | CCGAGATGAAGAAAGCCATCCTGCACGTGTACTGGGAAGAGTTC | |
| | | CAGAAAGACCCCGTGGGCCTGATGAGCAGAGTTGCTCAGCCTGC | |
| | | CAGCAAGAAGATCGACCAGAACAAGCTGAAGCCCGAGATGGACG | |
| | | AGAAGGGCAATCTGACCACAGCCGGCTTTGCCTGCTCTCAGTGT | |
| | | GGCCAGCCTCTGTTCGTGTACAAGCTGGAACAGGTGTCCGAGAA | |
| | | AGGCAAGGCCTACACCAACTACTTCGGCAGATGTAACGTGGCCG | |
| | | AGCACGAGAAGCTGATTCTGCTGGCCCAGCTGAAACCTGAGAAG | |
| | | GACTCTGATGAGGCCGTGACCTACAGCCTGGGCAAGTTTGGACA | |
| | | GAGAGCCCTGGACTTCTACAGCATCCACGTGACCAAAGAAAGCA | |
| | | CACACCCCGTGAAGCCCCTGGCTCAGATCGCCGGCAATAGATAC | |
| | | GCCTCTGGACCTGTGGGCAAAGCCCTGTCCGATGCCTGCATGGG | |
| | | AACAATCGCCAGCTTCCTGAGCAAGTACCAGGACATCATCATCG | |
| | | AGCACCAGAAGGTGGTCAAGGGCAACCAGAAGAGACTGGAAAGC | |
| | | CTGAGGGAGCTGGCCGGCAAAGAGAACCTGGAATACCCCAGCGT | |
| | | GACCCTGCCTCCTCAGCCTCACACAAAAGAAGGCGTGGACGCCT | |
| | | ACAACGAAGTGATCGCCAGAGTGAGAATGTGGGTCAACCTGAAC | |
| | | CTGTGGCAGAAGCTGAAACTGTCCAGGGACGACGCCAAGCCTCT | |
| | | GCTGAGACTGAAGGGCTTCCCTAGCTTCCCTCTGGTGGAAAGAC | |
| | | AGGCCAATGAAGTGGATTGGTGGGACATGGTCTGCAACGTGAAG | |
| | | AAGCTGATCAACGAGAAGAAAGAGGATGGCAAGGTTTTCTGGCA | |
| | | GAACCTGGCCGGCTACAAGAGACAAGAAGCCCTGAGGCCTTACC | |
| | | TGAGCAGCGAAGAGGACCGGAAGAAGGGCAAGAAGTTCGCCAGA | |
| | | TACCAGCTGGGCGACCTGCTGCTGCACCTGGAAAAGAAGCACGG | |
| | | CGAGGACTGGGGCAAAGTGTACGATGAGGCCTGGGAGAGAATCG | |
| | | ACAAGAAGGTGGAAGGCCTGAGCAAGCACATTAAGCTGGAAGAG | |
| | | GAAAGAAGGAGCGAGGACGCCCAATCTAAAGCCGCTCTGACCGA | |
| | | TTGGCTGAGAGCCAAGGCCAGCTTTGTGATCGAGGGCCTGAAAG | |
| | | AGGCCGACAAGGACGAGTTCTGCAGATGCGAGCTGAAGCTGCAG | |
| | | AAGTGGTACGGCGATCTGAGAGGCAAGCCCTTCGCCATTGAGGC | |
| | | CGAGAACAGCATCCTGGACATCAGCGGCTTCAGCAAGCAGTACA | |
| | | ACTGCGCCTTCATTTGGCAGAAAGACGGCGTCAAGAAACTGAAC | |
| | | CTGTACCTGATCATCAATTACTTCAAAGGCGGCAAGCTGCGGTT | |
| | | CAAGAAGATCAAACCCGAGGCCTTCGAGGCTAACAGATTCTACA | |
| | | CCGTGATCAACAAAAAGTCCGGCGAGATCGTGCCCATGGAAGTG | |
| | | AACTTCAACTTCGACGACCCCAACCTGATTATCCTGCCTCTGGC | |
| | | CTTCGGCAAGAGACAGGGCAGAGAGTTCATCTGGAACGATCTGC | |
| | | TGAGCCTGGAAACCGGCTCTCTGAAGCTGGCCAATGGCAGAGTG | |
| | | ATCGAGAAAACCCTGTACAACAGGAGAACCAGACAGGACGAGCC | |
| | | TGCTCTGTTTGTGGCCCTGACCTTCGAGAGAAGAGAGGTGCTGG | |
| | | ACAGCAGCAACATCAAGCCCATGAACCTGATCGGCGTGGACCGG | |
| | | GGCGAGAATATCCCTGCTGTGATCGCCCTGACAGACCCTGAAGG | |
| | | ATGCCCACTGAGCAGATTCAAGGACTCCCTGGGCAACCCTACAC | |
| | | ACATCCTGAGAATCGGCGAGAGCTACAAAGAGAAGCAGAGGACA | |
| | | ATCCAGGCCAAGAAAGAGGTGGAACAGCGCAGAGCCGGCGGATA | |
| | | CTCTAGGAAGTACGCCAGCAAGGCCAAGAATCTGGCCGACGACA | |
| | | TGGTCCGAAACACCGCCAGAGATCTGCTGTACTACGCCGTGACA | |
| | | CAGGACGCCATGCTGATCTTCGAGAATCTGAGCAGAGGCTTCGG | |
| | | CCGGCAGGGCAAGAGAACCTTTATGGCCGAGAGGCAGTACACCA | |
| | | GAATGGAAGATTGGCTCACAGCTAAACTGGCCTACGAGGGACTG | |
| | | AGCAAGACCTACCTGTCCAAAACACTGGCCCAGTATACCTCCAA | |
| | | GACCTGCAGCAATTGCGGCTTCACCATCACCAGCGCCGACTACG | |
| | | ACAGAGTGCTGGAAAAGCTCAAGAAAACCGCCACCGGCTGGATG | |
| | | ACCACCATCAACGGCAAAGAGCTGAAGGTTGAGGGCCAGATCAC | |
| | | CTACTACAACAGGTACAAGAGGCAGAACGTCGTGAAGGATCTGA | |
| | | GCGTGGAACTGGACAGACTGAGCGAAGAGAGCGTGAACAACGAC | |
| | | ATCAGCAGCTGGACAAAGGGCAGATCAGGCGAGGCTCTGAGCCT | |
| | | GCTGAAGAAGAGGTTTAGCCACAGACCTGTGCAAGAGAAGTTCG | |
| | | TGTGCCTGAACTGCGGCTTCGAGACACACGCCGATGAACAGGCT | |
| | | GCCCTGAACATTGCCAGAAGCTGGCTGTTCCTGAGAAGCCAAGA | |
| | | GTACAAGAAGTACCAGACCAACAAGACCACCGGCAACACCGACA | |
| | | AGAGGGCCTTTGTGGAAACCTGGCAGAGCTTCTACAGAAAAAAG | |
| | | CTGAAAGAAGTCTGGAAGCCCGCCGTG | |
| | linker | GGATCC | 44025 |
| | SV40 NLS | CCAAAAAAGAAGAGAAAGGTA | 44026 |
| | HA tag | TACCCATATGATGTCCCTGACTACGCT | 44027 |
| | linker + stop | GGATCCTAA | 44028 |
| | buffer sequence | GAATTCCTAGAGCTCGCTGATCAGCCTCGA | 44029 |
| | bGH poly(A) | CTGTGCCTTCTAGTTGCCAGCCATCTGTTGTTTGCCCCTCCCCC | 44030 |
| | | GTGCCTTCCTTGACCCTGGAAGGTGCCACTCCCACTGTCCTTTC | |
| | | CTAATAAAATGAGGAAATTGCATCGCATTGTCTGAGTAGGTGTC | |
| | | ATTCTATTCTGGGGGGTGGGGTGGGGCAGGACAGCAAGGGGGAG | |
| | | GATTGGGAAGAGAATAGCAGGCATGCTGGGGA | |
| | buffer sequence | GGTACCGT | 44031 |
| | U6 promoter | GAGGGCCTATTTCCCATGATTCCTTCATATTTGCATATACGATA | 44032 |
| | | CAAGGCTGTTAGAGAGATAATTGGAATTAATTTGACTGTAAACA | |
| | | CAAAGATATTAGTACAAAATACGTGACGTAGAAAGTAATAATTT | |
| | | CTTGGGTAGTTTGCAGTTTTAAAATTATGTTTTAAAATGGACTA | |
| | | TCATATGCTTACCGTAACTTGAAAGTATTTCGATTTCTTGGCTT | |
| | | TATATATCTTGTGGAAAGGAC | |
| | buffer sequence | GAAACACC | 44033 |
| | Scaffold 174 | ACTGGCGCTTTTATCTGATTACTTTGAGAGCCATCACCAGCGAC | 43983 |
| | | TATGTCGTAGTGGGTAAAGCTCCCTCTTCGGAGGGAGCATCAAA | |
| | | G | |
| | Spacer 1 | See specific dual guide combos below | |
| | buffer sequence | TTTTTTTTGGCTAGC | 44034 |
| | U6 promoter | GAGGGCCTATTTCCCATGATTCCTTCATATTTGCATATACGATA | 44032 |
| | | CAAGGCTGTTAGAGAGATAATTGGAATTAATTTGACTGTAAACA | |
| | | CAAAGATATTAGTACAAAATACGTGACGTAGAAAGTAATAATTT | |
| | | CTTGGGTAGTTTGCAGTTTTAAAATTATGTTTTAAAATGGACTA | |
| | | TCATATGCTTACCGTAACTTGAAAGTATTTCGATTTCTTGGCTT | |
| | | TATATATCTTGTGGAAAGGAC | |
| | buffer sequence | GAAACACC | 44033 |
| | Scaffold 174 | ACTGGCGCTTTTATCTGATTACTTTGAGAGCCATCACCAGCGAC | 43983 |
| | | TATGTCGTAGTGGGTAAAGCTCCCTCTTCGGAGGGAGCATCAAA | |
| | | G | |
| | Spacer 2 | See specific dual guide combos below | |
| | buffer sequence | TTTTTTTTGGCGGCCGC | 44035 |
| | 3′ ITR | AGGAACCCCTAGTGATGGAGTTGGCCACTCCCTCTCTGCGCGCT | 44036 |
| | | CGCTCGCTCACTGAGGCCGGGCGACCAAAGGTCGCCCGACGCCC | |
| | | GGGCTTTGCCCGGGCGGCCTCAGTGAGCGAGCGAGCGCGCAGCT | |
| | | GCCTGCAGG | |
| 1 | Spacer 1 (28.10) | CAGCGGGGATCCGACGAGCT | |
| | Spacer 2 (12.7) | CTGCATTCTAGTTGTGGTTT | 43978 |
| 2 | Spacer 1 (NT) | GGGTCTTCGAGAAGACCC | 43982 |
| | Spacer 2 (12.7) | CTGCATTCTAGTTGTGGTTT | 43978 |

### AAV Production:

Suspension-adapted HTEK293T cells were seeded in 20-30 mL of media at 1.5E6 cells/mL on the day of transfection. Endotoxin-free pAAV plasmids with the transgene flanked by ITR repeats were co-transfected with plasmids supplying the adenoviral helper genes for replication and AAV rep/cap genome using PEI Max (Polysciences) in serum-free Opti-MVEM media. Cultures were supplemented with 1000 CDM4HEK293 (HyClone) three hours post-transfection. Three days later, cultures were centrifuged to separate the supermatant from the cell pellet. The supernatant was mixed with 400% PEG 2.5M NaCl and incubated on ice to precipitate AAV viral particles. The cell pellet, containing majority of the AAV vectors, was resuspended in lysis media (0.15 M NaCl, 50 mM Tris HCl, 0.05% Tween, pH 8.5), sonicated on ice, and treated with Benzonase (250 U/μL, Novagen) for 30 minutes at 37° C. Crude lysate and PEG-treated supernatant were then centrifuged to resuspend the PEG-precipitated AAV with cell debris-free crude lysate, and then clarified further using a 0.45 μm filter.

To determine the viral genome (vg) titer, 1 μL from crude lysate virus was digested with DNase and ProtK, followed by quantitative PCR. 5 μL of digested virus was used in a 25 μL qPCR reaction composed of IDT primetime master mix and a set of primer and 6′FAM/Zen/IBFQ probe (IDT) amplifying the poly(A) signal region (Fwd 5′-GGCACCTTCCAGGGTCAAG-3′ (SEQ ID NO: 44040); Rev 5′-GCCTTCTAGTTGCCAGCCAT-3′ (SEQ ID NO: 44041), Probe 5′-TCCCCCGTGCCTTCCTTGACC-3′) (SEQ ID NO: 44042). Ten-fold serial dilutions of an AAV ITR plasmid were used as reference standards to calculate the titer (vg/mL) of viral samples. The qPCR program was set up as: initial denaturation step at 95° C. for 5 minutes, followed by 40 cycles of denaturation at 95° C. for 1 minute and annealing/extension at 60° C. for 1 minute.

### AAV Transduction of Astrocytes In Vitro:

tdTomato mouse midbrain astrocytes, derived from the Ai9 tdTomato reporter mouse, were grown in BrainBits Nb Astro media supplemented with Pen/Strep using 10 cm plates coated with PLF. Briefly, Ai9 is a Cre reporter strain designed to have a loxP-flanked STOP cassette preventing the transcription of a CAG promoter-driven tdTomato marker. Successful excision of the STOP cassette would drive expression of the tdTomato fluorescent reporter in edited cells. Once the midbrain astrocytes reached 90% confluency, they were trypsinized and seeded at a density of 20,000 cells/well on PLF-coated 96-well plates 24 hours before AAV transduction. The next day, seeded cells were treated with AAVs expressing CasX variant 491 (XAAVs) with the dual-guide system (i.e., spacers 28.10-12.7 or NT-12.7; refer to Table 17 for sequences). Viral infection conditions were performed in triplicate, with normalized number of viral genomes (vg) among experimental vectors, in a series of three-fold serial dilution of MOI ranging from 5E5 to 6E3 to vg/cell. Cells were fed with a 50% media exchange three days post-transduction. Four days post-transduction, cells were trypsinized for editing activity assessments: tdTomato+ cell quantification by flow cytometry and editing analysis at the PTBP1 locus by NGS. For editing analysis by NGS, amplicons were amplified from 200 ng of extracted gDNA with a set of primers targeting the mouse PTBP1 locus of interest and processed as described earlier in Example 14.

### Results:

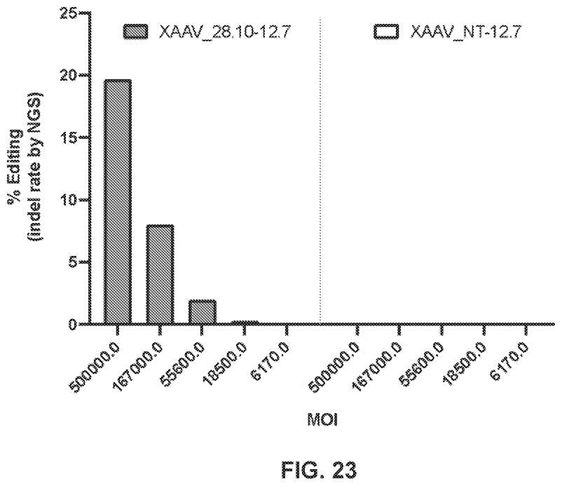

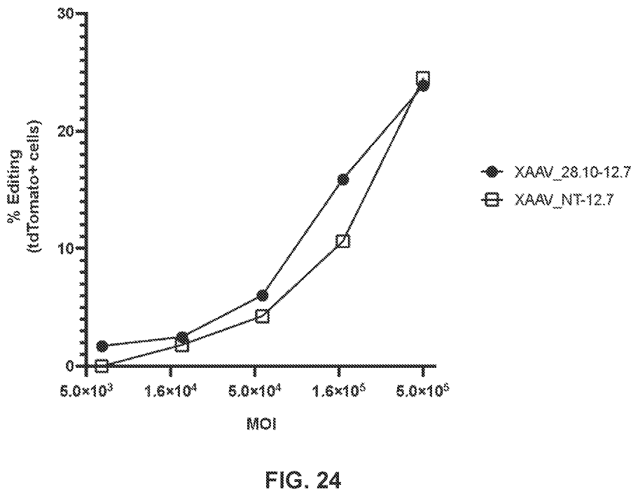

FIG. 23 shows the quantification of editing measured as indel rate detected by NGS at the mouse PTBP1 locus in primary astrocytes infected with dual-guide XAAVs harboring either the spacer combination of 28.10-12.7 (targeting PTBP1 and tdTomato) or NT-12.7 (non-targeting and tdTomato) at various MOIs. XAAV containing dual-guide 28.10-12.7 demonstrated on-target editing efficiency at the murine PTBP1 locus in a dose-dependent manner, attaining ˜20% editing at the highest MOI, while no editing was observed at the PTBP1 locus with the NT-12.7 dual-guide XAAV as expected (FIG. 23). FIG. 24 shows percent editing at the tdTomato locus within the same cultured primary astrocytes, which were marked by tdTomato expression, indicating successful editing and excision of the tdTomato STOP cassette by the targeting spacer 12.7. FIG. 24 also shows that both XAAV dual-guide systems were able to edit on-target at the tdTomato locus at comparative levels in a dose-dependent manner.

The results of the experiments demonstrate that CasX variant 491 and guide scaffold 174 with spacer targeting the mouse PTBP1 locus can edit on-target efficiently when packaged and delivered in vitro via XAAVs. Furthermore, the data show the ability to encode, package, and deliver CasX with a dual-guide system within a single AAV vector particle that can edit with on-target efficiency at different locations in the genome, an outcome not possible with Cas9-based systems. These findings justify additional studies to investigate in vivo editing after delivering AAV containing CasX with a PTBP1-targeting spacer directly into the substantia nigra of the mouse midbrain and enable accurate assessment of astrocyte conversion to neurons in vivo, as described later in Example 18.

### Example 16: CasX-Mediated Editing at the Mouse PTBP1 Locus Results in Significant Knockdown of PTBP1 and Subsequent Upregulation of Neuronal PTB In Vitro

PTBP1 has been previously shown to suppress neuronal fate in non-neuronal cells by repressing the activity of pro-neuronal factors such as neuronal PTB (nPTB, also known as PTBP2). During neural development, PTBP1 expression decreases, releasing inhibition of nPTB expression, which is critical for neuronal induction (Hu et al., 2018). Experiments were performed to demonstrate that CasX-mediated editing at the PTBP1 locus in primary mouse astrocytes, when delivered via XDPs in vitro, can induce substantial PTBP1 knockdown and consequent induction of nPTB.

### Materials and Methods:

XDP production and transduction of astrocytes in vitro were performed as described in Example 14. Briefly, for XDP transduction of cultured astrocytes, 120,000 tdTomato mouse midbrain astrocytes were seeded in each well of a 24-well plate coated with PLF. The next day, each well was treated with XDP particles containing RNPs of CasX protein 491 and a guide RNA at an MOI of 3E5. Cells were transduced with XDPs containing a spacer targeting either PTBP1 or tdTomato STOP cassette (the tdTomato spacer served as the non-targeting (NT) control; SEQ ID NO. 43978). Following transduction, cells were harvested at multiple time points and subjected to whole cell lysate extraction for western blotting analysis. Specifically, for one experiment, cells were harvested at 2 and 5 days post-transduction; in the second experiment, cells were harvested at 5, 12, and 21 days post-transduction.

### Results:

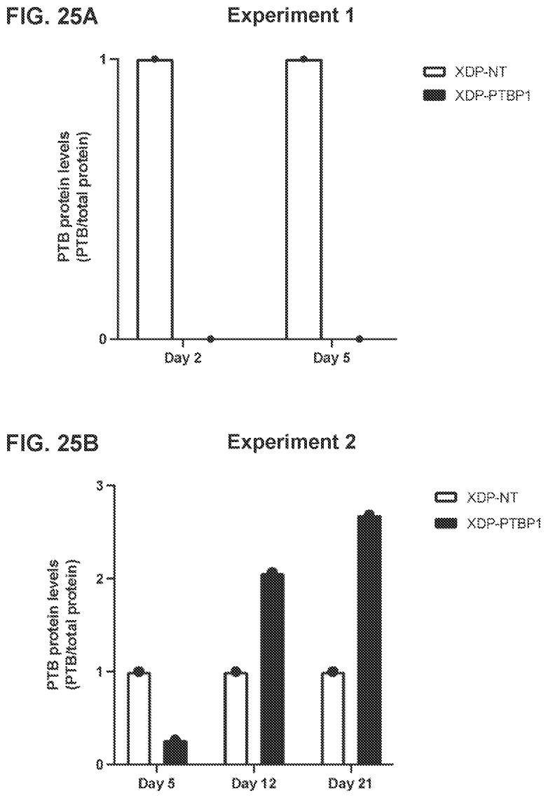

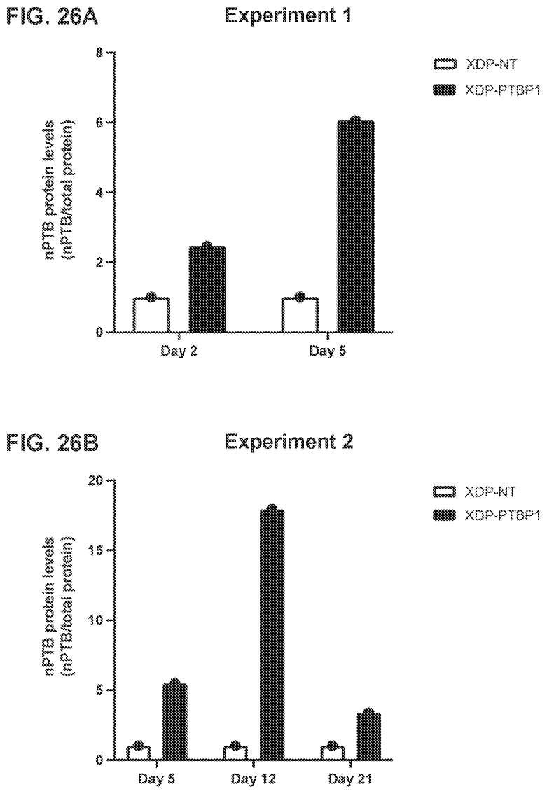

Extracted protein samples were resolved by SDS-PAGE followed by immunoblotting to analyze PTBP1 and nPTB levels in cells transduced with XDPs containing spacer targeting PTBP1 or tdTomato (NT control). PTBP1 and nPTB protein levels at the indicated time points, i.e., 2, 5, 12, and 21 days post-XDP transduction, were quantified by densitometry and illustrated in FIG. 25 and FIG. 26, respectively, as the ratio of detected protein over total protein normalized to NT control. The results from one experiment (depicted in FIG. 25A) demonstrate that XDPs harboring the PTBP1-targeting spacer (XDP-PTBP1) significantly decreased PTBP1 protein to undetectable levels as early as two days post-treatment, compared to PTBP1 levels from NT control treatment (XDP-NT). In a second experiment (results depicted in FIG. 25B), substantial reduction of PTBP1 protein levels was also observed five days post-transduction with XDP-PTBP1 compared to levels seen with XDP-NT. This decrease in PTBP1 protein levels was not observed at Day 12 and 21 in this experiment. In addition, nPTB upregulation was detected by five days post-XDP-PTBP1 treatment (FIGS. 26A & B). This nPTB induction was transient and decreased by Day 21 (FIG. 26B), an indication of a switch to a neuronal phenotype and consistent with reported findings (Qian et al., 2020).

These experiments demonstrate that XDPs containing CasX variant 491 with a gRNA having a PTBP1-targeting spacer was able to edit the mouse PTBP1 locus efficiently in vitro, resulting in a significant knockdown of PTBP1 and subsequent upregulation of nPTB, which would initiate the astrocyte-to-neuron conversion process.

### Example 17: CasX Editing at the Mouse PTBP1 Gene Results in Conversion of Mouse Astrocytes to Neurons in Cell Culture

Reducing the levels of PTBP1 in glial cells has been shown to be sufficient for converting glia to neurons in vitro and in vivo (Qian et al., 2020; Zhou et al., 2020). Experiments were performed to demonstrate induced conversion of mouse astrocytes to neurons in vitro by CasX editing at the mouse PTBP1 gene to down-regulate PTBP1 expression.

### Materials and Methods:

XDP production and transduction of astrocytes in vitro were performed as described in Example 14. Briefly, for XDP transduction of cultured astrocytes, 50,000 tdTomato mouse midbrain astrocytes were seeded in each well of a 24-well plate coated with PLF. The next day, wells were either untreated or treated with XDP particles containing CasX protein 491 and a guide RNA at an MOI of 3E5. Treated cells were transduced with XDPs containing a spacer targeting either PTBP1 (XDP-PTBP1) or tdTomato STOP cassette. The tdTomato spacer served as the non-targeting (NT) control (XDP-NT). Following transduction, cells were harvested at 5, 12, and 21 days post-transduction for gDNA extraction for editing analysis at the PTBP1 locus by NGS. For editing analysis by NGS, amplicons were amplified from 200 ng of extracted gDNA with a set of primers targeting the mouse PTBP1 locus of interest and processed as described earlier in Example 14.

### Immunocytochemistry:

Cultured mouse astrocytes that were untreated or treated with XDPs were also grown to 28 days post-treatment and harvested for immunocytochemistry analysis as previously described (Qian et al. 2020). Briefly, Day 28 cultured cells were fixed with 4% paraformaldehyde. After washing and blocking with 2% BSA/0.1% Triton X-100/2.5% normal goat serum in PBS for 1 hour at room temperature, fixed cells were incubated with the primary antibody mouse anti-MAP2 (Abcam, 1:1000) overnight at 4° C. The next day, cells were washed and then incubated with a secondary antibody conjugated to Alexa Fluor 647 (Thermo Fisher, 1:1000) for 1 hour. Cells were also counterstained with DAPI to label nuclei. Images were captured and quantified at 10× on the Pico fluorescent microscope, with ˜70,000 DAPI+ cells per experimental condition analyzed to determine the percentage of cells expressing the neuronal marker MAP2, which would indicate induced conversion of astrocytes to neurons upon editing at the mouse PTBP1 locus in vitro.

### Results:

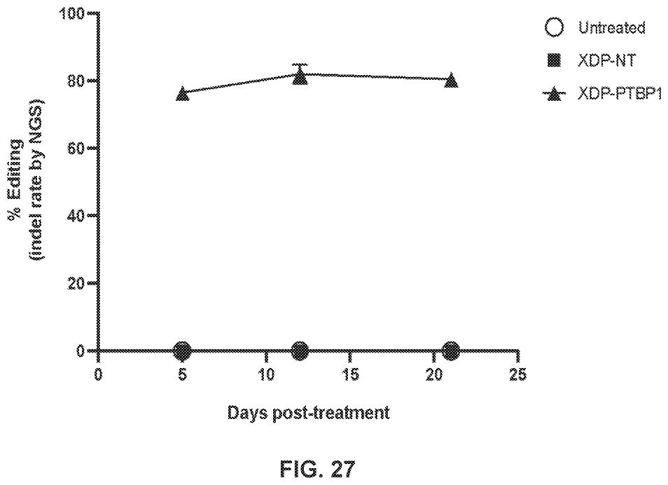

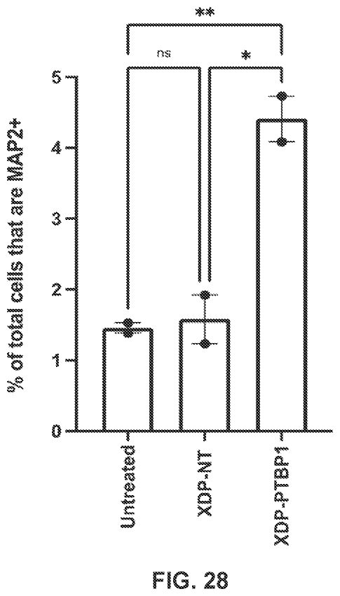

To assess conversion of astrocytes to neurons in vitro, primary mouse astrocytes were treated with XDP-PTBP1 or XDP-NT and subjected to editing assessment by NGS at 5, 12, and 21 days after XDP transduction and immunocytochemistry analysis at 28 days after transduction. FIG. 27 shows that XDP-PTBP1 was able to edit the mouse PTBP1 locus with ˜80% efficiency by Day 5 of XDP treatment, and this editing rate persisted in culture through Day 21. FIG. 28 shows that XDP-PTBP1 treatment resulted in >3-fold increase in the percentage of cells expressing the neuronal marker MAP2, relative to that observed from XDP-NT treatment, indicating astrocyte-to-neuron conversion was occurring by Day 28 post-XDP treatment.

The experiments demonstrate that XDP targeting PTBP1 was able to effectively edit the mouse PTBP1 locus at high efficiency and consequently induce in vitro glia conversion to neurons. These results are suggestive of progressive conversion of astrocytes to new neurons that would innervate into and repopulate endogenous neural circuits, a potential therapeutic strategy for replacing neurons lost as a result of neurodegenerative disease or acute brain injury.

### Example 18: AAV Delivery of CasX into the Substantia Nigra Induces Conversion of Mouse Astrocytes to Neurons In Vivo

Experiments were performed to demonstrate induced conversion of mouse astrocytes to neurons in vivo by CasX-mediated editing of the mouse PTBP1 locus to deplete PTBP1 expression after direct delivery of AAVs into the substantia nigra of the mouse midbrain.

### Materials and Methods:

AAV construct cloning and production were performed as described in Example 15.

In vivo administration of AAV-CasX (XAAVs) and immunohistochemistry:

XAAV particles containing CasX protein 491 downstream of an astrocyte-specific promoter with the dual-guide system (i.e., spacers 28.10-12.7 targeting PTBP1-tdTomato STOP cassette, or NT-12.7 as non-targeting control; refer to Table 17 for sequences) were administered directly into the substantia nigra of Ai9 reporter mice. Briefly, 5E10 XAAV particles were stereotaxically injected unilaterally into the substantia nigra of anesthetized 8-10 week old mice. At 12 weeks post-injection, mice were euthanized by terminal anesthesia followed by transcardiac perfusion. Brains were harvested, post-fixed in 4% paraformaldehyde at 4° C., and then transferred to 30% sucrose solution. Brains were then embedded in OCT compound and frozen on dry ice. OCT-embedded brains were cut into 20 μm thick sections prior to staining for immunohistochemistry. Sections were blocked for 1 hour at room temperature in blocking buffer (5% normal goat serum/2% BSA/0.3% Triton X-100) before antibody labeling. Antibodies used were as follows: rabbit anti-Sox9 (Millipore, 1:1000); rabbit anti-NeuN (Millipore 1:1000); chicken anti-Tyrosine Hydroxylase (Abcam, 1:500); goat anti-chicken Alexa Fluor 488 (Thermo Fisher, 1:1000); and goat anti-rabbit Alexa Fluor 647 (Thermo Fisher, 1:1000). Sections were counterstained with DAPI to label nuclei and imaged on the Echo Revolution fluorescent microscope.

### Results:

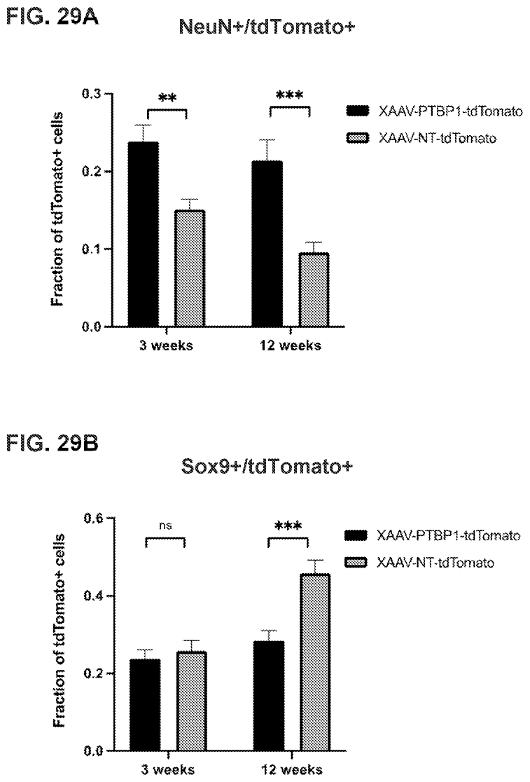

In this experiment, astrocytes were marked to enable tracing and accurate assessment of their conversion into neurons in vivo. Each AAV was packaged with CasX with expression driven by an astrocyte-specific promoter and a dual-gRNA system (i.e., PTBP1-tdTomato gRNAs or non-targeting-tdTomato gRNAs). Specific editing of both PTBP1 and tdTomato loci within astrocytes would induce conversion of edited astrocytes, as marked by tdTomato fluorescence, into neurons. The results depicted in FIG. 29 shows that at three weeks after treatment, there was a 59% increase in the number of tdTomato+ edited cells that co-stained with neuronal marker NeuN in the XAAV-PTBP1 sample relative to the level seen in XAAV-NT (p<0.01), and this percent change increases to 123% at 12 weeks (p<0.001). Contrarily, while no significant difference was observed in the proportion of tdTomato+ cells that co-labeled with astrocyte marker Sox9 at three weeks, the percent decrease was significant at 12 weeks post-treatment (38% reduction, p<0.001).

The results here indicate that in vivo conversion of astrocytes to neurons was successfully initiated in the substantia nigra of the mouse midbrain upon targeting the PTBP1 locus using CasX variant 491 delivered by AAVs. Furthermore, the protocol described herein may also be applicable to converting astrocytes from different regions of the brain (e.g., striatal, cortical) and spinal cord to region-specific neurons. Similar approaches may also be taken in other organ systems, for example, in the eye.

### Example 19: Use of CasX:gNA Systems to Edit the Rat PTBP1 Locus in Rat Astrocytes when Delivered Via XDPs In Vitro

Experiments were performed to demonstrate that CasX and gRNAs can also edit at the rat PTBP1 locus in cultured rat astrocytes when delivered via XDPs.

### Materials and Methods:

XDP construct cloning and production were performed as described in Example 14. Briefly, for XDP construct cloning, XDP plasmid constructs comprising sequences coding for CasX protein 491 with scaffold variant 174 and a spacer targeting PTBP1 (specifically, spacer 28.10, 28.11, 28.15, or 28.16; see Table 17 for sequences) were subjected to E. coli transformation and selection, followed by mini-prepping and Sanger-sequencing. Sequence-validated constructs were then cloned into an expression vector via Golden Gate assembly with the indicated spacers. PTBP1 spacers 28.10, 28.11, 28.15, and 28.16 were selected given their sequence consensus between the mouse and rat genomes. For XDP production, HEK293T Lenti-X cells were transfected with XDP structural plasmids, gRNA plasmid, and plasmid encoding for GP2 using PEI Max (Polypus). XDP-containing media were collected 72 hours post-transfection and filtered. The supernatant was then concentrated and purified via centrifugation, and XDPs were resuspended in a freezing buffer.

### XDP Transduction of Rat Astrocytes In Vitro:

SV40-immortalized rat astrocytes of the D1 TNC1 cell line (ATCC CRL-2005TM) were grown in BrainBits Nb Astro media on T75 flasks coated with poly-L-lysine (PLL). Cells were lifted at 90% confluency with 0.05% Trypsin/0.02% EDTA and seeded on a 24-well tissue culture plate, also coated with PLL, at a density of 100,000 cells per well for the assay. The next day, cells were either untreated (control) or treated with XDP particles containing CasX RNPs with PTBP1-targeting spacers (28.10, 28.11, 28.15, or 28.16), which were applied either at high or low dose (based on total volume of virion). Cells were fed with a 50% media exchange every other day. Two days post-transduction, cells were trypsinized for editing analysis by NGS as described in Example 14. Briefly, amplicons were amplified from 200 ng of extracted gDNA with primers targeting the rat PTBP1 locus of interest and processed as described in Example 14.

### Results:

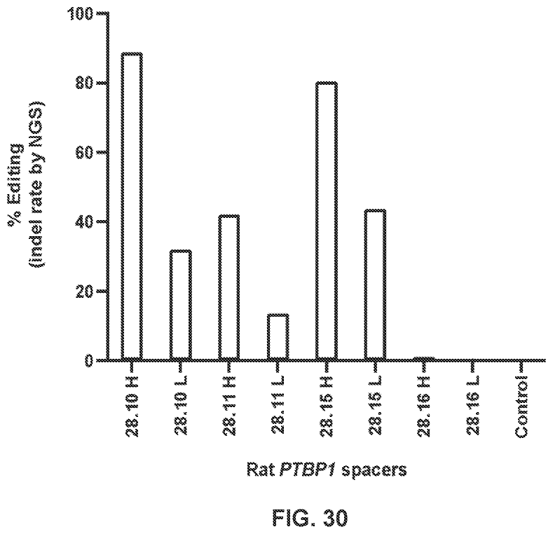

FIG. 30 shows the results of an editing assay assessing spacers 28.10, 28.11, 28.15, and 28.16 in targeting the rat PTBP1 locus. When administered at high dose, spacer 28.10 demonstrated the highest editing rate of ˜90%, followed by spacer 28.15 at ˜80%, spacer 28.11 at ˜42%, and then 28.16 at ˜1.1%. In addition, these spacers were able to edit in a dose-dependent manner. The level of editing achieved by spacer 28.10 in rat astrocytes is similar to the level observed in primary mouse astrocytes, as discussed earlier in Example 14, justifying the use of spacer 28.10 in planned efficacy studies using a rat model of Parkinson's disease.

The experiments demonstrate that CasX variant 491 and guide scaffold 174 with spacers targeting the rat PTBP1 locus can edit on-target efficiently in rat astrocytes when packaged as RNPs and delivered in vitro via XDPs and, therefore, should similarly be able to reprogram conversion of astrocytes into functional neurons.

### Example 20: Assessment of Editing by Engineered Variants of CasX Nucleases at the Mouse and Rat PTBP1 Loci

Here we investigated whether rationally-designed engineered CasX nucleases, with mutations predicted to increase on-target activity and specificity and reduce potentially off-target events, would improve editing at the endogenous mouse and rat PTBP1 loci when delivered via XDPs in vitro.

### Materials and Methods:

XDP construct cloning and production were performed as described in Example 14. Briefly, for XDP construct cloning, XDP plasmid constructs comprising sequences encoding for CasX variants 491, 668, 672, and 676 with scaffold variant 251 and PTBP1-targeting spacer 28.10 (see Table 17 for spacer 28.10 sequence) were subjected to E. coli transformation and selection, followed by mini-prepping and Sanger-sequencing. Sequence-validated constructs were then cloned into an expression vector via Golden Gate assembly with PTBP1 spacer 28.10. For XDP production, HEK293T Lenti-X cells were transfected with XDP structural plasmids, gRNA plasmid, and plasmid encoding for GP2 using PEI Max (Polypus). XDP-containing media were collected 72 hours post-transfection and filtered. The supernatant was then concentrated and purified via centrifugation, and XDPs were resuspended in Nb Astro media. XDP transduction of primary mouse astrocytes and immortalized rat astrocytes in vitro:

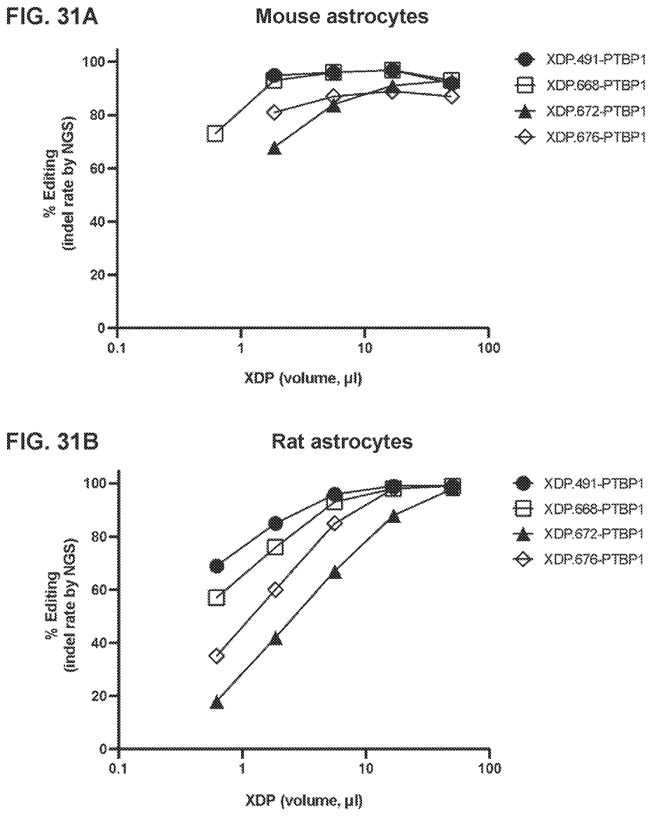

Primary mouse midbrain astrocytes and SV40-immortalized rat astrocytes were separately seeded at 15,000 cells per well in 96-well plates coated with PLF or PLL, respectively. The next day, XDP particles containing RNPs of CasX variants with PTBP1-targeting spacer 28.10 were applied at increasing doses (fixed volumes of XDPs as depicted in FIG. 31). Infection conditions were performed in duplicate. Cells were fed with a 50% media exchange every other day. Five days post-transduction, cells for each sample were harvested for gDNA extraction for editing analysis by NGS as described in Example 14. Briefly, amplicons were amplified from 200 ng of extracted gDNA with primers targeting either the mouse or rat PTBP1 locus of interest and processed as described in Example 14.

### Results:

FIGS. 31A & B illustrate the editing levels of engineered CasX variants 491, 668, 672, and 676 at the mouse (FIG. 31A) or rat (FIG. 31B) PTBP1 locus when delivered in vitro via XDPs at the indicated volumes. All four CasX variants were able to edit at the mouse and rat PTBP1 loci with varying efficiencies. FIG. 31A shows that CasX variants 491 and 668 exhibited similar levels of editing at the PTBP1 locus in primary mouse astrocytes, and both proteins demonstrated improved editing efficiency over CasX variants 672 and 676 at the different volumes of XDP treatment, eventually reaching saturation levels in editing at the highest dose. These editing outcomes were recapitulated in the immortalized rat astrocytes: both CasX 491 and 668 were able to edit at the rat PTBP1 locus with higher efficiency compared to CasX 672 and 676 at the varying MOIs, also reaching saturation at higher doses, although CasX 491 appeared to exhibit slightly better editing over CasX 668 at lower doses (FIG. 31B).

The experiments demonstrate that when packaged as RNPs and delivered in vitro via XDPs, CasX variants 491 and 668 with guide scaffold 251 and PTBP1-targeting spacer 28.10 demonstrated superior editing efficiency over CasX variants 672 and 676 in targeting the mouse and rat PTBP1 loci, although all constructs demonstrated the ability to edit the locus.

### Example 21: Use of CasX:gNA Systems to Edit the Human PTBP1 Locus in Human Fibroblasts when Delivered Via XDPs or Lentiviral Particles In Vitro

Experiments were performed to demonstrate that CasX and guide RNAs can edit at the human PTBP1 locus in cultured human fibroblasts when delivered via XDPs.

### Materials and Methods:

XDP construct cloning and production were performed as described in Example 14. Human PTBP1 spacers were designed based on PAM availability (listed in Table 19). Spacers were made by annealing two oligos and cloned via Golden Gate assembly with the appropriate restriction enzymes. Cloned spacers were subjected to transformation, mini-prepping, and Sanger-sequencing for verification as previously described.

### XDP Transduction of Human Fibroblasts In Vitro:

An initial screen of human PTBP spacers (marked in bold in Table 19) was performed to assess editing levels at the human PTBP1 locus. Human fibroblast cells (CL043 line) were seeded at 75,000 cells per well in a 24-well plate in fibroblast medium (DMEM supplemented with 10% fetal bovine serum, 100 sodium pyruvate, 1% NEAA, 0.2% BME, and Pen/Strep). The following day, XDP particles containing CasX RNPs with the indicated PTBP1-targeting spacers were applied at two fixed volumes, denoted as high (H) and low (L), followed by spinfection with polybrene. Two days post-transduction, cells were harvested for editing analysis by NGS as described in Example 14. Briefly, amplicons were amplified from 200 ng of extracted gDNA with primers targeting the human PTBP1 locus of interest and processed as described in Example 14.

TABLE 19
Spacers for targeting human PTBP1

| | | SEQ | | | SEQ |
|---|---|---|---|---|---|
| Spacer | Sequence | ID NO | Spacer | Sequence | ID NO |
| 30.01 | UGCUAUUCCGGCGCCUCCAC | 37978 | 30.50 | AGAACAUAUUCCCGCCCUCG | 38265 |
| 30.02 | CCCCCUCCCCCGACUACACA | 37972 | 30.51 | CGCCCUCGGCCACGCUGCAC | 38267 |
| 30.03 | UCAAGGCCGAAGGCGGAAAC | 37975 | 30.52 | AGCAAUGGGGGCGUCGUCAA | 38306 |
| 30.04 | GCCUUCGGCCUUGAGGAAUA | 37973 | 30.53 | CCCCGAGGUCGUGGUUGUGC | 38311 |
| 30.05 | GCCUUGAGGAAUAACCGCCU | 37974 | 30.54* | CCAAGUCCACCAUCUAGGGG | 38312 |
| 30.06 | CUGGGUCCCGCCCCCGGGCG | 37976 | 30.55 | CUGGAAUGAUGGAAGUUGUC | 38315 |
| 30.07 | GGCCAGUGGGAGGUGCUGGC | 37977 | 30.56 | GCUGUUUUUAAAGUGGCUUU | 38316 |
| 30.08* | CGCCGCCUGACUCGCCACGU | 37971 | 30.57 | AUCAUUCCAGAGAAAAGCCA | 38313 |
| 30.09* | GGCGCCUCCACUCCGUCCCC | 37979 | 30.58 | AGAGAAAAGCCACUUUAAAA | 38314 |
| 30.10 | CUACUUGUGUCACUAACGGA | 38137 | 30.59* | CUUUAAAAAAAUAAAAUCUC | 38317 |
| 30.11 | GCUGUCACCUUUGAACUUCU | 38153 | 30.60 | CCUUGCUCACCCUGCGGUGA | 38318 |
| 30.12 | AAGGUGACAGCCGAAGUGCA | 38152 | 30.61 | CGGCCCUCCACACCCGGGGC | 38319 |
| 30.13 | GGAUGUGGAUCACUCUAGAG | 38154 | 30.62 | UUGUGCCUUAAAAAACCUGC | 38320 |
| 30.14 | CCCUCCGUGACGUCGAUGGG | 38155 | 30.63 | UGCAGCCACACACCCACCCG | 38321 |
| 30.15 | CAAAGGGCAGCCCCAGGGAG | 38156 | 30.64 | CCCUUCACCCCGCCCCCAGG | 38322 |
| 30.16* | GCAUCAGGAGGUUGGUGACC | 38157 | 30.65 | CCCCGCCCCCAGGGCCUUCC | 38323 |
| 30.17* | CCUUCAGCAUCAGGAGGUUG | 38158 | 30.66 | CUUCUGCCCCCAGGCGGGCU | 38324 |
| 30.18* | CCAUGGUGUUGGCAGCCUCC | 38162 | 30.67 | GCCCCCAGGCGGGCUCCCCG | 38325 |
| 30.19* | UCGAGAUGAACACGGAGGAG | 38160 | 30.68* | AGUUGACCAAAUAUUCUAAU | 38326 |
| 30.20* | GCUCCUUGUGGUUGGAGAAC | 38164 | 30.69 | UUUGCAUAUAAAUGAAAAAA | 38329 |
| 30.21* | CCAACCACAAGGAGCUGAAG | 38163 | 30.70 | AAUCUUUUUUCAUUUAUAUG | 38327 |
| 30.22 | CCGCCUGCAGGGCCGCCUGG | 38165 | 30.71 | UUUAUAUGCAAAAGAAAUAG | 38328 |
| 30.23 | CCGACUGGACCGAGUUCACC | 38166 | 30.72* | UUGUGGUAUUACCUUGUAUG | 38330 |
| 30.24 | CCACGAUGAUCCUGAGCACG | 38167 | 30.73* | UUGUAAUUAAGUCACAGGCA | 38331 |
| 30.25 | ACCCUGUGACCCUGGAUGUG | 38168 | 30.74* | AGAGAGCAGGCGGGGCCGCC | 38332 |
| 30.26 | ACACUGUGCCGAACUUGGAG | 38171 | 30.75 | CUGAAGCUCAGGGGCUCUAA | 38334 |
| 30.27 | CCAAGUUCGGCACAGUGUUG | 38169 | 30.76 | GGGAAGGGGCGGGCGUGUCG | 38333 |
| 30.28* | UGGUGAAGGUGAUGAUCUUC | 38173 | 30.77 | AGGCGACUGCAGGUGAAGGC | 38336 |
| 30.29 | GCACAGUGUUGAAGAUCAUC | 38170 | 30.78 | CCUGCAGUCGCCUAGAAAAC | 38335 |
| 30.30* | CCAAGAACAACCAGUUCCAG | 38172 | 30.79 | GGGUUUUUUCUUCCUUCAAA | 38337 |
| 30.31 | AGGCCCUGCUGCAGUAUGCG | 38174 | 30.80 | UCCUUCAAAUUUUGGACCAA | 38338 |
| 30.32* | AAGCUCACCAGCCUCAACGU | 38176 | 30.81 | UUCAAAUUUUGGACCAAAGU | 38339 |
| 30.33 | GGGGACAGCCAGCCCUCGCU | 38177 | 30.82* | AAUUUUGGACCAAAGUCUCA | 38340 |
| 30.34 | CUCCCACCUUUGCCAUUCCU | 38181 | 30.83 | GGGUCCCAGCAUCAGAGGCA | 38342 |
| 30.35 | GUUCCGAACGUCCACGGCGC | 38195 | 30.84 | GUGUUUUGCCUGCCUCUGAU | 38341 |
| 30.36 | GAACGUCCACGGCGCCCUGG | 38196 | 30.85 | CUGUGCUCUUUCUACCGCCC | 38343 |
| 30.37* | UGCCCCCGCCAGGCCCGGGA | 38198 | 30.86 | ACCGCCCCCGCGUCCUGUCC | 38344 |
| 30.38* | GUAUUGCUGGUCAGCAACCU | 38199 | 30.87 | GUAAAAGCGUGUAACAAGGG | 38345 |
| 30.39 | CGCGCUGCACGUCACCGUAG | 38253 | 30.88 | AGGCUCAGUAUUGUGACCGC | 38346 |
| 30.40* | UAUUGAACAGGAUCUUCACG | 38255 | 30.89 | CUCCGCGUCACAAGCCAUCG | 38348 |
| 30.41* | CCUUCUUAUUGAACAGGAUC | 38256 | 30.90 | GUUGCCUUACCCGAUGGCUU | 38347 |
| 30.42 | AUAAGAAGGAGAACGCCCUA | 38254 | 30.91 | CAAACGGUUUUAAUCGGUUC | 38349 |
| 30.43 | CGUGCAGCUUGUGCCCGUUC | 38259 | 30.92* | CUGUGGACGCUGUAGAGGCA | 38350 |
| 30.44 | AGAGCGUGAUGCGGAUGGGC | 38260 | 30.93 | AAGUCCAGGUACAGACUGGC | 38352 |
| 30.45 | GGUGCUUCGAGAGCGUGAUG | 38261 | 30.94 | AAUAAAUCUUCUGUAUCCUC | 38351 |
| 30.46* | UGAAGCGGUGCAGGGGUGAG | 38263 | 30.95 | GUAUCCUCGCUCCGUUCCGC | 38353 |
| 30.47* | UGGAGCCCGGCUUCUUGAAG | 38264 | 30.97 | CUCUACAGCAUUGUCCCAGA | 38027 |
| 30.48* | GGAAGUUCUUGGAGCCCGGC | 38266 | 30.98 | CUUGCAGCGGGGAUCUGACG | 38136 |
| 30.49* | AGAAGCCGGGCUCCAAGAAC | 38262 | | | |

Bolded spacers in Table 19 were initially selected for editing assessment of human PTBPI spacers.

Spacers marked by * were subsequently tested given their sequence consensus between human and non-human primate genomes.

### Lentiviral Cloning:

Lentiviral plasmid constructs comprising sequences encoding for CasX variant 491, guide scaffold variant 174, and select spacers (marked with * in Table 19) were transformed into chemically-competent E. coli cells, which were plated on kanamycin LB-agar plates following recovery at 37° C. for 1 hour. Single colonies were picked, mini-prepped, and Sanger-sequenced. Sequence-verified constructs were then cloned into a lentiviral expression vector via BbsI Golden Gate assembly with targeting spacers against the human PTBP1 locus (Table 19). Spacers (marked with * in Table 19) were selected based on sequence conservation between human and non-human primate (i.e., consensus spacers). As previously described, spacers were made by annealing two oligos and cloned via Golden Gate assembly with the appropriate restriction enzymes. Cloned spacers were then subjected to transformation, mini-prepping, and Sanger-sequencing.

### Lentivirus Production:

Lentiviral particles packaging CasX variant 491 and gRNA constructs with scaffold 174 and consensus spacers targeting human PTBP1 were produced by transfecting HEK293T Lenti-X cells with plasmids encoding CasX, guide RNA, lentiviral packaging vector, and VSV-G envelope using PEI Max (Polypus). Media were changed 12 hours post-transfection, and viral supernatants were harvested at 72 hours post-transfection and filtered using a 0.45 m PES filter. Lentiviral transduction:

To assess the editing efficiency of CasX:gRNAs with consensus PTBP1 spacers, human fibroblast cells were seeded at 50,000 cells per well in a 24-well plate in fibroblast medium. The following day, lentiviral particles containing CasX with the indicated consensus PTBP1 spacers were applied at two fixed volumes, denoted as high and low dose, followed by spinfection with polybrene. Viral infection conditions were performed in duplicate. Five days post-transduction, cells were harvested to determine PTBP1 expression knockdown by quantitative PCR (qPCR).

Quantification of PTBP1 mRNA by qPCR:

200 ng of RNA was extracted from lentiviral-transduced human fibroblasts using the Zymo Quick RNA 96 kit and used as input for reverse transcription. The resulting cDNA served as input for qPCR reactions to measure PTBP1 expression using SYBR Green-based detection. Expression of the GAPDH housekeeping gene was used for normalization. Expression data were analyzed according to the double delta Ct method and normalized relative to the non-targeting spacer (NT; AGGGGUCUUCGAGAAGACCC (SEQ ID NO: 44043)).

### Results:

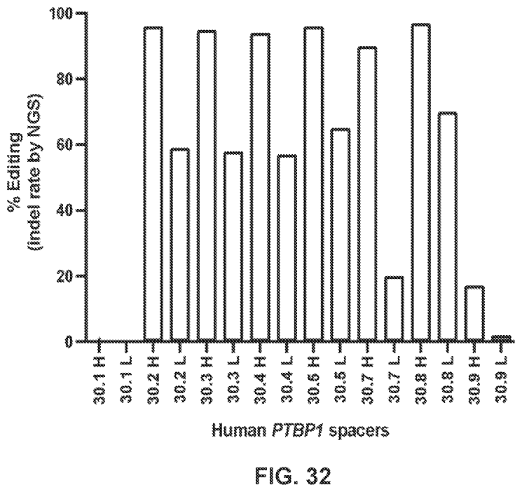

FIG. 32 shows the results of an editing assay assessing spacers 30.1, 30.2, 30.3, 30.4, 30.5, 30.7, 30.8, and 30.9 in targeting the human PTBP1 locus. Of the 8 human PTBP1 spacers tested in this initial screen, 7 spacers demonstrated significant dose-dependent editing at the human PTBP1 gene, 6 of which exhibited >90% editing efficiency when administered at high dose (FIG. 32). Of the 7 spacers that demonstrated editing activity in the preliminary screen, spacer 30.9 has 100% sequence identity to the non-human primate species. Subsequently, human PTBP1 spacers that showed consensus with the non-human primate species (marked with * in Table 19) were selectively tested to determine the effects of editing at the PTBP1 locus in reducing PTBP1 transcript expression. Table 20 shows PTBP1 expression at high or low dose for each consensus PTBP1 spacer relative to the expression from the non-targeting (NT) spacer and the relative rankings for each spacer based on the resulting PTBP1 knockdown. As exhibited in Table 20, all consensus spacers were able to edit at the human PTBP1 locus when administered at high dose, and nearly ⅔ of the spacers were able to edit when administered at low dose. The top performing spacer in the high dose cohort was spacer 30.17; for the low dose cohort, it was spacer 30.19. In addition, these spacers also demonstrated editing activity at the PTBP1 locus in a dose-dependent manner (Table 20).

TABLE 20
PTBP1 gene expression knockdown upon CasX targeting in human
fibroblasts.
Bolded spacers were selected for follow-up characterization

| | Relative | Relative | | |
|---|---|---|---|---|
| | Expression | Expression | | |
| | with High | with Low | High | Low |
| Spacer | Viral Dose | Viral Dose | Dose Rank | Dose Rank |
| 30.08 | 0.665 | 1.034 | 23 | 22 |
| 30.09 | 0.707 | 0.922 | 25 | 13 |
| 30.16 | 0.394 | 1.022 | 5 | 20 |
| 30.17 | 0.335 | 0.933 | 1 | 15 |
| 30.18 | 0.466 | 0.807 | 12 | 4 |
| 30.19 | 0.366 | 0.683 | 3 | 1 |
| 30.20 | 0.425 | 0.7 | 8 | 2 |
| 30.21 | 0.519 | 0.86 | 19 | 7 |
| 30.28 | 0.374 | 0.83 | 4 | 5 |
| 30.30 | 0.714 | 0.891 | 27 | 8 |
| 30.32 | 0.554 | 0.908 | 20 | 10 |
| 30.37 | 0.349 | 0.849 | 2 | 6 |
| 30.38 | 0.619 | 1.096 | 21 | 26 |
| 30.40 | 0.411 | 0.98 | 7 | 18 |
| 30.41 | 0.66 | 1.056 | 22 | 24 |
| 30.46 | 0.508 | 1.046 | 17 | 23 |
| 30.47 | 0.509 | 0.965 | 18 | 17 |
| 30.48 | 0.504 | 0.897 | 15 | 9 |
| 30.49 | 0.678 | 0.933 | 24 | 14 |
| 30.54 | 0.461 | 0.909 | 11 | 11 |
| 30.59 | 0.475 | 0.794 | 13 | 3 |
| 30.68 | 0.436 | 1.087 | 10 | 25 |
| 30.72 | 0.505 | 0.921 | 16 | 12 |
| 30.73 | 0.436 | 1.029 | 9 | 21 |
| 30.74 | 0.403 | 0.952 | 6 | 16 |
| 30.82 | 0.478 | 1.104 | 14 | 28 |
| 30.92 | 0.712 | 1.097 | 26 | 27 |
| NT | 1 | 1 | 28 | 19 |

The experiments demonstrate that CasX variant 491 and guide scaffold 174 with human PTBP1 spacers can edit on-target efficiently in human fibroblasts when delivered in vitro via XDPs. In addition, lentiviral delivery of human PTBP1 spacers having consensus sequence with the non-human primate species can effectively downregulate PTBP1 expression. These findings also support the use of select spacers in preclinical efficacy studies using non-human primate models of Parkinson's disease.

### Example 22: CasX-Mediated Editing at the Human PTBP1 Locus Reduces PTBP1 RNA Expression and Upregulates nPTB Expression In Vitro

Experiments were performed to demonstrate CasX-mediated editing at the PTBP1 locus in human fibroblasts, when delivered via lentiviral particles in vitro, that CasX can induce effective PTBP1 transcript knockdown and consequent upregulation of nPTB gene expression.

### Materials and Methods:

Lentiviral production and transduction of human fibroblasts in vitro were performed as described in Example 21. Briefly, for lentiviral transduction, 100,000 human fibroblasts were seeded in each well of a 12-well plate. The next day, each well was infected with filtered lentiviral particles containing CasX with PTBP1-targeting spacer 30.17 or 30.19 or a non-targeting spacer. Spacers 30.17 and 30.19 were selected for this experiment because they demonstrated the highest editing activity described in Example 21. Viral infection conditions were performed with a normalized number of viral genomes among experimental vectors, in a series of 3-fold serial dilution of MOI ranging from 0.02 to 0.6 vg/cell. Five days post-transduction, cells were harvested for editing activity assessments: editing analysis at the PTBP1 locus by NGS and quantification of PTBP1 and nPTB RNA expression by qPCR. DNA and RNA were extracted from harvested cells using the Zymo Quick-DNA/RNA Miniprep kit. For editing analysis by NGS, target amplicons were amplified from 200 ng of extracted gDNA and processed as described in Example 14. Quantification of PTBP1 and nPTB mRNA by qPCR was also performed as described in Example 21.

### Results:

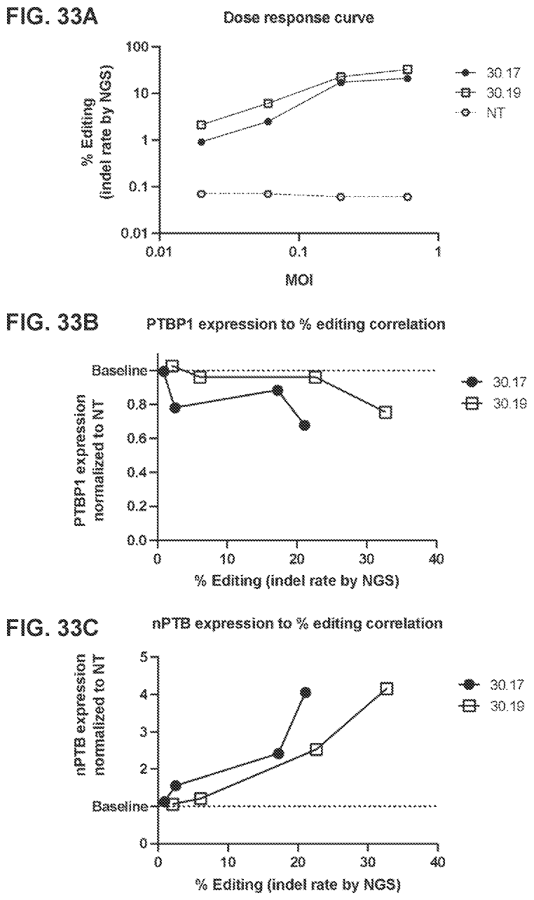

FIG. 33A shows the results of an editing assay assessing spacers 30.17 and 30.19 in targeting the human PTBP1 locus. When administered at the highest MOI of 0.6, spacers 30.17 and 30.19 demonstrated an editing rate of ˜21% and ˜33% respectively. Both spacers were also able to edit in a dose-dependent manner. FIG. 33B illustrates an overall inverse relationship between editing events at the PTBP1 locus and PTBP1 transcript expression in cultured human fibroblasts for PTBP1 spacers 30.17 and 30.19. Each data point on the line graph corresponds to an MOI, with increasing MOI from left to right (see FIG. 33A). At the highest dose, editing at the PTBP1 locus by either spacer resulted in decreased PTBP1 RNA expression by ˜30% (FIG. 33B). These editing effects also corresponded with nearly a four-fold induction of nPTB expression at the highest dose (FIG. 33C). Between the two characterized spacers, spacer 30.17 demonstrated higher potency upon editing the human PTBP1 locus.

The experiments demonstrate that lentiviral particles targeting PTBP1 were able to effectively edit the human PTBP1 locus efficiently in vitro and consequently downregulate PTBP1 expression to induce nPTB expression, which would be able to initiate astrocyte-to-neuron conversion.

### Example 23: Proof-of-Concept Demonstrating In Vivo Editing in the Substantia Nigra of the Mouse Midbrain Using XDPs

Experiments were performed to demonstrate an in vivo proof-of-concept that engineered XDPs could specifically target astrocytes for editing in the substantia nigra, a region in the midbrain prone to neuronal degeneration in Parkinson's disease.

### Materials and Methods:

### In Vivo Administration of XDPs and 2D Immunohistochemistry:

XDP particles containing CasX protein 491 with gRNA targeting the tdTomato STOP cassette were administered into the substantia nigra of Ai9 mice. Briefly, 3.15-3.5E8 XDP particles were stereotaxically injected unilaterally into the substantia nigra of anesthetized 8-10 week-old mice. At 3 weeks post-injection, mice were euthanized by terminal anesthesia followed by transcardiac perfusion. Brains were harvested, post-fixed in 4% paraformaldehyde at 4° C., and then transferred to 30% sucrose solution. Brains were then embedded in OCT compound and frozen on dry ice. OCT-embedded brains were cut sagittally into 20 μm thick sections using a cryostat prior to staining for immunohistochemistry. Sections were blocked for 1 hour at room temperature in blocking buffer (5% NGS, 2% BSA, 0.3% Triton X-100 in PBS) before antibody labeling. Antibodies used were as follows: rabbit anti-Sox9 (Millipore, 1:1000); rabbit anti-NeuN (Thermo Fisher, 1:1000); chicken anti-Tyrosine Hydroxylase (Abcam, 1:500); goat anti-chicken Alexa Fluor 488 (Thermo Fisher, 1:1000); and goat anti-rabbit Alexa Fluor 647 (Thermo Fisher, 1:1000). Sections were counterstained with DAPI to label nuclei and imaged on the Echo Revolve fluorescent microscope. Images were analyzed using a custom computational pipeline.

### Results:

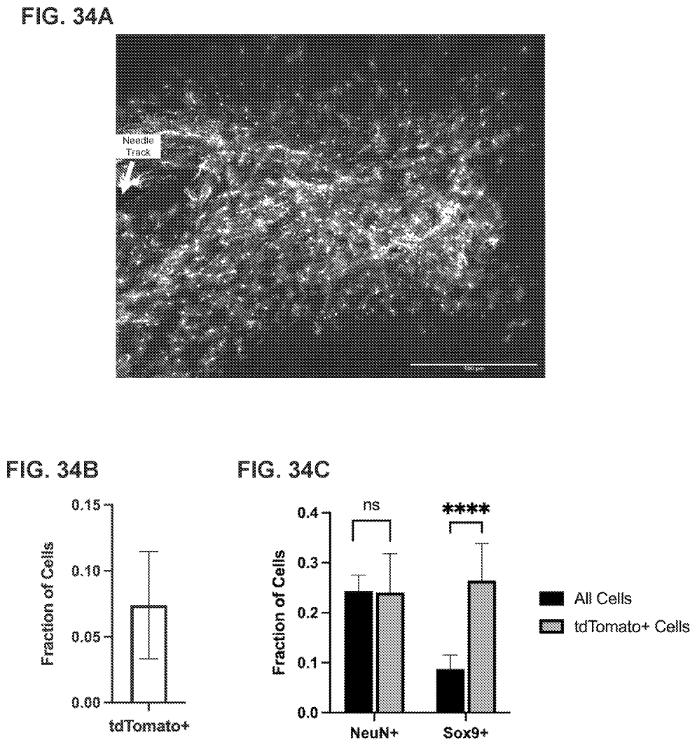

In this experiment, the Ai9 tdTomato reporter mouse model was used to demonstrate a proof-of-concept for CasX delivery using engineered XDPs into the substantia nigra. Ai9 is a Cre reporter strain designed to have a loxP-flanked STOP cassette preventing the transcription of a CAG promoter-driven tdTomato marker. Successful XDP delivery and editing would result in excision of the STOP cassette to drive expression of the tdTomato fluorescent reporter in edited cells. As illustrated in FIG. 34A, efficient editing, marked by tdTomato fluorescence, was observed at three weeks post-XDP injection into the substantia nigra of the mouse midbrain. The robust tdTomato expression observed in the substantia nigra also highlights that the XDP delivery modality was able to achieve sufficient biodistribution in the mouse midbrain. FIGS. 34B and C show that of the tdTomato+ edited cells that were quantified (˜7% of total cells) within the substantia nigra, ˜27% co-labeled with astrocyte marker Sox9, while ˜24% co-stained with neuronal marker NeuN. When normalized to the total number of Sox9+ astrocytes and NeuN+ neurons in the imaged region, XDPs were able to edit astrocytes at ˜2.5-fold greater rate compared to editing in neurons (FIG. 34C), illustrating the ability to engineer XDPs that displayed a biased tropism for astrocytes.

The results demonstrate that effective in vivo editing could be achieved in the substantia nigra using engineered XDPs. In addition, the XDPs were able to exhibit tropism for astrocytes, highlighting the ability to design and engineer XDPs that could be targeted to specific cell types when necessary. These experiments also justify the use of XDP in studies to examine the efficiency of editing and astrocyte conversion to neurons in the substantia nigra via delivery of XDPs to target the mouse PTBP1 locus in vivo, which are described in Example 24.

### Example 24: XDP Delivery of CasX into the Substantia Nigra Results in Conversion of Mouse Astrocytes to Neurons In Vivo

Experiments will be performed to demonstrate induced conversion of mouse astrocytes to neurons in vivo by CasX-mediated editing of the mouse PTBP1 locus to deplete PTBP1 expression after direct delivery of XDPs into the substantia nigra of the mouse midbrain.

### Materials and Methods:

In vivo administration of XDPs and 2D immunohistochemistry analysis will be performed as described in Example 23. Briefly, 3.15-3.5E8 XDP particles containing CasX protein 491 with gRNAs targeting PTBP1 and the tdTomato STOP cassette will be stereotaxically injected into the substantia nigra of anesthetized 8-10 week old Ai9 mice. At 3 and 12 weeks post-injection, mice will be euthanized by terminal anesthesia followed by transcardiac perfusion, and brains will be harvested, fixed, sectioned, and subjected to immunohistochemistry as described in Example 23. Sections will be counterstained with DAPI to label nuclei and imaged on the Echo Revolve fluorescent microscope, and images will be analyzed using a custom computational pipeline. Cell-types will be classified in an unbiased manner using a trained machine learning model. 3D whole mount tissue clearing and analysis:

XDP particles containing CasX protein 491 with gRNAs targeting PTBP1 and tdTomato STOP cassette will be administered as described previously with 2D immunohistochemical analysis. At 12 weeks post-injection, whole brains will be chemically cleared according to the iDISCO+ protocol (Renier et al., 2016). The same antibodies against NeuN, Sox9, and Tyrosine Hydroxlyase will be used to mark specific cell types as described in the 2D immunohistochemistry experiments. Cleared whole brains will be imaged using a light-sheet microscope (Ultramicroscope II; Miltenyi Biotech) at sufficient resolution to resolve cellular markers and delineate major neuronal processes. Cell counts will be quantified using the NuMorph software package (Krupa et al., 2021) to determine viral tropism and total cell count differences upon PTBP1 knockdown. Differences in axonal projections will also be measured using the TrailMap software package (Friedmann et al., 2020) to map the projections of newly formed neurons within specific brain structures.

### Results:

The 2D immunohistochemical results are expected to show that CasX-mediated editing of the mouse PTBP1 gene will initiate conversion of mouse astrocytes to neurons in vivo. The tdTomato reporter system will enable tracing for accurate assessment of this astrocyte-to-neuron conversion, such that edited astrocytes will be expected to be marked by tdTomato fluorescence. In addition to 2D immunohistochemical analysis, 3D whole mount tissue clearing and analysis will be performed to determine differences in cell count and axonal projections upon PTBP1 knockdown. Tracing of neuronal processes within 3D whole brain images will further validate the correct axonal localization and downstream targets of newly converted neurons. Furthermore, stereotaxic injections may be similarly performed in other regions of the brain (e.g., striatum, cortex, hippocampus) to induce astrocyte conversion into region-specific neurons, and 2D immunohistochemistry and 3D whole mount tissue clearing and analysis will be conducted to analyze region-specific conversion of astrocytes to neurons.

### Example 25: CasX Mediated Editing of Mouse PTBP1 Gene in the CNS in a Parkinson's Disease Mouse Model Results in Alleviation of Disease Symptoms

Experiments will be conducted to demonstrate conversion of mouse midbrain astrocytes to functional neurons by depleting the RNA-binding protein PTBP1 in the mouse brain in a model of Parkinson's disease, demonstrating progressive conversion of astrocytes to new neurons that innervate into and repopulate endogenous neural circuits, and consequent alleviation of disease symptoms.

### Materials and Methods:

In this experiment, CasX 491 and guide 174 with spacers targeting the mouse PTBP1 gene, including the spacers of Table 17, will be administered into the 6-OHDA lesioned Parkinson's disease model mice. Adult WT mice at P30-P40 will be used to generate a well-established chemically-induced Parkinson's disease model. Animals will be anaesthetized and then placed in a stereotaxic mouse frame. Before injecting 6-hydroxydopamine (6-OHDA, Sigma), mice will be treated with a mix of desipramine (25 mg/kg) and pargyline (5 mg/kg). 6-OHDA (3.6 μg per mouse) will be dissolved in 0.02% ice-cold ascorbate/saline solution at a concentration of 15 mg/ml and the solution will be injected into the medial forebrain bundle using a 5 μl Hamilton syringe with a 33G needle at the speed of 0.1 μl/min. The needle will be slowly removed 3 min after injection. CasX and guide with PTBP1 or control spacers delivered by AAV or XDP will be stereotaxically injected into substantia nigra ˜30 days after 6-OHDA induced lesion.

Parkinson's disease phenotype will be assessed by measuring apomorphine (or amphetamine) induced rotations. All behavioral tests will be carried out at pre-determined time points, for example 21-28 days after 6-OHDA induced lesion or 2, 3 and 5 months after the delivery of AAVs or XDPs. For the rotation test, apomorphine-induced rotations in mice will be recorded after intraperitoneal injection of apomorphine (Sigma, 0.5 mg kg−1). Mice will be injected with apomorphine (0.5 mg kg) on two separate days before performing the rotation test to prevent a ‘wind-up’ effect that could obscure the final results. Rotation of full-body turns will be measured 5 min following the injection for 10-60 min. The treated group is expected to show significantly lower level of net rotation compared to control group.

At the end of the experimental period, brain tissue will be harvested from experimental animals for analysis by immunohistochemistry. Antibodies against pan-neuronal markers MAP2, Tuj 1 and midbrain dopaminergic markers TH will be used, with nuclei counterstaining by DAPI.

The results of this experiment are expected to show that CasX mediated editing of the PTBP1 gene in midbrain astrocytes of a Parkinson's disease mouse model alleviates motor symptoms of Parkinson's disease. The lesioned striatum in animals receiving the experimental treatment (PTBP1 targeting guide) is expected to show higher levels of TH labeling, compared to animals receiving the control treatment (non-targeting guide).

### REFERENCES

Friedmann, D., Pun, A., Adams, E. L., Lui, J. H., Kebschull, J. M., Grutzner, S. M., Castagnola, C., Tessier-Lavigne, M. and Luo, L., 2020. Mapping mesoscale axonal projections in the mouse brain using a 3D convolutional network. Proceedings of the National Academy of Sciences, 117(20), pp. 11068-11075.
Krupa, O., Fragola, G., Hadden-Ford, E., Mory, J. T., Liu, T., Humphrey, Z., Rees, B. W., Krishnamurthy, A., Snider, W. D., Zylka, M. J. and Wu, G., 2021. NuMorph: Tools for cortical cellular phenotyping in tissue-cleared whole-brain images. Cell reports, 37(2), p. 109802.
Qian, H., Kang, X., Hu, J. et al. Reversing a model of Parkinson's disease with in situ converted nigral neurons. Nature 582, 550-556 (2020). https://doi.org/10.1038/s41586-020-2388-4
Renier, N., Adams, E. L., Kirst, C., Wu, Z., Azevedo, R., Kohl, J., Autry, A. E., Kadiri, L., Venkataraju, K. U., Zhou, Y. and Wang, V. X., 2016. Mapping of brain activity by automated volume analysis of immediate early genes. Cell, 165(7), pp. 1789-1802.
Zhou H, Su J, Hu X, Zhou C, Li H, Chen Z, Xiao Q, Wang B, Wu W, Sun Y, Zhou Y, Tang C, Liu F, Wang L, Feng C, Liu M, Li S, Zhang Y, Xu H, Yao H, Shi L, Yang H. Glia-to-Neuron Conversion by CRISPR-CasRx Alleviates Symptoms of Neurological Disease in Mice. Cell. 2020 Apr. 30; 181(3):590-603.e16. doi: 10.1016/j.cell.2020.03.024. Epub 2020 Apr. 8. PMID: 32272060.

What is claimed is:

1. A system comprising a Class 2, Type V CRISPR protein and a first guide ribonucleic acid (gRNA), wherein the gRNA comprises a targeting sequence complementary to a polypyrimidine tract-binding protein 1 (PTBP1) gene target nucleic acid sequence.

2. The system of claim 1, wherein the gRNA comprises a targeting sequence complementary to a target nucleic acid sequence selected from the group consisting of:
a. a PTBP1 intron;
b. a PTBP1 exon;
c. a PTBP1 intron-exon junction;
d. a PTBP1 regulatory element; and
e. an intergenic region.

3. The system of claim 1 or claim 2, wherein the PTBP1 gene comprises a wild-type sequence.

4. The system of any one of claims 1-3, wherein the gRNA is a guide RNA (gRNA).

5. The system of any one of claims 1-3, wherein the gRNA is a chimera comprising DNA and RNA.

6. The system of any one of claims 1-5, wherein the gRNA is a single-molecule gRNA (sgRNA).

7. The system of any one of claims 1-5, wherein the gRNA is a dual-molecule gRNA (dgRNA).

8. The system of any one of claims 1-7, wherein the targeting sequence of the gRNA comprises a sequence selected from the group consisting of the sequences of SEQ ID NOS: 492-2100 and 2286-43569, or a sequence having at least about 65%, at least about 75%, at least about 85%, or at least about 95% identity thereto.

9. The system of any one of claims 1-7, wherein the targeting sequence of the gRNA comprises a sequence selected from the group consisting of the sequences of SEQ ID NOS: 492-2100 and 2286-43569.

10. The system of any one of claims 1-7, wherein the targeting sequence of the gRNA comprises a sequence of SEQ ID NOS: 492-2100 and 2286-43569 with a single nucleotide removed from the 3′ end of the sequence.

11. The system of any one of claims 1-7, wherein the targeting sequence of the gRNA comprises a sequence of SEQ ID NOS: 492-2100 and 2286-43569 with two nucleotides removed from the 3′ end of the sequence.

12. The system of any one of claims 1-7, wherein the targeting sequence of the gRNA comprises a sequence of SEQ ID NOS: 492-2100 and 2286-43569 with three nucleotides removed from the 3′ end of the sequence.

13. The system of any one of claims 1-7, wherein the targeting sequence of the gRNA comprises a sequence of SEQ ID NOS: 492-2100 and 2286-43569 with four nucleotides removed from the 3′ end of the sequence.

14. The system of any one of claims 1-7, wherein the targeting sequence of the gRNA comprises a sequence of SEQ ID NOS: 492-2100 and 2286-43569 with five nucleotides removed from the 3′ end of the sequence.

15. The system of any one of claims 1-7, wherein the targeting sequence of the gRNA comprises a sequence of SEQ ID NOS: 37971-37979, 38027, 38136, 38137, 38152-38158, 38160, 38162-38174, 38176, 38177, 38181, 38195, 38196, 38198, 38199, 38253-38256, 38259-38267, 38306 and 38311-38353.

16. The system of claim 15, wherein the targeting sequence of the gRNA is complementary to a sequence selected from the group consisting of PTBP1 exon 1, PTBP1 exon 2, PTBP1 exon 3, PTBP1 exon 4, PTBP1 exon 5, PTBP1 exon 6, PTBP1 exon 7, PTBP1 exon 8, PTBP1 exon 9, PTBP1 exon 10, PTBP1 exon 11, PTBP1 exon 12, PTBP1 exon 13, PTBP1 exon 14, PTBP1 exon 15, and PTBP1 exon 16.

17. The system of claim 16, wherein the targeting sequence of the gRNA is complementary to a sequence selected from the group consisting of PTBP1 exon 1, PTBP1 exon 2, and PTBP1 exon 3.

18. The system of any one of claims 1-17, further comprising a second gRNA, wherein the second gRNA has a targeting sequence complementary to a different or overlapping portion of the PTBP1 target nucleic acid compared to the targeting sequence of the gRNA of the first gRNA.

19. The system of claim 18, wherein the second gRNA has a targeting sequence complementary to the same exon targeted by the first gRNA.

20. The system of claim 18 or claim 19, wherein the first or second gRNA scaffold comprises a sequence having at least one modification relative to a reference gRNA sequence selected from the group consisting of SEQ ID NOS: 4-16.

21. The system of claim 20, wherein the at least one modification of the reference gRNA comprises;
a. at least one nucleotide substitution in a region of the gRNA variant;
b. at least one nucleotide deletion in a region of the gRNA variant;
c. at least one nucleotide insertion in a region of the gRNA variant;
d. a substitution of all or a portion of a region of the gRNA variant;
e. a deletion of all or a portion of a region of the gRNA variant; or
f. any combination of (a)-(e).

22. The system of claim 21, wherein the modified region of the gRNA variant is selected from the group consisting of extended stem loop, scaffold stem loop, triplex, and pseudoknot.

23. The gRNA variant of claim 22, wherein the scaffold stem further comprises a bubble.

24. The gRNA variant of claim 22 or claim 23, wherein the triplex further comprises a loop region.

25. The gRNA variant of any one of claims 21-24, wherein the scaffold further comprises a 5′ unstructured region.

26. The gRNA variant of any one of claims 21-25, wherein the at least one modification comprises:
a. a substitution of 1 to 15 consecutive or non-consecutive nucleotides in the gRNA variant in one or more regions;
b. a deletion of 1 to 10 consecutive or non-consecutive nucleotides in the gRNA variant in one or more regions;
c. an insertion of 1 to 10 consecutive or non-consecutive nucleotides in the gRNA variant in one or more regions;
d. a substitution of the scaffold stem loop or the extended stem loop with an RNA stem loop sequence from a heterologous RNA source; or
e. any combination of (a)-(d).

27. The gRNA variant of any one of claims 21-26, wherein the heterologous extended stem loop region comprises at least 10, at least 20, at least 100, at least 500, at least 1000, or at least 10,000 nucleotides.

28. The gRNA variant of claim 27, wherein the heterologous extended stem loop sequence increases the stability of the gRNA.

29. The gRNA variant of claim 27 or claim 28, wherein the heterologous RNA stem loop sequence is selected from one or more of MS2 hairpin, Qβ hairpin, U1 hairpin II, Uvsx, PP7 stem loop, or Rev Response Element (RRE), or a sequence variant thereof.

30. The gRNA variant of claim 29, wherein the heterologous RNA stem loop is capable of binding a protein, an RNA structure, a DNA sequence, or a small molecule.

31. The system of any one of claims 1-30, wherein the first or second gRNA has a scaffold comprising a sequence having at least about 50%, at least about 60%, at least about 70%, at least about 80%, at least about 90%, at least about 95%, at least about 96%, at least about 97%, at least about 98%, at least about 99%, or 100% sequence identity to a sequence selected from the group consisting of SEQ ID NOS: 2238-2285, 43571-43661, 44045, and 44047.

32. The system of any one of claims 1-30, wherein the first or second gRNA has a scaffold comprising a sequence selected from the group consisting of SEQ ID NOs: 2238-2285, 43571-43661, 44045, and 44047.

33. The system of any one of claims 1-30, wherein the first or second gRNA has a scaffold consisting of a sequence selected from the group consisting of SEQ ID NOs: 2238-2285, 43571-43661, 44045, and 44047.

34. The system of any one of claims 1-33, wherein the Class 2, Type V CRISPR protein is a CasX variant protein having at least one modification relative to a reference CasX protein having a sequence selected from the group consisting of SEQ ID NOS: 1-3 wherein the CasX variant exhibits at least one improved characteristic as compared to the reference CasX protein.

35. The system of claim 34, wherein the at least one modification comprises at least one amino acid substitution, deletion, or substitution in a domain of the CasX variant protein relative to the reference CasX protein.

36. The system of claim 35, wherein the domain is selected from the group consisting of a non-target strand binding (NTSB) domain, a target strand loading (TSL) domain, a helical I domain, a helical II domain, an oligonucleotide binding domain (OBD), and a RuvC DNA cleavage domain.

37. The system of any one of claims 34-36, wherein the CasX variant comprises an NTSB domain derived from SEQ ID NO: 1 and TSL, helical I, helical II domain, OBD, and RuvC domains derived from SEQ ID NO: 2.

38. The system of claim 37, wherein the CasX variant comprises the sequence of SEQ ID NO: 127.

39. The system of claim 37, wherein the CasX variant comprises a helical 1B domain derived from SEQ ID NO: 1

40. The system of claim 39, wherein the CasX variant comprises the sequence of SEQ ID NOS: 132-148 or 43662-43907.

41. The system of any one of claims 34-36, wherein the Class 2, Type V CRISPR protein is a CasX variant protein comprising a sequence selected from the group consisting of SEQ ID NOS: 59, 72-99, 101-148, and 43662-43907, or a sequence having at least about 50%, at least about 60%, at least about 70%, at least about 80%, at least about 90%, or at least about 95%, or at least about 95%, or at least about 96%, or at least about 97%, or at least about 98%, or at least about 99% sequence identity thereto.

42. The system of any one of claims 34-36, wherein the Class 2, Type V CRISPR protein is a CasX variant protein comprising a sequence selected from the group consisting of SEQ ID NOS: 59, 72-99, 101-148, and 43662-43907.

43. The system of any one of claims 34-36, wherein the CasX variant protein consists of a sequence selected from the group consisting of SEQ ID NOS: 59, 72-99, 101-148, and 43662-43907.

44. The system of any one of claims 34-43, wherein the CasX variant protein further comprises one or more nuclear localization signals (NLS).

45. The system of claim 44, wherein the one or more NLS are selected from the group of sequences consisting of PKKKRKV (SEQ ID NO: 149), KRPAATKKAGQAKKKK (SEQ ID NO: 150), PAAKRVKLD (SEQ ID NO: 151), RQRRNELKRSP (SEQ ID NO: 152), NQSSNFGPMKGGNFGGRSSGPYGGGGQYFAKPRNQGGY (SEQ ID NO: 153), RMRIZFKNKGKDTAELRRRRVEVSVELRKAKKDEQILKRRNV (SEQ ID NO: 154), VSRKRPRP (SEQ ID NO: 155), PPKKARED (SEQ ID NO: 156), PQPKKKPL (SEQ ID NO: 185), SALIKKKKKMAP (SEQ ID NO: 157), DRLRR (SEQ ID NO: 158), PKQKKRK (SEQ ID NO: 159), RKLKKKIKKL (SEQ ID NO: 160), REKKKFLKRR (SEQ ID NO: 161), KRKGDEVDGVDEVAKKKSKK (SEQ ID NO: 162), RKCLQAGMNLEARKTKK (SEQ ID NO: 163), PRPRKIPR (SEQ ID NO: 164), PPRKKRTVV (SEQ ID NO: 165), NLSKKKKRKREK (SEQ ID NO: 166), RRPSRPFRKP (SEQ ID NO: 167), KRPRSPSS (SEQ ID NO: 168), KRGINDRNFWRGENERKTR (SEQ ID NO: 169), PRPPKMARYDN (SEQ ID NO: 170), KRSFSKAF (SEQ ID NO: 186), KLKIKRPVK (SEQ ID NO: 171), PKTRRRPRRSQRKRPPT (SEQ ID NO: 173), RRKKRRPRRKKRR (SEQ ID NO: 176), PKKKSRKPKKKSRK (SEQ ID NO: 177), HKKKHPDASVNFSEFSK (SEQ ID NO: 178), QRPGPYDRPQRPGPYDRP (SEQ ID NO: 179), LSPSLSPLLSPSLSPL (SEQ ID NO: 180), RGKGGKGLGKGGAKRHRK (SEQ ID NO: 181), PKRGRGRPKRGRGR (SEQ ID NO: 182), MSRRRKANPTKLSENAKKLAKEVEN (SEQ ID NO: 174), PKKKRKVPPPPAAKRVKLD (SEQ ID NO: 172), and PKKKRKVPPPPKKKRKV (SEQ ID NO: 184).

46. The system of claim 44 or claim 45, wherein the one or more NLS are located at or near the C-terminus of the CasX variant protein.

47. The system of claim 44 or claim 45, wherein the one or more NLS are located at or near the N-terminus of the CasX variant protein.

48. The system of claim 44 or claim 45, comprising one or more NLS located at or near the N-terminus and at or near the C-terminus of the CasX variant protein.

49. The system of any one of claims 34-48, wherein the CasX variant is capable of forming a ribonuclear protein complex (RNP) with a gRNA.

50. The system of claim 49, wherein an RNP of the CasX variant protein and the gRNA variant exhibit at least one or more improved characteristics as compared to an RNP of a reference CasX protein of SEQ ID NO: 1, SEQ ID NO: 2, or SEQ ID NO: 3 and a gRNA comprising a sequence of SEQ ID NOs: 4-16.

51. The system of claim 50, wherein the improved characteristic is selected from one or more of the group consisting of improved folding of the CasX variant; improved binding affinity to a guide ribonucleic acid (gRNA); improved binding affinity to a target DNA; improved ability to utilize a greater spectrum of one or more PAM sequences, including ATC, CTC, GTC, or TTC, in the editing of target DNA; improved unwinding of the target DNA; increased editing activity; improved editing efficiency; improved editing specificity; increased nuclease activity; increased target strand loading for double strand cleavage; decreased target strand loading for single strand nicking; decreased off-target cleavage; improved binding of non-target DNA strand; improved protein stability; improved protein solubility; improved protein:gRNA complex (RNP) stability; and improved fusion characteristics.

52. The system of claim 50 or claim 51, wherein the improved characteristic of the RNP of the CasX variant protein and the gRNA variant is at least about 1.1 to about 100-fold or more improved relative to the RNP of the reference CasX protein of SEQ ID NO: 1, SEQ ID NO: 2, or SEQ ID NO: 3 and the gRNA comprising a sequence of SEQ ID NOs: 4-16.

53. The system of claim 50 or claim 51, wherein the improved characteristic of the CasX variant protein is at least about 1.1, at least about 2, at least about 10, at least about 100-fold or more improved relative to the reference CasX protein of SEQ ID NO: 1, SEQ ID NO: 2, or SEQ ID NO: 3 and the gRNA comprising a sequence of SEQ ID NOs: 4-16.

54. The system of any one of claims 50-53, wherein the improved characteristic comprises editing efficiency, and the RNP of the CasX variant protein and the gRNA variant comprises a 1.1 to 100-fold improvement in editing efficiency compared to the RNP of the reference CasX protein of SEQ ID NO: 2 and the gRNA of SEQ ID NOs: 4-16.

55. The system of any one of claims 49-54, wherein the RNP comprising the CasX variant and the gRNA variant exhibits greater editing efficiency and/or binding of a target sequence in the target DNA when any one of the PAM sequences TTC, ATC, GTC, or CTC is located 1 nucleotide 5′ to the non-target strand of the protospacer having identity with the targeting sequence of the gRNA in a cellular assay system compared to the editing efficiency and/or binding of an RNP comprising a reference CasX protein and a reference gRNA in a comparable assay system.

56. The system of claim 55, wherein the PAM sequence is TTC.

57. The system of claim 55, wherein the PAM sequence is ATC.

58. The system of claim 55, wherein the PAM sequence is CTC.

59. The system of claim 55, wherein the PAM sequence is GTC.

60. The system of any one of claims 55-59, wherein the increased binding affinity for the one or more PAM sequences is at least 1.5-fold greater compared to the binding affinity of any one of the reference CasX proteins of SEQ ID NOS: 1-3 for the PAM sequences.

61. The system of any one of claims 49-60, wherein the CasX variant and the gRNA variant are able to form RNP having at least about a 5%, at least about a 10%, at least about a 15%, or at least about a 20% higher percentage of cleavage-competent conformation compared to an RNP of any one of the reference CasX proteins of SEQ ID NOS: 1-3 and the gRNA of SEQ ID NOs: 4-16.

62. The system of any one of claims 49-61, wherein the RNP comprising the CasX variant and the gRNA variant exhibit a cleavage rate for the target nucleic acid in a timed in vitro assay that is at least about 5-fold, at least about 10-fold, or at least about 20-fold higher compared to an RNP of any one of the reference CasX proteins of SEQ ID NOS: 1-3 and the gRNA of SEQ ID NOs: 4-16 in a comparable assay.

63. The system of any one of claims 49-62, wherein the RNP comprising the CasX variant and the gRNA variant exhibit higher percent editing of the target nucleic acid in a timed in vitro assay that is at least about 5-fold, at least about 10-fold, at least about 20-fold, or at least about 100-fold higher compared to an RNP of any one of the reference CasX proteins of SEQ ID NOS: 1-3 and the gRNA of SEQ ID NOs: 4-16 in a comparable assay.

64. The system of any one of claims 34-63, wherein the CasX variant protein comprises a RuvC DNA cleavage domain having nickase activity.

65. The system of any one of claims 34-63, wherein the CasX variant protein comprises a RuvC DNA cleavage domain having double-stranded cleavage activity.

66. The system of any one of claims 34-49, wherein the CasX variant protein is a catalytically inactive CasX variant protein (dCasX), and wherein the dCasX and the gRNA retain the ability to bind to the PTBP1 target nucleic acid.

67. The system of claim 66, wherein the dCasX comprises a mutation at residues:
a. D672, E769, and/or D935 corresponding to the CasX protein of SEQ ID NO:1; or
b. D659, E756 and/or D922 corresponding to the CasX protein of SEQ ID NO: 2.

68. The system of claim 67, wherein the mutation is a substitution of alanine for the residue.

69. The system of any one of claims 1-65, further comprising a donor template nucleic acid.

70. The system of claim 69, wherein the donor template comprises a nucleic acid comprising at least a portion of a PTBP1 gene selected from the group consisting of a PTBP1 exon, a PTBP1 intron, a PTBP1 intron-exon junction, and a PTBP1 regulatory element.

71. The system of claim 70, wherein the donor template sequence comprises one or more mutations relative to a corresponding portion of a wild-type PTBP1 gene.

72. The system of claim 70 or claim 71, wherein the donor template comprises a nucleic acid comprising at least a portion of a PTBP1 exon selected from the group consisting of PTBP1 exon 1, PTBP1 exon 2, PTBP1 exon 3, PTBP1 exon 4, PTBP1 exon 5, PTBP1 exon 6, PTBP1 exon 7, PTBP1 exon 8, PTBP1 exon 9, PTBP1 exon 10, PTBP1 exon 11, PTBP1 exon 12, PTBP1 exon 13, PTBP1 exon 14, PTBP1 exon 15, and PTBP1 exon 16.

73. The system of claim 72, wherein the donor template comprises a nucleic acid comprising at least a portion of a PTBP1 exon selected from the group consisting of PTBP1 exon 1, PTBP1 exon 2, and PTBP1 exon 3.

74. The system of any one of claims 69-73, wherein the donor template ranges in size from 10-15,000 nucleotides.

75. The system of any one of claims 69-74, wherein the donor template is a single-stranded DNA template or a single stranded RNA template.

76. The system of any one of claims 69-74, wherein the donor template is a double-stranded DNA template.

77. The system of any one of claims 69-76, wherein the donor template comprises homologous arms at or near the 5′ and 3′ ends of the donor template that are complementary to sequences flanking cleavage sites in the PTBP1 target nucleic acid introduced by the Class 2, Type V CRISPR protein.

78. A nucleic acid comprising the donor template of any one of claims 69-77.

79. A nucleic acid comprising a sequence that encodes the CasX variant of any one of claims 34-68.

80. A nucleic acid comprising a sequence that encodes the gRNA of any one of claims 1-33.

81. The nucleic acid of claim 79, wherein the sequence that encodes the CasX variant protein is codon optimized for expression in a eukaryotic cell.

82. A vector comprising the gRNA of any one of claims 1-33, the CasX variant protein of any one of claims 34-68, or the nucleic acid of any one of claims 78-81.

83. The vector of claim 82, wherein the vector further comprises a promoter.

84. The vector of claim 82, wherein the vector is selected from the group consisting of a retroviral vector, a lentiviral vector, an adenoviral vector, an adeno-associated viral (AAV) vector, a herpes simplex virus (HSV) vector, a CasX delivery particle (XDP), a plasmid, a minicircle, a nanoplasmid, a DNA vector, and an RNA vector.

85. The vector of claim 84, wherein the vector is an AAV vector.

86. The vector of claim 85, wherein the AAV vector is selected from AAV1, AAV2, AAV3, AAV4, AAV5, AAV6, AAV7, AAV8, AAV9, AAV10, AAV11, AAV12, AAV-Rh74, or AAVRh10.

87. The vector of claim 84, wherein the vector is a retroviral vector.

88. The vector of claim 84, wherein the vector is a XDP comprising one or more components of a gag polyprotein.

89. The vector of claim 88, wherein the one or more components of the gag polyprotein are selected from the group consisting of matrix protein (MA), a nucleocapsid protein (NC), a capsid protein (CA), a p1 peptide, a p6 peptide, a P2A peptide, a P2B peptide, a P10 peptide, a p12 peptide, a PP21/24 peptide, a P12/P3/P8 peptide, a P20 peptide, a protease cleavage site.

90. The vector of claim 88 or claim 89, comprising the CasX variant protein and the gRNA.

91. The vector of claim 90, wherein the CasX variant protein and the gRNA are associated together in an RNP.

92. The vector of any one of claims 88-91, further comprising a glycoprotein tropism factor.

93. The vector of any one of claims 88-92, wherein the glycoprotein tropism factor has binding affinity for a cell surface marker of a target cell and facilitates entry of the XDP into the target cell.

94. The vector of any one of claims 82-93, further comprising the donor template.

95. A host cell comprising the vector of any one of claims 82-94.

96. The host cell of claim 95, wherein the host cell is selected from the group consisting of Baby Hamster Kidney fibroblast (BHK) cells, human embryonic kidney 293 (HEK293), human embryonic kidney 293T (HEK293T) cells, NS0 cells, SP2/0 cells, YO myeloma cells, P3X63 mouse myeloma cells, PER cells, PER.C6 cells, hybridoma cells, NIH3T3 cells, CV-1 (simian) in Origin with SV40 genetic material (COS) cells, HeLa cells, Chinese hamster ovary (CHO) cells, or yeast cells.

97. A method of modifying a PTBP1 target nucleic acid sequence in a population of cells, the method comprising introducing into cells of the population:
a. the system of any one of claims 1-77;
b. the nucleic acid of any one of claims 78-81;
c. the vector as in any one of claims 82-87;
d. the XDP of any one of claims 89-93; or
e. combinations of two or more of (a)-(d),
wherein the PTBP1 gene target nucleic acid sequence of the cells targeted by the first gRNA is modified by the CasX variant protein.

98. The method of claim 97, wherein the modifying comprises introducing a single-stranded break in the PTBP1 gene target nucleic acid sequence of the cells of the population.

99. The method of claim 97, wherein the modifying comprises introducing a double-stranded break in the PTBP1 gene target nucleic acid sequence of the cells of the population.

100. The method of any one of claims 97-99, further comprising introducing into the cells of the population a second gRNA or a nucleic acid encoding the second gRNA, wherein the second gRNA has a targeting sequence complementary to a different or overlapping portion of the PTBP1 gene target nucleic acid compared to the first gRNA, and wherein introducing the second gRNA results in an additional break in the PTBP1 target nucleic acid of the cells of the population.

101. The method of any one of claims 97-100, wherein the modifying comprises introducing an insertion, deletion, substitution, duplication, or inversion of one or more nucleotides in the PTBP1 gene of the cells of the population.

102. The method of any one of claims 97-101, wherein the modifying comprises insertion of the donor template into the break site(s) of the PTBP1 gene target nucleic acid sequence of the cells of the population.

103. The method of claim 102, wherein the insertion of the donor template is mediated by homology-directed repair (HDR) or homology-independent targeted integration (HITI).

104. The method of any one of claims 97-102, wherein the modifying results in at least about 10%, at least about 20%, at least about 30%, at least about 40%, at least about 50%, at least about 60%, at least about 70%, at least about 80%, or at least about 90% edits in the PTBP1 gene in the modified cells of the population.

105. The method of any one of claims 97-104, wherein the modifying results in a knock-down or knock-out of the PTBP1 gene in the cells of the population.

106. The method of any one of claims 97-105, wherein the PTBP1 gene of the cells of the population is modified such that expression of the PTBP1 protein is reduced by at least about 10%, at least about 20%, at least about 30%, at least about 40%, at least about 50%, at least about 60%, at least about 70%, at least about 80%, or at least about 90% in comparison to cells in which the PTBP1 gene has not been modified.

107. The method of any one of claims 97-105, wherein the PTBP1 gene of the cells of the population is modified such that at least about 10%, at least about 20%, at least about 30%, at least about 40%, at least about 50%, at least about 60%, at least about 70%, at least about 80%, or at least about 90% of the cells do not express a detectable level of PTBP1 protein.

108. The method of any one of claims 97-107, wherein the cells are eukaryotic.

109. The method of claim 108, wherein the eukaryotic cells are selected from the group consisting of rodent cells, mouse cells, rat cells, and non-human primate cells.

110. The method of claim 108, wherein the eukaryotic cells are human cells.

111. The method of any one of claims 108-110, wherein the eukaryotic cells are selected from the group consisting of microglial cells, astrocytes, oligodendrocytes, and fibroblasts.

112. The method of claim 111, wherein the modification of the PTBP1 target nucleic acid sequence results in reprogramming of the eukaryotic cells into neurons.

113. The method of claim 112, wherein the modification of the PTBP1 target nucleic acid sequence results in an increase in expression of nPTB in the modified cells by at least about 10%, at least about 20%, at least about 30%, at least about 40%, at least about 50%, at least about 60%, at least about 70%, at least about 80%, or at least about 90% in comparison to cells in which the PTBP1 gene has not been modified.

114. The method of claim 112 or claim 113, wherein the PTBP1 gene of the cells of the population is modified such that at least about 10%, at least about 20%, at least about 30%, at least about 40%, at least about 50%, at least about 60%, at least about 70%, at least about 80%, or at least about 90% of the cells express a detectable level of nPTB protein.

115. The method of any one of claim 97-114, wherein the modification of the PTBP1 gene target nucleic acid sequence of the population of cells occurs in vitro or ex vivo.

116. The method of any one of claim 97-114, wherein the modification of the PTBP1 gene target nucleic acid sequence of the population of cells occurs in vivo in a subject.

117. The method of claim 116, wherein the subject is selected from the group consisting of a rodent, a mouse, a rat, and a non-human primate.

118. The method of claim 116, wherein the subject is a human.

119. The method of any one of claims 116-118, wherein the method comprises administering a therapeutically effective dose of an AAV vector to the subject.

120. The method of claim 119, wherein the AAV vector is administered to the subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

121. The method of claim 119, wherein the AAV vector is administered to the subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

122. The method of any one of claims 116-118, wherein the method comprises administering a therapeutically effective dose of a CasX delivery particle (XDP) to the subject.

123. The method of claim 122, wherein the XDP is administered to the subject at a dose of at least about 1×10⁵ particles/kg, at least about 1×10⁶ particles/kg, at least about 1×10⁷ particles/kg at least about 1×10⁸ particles/kg, at least about 1×10⁸ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, at least about 1×10¹⁶ particles/kg.

124. The method of claim 122, wherein the XDP is administered to the subject at a dose of at least about 1×10⁵ particles/kg to about 1×10¹⁶ particles/kg, or at least about 1×10⁶ particles/kg to about 1×10¹⁵ particles/kg, or at least about 1×10⁷ particles/kg to about 1×10¹⁴ particles/kg.

125. The method of any one of claims 116-124, wherein the vector or XDP is administered to the subject by a route of administration selected from intraparenchymal, intravenous, intra-arterial, intracerebroventricular, intracisternal, intrathecal, intracranial, lumbar, intraperitoneal, or combinations thereof.

126. The method of any one of claims 97-125, further comprising contacting the PTBP1 gene target nucleic acid sequence of the population of cells with:
a. an additional CRISPR nuclease and a gRNA targeting a different or overlapping portion of the PTBP1 target nucleic acid compared to the first gRNA;
b. a polynucleotide encoding the additional CRISPR nuclease and the gRNA of (a);
c. a vector comprising the polynucleotide of (b); or
d. a XDP comprising the additional CRISPR nuclease and the gRNA of (a),
wherein the contacting results in modification of the PTBP1 gene at a different location in the sequence compared to the sequence targeted by the first gRNA.

127. The method of claim 126, wherein the additional CRISPR nuclease is a CasX variant protein having a sequence different from the CasX variant protein of any of the preceding claims.

128. The method of claim 126, wherein the additional CRISPR nuclease is not a CasX protein.

129. The method of claim 128, wherein the additional CRISPR nuclease is selected from the group consisting of Cas9, Cas12a, Cas12b, Cas12c, Cas12d (CasY), Cas12J, Cas12k, Cas13a, Cas13b, Cas13c, Cas13d, Cas12j, Cas12k, CasY, Cas14, Cpf1, C2c1, Csn2, and sequence variants thereof.

130. A population of cells modified by the method of any one of claims 97-129, wherein the cells have been modified such that at least 70%, at least 75%, at least 80%, at least 85%, at least 90%, or at least 95% of the modified cells do not express a detectable level of PTBP1 protein.

131. A population of cells modified by the method of any one of claims 97-129, wherein the cells have been modified such that the expression of PTBP1 protein is reduced by at least 70%, at least 75%, at least 80%, at least 85%, at least 90%, or at least 95% compared to cells where the PTBP1 gene has not been modified.

132. A population of cells modified by the method of any one of claims 97-129, wherein the cells have been modified such that the expression of nPTB protein is increased by at least about 10%, at least about 20%, at least about 30%, at least about 40%, at least about 50%, at least about 60%, at least about 70%, at least about 80%, or at least about 90% in comparison to cells in which the PTBP1 gene has not been modified.

133. A method of treating a PTBP1-related disease in a subject in need thereof, the method comprising administering to the subject a therapeutically effective amount of the cells of any one of claims 130-132.

134. The method of claim 133, wherein the PTBP1-related disease is a neurologic disease or neurologic injury.

135. The method of claim 134, wherein the neurologic disease or neurologic injury is selected from the group consisting of Parkinson's disease, Huntington's disease, Alzheimer's, amyotrophic lateral sclerosis (ALS), traumatic brain injury, and traumatic spinal cord injury.

136. The method of any one of claims 133-135, wherein the cells are autologous with respect to the subject to be administered the cells.

137. The method of any one of claims 133-135, wherein the cells are allogeneic with respect to the subject to be administered the cells.

138. The method of any one of claims 133-137, wherein the cells or their progeny persist in the subject for at least one month, two month, three months, four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the modified cells to the subject.

139. The method of any one of claims 133-138, wherein the method further comprises administering a chemotherapeutic agent.

140. The method of any one of claims 133-139, wherein the subject is selected from the group consisting of a rodent, a mouse, a rat, and a non-human primate.

141. The method of any one of claims 133-139, wherein the subject is a human.

142. A method of treating a PTBP1-related disease in a subject in need thereof, comprising modifying a PTBP1 gene in cells of the subject, the modifying comprising contacting said cells with a therapeutically effective dose of:
a. the system of any one of claims 1-77;
b. the nucleic acid of any one of claims 78-81;
c. the vector as in any one of claims 82-87;
d. the XDP of any one of claims 88-93; or
e. combinations of two or more of (a)-(d),
wherein the PTBP1 gene of the cells targeted by the first gRNA is modified by the CasX variant protein.

143. The method of claim 142, wherein the modifying comprises introducing a single-stranded break in the PTBP1 gene of the cells.

144. The method of claim 142, wherein the modifying comprises introducing a double-stranded break in the PTBP1 gene of the cells.

145. The method of any one of claims 142-144, further comprising introducing into the cells of the subject a second gRNA or a nucleic acid encoding the second gRNA, wherein the second gRNA has a targeting sequence complementary to a different or overlapping portion of the target nucleic acid compared to the first gRNA, resulting in an additional break in the PTBP1 target nucleic acid of the cells of the subject.

146. The method of any one of claims 142-145, wherein the modifying comprises introducing an insertion, deletion, substitution, duplication, or inversion of one or more nucleotides in the PTBP1 gene of the cells.

147. The method of any one of claims 142-145, wherein the modifying comprises insertion of the donor template into the break site(s) of the PTBP1 gene target nucleic acid sequence of the cells.

148. The method of claim 147, wherein the insertion of the donor template is mediated by homology-directed repair (HDR) or homology-independent targeted integration (HITI).

149. The method of any one of claims 142-148, wherein the modifying results in edits in the PTBP1 gene in at least about 10%, at least about 20%, at least about 30%, at least about 40%, at least about 50%, at least about 60%, at least about 70%, at least about 80%, or at least about 90% edits of the modified cells of the subject.

150. The method of any one of claims 142-149, wherein the modifying results in a knock-down or knock-out of the PTBP1 gene in the modified cells of the subject.

151. The method of any one of claims 142-149, wherein the PTBP1 gene of the cells of the subject are modified such that expression of the PTBP1 protein by the modified cells is reduced by at least about 10%, at least about 20%, at least about 30%, at least about 40%, at least about 50%, at least about 60%, at least about 70%, at least about 80%, or at least about 90% in comparison to cells that have not been modified.

152. The method of any one of claims 142-149, wherein the PTBP1 gene of the cells of the subject are modified such that at least 50%, at least 60%, at least 70%, at least 75%, at least 80%, at least 85%, at least 90%, or at least 95% of the modified cells do not express a detectable level of PTBP1 protein.

153. The method of any one of claims 142-152, wherein the cells modified by the method are selected from the group consisting of microglial cells, astrocytes, oligodendrocytes, and fibroblasts.

154. The method of claim 153, wherein the modification results in reprogramming of the modified cells into neurons.

155. The method of any one of claims 142-154, wherein the modification results in an increase in expression of nPTB in the modified cells by at least about 10%, at least about 20%, at least about 30%, at least about 40%, at least about 50%, at least about 60%, at least about 70%, at least about 80%, or at least about 90% in comparison to cells in which the PTBP1 gene has not been modified.

156. The method of any one of claims 142-155, wherein at least about 10%, at least about 20%, at least about 30%, at least about 40%, at least about 50%, at least about 60%, at least about 70%, at least about 80%, or at least about 90% of the modified cells express a detectable level of nPTB protein.

157. The method of any one of claims 142-156, wherein the PTBP1-related disease is a neurologic disease or neurologic injury.

158. The method of claim 157, wherein the neurologic disease or neurologic injury is selected from the group consisting of Parkinson's disease, Huntington's disease, Alzheimer's, amyotrophic lateral sclerosis (ALS), traumatic brain injury, and traumatic spinal cord injury.

159. The method of any one of claims 142-152, wherein the PTBP1-related disease is a cancer.

160. The method of claim 159, wherein the cancer is selected from the group consisting of ovarian cancer, glioblastoma, bladder cancer, colon cancer and breast cancer.

161. The method of claim 159 or claim 160, wherein the modification of the PTBP1 gene results in prevention or reduction of tumorigenesis of the cells.

162. The method of claim 159 or claim 160, wherein the modification of the PTBP1 target nucleic acid sequence results in stasis of an existing tumor in a subject.

163. The method of any one of claims 142-162, wherein the subject is selected from the group consisting of rodent, mouse, rat, and non-human primate.

164. The method of any one of claims 142-162, wherein the subject is a human.

165. The method of any one of claims 142-164, wherein the vector is AAV and is administered to the subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

166. The method of any one of claims 142-164, wherein the vector is AAV and is administered to the subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

167. The method of any one of claims 142-164, wherein the XDP is administered to the subject at a dose of at least about 1×10⁵ particles/kg, at least about 1×10⁶ particles/kg, at least about 1×10⁷ particles/kg at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, at least about 1×10¹⁶ particles/kg.

168. The method of any one of claims 142-164, wherein the XDP is administered to the subject at a dose of at least about 1×10⁵ particles/kg to about 1×10¹⁶ particles/kg, or at least about 1×10⁶ particles/kg to about 1×10¹⁵ particles/kg, or at least about 1×10⁷ particles/kg to about 1×10¹⁴ particles/kg.

169. The method of any one of claims 142-168, wherein the vector or XDP is administered to the subject by a route of administration selected from intraparenchymal, intravenous, intra-arterial, intracerebroventricular, intracisternal, intrathecal, intracranial, lumbar, intraperitoneal, or combinations thereof.

170. The method of any one of claims 142-169, wherein the method results in improvement in at least one clinically-relevant endpoint in the subject.

171. The method of claim 170, wherein the disease is Parkinson's disease and the clinically-relevant endpoint is selected from the group consisting of disease progression, Unified Parkinson's Disease Rating Scale (UPDRS), Unified Dyskinesia Rating Scale (UDysRS), Parkinson's Disease Quality of Life Questionnaire (PDQ-39) score, Movement Disorder Society-Sponsored Unified Parkinson's Disease Rating Scale (MDS-UPDRS), changes from baseline of motor score as measured by Inertial Measurement Unit (IMU) on Finger taping (FT) and Pronation-supination movement of the hands (PSH), delay in time to clinically meaningful worsening of motor progression, levodopa's duration of effect (“on time”), Clinical Global Impression—Improvement (CGI-I), change from baseline in Zarit Burden Interview score (ZBI), EQ-5D summary index, total disease duration, patient cognitive status (MMSE), and change from baseline in fatigue.

172. The method of claim 170, wherein the disease is Huntington's disease and the clinically-relevant endpoint is selected from the group consisting of Unified Huntington's Disease Rating Scale (UHDRS), cognitive decline, psychiatric abnormalities, motor impairment, changes in baseline in striatal volume, Stroop word test, total motor score (TMS), bradykinesia, dystonia, Symbol Digit Modalities Test, University of Pennsylvania Smell Identification Test, emotion recognition, speeded tapping, paced tapping, the Trail Making Test, intracranial-corrected volumes (ICV), and the Everyday Cognition Rating Scale (ECOG).

173. The method of claim 170, wherein the disease is ALS and the clinically-relevant endpoint is selected from the group consisting of ALS Functional Rating Scale (ALSFRS-(R)), combined assessment of function and survival, time to death, time to tracheostomy, time to persistent assisted ventilation (DTP), forced vital capacity (% FVC), manual muscle test, maximum voluntary isometric contraction, duration of response, progression-free survival, time to progression of disease, and time-to-treatment failure.

174. The method of claim 170, wherein the disease is Alzheimer's disease and the clinically-relevant endpoint is selected from the group consisting of change in Alzheimer's Disease Assessment Scale-Cognitive subscale (ADAS-Cog₁₄) score, change in the Cohen-Mansfield Agitation Inventory (CMAI) score, change in the Alzheimer's Disease Cooperative Study-Instrumental Activities of Daily Living (ADCS-iADL) score, Clinical Dementia Rating Scale-Sum of Boxes (CDR-SB) score, DIAN Multivariate Cognitive Endpoint, Preclinical Alzheimer Cognitive Composite 5 (PACC5) score, Mini-Mental State Exam (MMSE) score, cognitive impairment, functional impairment, brain amyloid levels measured by amyloid positron emission tomography (PET), brain tau levels measured by PET, spinal fluid amyloid-β levels, and spinal fluid tau levels.

175. The method of claim 170, wherein the disease is cancer and the clinically-relevant endpoint is selected from the group consisting of tumor shrinkage as a complete, partial or incomplete response; time-to-progression; time to treatment failure; biomarker response; progression-free survival; disease free-survival; time to recurrence; time to metastasis; time of overall survival; improvement of quality of life; and improvement of symptoms.

176. The system of any one of claims 1-77, the nucleic acid of any one of claims 78-81, the vector of any one of 82-87, the XDP of any one of claims 88-93, the host cell of claim 95 or claim 96, or the population of cells of any one of claims 130-132, for use as a medicament for the treatment of a PTBP1 related disease.

177. The system of any one of claims 1-77, wherein the target nucleic acid sequence is complementary to a non-target strand sequence located 1 nucleotide 3′ of a protospacer adjacent motif (PAM) sequence.

178. The system of claim 177, wherein the PAM sequence comprises a TC motif.

179. The system of claim 178, wherein the PAM sequence comprises ATC, GTC, CTC or TTC.

180. The system of any one of claims 177-179, wherein the Class 2 Type V CRISPR protein comprises a RuvC domain.

181. The system of claim 180, wherein the RuvC domain generates a staggered double-stranded break in the target nucleic acid sequence.

182. The system of any one of claims 177-181, wherein the Class 2 Type V CRISPR protein does not comprise an HNH nuclease domain.

183. A composition of the Class 2, type V CRISPR protein of any one of claims 34-65 and the gRNA of any one of claims 1-33 as gene editing pairs for use as a medicament for the treatment of a subject having a PTBP1-related disease.
